(19) United States
(12) Patent Application Publication
(10) Pub. No.: US 2016/0376240 A1
(43) Pub. Date: Dec. 29, 2016

(54) 6 Phenyl or 6 Pyridin 3 YL Indazole Derivatives and Methods of Use

(71) Applicant: ABBVIE INC., North Chicago, IL (US)

(72) Inventors: William Bunnelle, Mundelein, IL (US); Marlon Cowart, Round Lake Beach, IL (US); Irene Drizin, Wadsworth, IL (US); John Robert Koenig, Chicago, IL (US); Marina Pliushchev, Vernon Hills, IL (US); Marc Scanio, Libertyville, IL (US)

(73) Assignee: ABBVIE INC., North Chicago, IL (US)

(21) Appl. No.: 15/112,870

(22) PCT Filed: Jan. 16, 2015
(86) PCT No.: PCT/US15/11760

§ 371 Date: Jul. 20, 2016

Related U.S. Application Data
(60) Provisional application No. 61/931,137, filed on Jan. 24, 2014.

(51) Int. Cl. C07D 231/56; C07D 405/14; C07D 417/14; C07D 487/10; C07D 403/12; C07D 471/04; C07D 413/10; C07D 401/10; C07D 405/12; C07D 401/12

(52) U.S. Cl. CPC C07D 231/56; C07D 405/12; C07D 405/14; C07D 417/14; C07D 401/12; C07D 403/12; C07D 471/04; C07D 413/10; C07D 401/10; C07D 487/10

(57) ABSTRACT

Compounds of formula (I) and pharmaceutically acceptable salts, esters, amides, or radiolabelled forms thereof, wherein R¹, R², R³, R⁴, R⁵, R⁶, and X are as defined in the specification, are useful in treating conditions or disorders prevented by or ameliorated by Tropomysin receptor kinases (Trk). Methods for making the compounds are disclosed. Also disclosed are pharmaceutical compositions of compounds of formula (I), and methods for using such compounds and compositions.

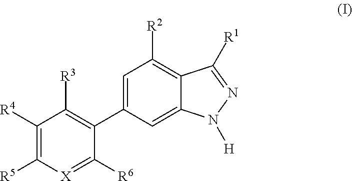

### BACKGROUND OF THE INVENTION

Technical Field

The invention relates to 6-phenyl- or 6-(pyridin-3-yl)indazoles that are inhibitors of TrkA (Tropomyosin receptor kinase isoform A), useful in treating diseases and conditions mediated and modulated by TrkA. Additionally, the invention relates to compositions containing compounds of the invention and processes of their preparation.

Description of Related Technology

TrkA is member of the Trk (Tropomyosin receptor) receptor family. Currently this family is known to include three highly homologous isoforms, called TrkA, TrkB, and TrkC. Trk receptors (Trks) are high affinity receptor tyrosine kinases. Trks bind adenosine triphosphate (ATP) and modulate intracellular signaling through their kinase enzymatic activity which is able to phosphorylate specific tyrosine residues of target proteins and peptides. Each Trk receptor isoform can be activated by endogenous peptidic factors known as neurotrophins (NT), which act as agonists of the Trk receptor. NGF (nerve growth factor) is a high affinity activator of TrkA. BDNF (brain-derived neurotrophic factor) and NT-4/5 are high affinity activators of TrkB (Tropomyosin receptor kinase isoform B). NT3 is a high affinity activator of TrkC (Tropomyosin receptor kinase isoform C). Trks are expressed in neurons, and have been implicated in the development and function of the nervous system, as well as other physiological processes.

Neurotrophins and their Trk receptors have been implicated in pain sensation and in inflammation. Pezet S, et al. Ann Rev Neuroscience 2006; 29:507-538; Mantyh P W, et al. Anesthesiology 2011; 115:189-204; and Patapoutian A, et al. Current Opinion in Neurobiology 2001; 11:272-280. Studies have shown that NGF, the agonist of TrkA, modulates pain in adult mammals. Dyck P J, et al. Neurology 1997; 48; 501-505; and Deising S, et al. Pain 2012; 153:1673-1679. Studies have also shown that inhibitors of the NGF/TrkA pathway are effective in blocking pain. Lane N E, et al. New England J Med 2010; 363:1521-1531; Schnitzer T J, et al. Osteoarthritis Cartilage 2011; 19:639-646; Katz N, et al. Pain 2011; 152:2248-2258; Evans R J, et al. J. Urology 2011; 185:1716-1721; Shelton D L, et al. Pain 2005; 116:8-16; Ro L S, et al. Pain 1999; 79:265-274; and Ugolini G, et al. Proceedings of the National Academy of Sciences of the USA 2007; 104:2985-2990. TrkA inhibitors block NGF signaling through its receptor (TrkA) and have been found effective in reducing pain in animal models. Ghilardi J R, et al. Bone 2011; 48:389-298; Ghilardi J R, et al. Molecular Pain 2010; 6:87; Mantyh, W G, et al, Neuroscience 2010; 17:588-598; and Hayashi K, et al. Journal of Pain 2011; 12:1059-1068. The TrkA, TrkB, and TrkC isoforms have high structural homology. Of the potent Trk inhibitor structural classes described, testing of isoform selectivity has revealed a lack of selectivity for any particular Trk isoform, hence they have been termed ‘pan-Trk’ inhibitors (Albaugh P, et al. ACS Medicinal Chemistry Letters 2012; 3:140-145), able to inhibit TrkA, TrkB, and TrkC. Wang T, et al. Expert Opinion on Therapeutic Patents 2009; 19:305-319.

Although compounds and mechanisms exist that are used clinically to treat pain, there is need for new compounds that can effectively treat different types of pain. Pain of various types (e.g., inflammatory pain, post-surgical pain, osteoarthritis pain, neuropathic pain) afflicts virtually all humans and animals at one time or another, and a substantial number of medical disorders and conditions produce some sort of pain as a prominent concern requiring treatment. As such, it would be particularly beneficial to identify new compounds for treating the various types of pain.

### SUMMARY

The invention is directed to 6-phenyl- or 6-(pyridin-3-yl)indazoles having a structure of Formula (I):

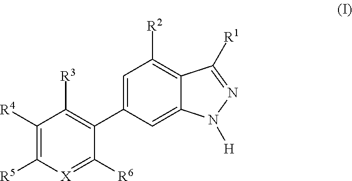

or a pharmaceutically acceptable salt, ester, amide, or radiolabelled form thereof, wherein:

R¹ is hydrogen, C₁-C₄-alkyl, C₁-C₄-fluoroalkyl, C₃-C₆-cycloalkyl or H₂N—;

R² is hydrogen or R⁷O—;

R³ and R⁶ are independently hydrogen or fluorine;

R⁴ is hydrogen, G¹-, G²-, or Y-L¹-(CR^{a}R^{b})_{f}-L²-;

R⁵ is hydrogen, R¹¹C(O)—, R¹⁰N(H)C(O)—, R¹¹C(O)NH—, R¹N(H)SO₂—, R¹¹SO₂NH—, R¹¹CH(OH)—, R¹¹C(O)C(O)NH—, or NC—;

R⁷ is C₁-C₆-alkyl, C₁-C₆-fluoroalkyl, C₃-C₈-cycloalkyl, C₃-C₈-cycloalkyl-C₁-C₄-alkyl, C₄-C₈-cycloalkenyl, C₄-C₈-cycloalkenyl-C₁-C₄-alkyl, M₄-M₇-heterocycle or M₄-M₇-heterocycle-C₁-C₄alkyl, wherein:
the C₁-C₆-alkyl and C₁-C₆-fluoroalkyl are optionally substituted with 1, 2, 3, 4, or 5 substituents selected from halogen, cyano, oxo, O₂N—, R^{s}O—, R^{s}O—(CR^{a}R^{b})ₘ—, R^{s}C(O)O—, (R^{s})(R^{t})NC(O)O—, R^{s}S—, R^{s}S(O)—, R^{s}S(O)₂—, (R^{s})(R^{t})NS(O)₂—, R^{s}C(O)—, R^{s}OC(O)—, (R^{s})(R^{t})NC(O)—, (R^{s})(R^{t})N—, R^{t}C(O)N(R^{s})—, R^{t}OC(O)N(R^{s})—, and (R^{t})S(O)₂N(R^{s})—;
the C₃-C₈-cycloalkyl, C₃-C₈-cycloalkyl-C₁-C₄-alkyl, C₄-C₈-cycloalkenyl, C₄-C₈-cycloalkenyl-C₁-C₄-alkyl, M₄-1\b-heterocycle and M₄-M₇-heterocycle-C₁-C₄alkyl are optionally substituted with 1, 2, 3, 4, or 5 substituents selected from C₁-C₄-alkyl, C₂-C₄-alkenyl, C₂-C₄-alkynyl, halogen, cyano, oxo, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{a})—, and C₁-C₄-haloalkyl;

R¹⁰ is hydrogen, C₁-C₆-alkyl, or C₃-C₇-cycloalkyl;

R¹¹ is C₁-C₆-alkyl or C₂-C₆-alkenyl;

R^{a} and R^{b}, at each occurrence, are each independently hydrogen, C₁-C₁-alkyl, or C₁-C₄-haloalkyl;

R^{c}, at each occurrence, is independently hydrogen, C₁-C₁-alkyl, C₃-C₆-cycloalkyl or C₁-C₄-haloalkyl;

R^{s}, R^{t}, R^{u}, and R^{v} are, at each occurrence, independently hydrogen, C₁-C₆-alkyl, C₂-C₆-alkenyl, C₂-C₆-alkynyl or C₁-C₆-haloalkyl;

G¹ is monocyclic M₄-M₇-heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, G^{1a}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}-(CR^{a}R^{b})ₚ—C(O)—, G^{1a}-OC(O)—, G^{1a}-(CR^{a}R^{b})ₚ—OC(O)—, G^{1b}-, G^{1b}C(O)—, G^{1b}-(CR^{a}R^{b})ₚ—, G^{1b}C(O)—(CR^{a}R^{b})ₚ—, G^{1b}-(CR^{a}R^{b})ₚ—OC(O)—, G^{1b}-(CR^{a}R^{b})ₚ—C(O)—, G^{1c}-, G^{1c}C(O)—, G^{1c}-(CR^{a}R^{b})ₕ—, G^{1c}C(O)—(CR^{a}R^{b})ₚ—, G^{1c}-(CR^{a}R^{b})ₚ—OC(O)—, G^{1c}-(CR^{a}R^{b})ₚ—C(O)—, (R^{a})(R^{v})N—, R^{v}C(O)N(R^{u})—, (R^{v})OC(O)N(R^{u})—, (R^{v})S(O)₂N(R^{u})—, and C₁-C₄-haloalkyl;

G^{1a} is aryl or heteroaryl wherein the aryl or heteroaryl are optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;

G^{1b} is C₃-C₈-cycloalkyl or C₄-C₈-cycloalkenyl, wherein the C₃-C₈-cycloalkyl or C₄-C₈-cycloalkenyl are optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;

G^{1c} is M₄-M₇-heterocycle wherein the M₄-M₇-heterocycle is optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;

G² is a fused-bicyclic heterocycle or spirocyclic heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})_{q}—, R^{u}O—(CR^{a}R^{b})—O—(CR^{a}R^{b})—, G^{1a}-, G^{1a}C(O)—, G^{1a}-(CR^{a}R^{b})_{q}—, G^{1b}-, G^{1b}C(O)—, G^{1b}-(CR^{a}R^{b})_{q}—, G^{1b}C(O)—(CR^{a}R^{b})_{q}—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;

L¹ and L² are independently selected from a bond, —O—, —NR^{c}—, —C(O)—, —R^{c}NC(O)—, —C(O)NR^{c}—, —R^{c}NC(O)O—, —OC(O)NR^{c}—, —NR^{c}C(O)NR^{c}—, —S(O)—, —S(O)₂—, —R^{c}NS(O)₂—, and —S(O)₂NR^{c}—;

X is N, CH, or CF;

Y is monocyclic C₃-C₈-cycloalkyl, monocyclic C₃-C₈-cycloalkenyl, or monocyclic M₄-M₂-heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₚ—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; or

Y is aryl or heteroaryl unsubstituted or optionally substituted with 1, 2, 3, or 4 substituents selected from C₁-C₈-alkyl, C₂-C₈-alkenyl, C₂-C₈-alkynyl, halogen, cyano, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₘ—, (R^{u})(R^{v})N—, R^{v}C(O)N(R^{u})—, (R^{v})OC(O)N(R¹¹)—, (R^{v})S(O)₂N(R^{u}), and C₁-C₄-haloalkyl; or

Y is C₁-C₆-alkyl or C₁-C₆-fluoroalkyl optionally substituted with 1, 2, 3, 4, or 5 substituents selected from halogen, cyano, oxo, O₂N—, R^{s}O—, R^{s}C(O)O—, (R^{s})(R^{t})NC(O)O—, R^{s}S—, R^{s}S(O)—, R^{s}S(O)₂—, (R^{s})(R^{t})NS(O)₂—, R^{s}C(O)—, R^{s}OC(O)—, (R^{s})(R^{t})NC(O)—, (R^{s})(R^{t})N—, R^{t}C(O)N(R^{s})—, (R^{t})OC(O)N(R^{s})—, and (R^{t})S(O)₂N(R^{s})—;

f is 1, 2, 3, or 4; or

f is 2, 3, or 4 when the moieties attaching to each side of —(CR^{a}R^{b})_{f}— are selected from O, NR^{c}, or a ring nitrogen atom of a monocyclic M₄-M₂-heterocycle when L¹ is a bond;

h, j, k and n are independently 2, 3, or 4; and

m, p and q are independently 1, 2, 3, or 4.

Another aspect of the invention relates to pharmaceutical compositions comprising compounds of the invention. Such compositions can be administered in accordance with a method of the invention, typically as part of a therapeutic regimen for treatment or prevention of conditions and disorders related to Trk receptor kinases (and particularly TrkA kinase) activity.

Yet another aspect of the invention relates to a method of selectively modulating TrkA receptor kinase activity. The method is useful for treating, or preventing conditions and disorders related to TrkA modulation in mammals. More particularly, the method is useful for treating or preventing conditions and disorders related to pain, neuropathy, inflammation, auto-immune disease, fibrosis, chronic kidney disease, and cancer. Accordingly, the compounds and compositions of the invention are useful as a medicament for treating or preventing TrkA receptor kinases modulated disease.

The compounds, compositions comprising the compounds, methods for making the compounds, and methods for treating or preventing conditions and disorders by administering the compounds are further described herein.

These and other objects of the invention are described in the following paragraphs. These objects should not be deemed to narrow the scope of the invention.

### DETAILED DESCRIPTION OF THE INVENTION

Compounds of formula (I) are disclosed in this invention

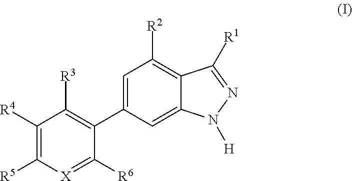

wherein R¹, R², R³, R⁴, R⁵, R⁶, and X are as defined above in the Summary. Compositions comprising such compounds and methods for treating conditions and disorders using such compounds and compositions are also disclosed.

In various embodiments, the present invention provides at least one variable that occurs more than one time in any substituent or in the compound of the invention or any other formulae herein. Definition of a variable on each occurrence is independent of its definition at another occurrence. Further, combinations of substituents are permissible only if such combinations result in stable compounds. Stable compounds are compounds which can be isolated from a reaction mixture.

### DEFINITION OF TERMS

Certain terms as used in the specification are intended to refer to the following definitions, as detailed below.

The term “alkenyl” as used herein, means a straight or branched hydrocarbon chain containing from 2 to 10 carbons and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkyl” as used herein, means a straight or branched, saturated hydrocarbon chain containing from 1 to 10 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene” denotes a divalent group derived from a straight or branched chain hydrocarbon containing from 1 to 10 carbon atoms. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkynyl” as used herein, means a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “amino” as used herein means an —NH₂ group.

The term “aryl” as used herein, means phenyl or a bicyclic aryl. The bicyclic aryl is naphthyl, or a phenyl fused to a monocyclic cycloalkyl, or a phenyl fused to a monocyclic cycloalkenyl. Representative examples of the aryl groups include, but are not limited to, dihydroindenyl, indenyl, naphthyl, dihydronaphthalenyl, and tetrahydronaphthalenyl. The bicyclic aryl is attached to the parent molecular moiety through any carbon atom contained within the bicyclic ring system. The aryl groups of the present invention can be unsubstituted or substituted.

The term “arylalkyl” as used herein, means an aryl group, as defined herein, appended to the parent molecular moiety through an alkylene group, as defined herein. Representative examples of arylalkyl include, but are not limited to, benzyl, 1-phenylethyl, 2-phenylethyl, 3-phenylpropyl, and 2-naphth-2-ylethyl.

The term “carbonyl” as used herein means a —C(═O)— group.

The term “cyano” as used herein, means a —CN group.

The term “cycloalkenyl” or “cycloalkene” as used herein, means a monocyclic or a bicyclic hydrocarbon ring system. The monocyclic cycloalkenyl has four-, five-, six-, seven- or eight carbon atoms and zero heteroatoms. The four-membered ring systems have one double bond, the five- or six-membered ring systems have one or two double bonds, and the seven- or eight-membered ring systems have one, two or three double bonds. Representative examples of monocyclic cycloalkenyl groups include, but are not limited to, cyclobutenyl, cyclopentenyl, cyclohexenyl, cycloheptenyl and cyclooctenyl. The bicyclic cycloalkenyl is a monocyclic cycloalkenyl fused to a monocyclic cycloalkyl group, or a monocyclic cycloalkenyl fused to a monocyclic cycloalkenyl group, or a bridged monocyclic ring system in which two non-adjacent carbon atoms of the monocyclic ring are linked by an alkylene bridge containing one, two, three, or four carbon atoms. Representative examples of the bicyclic cycloalkenyl groups include, but are not limited to, 4,5,6,7-tetrahydro-3aH-indene, octahydronaphthalenyl and 1,6-dihydro-pentalene. The monocyclic and bicyclic cycloalkenyl can be attached to the parent molecular moiety through any substitutable atom contained within the ring systems, and can be unsubstituted or substituted.

The term “cycloalkenylalkyl,” as used herein, refers to a cycloalkenyl group attached to the parent molecular moiety through an alkyl group.

The term “cycloalkyl” or “cycloalkane” as used herein, means a monocyclic, a bicyclic, or a tricyclic cycloalkyl. The monocyclic cycloalkyl is a carbocyclic ring system containing three to eight carbon atoms, zero heteroatoms and zero double bonds. Examples of monocyclic ring systems include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl ring, or a bridged monocyclic ring system in which two non-adjacent carbon atoms of the monocyclic ring are linked by an alkylene bridge containing one, two, three, or four carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic cycloalkyls are exemplified by a bicyclic cycloalkyl fused to a monocyclic cycloalkyl, or a bicyclic cycloalkyl in which two non-adjacent carbon atoms of the ring systems are linked by an alkylene bridge of 1, 2, 3, or 4 carbon atoms. Representative examples of tricyclic-ring systems include, but are not limited to, tricyclo[3.3.1.0³′⁷]nonane (octahydro-2,5-methanopentalene or noradamantane), and tricyclo[3.3.1.1³′⁷]decane (adamantane). The monocyclic, bicyclic, and tricyclic cycloalkyls can be unsubstituted or substituted, and are attached to the parent molecular moiety through any substitutable atom contained within the ring system.

The term “cycloalkylalkyl” as used herein, means a cycloalkyl group appended to the parent molecular moiety through an alkyl group, as defined herein.

The term “fluoroalkyl” as used herein, means an alkyl group, as defined herein, in which one, two, three, four, five, six, seven or eight hydrogen atoms are replaced by fluorine. Representative examples of haloalkyl include, but are not limited to, fluoromethyl, 2-fluoroethyl, 2,2-difluoroethyl, 2,2,2-trifluoroethyl, trifluoromethyl, difluoromethyl, pentafluoroethyl, 1,1,2-trifluoroisopropyl, and trifluoropropyl such as 3,3,3-trifluoropropyl.

The term “halo” or “halogen” as used herein, means Cl, Br, I, or F.

The term “haloalkyl” as used herein, means an alkyl group, as defined herein, in which one, two, three, four, five, six, seven or eight hydrogen atoms are replaced by halogen. Representative examples of haloalkyl include, but are not limited to, chloromethyl, 2-fluoroethyl, 2,2,2-trifluoroethyl, trifluoromethyl, difluoromethyl, pentafluoroethyl, 2-chloro-3-fluoropentyl, and trifluoropropyl such as 3,3,3-trifluoropropyl.

The term “heteroaryl” as used herein, means a monocyclic heteroaryl or a bicyclic heteroaryl. The monocyclic heteroaryl is a five- or six-membered ring. The five-membered ring contains two double bonds. The five-membered ring may contain one heteroatom selected from O or S; or one, two, three, or four nitrogen atoms and optionally one oxygen or sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryl groups include, but are not limited to, benzofuranyl, benzothienyl, benzoxazolyl, benzimidazolyl, benzoxadiazolyl, 6,7-dihydro-1,3-benzothiazolyl, imidazo[1,2-c]pyridinyl, indazolyl, indolyl, isoindolyl, isoquinolinyl, naphthyridinyl, pyridoimidazolyl, quinolinyl, thiazolo[5,4-b]pyridin-2-yl, thiazolo[5,4-d]pyrimidin-2-yl, and 5,6,7,8-tetrahydroquinolin-5-yl. The monocyclic and bicyclic heteroaryl groups of the present invention can be substituted or unsubstituted and are connected to the parent molecular moiety through any carbon atom or any nitrogen atom contained within the ring systems.

The term “heteroarylalkyl” as used herein, means a heteroaryl, as defined herein, appended to the parent molecular moiety through an alkyl group, as defined herein.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1³′]decane), and oxa-adamantane (2-oxatricyclo[3.3.1.1³′]decane). The monocyclic, bicyclic, and tricyclic heterocycles are connected to the parent molecular moiety through any carbon atom or any nitrogen atom contained within the rings, and can be unsubstituted or substituted.

The term “heterocyclealkyl”, as used herein, refers to refers to a heterocycle group attached to the parent molecular moiety through an alkyl group.

The term “heteroatom” as used herein, means a nitrogen, oxygen, or sulfur atom.

The term “hydroxyl” or “hydroxy” as used herein, means an —OH group.

The term “hydroxyalkyl” as used herein, means at least one hydroxy group, as defined herein, is appended to the parent molecular moiety through an alkylene group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “oxo” as used herein means (═O).

In some instances, the number of carbon atoms in a hydrocarbyl substituent (e.g., alkyl, alkenyl, alkynyl, or cycloalkyl) is indicated by the prefix “Cₓ-C_{y}-”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl means a saturated hydrocarbyl ring containing from 3 to 6 carbon ring atoms.

As used herein, the number of ring atoms in a heterocyclic moiety can be identified by the prefix “Mₓ-M_{y},” where x is the minimum and y is the maximum number of ring atoms in the heterocyclic moiety.

As used herein, the term “radiolabel” refers to a compound of the invention in which at least one of the atoms is a radioactive atom or radioactive isotope, wherein the radioactive atom or isotope spontaneously emits gamma rays or energetic particles, for example alpha particles or beta particles, or positrons. Examples of such radioactive atoms include, but are not limited to, ³H (tritium), ¹⁴C, ¹¹C, ¹⁵O, ¹⁸F ³⁵S, ¹²³I and ¹²⁵I.

### Compounds of the Invention

Compounds of the invention can have the Formula (I) as described in the Summary.

Particular values of variable groups in compounds of Formula (I) are as follows. Such values can be used where appropriate with any of the other values, definitions, claims or embodiments defined hereinbefore or hereinafter.

In one embodiment, R¹ is hydrogen, C₁-C₄-alkyl, C₁-C₄-fluoroalkyl, C₃-C₆-cycloalkyl or H₂N—.

In another embodiment, R¹ is hydrogen, C₁-C₄-alkyl, C₁-C₄-fluoroalkyl, or C₃-C₆-cycloalkyl.

In another embodiment, R¹ is hydrogen.

In another embodiment, R¹ is C₁-C₄-alkyl.

In a further embodiment, R¹ is methyl or ethyl.

In another embodiment, R¹ is C₁-C₄-fluoroalkyl.

In another embodiment, R¹ is C₃-C₆-cycloalkyl.

In another embodiment, R¹ is H₂N—.

In another embodiment, R¹ is methyl or ethyl.

In one embodiment, R² is hydrogen or R⁷O—.

In another embodiment, R² is hydrogen.

In another embodiment, R² is R⁷O—.

In one embodiment, R³ is hydrogen or fluorine.

In another embodiment, R³ is hydrogen.

In another embodiment, R³ is fluorine.

In one embodiment, R⁴ is hydrogen, G¹-, G²-, or Y-L¹-(CR^{a}R^{b})_{f}-L²-.

In another embodiment, R⁴ is G¹- or G²-.

In another embodiment, R⁴ is hydrogen.

In another embodiment, R⁴ is G¹-.

In another embodiment, R⁴ is G²-.

In another embodiment, R⁴ is Y-L¹-(CR^{a}R^{b})_{f}-L²-.

In one embodiment, R⁵ is hydrogen, R¹¹C(O), —R¹⁰N(H)C(O)—, R¹¹C(O)NH—, R¹⁰N(H)SO₂—, R¹¹SO₂NH—, R¹¹CH(OH)—, R¹¹C(O)C(O)NH—, or NC—.

In another embodiment, R⁵ is hydrogen.

In another embodiment, R⁵ is R¹¹C(O)—.

In another embodiment, R⁵ is R¹⁰N(H)C(O)—.

In another embodiment, R⁵ is R¹¹C(O)NH—.

In another embodiment, R⁵ is R¹⁰N(H)SO₂—.

In another embodiment, R⁵ is R¹¹SO₂NH—.

In another embodiment, R⁵ is R¹¹CH(OH)—.

In another embodiment, R⁵ is R¹¹C(O)C(O)NH—.

In another embodiment, R⁵ is NC—.

In one embodiment, R⁷ is C₁-C₆-alkyl, C₁-C₆-fluoroalkyl, C₃-C₈-cycloalkyl, C₃-C₈-cycloalkyl-C₁-C₄-alkyl, C₄-C₈-cycloalkenyl, C₄-C₈-cycloalkenyl-C₁-C₄-alkyl, M₄-M₇-heterocycle or M₄-M₇-heterocycle-C₁-C₄alkyl, wherein: the C₁-C₆-alkyl and C₁-C₆-fluoroalkyl are optionally substituted with 1, 2, 3, 4, or 5 substituents selected from halogen, cyano, oxo, O₂N—, R^{s}O—, R^{s}O—(CR^{a}R^{b})ₘ—, R^{s}C(O)O—, (R^{s})(R^{t})NC(O)O—, R^{s}S—, R^{s}S(O)—, R^{s}S(O)₂—, (R^{s})(R^{t})NS(O)₂—, R^{s}C(O)—, R^{s}OC(O)—, (R^{s})(R^{t})NC(O)—, (R^{s})(R^{t})N—, R^{t}C(O)N(R^{s})—, R^{t}OC(O)N(R^{s})—, and (R^{t})S(O)₂N(R^{s})—; the C₃-C₈-cycloalkyl, C₃-C₈-cycloalkyl-C₁-C₄-alkyl, C₄-C₈-cycloalkenyl, C₄-C₈-cycloalkenyl-C₁-C₄-alkyl, M₄-M₇-heterocycle and M₄-M₇-heterocycle-C₁-C₄alkyl are optionally substituted with 1, 2, 3, 4, or 5 substituents selected from C₁-C₄-alkyl, C₂-C₄-alkenyl, C₂-C₄-alkynyl, halogen, cyano, oxo, O₂N—, R^{u}O—, R^{u}O—(CR^{a}R^{b})—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl, wherein R^{a}, R^{b}, R^{s}, R^{t}, R^{u}, R^{v} and m are as defined in the Summary.

In another embodiment, R⁷ is C₁-C₆-alkyl.

In a further embodiment, R⁷ is isopropyl.

In another embodiment, R⁷ is C₁-C₆-fluoroalkyl.

In a further embodiment, R⁷ is 1,1,1-trifluoropropan-2-yl, (2S)-1,1,1-trifluoropropan-2-yl, or (2R)-1,1,1-trifluoropropan-2-yl.

In another embodiment, R⁷ is C₃-C₈-cycloalkyl.

In a further embodiment, R⁷ is cyclopentyl.

In another embodiment R⁷ is C₃-C₈-cycloalkyl-C₁-C₄-alkyl.

In another embodiment R⁷ is C₄-C₈-cycloalkenyl.

In another embodiment R⁷ is C₄-C₈-cycloalkenyl-C₁-C₄-alkyl.

In another embodiment R⁷ is M₄-M₇-heterocycle.

In a further embodiment, R⁷ is tetrahydro-2H-pyran-4-yl, (3S)-tetrahydrofuran-3-yl, (3R)-tetrahydrofuran-3-yloxy, piperidin-4-yl, 1-methylpiperidin-4-yl, or 1-acetylpiperidin-4-yl.

In another embodiment R⁷ is M₄-M₇-heterocycle-C₁-C₄alkyl.

In one embodiment, R¹⁰ is hydrogen, C₁-C₆-alkyl, or C₃-C₇-cycloalkyl.

In another embodiment, R¹⁰ is hydrogen.

In another embodiment, R¹⁰ is C₁-C₆-alkyl.

In another embodiment, R¹⁰ is C₃-C₇-cycloalkyl.

In one embodiment, R¹¹ is C₁-C₆-alkyl, C₂-C₆-alkenyl.

In another embodiment, R¹¹ is C₁-C₆-alkyl.

In another embodiment, R¹¹ is C₂-C₆-alkenyl.

In one embodiment, G¹ is monocyclic M₄-M₇-heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, G^{1a}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}-(CR^{a}R^{b})ₚ—C(O)—, G^{1a}-OC(O)—, G^{1a}-(CR^{a}R^{b})ₚ—OC(O)—, G^{1b}-, G^{1b}C(O)—, G^{1b}-(CR^{a}R^{b})ₚ—, G^{1b}C(O)—(CR^{a}R^{b})ₚ—, G^{1b}-(CR^{a}R^{b})ₚ—OC(O)—, G^{1b}-(CR^{a}R^{b})ₚ—C(O)—, G^{1c}-, G^{1c}C(O)—, G^{1c}-(CR^{a}R^{b})ₕ—, G^{1c}C(O)—(CR^{a}R^{b})ₚ—, G^{1c}-(CR^{a}R^{b})ₚ—OC(O)—, G^{1c}-(CR^{a}R^{b})ₚ—C(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; G^{1a} is aryl or heteroaryl wherein the aryl or heteroaryl are optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; G^{1b} is C₃-C₈-cycloalkyl or C₄-C₈-cycloalkenyl, wherein the C₃-C₈-cycloalkyl or C₄-C₈-cycloalkenyl are optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; G^{1c} is M₄-M₇-heterocycle wherein the M₄-M₇-heterocycle is optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl, wherein Ra, Rb, Ru, Rv, h, j, k, m and p are as defined in the Summary.

In another embodiment, G¹ is monocyclic M₄-M₇-heterocycle unsubstituted or optionally substituted with 1, 2, 3, or 4 substituents selected from C₁-C₈-alkyl, oxo, G^{1a}S(O)₂—, R^{u}C(O)—, R^{u}OC(O)—, (R^{u})(R^{v})NC(O)—, R^{u}O—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}-(CR^{a}R^{b})ₚ—OC(O)—, G^{1b}-, G^{1b}C(O)—, G^{1b}-(CR^{a}R^{b})ₚ—, G^{1b}-(CR^{a}R^{b})ₚ—C(O)—, G^{1c}-, G^{1c}C(O)—, G^{1c}-(CR^{a}R^{b})ₕ—, G^{1c}C(O)—(CR^{a}R^{b})ₚ—, and C₁-C₄-haloalkyl; G^{1a} is aryl or heteroaryl wherein the aryl or heteroaryl are optionally substituted with 1, 2, 3, 4, or 5 substituents selected from C₁-C₄-alkyl, halogen, and R^{u}O—; G^{1b} is C₃-C₈-cycloalkyl, wherein the C₃-C₈-cycloalkyl is optionally substituted with 1, 2, 3, 4, or 5 substituents selected from halogen; G^{1c} is M₄-M₇-heterocycle wherein the M₄-M₇-heterocycle is optionally substituted with 1, 2, 3, 4, or 5 substituents selected from C₁-C₄-alkyl, wherein R^{a}, R^{b}, R^{u}, R^{v}, h, j, k and p are as defined in the Summary.

In a further embodiment, G¹ is 4-phenylpiperazin-1-yl, 4-(pyridin-3-yl)piperazin-1-yl, 4-cyclopropylpiperazin-1-yl, 4-acetylpiperazin-1-yl, 4-[2-(morpholin-4-yl)ethyl]piperazin-1-yl, 4-(5-methoxypyrimidin-4-yl)piperazin-1-yl, 4-(3-hydroxypropyl)piperazin-1-yl, 4-(5-chloropyridin-3-yl)piperazin-1-yl, 4-(1-methylpiperidin-4-yl)piperazin-1-yl, 4-[2-(pyridin-2-yl)ethyl]piperazin-1-yl, 4-[2-(2-hydroxyethoxy)ethyl]piperazin-1-yl, 4-(3-chlorophenyl)piperazin-1-yl, 4-(pyrimidin-2-yl)piperazin-1-yl, 4-(4-fluorophenyl)piperazin-1-yl, 4-(3,3-dimethylbutyl)piperazin-1-yl, 4-cyclohexylpiperazin-1-yl, 4-(cyclopropylcarbonyl)piperazin-1-yl, 4-(2,2-dimethylpropanoyl)piperazin-1-yl, 4-(pyrazin-2-yl)piperazin-1-yl, 4-cyclobutylpiperazin-1-yl, 4-cyclopentylpiperazin-1-yl, 2-isobutylmorpholin-4-yl, 4-(morpholin-4-yl)piperidin-1-yl, 4-hydroxy-4-phenylpiperidin-1-yl, 4-cyclohexylpiperidin-1-yl, tert-butyl piperazin-1-yl carboxylate, 4-(pyridin-2-yl)piperazin-1-yl, 4-acetylpiperazin-1-yl, 1-piperidinyl-4-carboxamide, 4-benzoylpiperazin-1-yl, 4-[2-oxo-2-(pyrrolidin-1-yl)ethyl]piperazin-1-yl, 4-benzylpiperazin-1-yl, 4-(pyridin-4-yl)piperazin-1-yl, 4-isopropylpiperazin-1-yl, 4-cyclopentylpiperazin-1-yl, 4-[2-oxo-2-(piperidin-1-yl)ethyl]piperazin-1-yl, 4-(2,2,2-trifluoroethyl)piperazin-1-yl, 4-(cyclopentylmethyl)piperazin-1-yl, piperazin-1-yl, 4-(5-fluoropyrimidin-2-yl)piperazin-1-yl, isopropyl 4piperazin-1-yl-carboxylate, 4-(pyridin-3-ylmethyl)piperazin-1-yl, isobutyl 4-piperazin-1-yl-carboxylate, benzyl 4-piperazin-1-yl-carboxylate, ethyl 4-piperazin-1-yl-carboxylate, propyl 4-piperazin-1-yl-carboxylate, 2,2,2-trifluoroethyl 4-piperazin-1-yl-carboxylate, 2,2-dimethylpropyl 4-piperazin-1-yl-carboxylate, 2-methoxyethyl 4-piperazin-1-yl-carboxylate, 4-(cyclobutylcarbonyl)piperazin-1-yl, 4-(pyridin-3-ylmethyl)piperazin-1-yl, 4-(3-fluorobenzyl)piperazin-1-yl, 4-[(5-chloro-2-furyl)methyl]piperazin-1-yl, 4-[(4-methylphenyl)sulfonyl]piperazin-1-yl, methyl 4-piperazin-1-yl-carboxylate, 4-(3-fluorobenzyl)piperazin-1-yl, N-isopropyl-4-piperazin-1-yl-carboxamide, 4-benzyl-3-oxopiperazin-1-yl, 4-(3-methylbutanoyl)piperazin-1-yl, 1,1,1-trifluoropropan-2-yl 4-piperazin-1-yl-carboxylate, 4-(cyclopropylacetyl)piperazin-1-yl, 4-(2,2,3,3-tetrafluoropropyl)piperazin-1-yl, 4-(3,3,3-trifluoropropyl)piperazin-1-yl, 4-(2,2-difluoroethyl)piperazin-1-yl, 4-(2,2,3,3,3-pentafluoropropyl)piperazin-1-yl, 4-[(3,3-difluorocyclobutyl)methyl]piperazin-1-yl, 4-(2,2-difluoropropyl)piperazin-1-yl, or 4-[(2,2-difluorocyclopropyl)methyl]piperazin-1-yl.

In one embodiment, G² is a fused-bicyclic heterocycle or spirocyclic heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₘ—, R^{u}O—(CR^{a}R^{b})ₙ—O—(CR^{a}R^{b})ₘ—, G^{1a}-, G^{1a}C(O)—, G^{1a}-(CR^{a}R^{b})_{q}—, G^{1b}-, G^{1b}C(O)—, G^{1b}-(CR^{a}R^{b})_{q}—, G^{1b}C(O)—(CR^{a}R^{b})_{q}—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; G^{1a} is aryl or heteroaryl wherein the aryl or heteroaryl are optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; G^{1b} is C₃-C₈-cycloalkyl or C₄-C₈-cycloalkenyl, wherein the C₃-C₈-cycloalkyl or C₄-C₈-cycloalkenyl are optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; wherein R^{a}, R^{b}, R^{u}, R^{v}, m, n, and q are as defined in the Summary.

In another embodiment, G² is a fused-bicyclic heterocycle or spirocyclic heterocycle unsubstituted or optionally substituted with 1, 2, 3, or 4 substituents selected from G^{1a}-(CR^{a}R^{b})_{q}— and C₁-C₄-haloalkyl; G^{1a} is aryl or heteroaryl wherein the aryl or heteroaryl are optionally substituted with 1, 2, 3, 4, or 5 substituents selected from C₁-C₄-alkyl, halogen, and R^{u}O—; and G^{1b} is C₃-C₈-cycloalkyl, wherein the C₃-C₈-cycloalkyl is optionally substituted with 1, 2, 3, 4, or 5 substituents selected from C₁-C₄-alkyl or halogen.

In a further embodiment, G² is octahydro-2H-pyrido[1,2-a]pyrazin-2-yl, (3aR*,6aS*)-5-benzylhexahydropyrrolo[3,4-c]pyrrol-2(1H)-yl, or 6-(2,2,2-trifluoroethyl)-2,6-diazaspiro[3.3]hept-2-yl.

In one embodiment, L¹ and L² are independently selected from a bond, —O—, —NR^{c}—, —C(O)—, —R^{c}NC(O)—, —C(O)NR^{c}—, —R^{c}NC(O)O—, —OC(O)NR^{c}—, —NR^{c}C(O)NR^{c}—, —S(O)—, —S(O)₂—, —R^{c}NS(O)₂—, and —S(O)₂NR^{c}—; wherein R^{c} is as described in the Summary.

In another embodiment, L¹ and L² are independently selected from a bond, —O—, —NR^{c}—, and —OC(O)NR^{c}—; wherein R^{c} is as described in the Summary.

In another embodiment, L¹ and L² are independently selected from a bond, —O—, —NH—, and —OC(O)NH—.

In one embodiment, Y is monocyclic C₃-C₈-cycloalkyl, monocyclic C₃-C₈-cycloalkenyl, or monocyclic M₄-M₇-heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₚ—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; wherein R^{a}, R^{b}, R^{u}, R^{v}, and p are as described in the Summary.

In another embodiment, Y is monocyclic C₃-C₈-cycloalkyl unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₚ—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; wherein R^{a}, R^{b}, R^{u}, R^{v}, and p are as described in the Summary.

In a further embodiment, Y is cyclohexyl.

In one embodiment, Y is aryl or heteroaryl unsubstituted or optionally substituted with 1, 2, 3, or 4 substituents selected from C₁-C₈-alkyl, C₂-C₈-alkenyl, C₂-C₈-alkynyl, halogen, cyano, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₚ—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; wherein R^{a}, R^{b}, R^{u}, R^{v}, and m are as described in the Summary.

In another embodiment, Y is aryl unsubstituted or optionally substituted with 1, 2, 3, or 4 substituents selected from C₁-C₈-alkyl, C₂-C₈-alkenyl, C₂-C₈-alkynyl, halogen, cyano, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₘ—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; wherein R^{a}, R^{b}, R^{y}, and m are as described in the Summary.

In a further embodiment, Y is phenyl.

In one embodiment, Y is C₁-C₆-alkyl or C₁-C₆-fluoroalkyl optionally substituted with 1, 2, 3, 4, or 5 substituents selected from halogen, cyano, oxo, O₂N—, R^{s}O—, R^{s}C(O)O—, (R^{s})(R^{t})NC(O)O—, R^{s}S—, R^{s}S(O)—, R^{s}S(O)₂—, (R^{s})(R^{t})NS(O)₂—, R^{s}C(O)—, R^{s}OC(O)—, (R^{s})(R^{t})NC(O)—, (R^{s})(R^{t})N—, R^{t}C(O)N(R^{s})—, (R^{t})OC(O)N(R^{s})—, and (R^{t})S(O)₂N(R^{s})—; wherein Rs and Rt are as described in the Summary.

In another embodiment, Y is C₁-C₆-alkyl optionally substituted with 1, 2, 3, 4, or 5 substituents selected from halogen, cyano, oxo, O₂N—, R^{s}O—, R^{s}C(O)O—, (R^{s})(R^{t})NC(O)O—, R^{s}S—, R^{s}S(O)—, R^{s}S(O)₂—, (R^{s})(R^{t})NS(O)₂—, R^{s}C(O)—, R^{s}OC(O)—, (R^{s})(R^{t})NC(O)—, (R^{s})(R^{t})N—, R^{t}C(O)N(R^{s})—, (R^{t})OC(O)N(R^{s})—, and (R^{t})S(O)₂N(R^{s})—; wherein Rs and Rt are as described in the Summary.

In a further embodiment, Y is isopropyl or tert-butyl.

In one embodiment, Y-L¹-(CR^{a}R^{b})_{f}-L²-is 2-(cyclohexyloxy)ethoxy, 2-phenoxyethoxy, (2-phenoxyethyl)amino, or (CH₃)₂CH—OC(O)NHCH₂CH₂O—.

In one embodiment, X is N, CH, or CF.

In another embodiment, X is N.

In another embodiment, X is CH.

In another embodiment, X is CF.

In one embodiment; R¹ is H₂N—; R² is R⁷O—; R⁴ is G¹-; R⁵ is hydrogen, R¹¹C(O)—, R¹⁰N(H)C(O)—, R¹¹C(O)NH—, R¹¹SO₂NH—, R¹N(H)SO₂—, R¹¹CH(OH)—, or NC—; R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₄-alkyl or R^{v}C(O)—; G¹ is monocyclic M₄-M₇-heterocycle unsubstituted or optionally substituted with 1 or 2 substituents selected from C₁-C₈-alkyl, C₂-C₈-alkenyl, C₂-C₈-alkynyl, halogen, cyano, oxo, R^{u}S(O)₂—, G^{1a}S(O)₂—, (R^{u})(R^{v})NS(O)₂—, R^{v}C(O)—, R^{u}OC(O)—, (R^{u})(R^{v})NC(O)—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}-(CR^{a}R^{b})ₚ—OC(O)—, G^{1b}G^{1b}C(O)—, G^{1b}-(CR^{a}R^{b})ₚ—, G^{1b}-(CR^{a}R^{b})ₚ—C(O)— G^{1c}G^{1c}C(O)—, G^{1c}-(CR^{a}R^{b})ₕ—, G^{1c}C(O)—(CR^{a}R^{b})ₚ—, and C₁-C₄-haloalkyl; G^{1a} is aryl or heteroaryl wherein the aryl or heteroaryl are optionally substituted with 1 substituents selected from C₁-C₄-alkyl, halogen, or R^{u}O—; G^{1b} is C₃-C₈-cycloalkyl, wherein the C₃-C₈-cycloalkyl is optionally substituted with for 2 halogen; G^{1c} is M₄-M₇-heterocycle wherein the M₄-M₇-heterocycle is optionally substituted with 1 C₁-C₄-alkyl; and R³, R⁶, R¹⁰, R¹¹, R^{a}, R^{b}, R^{u}, R^{v}, h, j, k, p and X are as defined in the Summary.

In one embodiment; R¹ is H₂N—; R² is R⁷O—; R⁴ is G²-; R⁵ is hydrogen, R¹¹C(O)—, R¹⁰N(H)C(O)—, R¹¹C(O)NH—, R¹¹SO₂NH—, R¹¹CH(OH)—, or NC—; R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₄-alkyl or R^{v}C(O)—; G² is a fused-bicyclic heterocycle or spirocyclic heterocycle unsubstituted or optionally substituted with 1 substituent selected from C₁-C₈-alkyl, G^{1a}-(CR^{a}R^{b})_{q}—, and C₁-C₄-haloalkyl; and R³, and X are as defined in the Summary.

In one embodiment; R¹ is H₂N—; R² is R⁷O—; R⁴ is hydrogen or Y-L¹-(CR^{a}R^{b})_{f}-L²-; R⁵ is hydrogen, R¹¹C(O)—, R¹⁰N(H)C(O)—, R¹¹C(O)NH—, R¹¹SO₂NH—, R¹¹CH(OH)—, or NC—; R⁷ is C₁-C₆ alkyl C₁-C₆-fluoroalkyl, C₃-C₈-cycloalkyl, or M₄-M₇-heterocycle, wherein the M₄-M₇-heterocycle is optionally substituted with C₁-C₄-alkyl or R^{v}C(O)—; L¹ and L² are independently selected from —O—, —NR^{c}—, and —OC(O)NR^{c}—; Y is C₃-C₈-cycloalkyl, aryl, or C₁-C₆-alkyl; and R³, R⁶, R¹⁰, R¹¹, R^{a}, R^{b}, R^{c}, R^{u}, f and X are as defined in the Summary.

In one embodiment; R¹ is H₂N—; R² is hydrogen; R⁴ is G¹-; R⁵ is hydrogen, R¹¹C(O)—, R¹⁰N(H)C(O)—, R¹¹C(O)NH—, R¹¹SO₂NH—, R¹¹CH(OH)—, or NC—; G¹ is monocyclic M₄-M₇-heterocycle unsubstituted or optionally substituted with 1 or 2 substituents selected from C₁-C₈-alkyl, C₂-C₈-alkenyl, C₂-C₈-alkynyl, halogen, cyano, oxo, R^{u}S(O)₂—, G^{1a}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}-(CR^{a}R^{b})ₚ—OC(O)—, G^{1b}-, G^{1b}C(O), G^{1b}-(CR^{a}R^{b})ₚ, G^{1b}-(CR^{a}R^{b})ₚ—C(O)—, G^{1c}-, G^{1c}- C(O)—, G^{1c}-(CR^{a}R^{b})ₕ—, G^{1c}C(O)—(CR^{a}R^{b})ₚ—, and C₁-C₄-haloalkyl; G^{1a} is aryl or heteroaryl wherein the aryl or heteroaryl are optionally substituted with 1 substituents selected from C₁-C₄-alkyl, halogen, or R^{u}O—; G^{1b} is C₃-C₈-cycloalkyl, wherein the C₃-C₈-cycloalkyl is optionally substituted with for 2 halogen; G^{1c} is M₄-M₇-heterocycle wherein the M₄-M₇-heterocycle is optionally substituted with 1 C₁-C₄-alkyl; and R³, R⁶, R¹⁰, R¹¹, R^{a}, R^{b}, R^{u}, R^{v}, h, j, k, p and X are as defined in the Summary.

In one embodiment; R¹ is hydrogen, C₁-C₄-alkyl, C₁-C₄-fluoroalkyl, or C₃-C₆-cycloalkyl; R² is R⁷O—; R⁴ is G¹-; R⁵ is hydrogen, R¹¹C(O)—, R¹¹N(H)C(O)—, R¹¹C(O)NH—, R¹¹SO₂NH—, R¹¹CH(OH)—, R¹¹C(O)C(O)NH—, or NC—; R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₁-alkyl or R^{u}C(O)—; G¹ is monocyclic M₄-M₇-heterocycle unsubstituted or optionally substituted with 1 or 2 substituents selected from C₁-C₈-alkyl, C₂-C₈-alkenyl, C₂-C₈-alkynyl, halogen, cyano, oxo, R^{u}S(O)₂—, G^{1a}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}-(CR^{a}R^{b})ₚ—OC(O)—, G^{1b}-, G^{1b}C(O)—, G^{1b}-(CR^{a}R^{b})ₚ—, G^{1b}-(CR^{a}R^{b})ₚ—C(O)—, G^{1c}-, G^{1c}C(O)—, G^{1c}-(CR^{a}R^{b})ₕ—, G^{1c}C(O)—(CR^{a}R^{b})ₚ—, and C₁-C₄-haloalkyl; G^{1a} is aryl or heteroaryl wherein the aryl or heteroaryl are optionally substituted with 1 substituents selected from C₁-C₁-alkyl, halogen, or R^{u}O—; G^{1b} is C₃-C₈-cycloalkyl, wherein the C₃-C₈-cycloalkyl is optionally substituted with for 2 halogen; G^{1c} is M₄-M₇-heterocycle wherein the M₄-M₇-heterocycle is optionally substituted with 1 C₁-C₁-alkyl; and R³, R⁶, R¹⁰, R¹¹, R^{a}, R^{b}, R^{u}, R^{v}, h, j, k, p and X are as defined in the Summary.

In one embodiment; R¹ is hydrogen, C₁-C₁-alkyl, C₁-C₄-fluoroalkyl, or C₃-C₆-cycloalkyl; R² is R⁷O—; R⁴ is G²-; R⁵ is hydrogen, R¹¹C(O)—, R¹⁰N(H)C(O)—, R¹¹C(O)NH—, R¹¹SO₂NH—, R¹¹CH(OH)—, or NC—; R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₁-alkyl or R^{u}C(O)—; G² is a fused-bicyclic heterocycle or spirocyclic heterocycle unsubstituted or optionally substituted with 1 substituent selected from C₁-C₈-alkyl, G^{1a}-(CR^{a}R^{b})_{q}—, and C₁-C₄-haloalkyl; and R³, R⁶, R¹⁰, R¹¹, R^{a}, R^{b}, R^{u}, G^{1a}, q and X are as defined in the Summary. In one embodiment, R¹ is hydrogen, C₁-C₁-alkyl, C₁-C₄-fluoroalkyl, or C₃-C₆-cycloalkyl; R² is R⁷O—; R⁴ is hydrogen or Y-L¹-(CR^{a}R^{b})_{f}-L²-; R⁵ is hydrogen, R¹¹C(O)—, R¹⁰N(H)C(O)—, R¹¹C(O)NH—, R¹¹SO₂NH—, R¹¹CH(OH)—, or NC—; R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₁-alkyl or R^{u}C(O)—; L¹ and L² are independently selected from —O—, —NR^{c}—, and —OC(O)NR^{c}—; Y is C₃-C₈-cycloalkyl, aryl, or C₁-C₆-alkyl; and R³, R⁶, R¹⁰, R¹¹, R^{a}, R^{b}, R^{c}, R^{u}, f and X are as defined in the Summary.

In one embodiment, a compound of Formula (I) is selected from (1), (2), (3), (4), (5), (6), (7), (8), or (9):

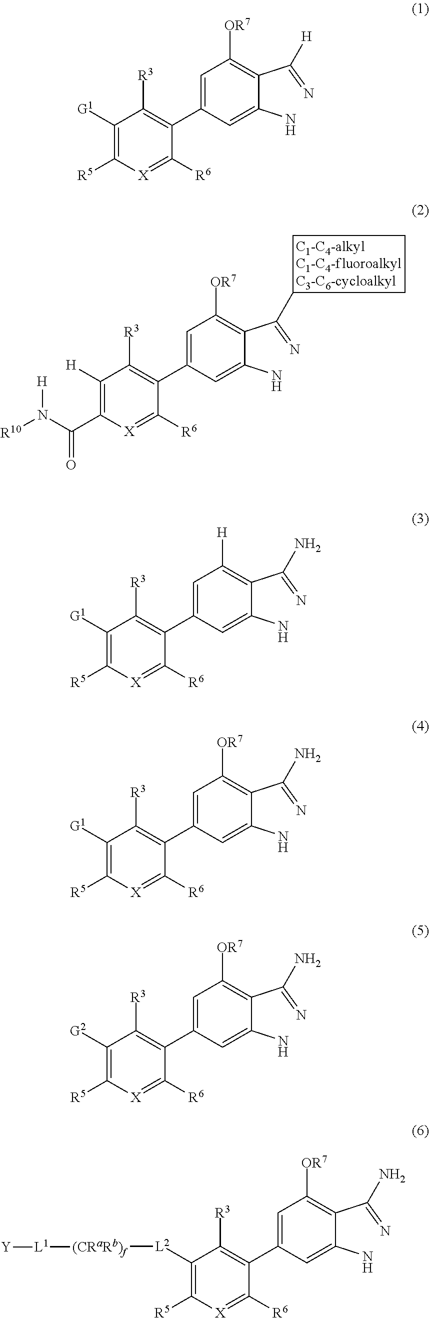

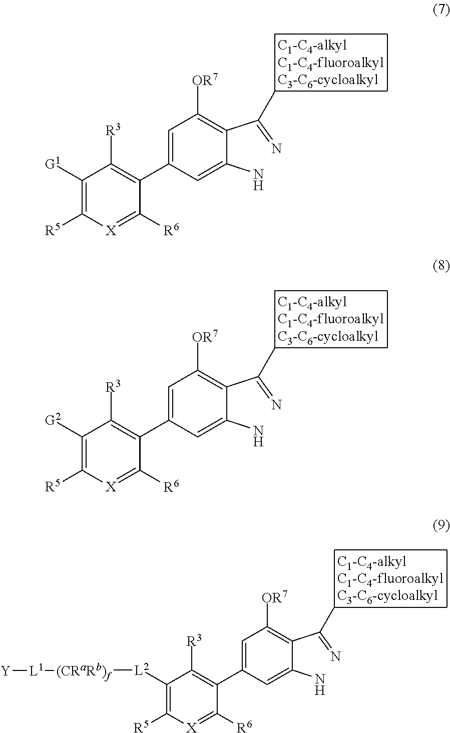

wherein R³, R⁵, R⁶, R⁷, R¹⁰, R^{a}, R^{b}, G¹, G², L¹, L², and f are as defined above.

Specific embodiments contemplated as part of the invention also include, but are not limited to, compounds of Formula (I), as defined, for example:
4-[3-amino-4-(tetrahydro-2H-pyran-4-yloxy)-1H-indazol-6-yl]-2-(4-phenylpiperazin-1-yl)benzamide;
4-[3-amino-4-(tetrahydro-2H-pyran-4-yloxy)-1H-indazol-6-yl]-2-[4-(pyridin-3-yl)piperazin-1-yl]benzamide;
4-[3-amino-4-(tetrahydro-2H-pyran-4-yloxy)-1H-indazol-6-yl]-2-(4-cyclopropylpiperazin-1-yl)benzamide;
2-(4-acetylpiperazin-1-yl)-4-[3-amino-4-(tetrahydro-2H-pyran-4-yloxy)-1H-indazol-6-yl]benzamide;
4-[3-amino-4-(tetrahydro-2H-pyran-4-yloxy)-1H-indazol-6-yl]-2-{4-[2-(morpholin-4-yl)ethyl]piperazin-1-yl}benzamide;
4-[3-amino-4-(tetrahydro-2H-pyran-4-yloxy)-1H-indazol-6-yl]-2-[4-(5-methoxypyrimidin-4-yl)piperazin-1-yl]benzamide;
4-[3-amino-4-(tetrahydro-2H-pyran-4-yloxy)-1H-indazol-6-yl]-2-[4-(3-hydroxypropyl)piperazin-1-yl]benzamide;
4-[3-amino-4-(tetrahydro-2H-pyran-4-yloxy)-1H-indazol-6-yl]-2-[4-(5-chloropyridin-3-yl)piperazin-1-yl]benzamide;
4-[3-amino-4-(tetrahydro-2H-pyran-4-yloxy)-1H-indazol-6-yl]-2-[4-(1-methylpiperidin-4-yl)piperazin-1-yl]benzamide;
4-[3-amino-4-(tetrahydro-2H-pyran-4-yloxy)-1H-indazol-6-yl]-2-{4-[2-(pyridin-2-yl)ethyl]piperazin-1-yl}benzamide;
4-[3-amino-4-(tetrahydro-2H-pyran-4-yloxy)-1H-indazol-6-yl]-2-{4-[2-(2-hydroxyethoxy)ethyl]piperazin-1-yl}benzamide;
4-[3-amino-4-(tetrahydro-2H-pyran-4-yloxy)-1H-indazol-6-yl]-2-[4-(3-chlorophenyl)piperazin-1-yl]benzamide;
1-{4-[3-amino-4-(tetrahydro-2H-pyran-4-yloxy)-1H-indazol-6-yl]-2-(4-phenylpiperazin-1-yl)phenyl}ethanone;
1-{4-[3-amino-4-(tetrahydro-2H-pyran-4-yloxy)-1H-indazol-6-yl]-2-(4-cyclopropylpiperazin-1-yl)phenyl}ethanone;
1-{4-[3-amino-4-(tetrahydro-2H-pyran-4-yloxy)-1H-indazol-6-yl]-2-[4-(pyrimidin-2-yl)piperazin-1-yl]phenyl}ethanone;
1-{4-[3-amino-4-(tetrahydro-2H-pyran-4-yloxy)-1H-indazol-6-yl]-2-[4-(pyridin-3-yl)piperazin-1-yl]phenyl}ethanone;
1-{4-[3-amino-4-(tetrahydro-2H-pyran-4-yloxy)-1H-indazol-6-yl]-2-[4-(4-fluorophenyl)piperazin-1-yl]phenyl}ethanone;
1-{4-[3-amino-4-(tetrahydro-2H-pyran-4-yloxy)-1H-indazol-6-yl]-2-[4-(3,3-dimethylbutyl)piperazin-1-yl]phenyl}ethanone;
1-{4-[3-amino-4-(tetrahydro-2H-pyran-4-yloxy)-1H-indazol-6-yl]-2-(4-cyclohexylpiperazin-1-yl)phenyl}ethanone;
1-{4-[3-amino-4-(tetrahydro-2H-pyran-4-yloxy)-1H-indazol-6-yl]-2-[4-(cyclopropylcarbonyl)piperazin-1-yl]phenyl}ethanone;
1-{4-[3-amino-4-(tetrahydro-2H-pyran-4-yloxy)-1H-indazol-6-yl]-2-[4-(1,3-thiazol-2-yl)piperazin-1-yl]phenyl}ethanone;
1-{4-[3-amino-4-(tetrahydro-2H-pyran-4-yloxy)-1H-indazol-6-yl]-2-[4-(pyridin-3-yl)piperazin-1-yl]phenyl}ethanol;
4-[3-amino-4-(tetrahydro-2H-pyran-4-yloxy)-1H-indazol-6-yl]-N-cyclopropyl-2-(4-phenylpiperazin-1-yl)benzamide;
4-[3-amino-4-(tetrahydro-2H-pyran-4-yloxy)-1H-indazol-6-yl]-N-cyclopropyl-2-[4-(pyridin-3-yl)piperazin-1-yl]benzamide;
4-[3-amino-4-(tetrahydro-2H-pyran-4-yloxy)-1H-indazol-6-yl]-N-cyclopropyl-2-[4-(2,2-dimethylpropanoyl)piperazin-1-yl]benzamide;
4-{3-amino-4-[(1,1,1-trifluoropropan-2-yl)oxy]-1H-indazol-6-yl}-N-cyclopropyl-2-(4-phenylpiperazin-1-yl)benzamide;
4-{3-amino-4-[(1,1,1-trifluoropropan-2-yl)oxy]-1H-indazol-6-yl}-N-cyclopropyl-2-(4-cyclopropylpiperazin-1-yl)benzamide;
4-{3-amino-4-[(1,1,1-trifluoropropan-2-yl)oxy]-1H-indazol-6-yl}-N-cyclopropyl-2-[4-(pyridin-3-yl)piperazin-1-yl]benzamide;
4-[3-amino-4-(tetrahydro-2H-pyran-4-yloxy)-1H-indazol-6-yl]-2-(4-cyclohexylpiperazin-1-yl)-N-methylbenzamide;
4-[3-amino-4-(tetrahydro-2H-pyran-4-yloxy)-1H-indazol-6-yl]-N-methyl-2-(4-phenylpiperazin-1-yl)benzamide;
4-[3-amino-4-(tetrahydro-2H-pyran-4-yloxy)-1H-indazol-6-yl]-N-methyl-2-[4-(pyridin-3-yl)piperazin-1-yl]benzamide;
4-[3-amino-4-(tetrahydro-2H-pyran-4-yloxy)-1H-indazol-6-yl]-2-[4-(cyclopropylcarbonyl)piperazin-1-yl]-N-methylbenzamide;
4-{3-amino-4-[(1,1,1-trifluoropropan-2-yl)oxy]-1H-indazol-6-yl}-N-methyl-2-(4-phenylpiperazin-1-yl)benzamide;
4-{3-amino-4-[(1,1,1-trifluoropropan-2-yl)oxy]-1H-indazol-6-yl}-2-(4-cyclopropylpiperazin-1-yl)-N-methylbenzamide;
4-{3-amino-4-[(1,1,1-trifluoropropan-2-yl)oxy]-1H-indazol-6-yl}-2-(4-cyclohexylpiperazin-1-yl)-N-methylbenzamide;
4-{3-amino-4-[(1, 1,1-trifluoropropan-2-yl)oxy]-1H-indazol-6-yl}-N-methyl-2-[4-(pyridin-3-yl)piperazin-1-yl]benzamide;
4-{3-amino-4-[(1, 1,1-trifluoropropan-2-yl)oxy]-1H-indazol-6-yl}-N-methyl-2-{4-[2-(morpholin-4-yl)ethyl]piperazin-1-yl}benzamide;
4-{3-amino-4-[(1, 1,1-trifluoropropan-2-yl)oxy]-1H-indazol-6-yl}-N-methyl-2-[4-(pyrazin-2-yl)piperazin-1-yl]benzamide;
4-(3-amino-4-{[(2S)-1,1,1-trifluoropropan-2-yl]oxy}-1H-indazol-6-yl)-N-methyl-2-(4-phenylpiperazin-1-yl)benzamide;
4-(3-amino-4-{[(2S)-1,1,1-trifluoropropan-2-yl]oxy}-1H-indazol-6-yl)-2-[4-(3,3-dimethylbutyl)piperazin-1-yl]-N-methylbenzamide;
4-(3-amino-4-{[(2S)-1,1,1-trifluoropropan-2-yl]oxy}-1H-indazol-6-yl)-N-methyl-2-[4-(pyridin-3-yl)piperazin-1-yl]benzamide;
4-(3-amino-4-{[(2S)-1,1,1-trifluoropropan-2-yl]oxy}-1H-indazol-6-yl)-2-(4-cyclobutylpiperazin-1-yl)-N-methylbenzamide;
4-(3-amino-4-{[(2S)-1,1,1-trifluoropropan-2-yl]oxy}-1H-indazol-6-yl)-N-methyl-2-[4-(pyrimidin-2-yl)piperazin-1-yl]benzamide;
4-(3-amino-4-{[(2S)-1,1,1-trifluoropropan-2-yl]oxy}-1H-indazol-6-yl)-2-(4-cyclopentylpiperazin-1-yl)-N-methylbenzamide;
4-(3-amino-4-{[(2S)-1,1,1-trifluoropropan-2-yl]oxy}-1H-indazol-6-yl)-2-[4-(2,2-dimethylpropanoyl)piperazin-1-yl]-N-methylbenzamide;
4-(3-amino-4-{[(2S)-1,1,1-trifluoropropan-2-yl]oxy}-1H-indazol-6-yl)-N-methyl-2-[4-(pyrazin-2-yl)piperazin-1-yl]benzamide;
4-(3-amino-4-{[(2S)-1,1,1-trifluoropropan-2-yl]oxy}-1H-indazol-6-yl)-2-(4-cyclohexylpiperazin-1-yl)-N-methylbenzamide;
4-(3-amino-4-{[(2R)-1,1,1-trifluoropropan-2-yl]oxy}-1H-indazol-6-yl)-N-methyl-2-(4-phenylpiperazin-1-yl)benzamide;
4-(3-amino-4-{[(2R)-1,1,1-trifluoropropan-2-yl]oxy}-1H-indazol-6-yl)-N-methyl-2-[4-(pyridin-3-yl)piperazin-1-yl]benzamide;
4-(3-amino-4-{[(2R)-1,1,1-trifluoropropan-2-yl]oxy}-1H-indazol-6-yl)-2-(4-cyclobutylpiperazin-1-yl)-N-methylbenzamide;
4-(3-amino-4-{[(2R)-1,1,1-trifluoropropan-2-yl]oxy}-1H-indazol-6-yl)-2-[4-(3,3-dimethylbutyl)piperazin-1-yl]-N-methylbenzamide;
4-(3-amino-4-{[(2R)-1,1,1-trifluoropropan-2-yl]oxy}-1H-indazol-6-yl)-N-methyl-2-[4-(pyrimidin-2-yl)piperazin-1-yl]benzamide;
4-(3-amino-4-{[(2R)-1,1,1-trifluoropropan-2-yl]oxy}-1H-indazol-6-yl)-2-(4-cyclopentylpiperazin-1-yl)-N-methylbenzamide;
4-(3-amino-4-{[(2R)-1,1,1-trifluoropropan-2-yl]oxy}-1H-indazol-6-yl)-2-[4-(2,2-dimethylpropanoyl)piperazin-1-yl]-N-methylbenzamide;
4-(3-amino-4-{[(2R)-1,1,1-trifluoropropan-2-yl]oxy}-1H-indazol-6-yl)-N-methyl-2-[4-(pyrazin-2-yl)piperazin-1-yl]benzamide;
4-(3-amino-4-{[(2R)-1,1,1-trifluoropropan-2-yl]oxy}-1H-indazol-6-yl)-2-(4-cyclohexylpiperazin-1-yl)-N-methylbenzamide;
4-(3-amino-4-{[(2R)-1,1,1-trifluoropropan-2-yl]oxy}-1H-indazol-6-yl)-N-methyl-2-(octahydro-2H-pyrido[1,2-a]pyrazin-2-yl)benzamide;
4-(3-amino-4-{[(2R)-1,1,1-trifluoropropan-2-yl]oxy}-1H-indazol-6-yl)-2-(2-isobutylmorpholin-4-yl)-N-methylbenzamide;
4-(3-amino-4-{[(2R)-1,1,1-trifluoropropan-2-yl]oxy}-1H-indazol-6-yl)-N-methyl-2-[4-(morpholin-4-yl)piperidin-1-yl]benzamide;
4-(3-amino-4-{[(2R)-1,1,1-trifluoropropan-2-yl]oxy}-1H-indazol-6-yl)-2-(4-hydroxy-4-phenylpiperidin-1-yl)-N-methylbenzamide;
4-(3-amino-4-{[(2R)-1,1,1-trifluoropropan-2-yl]oxy}-1H-indazol-6-yl)-2-(4-cyclohexylpiperidin-1-yl)-N-methylbenzamide;
4-{3-amino-4-[(1-methylpiperidin-4-yl)oxy]-1H-indazol-6-yl}-N-cyclopropyl-2-(4-phenylpiperazin-1-yl)benzamide;
4-[3-amino-4-(piperidin-4-yloxy)-1H-indazol-6-yl]-2-(4-phenylpiperazin-1-yl)benzamide;
4-{4-[(1-acetylpiperidin-4-yl)oxy]-3-amino-1H-indazol-6-yl}-2-(4-phenylpiperazin-1-yl)benzamide;
4-{3-amino-4-[(3S)-tetrahydrofuran-3-yloxy]-1H-indazol-6-yl}-2-(4-cyclopropylpiperazin-1-yl)benzamide;
4-{3-amino-4-[(3S)-tetrahydrofuran-3-yloxy]-1H-indazol-6-yl}-2-(4-phenylpiperazin-1-yl)benzamide;
4-{3-amino-4-[(3S)-tetrahydrofuran-3-yloxy]-1H-indazol-6-yl}-2-[4-(pyridin-3-yl)piperazin-1-yl]benzamide;
4-{3-amino-4-[(3R)-tetrahydrofuran-3-yloxy]-1H-indazol-6-yl}-2-[4-(pyridin-3-yl)piperazin-1-yl]benzamide;
4-[3-amino-4-(cyclopentyloxy)-1H-indazol-6-yl]-2-[4-(pyridin-3-yl)piperazin-1-yl]benzamide;
4-[3-amino-4-(cyclopentyloxy)-1H-indazol-6-yl]-2-{4-[2-(morpholin-4-yl)ethyl]piperazin-1-yl}benzamide;
4-[3-amino-4-(cyclopentyloxy)-1H-indazol-6-yl]-2-(4-phenylpiperazin-1-yl)benzamide;
4-[3-amino-4-(cyclopentyloxy)-1H-indazol-6-yl]-2-[4-(4-fluorophenyl)piperazin-1-yl]benzamide;
tert-butyl 4-{5-[3-amino-4-(cyclopentyloxy)-1H-indazol-6-yl]-2-carbamoylphenyl}piperazine-1-carboxylate;
4-[3-amino-4-(cyclopentyloxy)-1H-indazol-6-yl]-2-(4-cyclohexylpiperazin-1-yl)benzamide;
4-[3-amino-4-(cyclopentyloxy)-1H-indazol-6-yl]-2-[4-(pyridin-2-yl)piperazin-1-yl]benzamide;
4-[3-amino-4-(cyclopentyloxy)-1H-indazol-6-yl]-2-(4-phenylpiperidin-1-yl)benzamide;
2-(4-acetylpiperazin-1-yl)-4-[3-amino-4-(cyclopentyloxy)-1H-indazol-6-yl]benzamide;
4-[3-amino-4-(cyclopentyloxy)-1H-indazol-6-yl]-2-(4-cyclopropylpiperazin-1-yl)benzamide;
1-{5-[3-amino-4-(cyclopentyloxy)-1H-indazol-6-yl]-2-carbamoylphenyl}piperidine-4-carboxamide;
4-[3-amino-4-(cyclopentyloxy)-1H-indazol-6-yl]-2-(4-benzoylpiperazin-1-yl)benzamide;
4-[3-amino-4-(cyclopentyloxy)-1H-indazol-6-yl]-2-[4-(cyclopropylcarbonyl)piperazin-1-yl]benzamide;
4-[3-amino-4-(cyclopentyloxy)-1H-indazol-6-yl]-2-[4-(morpholin-4-ylcarbonyl)piperazin-1-yl]benzamide;
4-(3-amino-1H-indazol-6-yl)-2-{4-[2-oxo-2-(pyrrolidin-1-yl)ethyl]piperazin-1-yl}benzamide;
4-(3-amino-1H-indazol-6-yl)-2-(4-phenylpiperazin-1-yl)benzamide;
4-(3-amino-1H-indazol-6-yl)-2-(4-cyclohexylpiperazin-1-yl)benzamide;
4-(3-amino-1H-indazol-6-yl)-2-[4-(pyridin-2-yl)piperazin-1-yl]benzamide;
4-(3-amino-1H-indazol-6-yl)-2-(4-benzylpiperazin-1-yl)benzamide;
4-(3-amino-1H-indazol-6-yl)-2-(4-cyclopropylpiperazin-1-yl)benzamide;
4-(3-amino-1H-indazol-6-yl)-2-{4-[2-(morpholin-4-yl)ethyl]piperazin-1-yl}benzamide;
4-(3-amino-1H-indazol-6-yl)-2-[4-(pyridin-3-yl)piperazin-1-yl]benzamide;
4-{3-amino-4-[(1,1,1-trifluoropropan-2-yl)oxy]-1H-indazol-6-yl}-2-[4-(pyridin-3-yl)piperazin-1-yl]benzamide;
4-{3-amino-4-[(1,1,1-trifluoropropan-2-yl)oxy]-1H-indazol-6-yl}-2-(4-phenylpiperazin-1-yl)benzamide;
4-{3-amino-4-[(1,1,1-trifluoropropan-2-yl)oxy]-1H-indazol-6-yl}-2-(4-cyclohexylpiperazin-1-yl)benzamide;
4-{3-amino-4-[(1,1,1-trifluoropropan-2-yl)oxy]-1H-indazol-6-yl}-2-[4-(pyridin-2-yl)piperazin-1-yl]benzamide;
4-{3-amino-4-[(1,1,1-trifluoropropan-2-yl)oxy]-1H-indazol-6-yl}-2-{4-[2-(morpholin-4-yl)ethyl]piperazin-1-yl}benzamide;
2-(4-acetylpiperazin-1-yl)-4-{3-amino-4-[(1,1,1-trifluoropropan-2-yl)oxy]-1H-indazol-6-yl}benzamide;
4-{3-amino-4-[(1,1,1-trifluoropropan-2-yl)oxy]-1H-indazol-6-yl}-2-(4-cyclopropylpiperazin-1-yl)benzamide;
4-{3-amino-4-[(1,1,1-trifluoropropan-2-yl)oxy]-1H-indazol-6-yl}-2-[4-(pyridin-4-yl)piperazin-1-yl]benzamide;
4-{3-amino-4-[(1, 1,1-trifluoropropan-2-yl)oxy]-1H-indazol-6-yl}-2-[(3 aR*,6aS*)-5-benzylhexahydropyrrolo[3,4-c]pyrrol-2(1H)-yl]benzamide;
4-{3-amino-4-[(1,1,1-trifluoropropan-2-yl)oxy]-1H-indazol-6-yl}-2-[4-(3,3-dimethylbutyl)piperazin-1-yl]benzamide;
4-{3-amino-4-[(1,1,1-trifluoropropan-2-yl)oxy]-1H-indazol-6-yl}-2-(4-isopropylpiperazin-1-yl)benzamide;
4-{3-amino-4-[(1,1,1-trifluoropropan-2-yl)oxy]-1H-indazol-6-yl}-2-(4-cyclopentylpiperazin-1-yl)benzamide;
4-{3-amino-4-[(1, 1,1-trifluoropropan-2-yl)oxy]-1H-indazol-6-yl}-2-[4-(cyclopropylcarbonyl)piperazin-1-yl]benzamide;
4-{3-amino-4-[(1,1,1-trifluoropropan-2-yl)oxy]-1H-indazol-6-yl}-2-[4-(2,2-dimethylpropanoyl)piperazin-1-yl]benzamide;
4-(3-amino-4-{[(2R)-1,1,1-trifluoropropan-2-yl]oxy}-1H-indazol-6-yl)-2-[2-(cyclohexyloxy)ethoxy]-N-methylbenzamide;
4-(3-amino-4-{[(2R)-1,1,1-trifluoropropan-2-yl]oxy}-1H-indazol-6-yl)-N-methyl-2-(2-phenoxyethoxy)benzamide;
4-[3-amino-4-(cyclopentyloxy)-1H-indazol-6-yl]-2-{4-[2-oxo-2-(piperidin-1-yl)ethyl]piperazin-1-yl}benzamide;
4-[3-amino-4-(tetrahydro-2H-pyran-4-yloxy)-1H-indazol-6-yl]-N-methyl-2-[4-(2,2,2-trifluoroethyl)piperazin-1-yl]benzamide;
4-(3-amino-4-isopropoxy-1H-indazol-6-yl)-2-(4-cyclohexylpiperazin-1-yl)-N-methylbenzamide;
4-[3-amino-4-(tetrahydro-2H-pyran-4-yloxy)-1H-indazol-6-yl]-2-[4-(cyclopentylmethyl)piperazin-1-yl]-N-methylbenzamide;
tert-butyl 4-{5-[3-amino-4-(tetrahydro-2H-pyran-4-yloxy)-1H-indazol-6-yl]-2-(methylcarbamoyl)phenyl}piperazine-1-carboxylate;
4-[3-amino-4-(tetrahydro-2H-pyran-4-yloxy)-1H-indazol-6-yl]-N-methyl-2-(piperazin-1-yl)benzamide;
4-[3-amino-4-(tetrahydro-2H-pyran-4-yloxy)-1H-indazol-6-yl]-2-[4-(5-fluoropyrimidin-2-yl)piperazin-1-yl]-N-methylbenzamide;
isopropyl 4-{5-[3-amino-4-(tetrahydro-2H-pyran-4-yloxy)-1H-indazol-6-yl]-2-(methylcarbamoyl)phenyl}piperazine-1-carboxylate;
N-ethyl-4-[3-methyl-4-(tetrahydro-2H-pyran-4-yloxy)-1H-indazol-6-yl]-2-(4-phenylpiperazin-1-yl)benzamide;
2-(4-cyclohexylpiperazin-1-yl)-N-methyl-4-[4-(tetrahydro-2H-pyran-4-yloxy)-1H-indazol-6-yl]benzamide;
N-cyclopropyl-4-[3-methyl-4-(tetrahydro-2H-pyran-4-yloxy)-1H-indazol-6-yl]-2-(4-phenylpiperazin-1-yl)benzamide;
N-cyclobutyl-2-(4-cyclohexylpiperazin-1-yl)-4-[3-methyl-4-(tetrahydro-2H-pyran-4-yloxy)-1H-indazol-6-yl]benzamide;
N-{2-(4-cyclohexylpiperazin-1-yl)-4-[3-methyl-4-(tetrahydro-2H-pyran-4-yloxy)-1H-indazol-6-yl]phenyl}acetamide;
4-[4-(cyclopentyloxy)-1H-indazol-6-yl]-N-ethyl-2-(4-phenylpiperazin-1-yl)benzamide;
2-(4-cyclohexylpiperazin-1-yl)-4-[4-(cyclopentyloxy)-3-methyl-1H-indazol-6-yl]-N-methylbenzamide;
tert-butyl 4-{2-(methylcarbamoyl)-5-[3-methyl-4-(tetrahydro-2H-pyran-4-yloxy)-1H-indazol-6-yl]phenyl}piperazine-1-carboxylate;
N-methyl-4-[3-methyl-4-(tetrahydro-2H-pyran-4-yloxy)-1H-indazol-6-yl]-2-[4-(pyridin-3-ylmethyl)piperazin-1-yl]benzamide;
2-[4-(cyclopropylcarbonyl)piperazin-1-yl]-N-methyl-4-[3-methyl-4-(tetrahydro-2H-pyran-4-yloxy)-1H-indazol-6-yl]benzamide;
isopropyl 4-{2-(methylcarbamoyl)-5-[3-methyl-4-(tetrahydro-2H-pyran-4-yloxy)-1H-indazol-6-yl]phenyl}piperazine-1-carboxylate;
2-[2-(cyclohexyloxy)ethoxy]-N-methyl-4-[3-methyl-4-(tetrahydro-2H-pyran-4-yloxy)-1H-indazol-6-yl]benzamide;
N-{4-[4-(cyclopentyloxy)-3-methyl-1H-indazol-6-yl]-2-[(2-phenoxyethyl)amino]phenyl}acetamide;
isobutyl 4-{5-[4-(cyclopentyloxy)-3-methyl-1H-indazol-6-yl]-2-(methylcarbamoyl)phenyl}piperazine-1-carboxylate;
benzyl 4-{5-[4-(cyclopentyloxy)-3-methyl-1H-indazol-6-yl]-2-(methylcarbamoyl)phenyl}piperazine-1-carboxylate;
N-{4-[3-methyl-4-(tetrahydro-2H-pyran-4-yloxy)-1H-indazol-6-yl]-2-(2-phenoxyethoxy)phenyl}acetamide;
N-{2-(4-cyclohexylpiperazin-1-yl)-4-[3-methyl-4-(tetrahydro-2H-pyran-4-yloxy)-1H-indazol-6-yl]phenyl}acrylamide;
4-[3-methyl-4-(tetrahydro-2H-pyran-4-yloxy)-1H-indazol-6-yl]benzamide;
N-{2-(4-cyclohexylpiperazin-1-yl)-4-[3-methyl-4-(tetrahydro-2H-pyran-4-yloxy)-1H-indazol-6-yl]phenyl}-2-oxopropanamide;
ethyl 4-{2-(methylcarbamoyl)-5-[3-methyl-4-(tetrahydro-2H-pyran-4-yloxy)-1H-indazol-6-yl]phenyl}piperazine-1-carboxylate;
propyl 4-{2-(methylcarbamoyl)-5-[3-methyl-4-(tetrahydro-2H-pyran-4-yloxy)-1H-indazol-6-yl]phenyl}piperazine-1-carboxylate;
2,2,2-trifluoroethyl 4-{2-(methylcarbamoyl)-5-[3-methyl-4-(tetrahydro-2H-pyran-4-yloxy)-1H-indazol-6-yl]phenyl}piperazine-1-carboxylate;
2,2-dimethylpropyl 4-{2-(methylcarbamoyl)-5-[3-methyl-4-(tetrahydro-2H-pyran-4-yloxy)-1H-indazol-6-yl]phenyl}piperazine-1-carboxylate;
2-methoxyethyl 4-{2-(methylcarbamoyl)-5-[3-methyl-4-(tetrahydro-2H-pyran-4-yloxy)-1H-indazol-6-yl]phenyl}piperazine-1-carboxylate;
tert-butyl 4-{2-cyano-5-[3-methyl-4-(tetrahydro-2H-pyran-4-yloxy)-1H-indazol-6-yl]phenyl}piperazine-1-carboxylate;
tert-butyl 4-{2-carbamoyl-5-[3-methyl-4-(tetrahydro-2H-pyran-4-yloxy)-1H-indazol-6-yl]phenyl}piperazine-1-carboxylate;
tert-butyl 4-{2-acetamido-5-[3-methyl-4-(tetrahydro-2H-pyran-4-yloxy)-1H-indazol-6-yl]phenyl}piperazine-1-carboxylate;
isopropyl 4-{2-carbamoyl-5-[3-methyl-4-(tetrahydro-2H-pyran-4-yloxy)-1H-indazol-6-yl]phenyl}piperazine-1-carboxylate;
N-methyl-4-[3-methyl-4-(tetrahydro-2H-pyran-4-yloxy)-1H-indazol-6-yl]-2-[4-(2,2,2-trifluoroethyl)piperazin-1-yl]benzamide;
N-{2-[4-(cyclobutylcarbonyl)piperazin-1-yl]-4-[3-methyl-4-(tetrahydro-2H-pyran-4-yloxy)-1H-indazol-6-yl]phenyl}acetamide;
N-{2-(4-benzylpiperazin-1-yl)-4-[3-methyl-4-(tetrahydro-2H-pyran-4-yloxy)-1H-indazol-6-yl]phenyl}acetamide;
isopropyl 4-{2-acetamido-5-[3-methyl-4-(tetrahydro-2H-pyran-4-yloxy)-1H-indazol-6-yl]phenyl}piperazine-1-carboxylate;
isopropyl (2-{2-acetamido-5-[3-methyl-4-(tetrahydro-2H-pyran-4-yloxy)-1H-indazol-6-yl]phenoxy}ethyl)carbamate;
N-{4-[3-methyl-4-(tetrahydro-2H-pyran-4-yloxy)-1H-indazol-6-yl]-2-[4-(pyridin-3-ylmethyl)piperazin-1-yl]phenyl}acetamide;
N-{2-[4-(3-fluorobenzyl)piperazin-1-yl]-4-[3-methyl-4-(tetrahydro-2H-pyran-4-yloxy)-1H-indazol-6-yl]phenyl}acetamide;
N-(2-{4-[(5-chloro-2-furyl)methyl]piperazin-1-yl}-4-[3-methyl-4-(tetrahydro-2H-pyran-4-yloxy)-1H-indazol-6-yl]phenyl)acetamide;
4-{2-acetamido-5-[3-methyl-4-(tetrahydro-2H-pyran-4-yloxy)-1H-indazol-6-yl]phenyl}-N-isopropylpiperazine-1-carboxamide;
N-methyl-2-{4-[(4-methylphenyl)sulfonyl]piperazin-1-yl}-4-[3-methyl-4-(tetrahydro-2H-pyran-4-yloxy)-1H-indazol-6-yl]benzamide;
methyl 4-{2-(methylcarbamoyl)-5-[3-methyl-4-(tetrahydro-2H-pyran-4-yloxy)-1H-indazol-6-yl]phenyl}piperazine-1-carboxylate;
2-[4-(3-fluorobenzyl)piperazin-1-yl]-N-methyl-4-[3-methyl-4-(tetrahydro-2H-pyran-4-yloxy)-1H-indazol-6-yl]benzamide;
N-isopropyl-4-{2-(methylcarbamoyl)-5-[3-methyl-4-(tetrahydro-2H-pyran-4-yloxy)-1H-indazol-6-yl]phenyl}piperazine-1-carboxamide;
4-[3-methyl-4-(tetrahydro-2H-pyran-4-yloxy)-1H-indazol-6-yl]-2-[4-(2,2,2-trifluoroethyl)piperazin-1-yl]benzamide;
4-[3-methyl-4-(tetrahydro-2H-pyran-4-yloxy)-1H-indazol-6-yl]-2-[4-(2,2,2-trifluoroethyl)piperazin-1-yl]benzonitrile;
tert-butyl 4-{5-[3-ethyl-4-(tetrahydro-2H-pyran-4-yloxy)-1H-indazol-6-yl]-2-(methylcarbamoyl)phenyl}piperazine-1-carboxylate;
2-(4-benzyl-3-oxopiperazin-1-yl)-N-methyl-4-[3-methyl-4-(tetrahydro-2H-pyran-4-yloxy)-1H-indazol-6-yl]benzamide;
N-methyl-2-[4-(3-methylbutanoyl)piperazin-1-yl]-4-[3-methyl-4-(tetrahydro-2H-pyran-4-yloxy)-1H-indazol-6-yl]benzamide;
4-(cyclopentyloxy)-3-methyl-6-[5-(4-phenylpiperazin-1-yl)pyridin-3-yl]-1H-indazole;
tert-butyl 4-{2-[(methylsulfonyl)amino]-5-[3-methyl-4-(tetrahydro-2H-pyran-4-yloxy)-1H-indazol-6-yl]phenyl}piperazine-1-carboxylate;
1,1,1-trifluoropropan-2-yl 4-{2-(methylcarbamoyl)-5-[3-methyl-4-(tetrahydro-2H-pyran-4-yloxy)-1H-indazol-6-yl]phenyl}piperazine-1-carboxylate;
2-[4-(cyclopropylacetyl)piperazin-1-yl]-N-methyl-4-[3-methyl-4-(tetrahydro-2H-pyran-4-yloxy)-1H-indazol-6-yl]benzamide;
2-[4-(5-fluoropyrimidin-2-yl)piperazin-1-yl]-N-methyl-4-[3-methyl-4-(tetrahydro-2H-pyran-4-yloxy)-1H-indazol-6-yl]benzamide;
4-[3-methyl-4-(tetrahydro-2H-pyran-4-yloxy)-1H-indazol-6-yl]-2-[6-(2,2,2-trifluoroethyl)-2,6-diazaspiro[3.3]hept-2-yl]benzamide;
4-[3-amino-4-(tetrahydro-2H-pyran-4-yloxy)-1H-indazol-6-yl]-N-methyl-2-[4-(2,2,3,3-tetrafluoropropyl)piperazin-1-yl]benzamide;
4-[3-amino-4-(tetrahydro-2H-pyran-4-yloxy)-1H-indazol-6-yl]-N-methyl-2-[4-(3,3,3-trifluoropropyl)piperazin-1-yl]benzamide;
4-[3-amino-4-(tetrahydro-2H-pyran-4-yloxy)-1H-indazol-6-yl]-2-[4-(2,2-difluoroethyl)piperazin-1-yl]-N-methylbenzamide;
4-[3-amino-4-(tetrahydro-2H-pyran-4-yloxy)-1H-indazol-6-yl]-N-methyl-2-[4-(2,2,3,3,3-pentafluoropropyl)piperazin-1-yl]benzamide;
4-[3-amino-4-(tetrahydro-2H-pyran-4-yloxy)-1H-indazol-6-yl]-2-{4-[(3,3-difluorocyclobutyl)methyl]piperazin-1-yl}-N-methylbenzamide;
4-[3-amino-4-(tetrahydro-2H-pyran-4-yloxy)-1H-indazol-6-yl]-2-[4-(2,2-difluoropropyl)piperazin-1-yl]-N-methylbenzamide; and
4-[3-amino-4-(tetrahydro-2H-pyran-4-yloxy)-1H-indazol-6-yl]-2-{4-[(2,2-difluorocyclopropyl)methyl]piperazin-1-yl}-N-methylbenzamide.

Compound names are assigned by using Name Release 12.00 v. 12.5 naming algorithm by Advanced Chemical Development or Struct=Name naming algorithm as part of CHEMDRAW® ULTRA v. 12.0

Compounds of the invention may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The invention contemplates various stereoisomers and mixtures thereof and these are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

On occasion, the relative stereochemistry of an enantiomeric pair is known, however, the absolute configuration is not known. In that circumstance, the relative stereochemistry descriptor terms “R*” and “S*” are used. The terms “R*” and “S*” used herein are defined in Eliel, E. L.; Wilen, S. H. Stereochemistry of Organic Compounds; John Wiley & Sons, Inc.: New York, 1994; pp 119-120 and 1206.

Compounds of the invention may exist as cis or trans isomers, wherein substituents on a ring may attached in such a manner that they are on the same side of the ring (cis) relative to each other, or on opposite sides of the ring relative to each other (trans). For example, cyclobutane may be present in the cis or trans configuration, and may be present as a single isomer or a mixture of the cis and trans isomers. Individual cis or trans isomers of compounds of the invention may be prepared synthetically from commercially available starting materials using selective organic transformations, or prepared in single isomeric form by purification of mixtures of the cis and trans isomers. Such methods are well-known to those of ordinary skill in the art, and may include separation of isomers by recrystallization or chromatography.

It should be understood that the compounds of the invention may possess tautomeric forms, as well as geometric isomers, and that these also constitute an aspect of the invention.

The present invention also includes isotopically-labeled compounds, which are identical to those recited in formula (I) or formula (II), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Compounds incorporating positron-emitting isotopes are useful in medical imaging and positron-emitting tomography (PET) studies for determining the distribution of receptors. Suitable positron-emitting isotopes that can be incorporated in compounds of formula (I) or formula (II) are ¹¹C, ¹³N, ¹⁵O, and ¹⁸F. Isotopically-labeled compounds of formula (I) or formula (II) can generally be prepared by conventional techniques known to those skilled in the art or by processes analogous to those described in the accompanying Examples using appropriate isotopically-labeled reagent in place of non-isotopically-labeled reagent.

### Methods for Preparing Compounds of the Invention

The compounds of the invention can be better understood in connection with the following synthetic schemes and methods which illustrate a means by which the compounds can be prepared.

The compounds of this invention can be prepared by a variety of synthetic procedures. Representative procedures are shown in, but are not limited to, Schemes 1-18.

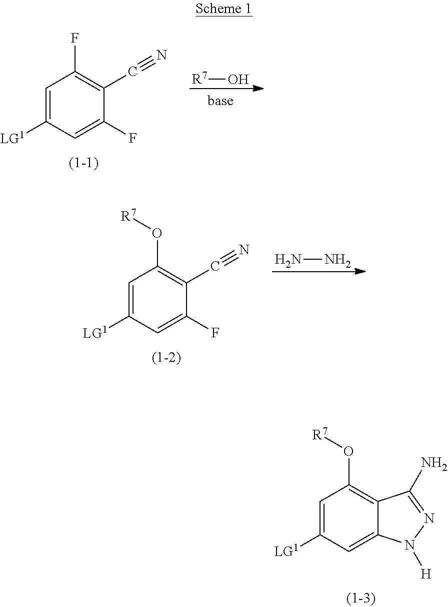

As shown in Scheme 1, compounds of Formula (1-3) can be prepared from compounds of Formula (1-1), wherein R⁷ is as defined in the Summary. To that end, compounds of Formula (1-1), wherein LG¹ is a leaving group such as chlorine, bromine or iodine, can be reacted with alkoxides formed from alcohols, R⁷—OH, in a suitable solvent and at a suitable temperature to deliver compounds of Formula (1-2). Compounds of Formula (1-2) can be reacted with hydrazine in an alcohol solvent at ambient or elevated temperatures to supply compounds of Formula (1-3).

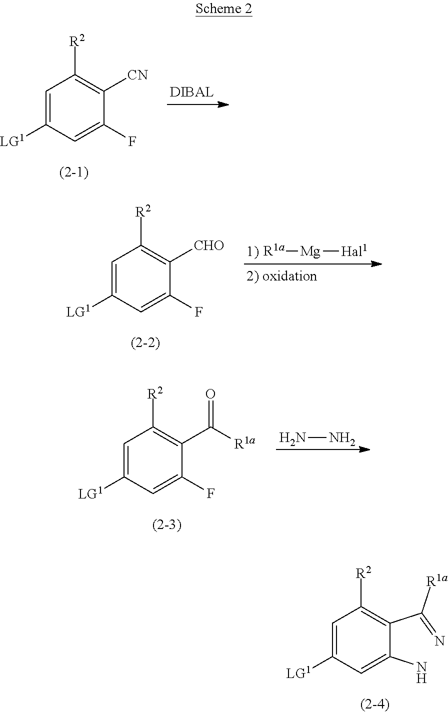

As shown in Scheme 2, compounds of Formula (2-4) can be prepared from compounds of Formula (2-1), wherein R² is as defined in the Summary. To that end, compounds of Formula (2-1), wherein LG¹ is a leaving group such as chlorine, bromine or iodine, can be reacted with diisobutylaluminum hydride (DIBAL) in a suitable solvent such as dichloromethane and at a suitable temperature to deliver compounds of Formula (2-2). Compounds of Formula (2-2) can be reacted initially with a Grignard reagent, R^{1a}—Mg-Hal¹, wherein R^{1a} is C₁-C₄-alkyl, C₁-C₄-fluoroalkyl, or C₃-C₆-cycloalkyl and Hal¹ is chlorine, bromine or iodine, in a suitable solvent such as tetrahydrofuran at −78° C. to ambient temperature to give an intermediate alcohol. The alcohol can be subsequently oxidized to give ketones of Formula (2-3). The oxidation step can be achieved with such reagents as tetrapropylammonium perruthenate and 4-methylmorpholine 4-oxide in the presence of molecular sieves in dichloromethane or pyridinium chlorochromate in dichloromethane. Compounds of Formula (2-3) can be reacted with hydrazine in solvents such as dimethoxyethane or dioxane at elevated temperatures to supply compounds of Formula (2-4).

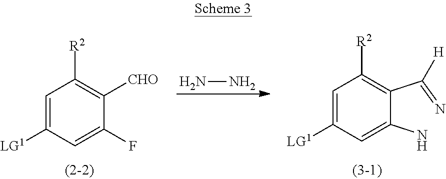

As shown in Scheme 3, compounds of Formula (3-1) can be prepared from compounds of Formula (2-2), wherein R² is as defined in the Summary. To that end, compounds of Formula (2-3), wherein LG¹ is a leaving group such as chlorine, bromine or iodine, can be reacted with hydrazine in solvents such as dimethoxyethane or dioxane at elevated temperatures to supply compounds of Formula (3-1).

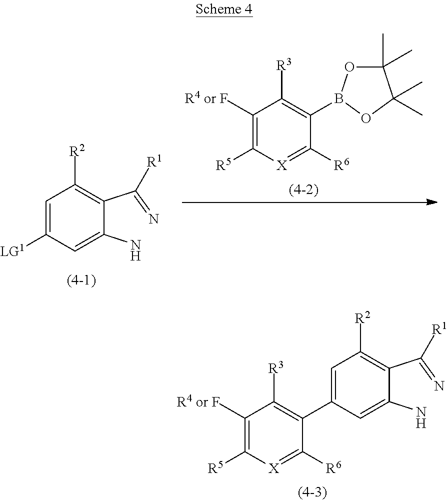

As depicted in Scheme 4, compounds of Formula (4-1), wherein R¹ and R² are as defined in the Summary and LG¹ is a leaving group such as chlorine, bromine or iodine, can be reacted with compounds of Formula (4-2) in a cross-coupling reaction under conditions known to one of skill in the art to give compounds of Formula (4-3). Although a borolane is shown in compounds of Formula (4-2), other suitable boron functional groups such as boronic acids may be substituted in the cross-coupling reaction. In compounds of Formula (4-2), R³, R⁴, R⁵, R⁶ and X are as defined in the Summary. The F, R⁴ and R⁵ of compounds of Formula (4-3) can be further derivatized.

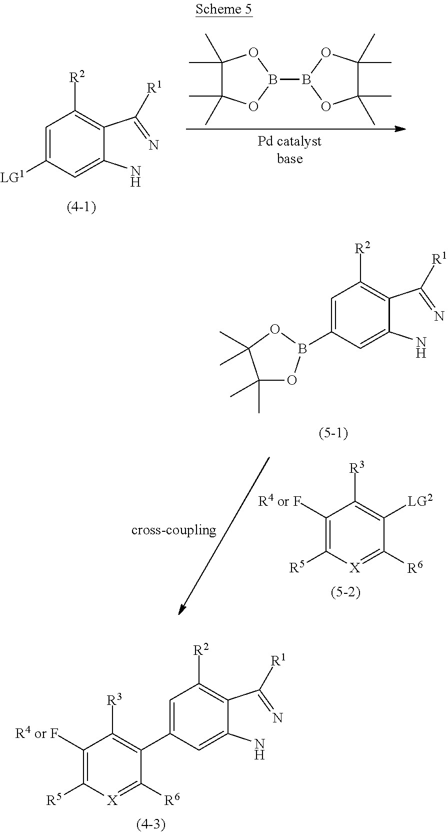

As shown in Scheme 5, compounds of Formula (4-1), wherein R¹ and R² are as defined in the Summary and LG¹ is a leaving group such as chlorine, bromine or iodine, can be reacted with bis(pinacolato)diboron in the presence of a palladium catalyst and base to give compounds of Formula (5-1). Although a borolane is shown in compounds of Formula (5-1), other suitable boron functional groups such as boronic acids may be suitable for use in the subsequent cross-coupling reaction. Compounds of Formula (5-1) can be cross-coupled with compounds of Formula (5-2), wherein R³, R⁴, R⁵, R⁶ and X are as defined in the Summary and LG² is a leaving group such as chlorine, bromine, or iodine, under conditions known to one of skill in the art to give compounds of Formula (4-3).

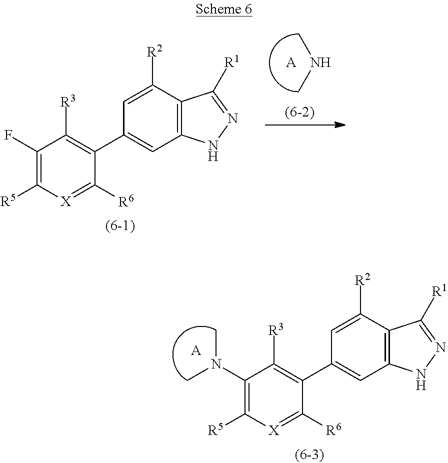

As depicted in Scheme 6, compounds of Formula (6-3), wherein R¹, R², R³, R⁵, R⁶ and X are as described in the Summary and ring A represents heterocycles as defined by G¹ and G² in the Summary, can be prepared from compounds of Formula (6-1). Compounds of Formula (6-1) can be reacted with heterocycles of Formula (6-2) containing a secondary amine moiety within the heterocycle under optionally heated conditions to deliver compounds of Formula (6-3). Compounds of Formula (6-3) which are representative of compounds of Formula (I) can be further derivatized.

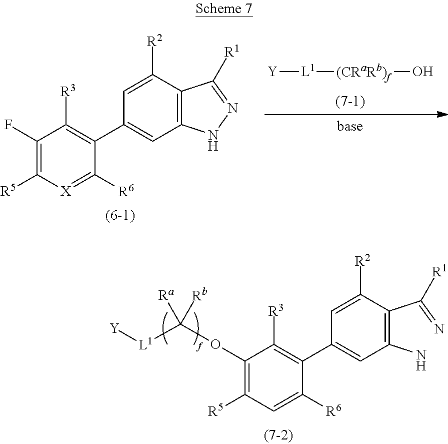

As depicted in Scheme 4, compounds of Formula (6-1) can be transformed to compounds of Formula (7-2), wherein R¹, R², R³, R⁵, R⁶, R^{a}, R^{b}, f, L¹, X and Y are as defined in the Summary. Compounds of Formula (6-1) can be reacted with compounds of Formula (7-1) in the presence of a base and in a suitable solvent at or near ambient temperature to give compounds of Formula (7-2). Compounds of Formula (7-2) are representative of compounds of Formula (I).

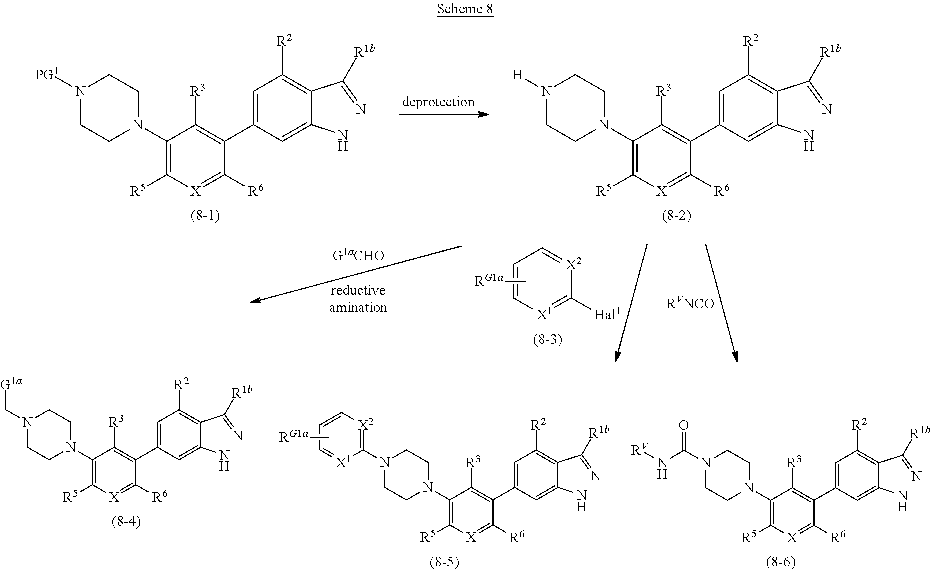

As shown in Scheme 8, compounds of Formula (8-4), (8-5), and (8-6), wherein R², R³, R⁵, R⁶, R^{v}, G^{1a}, and X are as described in the Summary and R^{1b} is hydrogen, C₁-C₄-alkyl, C₁-C₄-fluoroalkyl or C₃-C₆-cycloalkyl can be prepared from compounds of Formula (8-1). Compounds of Formula (8-1), wherein PG¹ is a protecting group such as tert-butoxycarbonyl, can be deprotected by conditions known to one of skill in the art to give compounds of Formula (8-2). Compounds of Formula (8-2) can be reductively aminated with aldehydes, G^{1a}CHO, to give compounds of Formula (8-4). Compounds of Formula (8-2) can be reacted with compounds of Formula (8-3), wherein R^{G1a} are substituents defined in the Summary for G^{1a}; Hal¹ is chlorine, bromine, or iodine; and wherein X¹ and X² are N or CH provided that at least one of X¹ or X² is N; in the presence of a base in an optionally heated solvent such as dimethyl sulfoxide to give compounds of Formula (8-5). Compounds of Formula (8-2) can also be reacted with isocyanates, R^{v}NCO, in the presence of a base to give compounds of Formula (8-6). Compounds of Formula (8-4), (8-5), and (8-6) are representative of compounds of Formula (I).

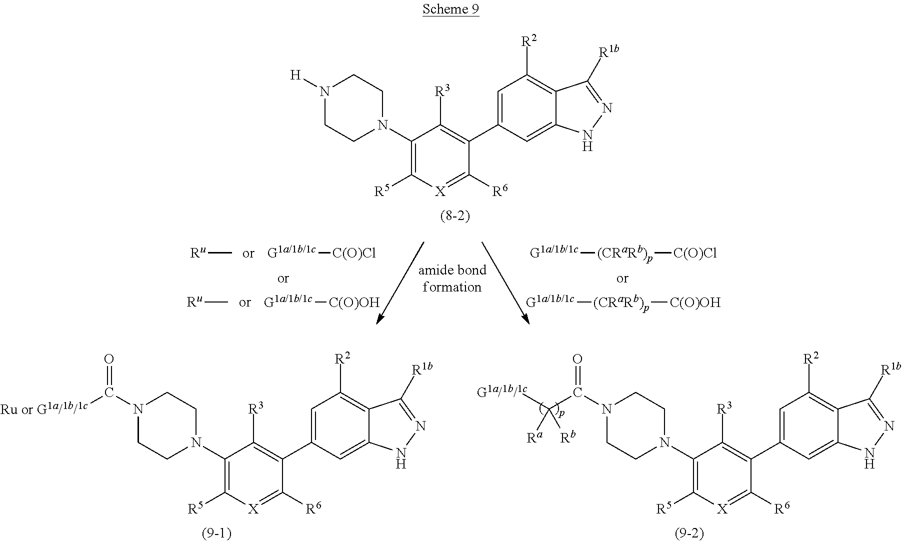

As shown in Scheme 9, compounds of Formula (8-2) can be transformed to compounds of Formula (9-1) and Formula (9-2), wherein R², R³, R⁵, R⁶, R^{a}, R^{b}, R^{u}, G^{1a}, G^{1b}, G^{1c}, p and X are as described in the Summary and R^{1b} is hydrogen, C₁-C₄-alkyl, C₁-C₄-fluoroalkyl or C₃-C₆-cycloalkyl. Compounds of Formula (9-1) and Formula (9-2) are representative of compounds of Formula (I). Compounds of Formula (8-2) are converted to compounds of the Formula (9-1) by two different methods. One method involves treatment of a compound with the formula of (8-2) with an acid chloride of formula R^{u}C(O)Cl, G^{1a}-C(O)Cl, G^{1b}-C(O)Cl, or G^{1c}-C(O)Cl in a solvent, such as dichloromethane, in the presence of triethylamine at room temperature. Alternatively, compounds of Formula (9-1) can be prepared from compounds of Formula (8-2) by reacting compounds of Formula (8-2) with a carboxylic acid of formula R^{u}C(O)OH, G^{1a}-C(O)OH, G^{1b}-C(O)OH, or G^{1c}-C(O)OH under amide coupling conditions. Examples of conditions known to generate amides from a mixture of a carboxylic acid and an amine include but are not limited to adding a coupling reagent such as but not limited to N-(3-dimethylaminopropyl)-N′-ethylcarbodiimide (EDC or EDCI), 1,3-dicyclohexylcarbodiimide (DCC), bis(2-oxo-3-oxazolidinyl)phosphinic chloride (BOPC1), 0-(7-azabenzotriazol-1-yl)-N,N,N′,N′-tetramethyluronium hexafluorophosphate (HATU), 0-benzotriazol-1-yl-N,N,N′,N′-tetramethyluronium tetrafluoroborate (TBTU), and 2-(1H-benzo[d][1,2,3]triazol-1-yl)-1,1,3,3-tetramethylisouronium hexafluorophosphate(V) (HBTU). The coupling reagents may be added as a solid, a solution, or as the reagent bound to a solid support resin. In addition to the coupling reagents, auxiliary-coupling reagents may facilitate the coupling reaction. Auxiliary coupling reagents that are often used in the coupling reactions include but are not limited to (dimethylamino)pyridine (DMAP), 1-hydroxy-7-azabenzotriazole (HOAT) and 1-hydroxybenzotriazole (HOBT). The reaction may be carried out optionally in the presence of a base such as triethylamine or diisopropylethylamine. The coupling reaction may be carried out in solvents such as but not limited to tetrahydrofuran, N,N-dimethylformamide, dichloromethane, and ethyl acetate. The reaction may be conducted at ambient or elevated temperatures. Similarly, compounds of Formula (8-2) are converted to compounds of Formula (9-2) by reaction of compounds of Formula (8-2) with carboxylic acid chlorides; G^{1a}-(CR^{a}R^{b})ₚ—C(O)Cl, G^{1b}-(CR^{a}R^{b})ₚ—C(O)Cl, or G^{1c}-(CR^{a}R^{b})ₚ—C(O)Cl; or carboxylic acids; G^{1a}-(CR^{a}R^{b})ₚ—C(O)OH, G^{1b}-(CR^{a}R^{b})ₚ—C(O)OH, or G^{1c}-(CR^{a}R^{b})ₚ—C(O)OH under the reaction conditions described above.

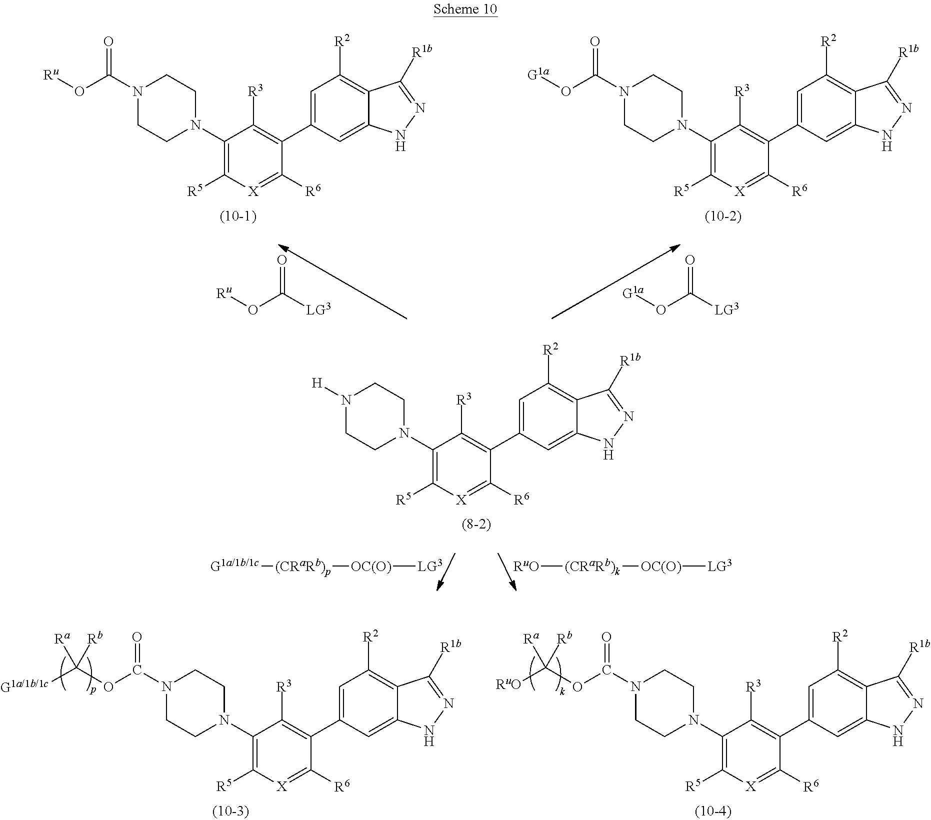

As shown in Scheme 10, compounds of Formula (8-2) can be transformed to carbamates of Formula (10-1), Formula (10-2), Formula (10-3), and Formula (10-4), wherein R², R³, R⁵, R⁶, R^{a}, R^{b}, R^{u}, G^{1a}, G^{1b}, G^{1c}, k, p and X are as described in the Summary and R^{1b} is hydrogen, C₁-C₄-alkyl, C₁-C₄-fluoroalkyl or C₃-C₆-cycloalkyl. Compounds of Formula (10-1), Formula (10-2), Formula (10-3), and Formula (10-4) are representative of compounds of Formula (I). Compounds of Formula (8-2) are converted to compounds of the Formula (10-1), Formula (10-2), Formula (10-3), and Formula (10-4) by two different methods. One method involves treatment of a compound with the formula of (8-2) with chloroformates of formula R^{u}O—C(O)Cl, G^{1a}O—C(O)Cl, G^{1a}(CR^{a}R^{b})ₚ—OC(O)Cl, G^{1b}(CR^{a}R^{b})ₚ—OC(O)Cl, G^{1c}(CR^{a}R^{b})ₚ—OC(O)Cl, or R^{u}O—(CR^{a}R^{b})ₖ—OC(O)Cl in a solvent such as dichloromethane, in the presence of a base such as triethylamine, diisopropylethylamine, aqueous potassium carbonate or aqueous sodium bicarbonate at room temperature to give compounds of Formula (10-1), Formula (10-2), Formula (10-3), or Formula (10-4), respectively. Under these conditions, LG³ is chlorine. Alternatively, compounds of Formula (8-2) react with compounds of Formula R^{u}O—C(O)LG³, G^{1a}O—C(O)LG³, G^{1a}(CR^{a}R^{b})ₚ—OC(O)LG³, G^{1b}(CR^{a}R^{b})ₚ—OC(O)LG³, G^{1c}(CR^{a}R^{b})ₚ—OC(O)LG³, or R^{u}O—(CR^{a}R^{b})ₖ—OC(O)LG³ in a solvent such as dichloromethane, in the presence of a base such as triethylamine or diisopropylethylamine at room temperature to give compounds of Formula (10-1), Formula (10-2), Formula (10-3), or Formula (10-4), respectively. Under these conditions, LG³ is 4-nitrophenoxy moiety.

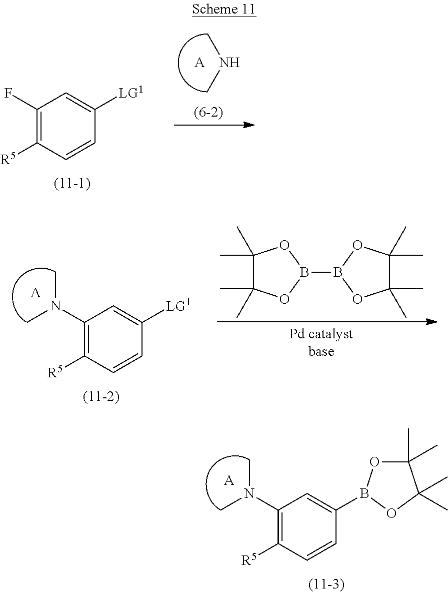

As shown in Scheme 11, compounds of Formula (11-2) and Formula (11-3), wherein R⁵ is as described in the Summary and ring A represents heterocycles as defined by G¹ and G² in the Summary, can be prepared from compounds of Formula (11-1). Compounds of Formula (11-1), wherein LG¹ is a leaving group such as chlorine, bromine or iodine and wherein R⁵ is as defined in the Summary, can be reacted with heterocycles of Formula (6-2) in the presence of a base in an optionally heated solvent to give compounds of Formula (11-2). Compounds of Formula (11-2) can be cross-coupled with bis(pinacolato)diboron to give compounds of Formula (11-3). Compounds of Formula (11-2) can be used in the methodology of Scheme 5, and compounds of Formula (11-3) can be used in the methodology shown in Scheme 4.

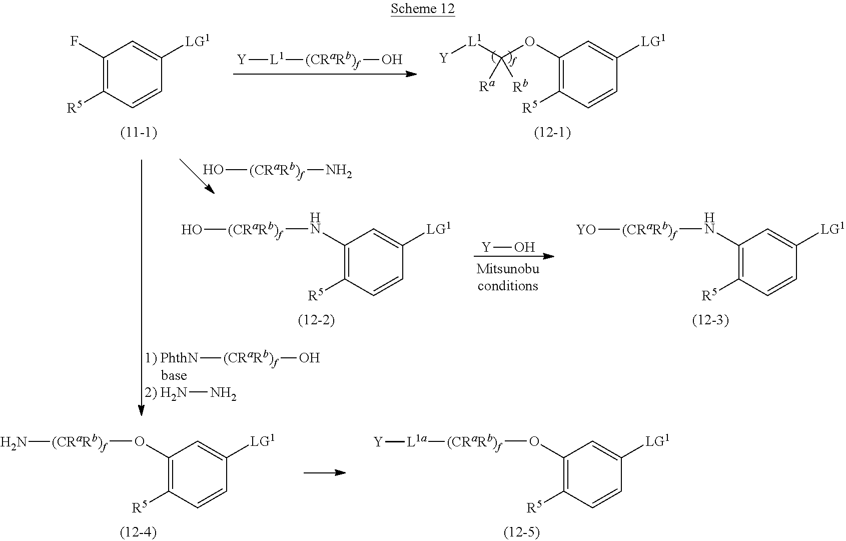

Compounds of Formula (11-1) can be converted into compounds of Formula (12-1), Formula (12-3), and Formula (12-5) for use transformations described in Schemes 5 and 11 and after conversion to the corresponding boronic acid or boronate, in Scheme 4. Compounds of Formula (11-1), wherein LG¹ is a leaving group such as chlorine, bromine or iodine, can be reacted with Y-L¹-(CR^{a}R^{b})_{f}—OH in the presence of a base such as potassium tert-butoxide in solvents such as dimethyl sulfoxide or tetrahydrofuran to give compounds of Formula (12-1), wherein R⁵, R^{a}, R^{b}, Y, and f are as described in the Summary. Compounds of Formula (11-1) can reacted with HO—(CR^{a}R^{b})_{f}—NH₂ in a heated solvent such as ethanol to give compounds of Formula (12-2). The heating may either be conventional or achieved with microwave irradiation. Compounds of Formula (12-2) can be treated with Y—OH under Mitsunobu reaction conditions to give compounds of Formula (12-3). Compounds of Formula (11-1) can also be reacted initially with PhthN—(CR^{a}R^{b})_{f}—OH, wherein PhthN represents a phthalimide moiety, and then reacted with hydrazine to give compounds of Formula (12-4). The amine of compounds of Formula (12-4) can then be functionalized to give compounds of Formula (12-5), wherein L^{1a} is —NR^{c}—, —OC(O)NR^{c}—, or —S(O)₂NR^{c}—, wherein R^{c} is as described in the Summary.

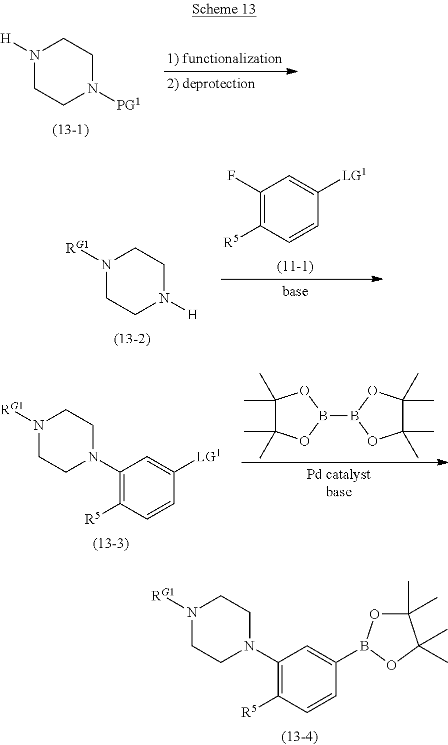

As shown in Scheme 13, compounds of Formula (13-1) can be converted to compounds of Formula (13-4) and Formula (13-5). To that end, compounds of Formula (13-1), wherein PG¹ is a protecting group such as tert-butoxycarbonyl, can be functionalized on the piperazine amine functionality with reaction known to one of skill in the art to introduce R^{G1}, the moieties that are substituents of G¹ that can be introduced and are stable on the piperazine amine Subsequent removal of the protecting group under reaction conditions known to one of skill in the art gives compounds of Formula (13-2). Reaction of compounds of Formula (13-2) with Compounds of Formula (11-1), wherein LG¹ is a leaving group such as chlorine, bromine or iodine and wherein R⁵ is as defined in the Summary, in the presence of a base in an optionally heated solvent gives compounds of Formula (13-3). Compounds of Formula (13-3) can be cross-coupled with bis(pinacolato)diboron to give compounds of Formula (11-4). Compounds of Formula (13-3) can be used in the methodology of Scheme 5, and compounds of Formula (13-4) can be used in the methodology shown in Scheme 4.

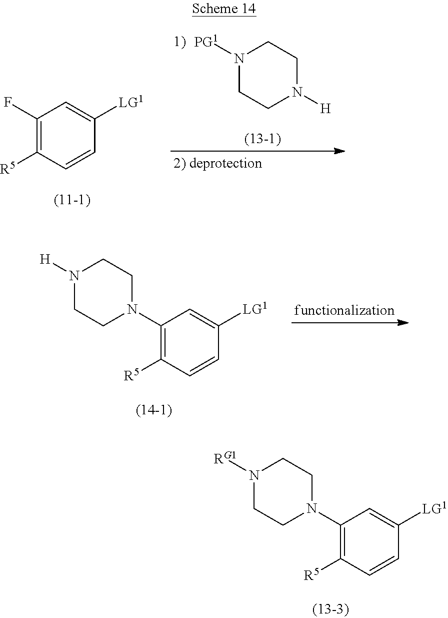

As shown in Scheme 14, compounds of Formula (11-1), wherein LG¹ is a leaving group such as chlorine, bromine or iodine and wherein R⁵ is as defined in the Summary, can be converted to compounds of Formula (13-3). Accordingly, compounds of Formula (11-1), can be reacted with compounds of Formula (13-1), wherein PG¹ is a protecting group such as tert-butoxycarbonyl, in the presence of a base in an optionally heated solvent and subsequently deprotected under conditions known to one of skill in the art to furnish compounds of Formula (14-1). Compounds of Formula (14-1) can be functionalized on the piperazine amine functionality with reactions known to one of skill in the art to introduce R^{G1}, the moieties that are substituents of G¹ that can be introduced and are stable on the piperazine amine Compounds of Formula (13-4) can be used as described in Scheme 13.

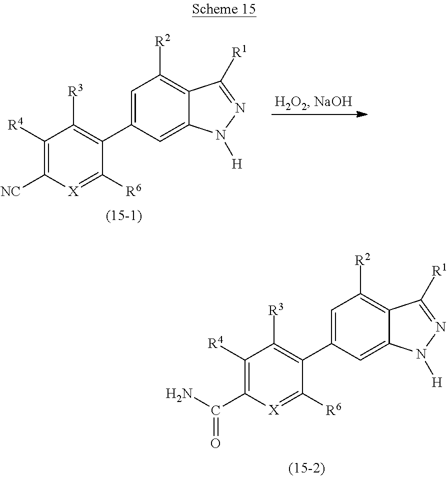

As shown in Scheme 15, compounds of Formula (15-1), wherein R¹, R², R³, R⁴, R⁶ and X are as defined in the Summary, can be transformed to compounds of Formula (15-2) which are representative of compounds of Formula (I). Compounds of Formula (15-1) can be treated with aqueous hydrogen peroxide and aqueous sodium hydroxide at or near room temperature to give compounds of Formula (15-2).

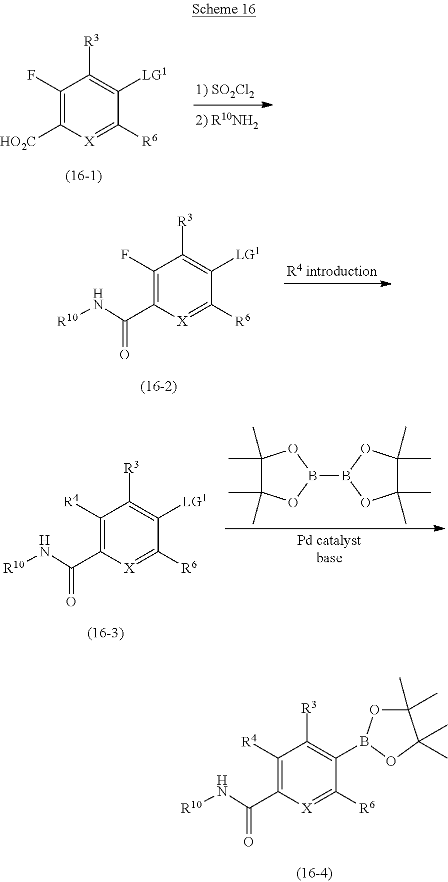

As depicted in Scheme 16, compounds of Formula (16-1) can be converted to compounds of Formula (16-3) and Formula (16-4), wherein R³, R⁴, R⁶, R¹⁰ and X are as defined in the Summary and LG¹ is a leaving group such as chlorine, bromine or iodine. Carboxylic acids of Formula (16-1) can be converted to the corresponding acid chloride by treatment with reagents such as thionyl chloride and then treated with amines, R¹⁰NH₂, to give compounds of Formula (16-2). Compounds of Formula (16-2) can be transformed to compounds of Formula (16-3) using the methodologies described in Schemes 6, 7, 11, 12, 13 or 14. Compounds of Formula (16-3) can be cross-coupled with bis(pinacolato)diboron to give compounds of Formula (16-4). Compounds of Formula (16-3) can be used in the methodology of Scheme 5, and compounds of Formula (16-4) can be used in the methodology shown in Scheme 4.

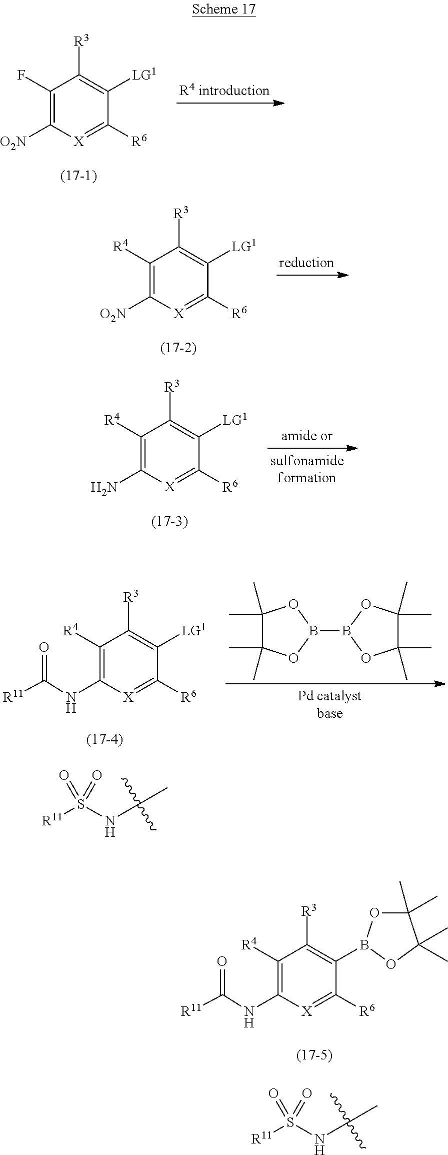

As shown in Scheme 17, compounds of Formula (17-1) can be converted to compounds of Formula (17-4) and Formula (17-5), wherein R³, R⁴, R⁶, R¹¹, and X are as defined in the Summary. Compounds of Formula (17-1), wherein LG¹ is a leaving group such as chlorine, bromine or iodine, are transformed to compounds of Formula (17-2) using the methodologies described in Schemes 6, 7, 11, 12, 13 or 14. Reduction of compounds of Formula (17-2) using for example zinc and ammonium chloride gives compounds of Formula (17-3). Compounds of Formula (17-3) can be reacted with carboxylic acids or acid chlorides and sulfonyl chlorides to give amides or sulfonamides of Formula (17-4). Compounds of Formula (17-4) can be cross-coupled with bis(pinacolato)diboron to give compounds of Formula (17-5). Compounds of Formula (17-4) can be used in the methodology of Scheme 5, and compounds of Formula (17-5) can be used in the methodology shown in Scheme 4.

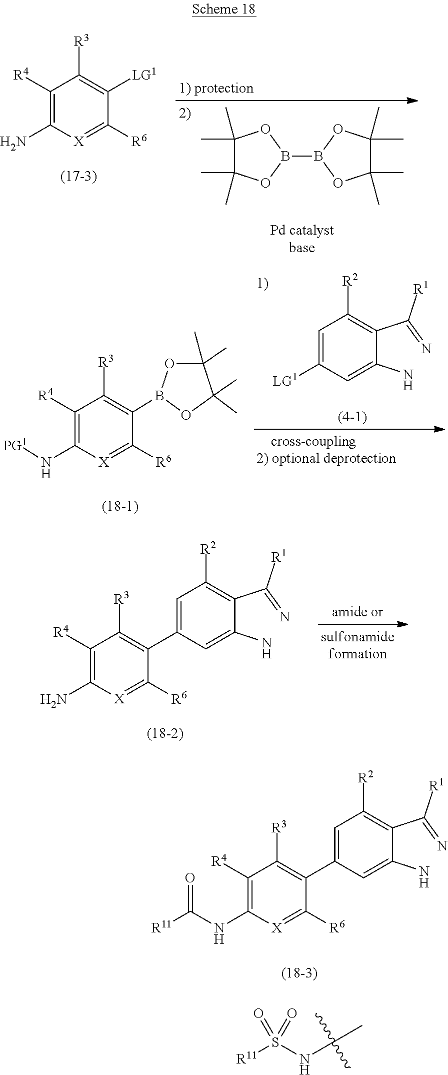

As shown in Scheme 18, compounds of Formula (17-3) can be converted to compounds of Formula (18-3), wherein R¹, R², R³, R⁴, R⁶, R¹¹, and X are as defined in the Summary Compounds of Formula (17-3), wherein LG¹ is a leaving group such as chlorine, bromine or iodine, can first be protected and then cross-coupled with bis(pinacolato)diboron to give compounds of Formula (18-1), wherein PG¹ is a protecting group. Compounds of Formula (18-1) can be cross-coupled with compounds of Formula (4-1) and then deprotected when the protecting group does not come off during the cross-coupling reaction to give compounds of Formula (18-2). Compounds of Formula (18-2) can be reacted with carboxylic acids or acid chlorides and sulfonyl chlorides to give amides or sulfonamides of Formula (18-3). Compounds of Formula (18-3) are representative of compounds of Formula (I).

The compounds and intermediates of the invention may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

Many of the compounds of the invention have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Unless otherwise specified, solvents, temperatures and other reaction conditions can be readily selected by one of ordinary skill in the art. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the invention is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the invention is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as illustrated in the Examples section are illustrative and are not to be read as limiting the scope of the invention as it is defined in the appended claims. All alternatives, modifications, and equivalents of the synthetic methods and specific examples are included within the scope of the claims.

### Compositions of the Invention

The invention also provides pharmaceutical compositions comprising a therapeutically effective amount of a compound of Formula (I) in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier”, as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally”, as used herein, refers to modes of administration which include intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of materials which can be useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants, which can be required. Ophthalmic formulations, eye ointments, powders and solutions are contemplated as being within the scope of this invention. Aqueous liquid compositions comprising compounds of the invention also are contemplated.

The compounds of the invention can be used in the form of pharmaceutically acceptable salts or esters, or amides derived from inorganic or organic acids. The term “pharmaceutically acceptable salts and esters and amides”, as used herein, refer to carboxylate salts, amino acid addition salts, zwitterions, and esters and amides of compounds of Formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid. An example of a suitable salt is a hydrochloride salt.

Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Preferred salts of the compounds of the invention are the tartrate and hydrochloride salts.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Examples of acids which can be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, ethylammonium and the like. Other representative organic amines useful for the formation of base addition salts include ethylenediamine, ethanolamine, diethanolamine, piperidine, and piperazine.

The term “pharmaceutically acceptable ester”, as used herein, refers to esters of compounds of the invention which hydrolyze in vivo and include those that break down readily in the human body to leave the parent compound or a salt thereof. Examples of pharmaceutically acceptable, non-toxic esters of the invention include C₁-to-C₆-alkyl esters and C₅-to-C₇-cycloalkyl esters, although C₁-to-C₄-alkyl esters are preferred. Esters of the compounds of Formula (I) may be prepared according to conventional methods. For example, such esters may be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, alkyl triflate, for example with methyl iodide, benzyl iodide, cyclopentyl iodide. They also may be prepared by reaction of the compound with an acid such as hydrochloric acid and an alcohol such as methanol or ethanol.

The term “pharmaceutically acceptable amide”, as used herein, refers to non-toxic amides of the invention derived from ammonia, primary C₁-to-C₆-alkyl amines and secondary C₁-to-C₆-dialkyl amines. In the case of secondary amines, the amine may also be in the form of a 5- or 6-membered heterocycle containing one nitrogen atom. Amides derived from ammonia, C₁-to-C₃-alkyl primary amides and C₁-to-C₂-dialkyl secondary amides are preferred. Amides of the compounds of Formula (I) may be prepared according to conventional methods. Pharmaceutically acceptable amides are prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also may be prepared by reaction of the compound with an acid such as sulfuric acid and an alkylcarboxylic acid such as acetic acid, or with acid and an arylcarboxylic acid such as benzoic acid under dehydrating conditions as with molecular sieves added. The composition can contain a compound of the invention in the form of a pharmaceutically acceptable prodrug.

The invention contemplates pharmaceutically active compounds either chemically synthesized or formed by in vivo biotransformation to compounds of Formula (I)

### Methods of the Invention

The compounds and compositions of the invention are useful for treating and preventing certain diseases and disorders in humans and animals. As an important consequence of the ability of the compounds of the invention to modulate the effects of Trk's in cells, the compounds described in the invention can affect physiological processes in humans and animals. In this way, the compounds and compositions described in the invention are useful for treating and preventing diseases and disorders modulated by Trk. Typically, treatment or prevention of such diseases and disorders can be effected by selectively modulating Trk's in a mammal, by administering a compound or composition of the invention, either alone or in combination with another active agent as part of a therapeutic regimen.

The compounds of the invention, including but not limited to those specified in the examples, possess an affinity for Trk and therefore, the compounds of the invention may be useful for the treatment and prevention of diseases or conditions such as pain, including osteoarthritis pain, joint pain, neuropathic pain, post-surgical pain, low back pain, and diabetic neuropathy, pain during surgery, cancer pain, chemotherapy induced pain, headaches, including cluster headache, tension headache, migraine pain, trigeminal neuralgia, shingles pain, post-herpetic neuralgia, carpal tunnel syndrome, inflammatory pain, pain from rheumatoid arthritis, colitis, pain of interstitial cystitis, visceral pain, pain from kidney stone, pain from gallstone, angina, fibromyalgia, chronic pain syndrome, thalamic pain syndrome, pain from stroke, phantom limb pain, sunburn, radiculopathy, complex regional pain syndrome, HIV sensory neuropathy, central neuropathic pain syndromes, multiple sclerosis pain, Parkinson disease pain, spinal cord injury pain, menstrual pain, toothache, pain from bone metastasis, pain from endometriosis, pain from uterine fibroids, nociceptive pain, hyperalgesias, and temporomandibular joint pain, inflammation, auto-immune disease, rheumatoid arthritis, psoriasis, psoriatic arthritis, asthma, Crohn's disease, inflammatory bladder cystitis, inflammatory bowel disease, joint swelling, diabetic nephropathy, kidney fibrosis, chronic kidney disease, cancer, neuroblastoma, melanoma, myeloma, cancers of the pancreas, prostate, ovary, colon, thyroid, lung, brain, esophagus, kidney, of bone, and blood.

Compounds of the invention are particularly useful for treating and preventing a condition or disorder affecting pain.

The ability of the compounds of the invention, including, but not limited to, those specified in the examples, to treat the pain of osteoarthritis may be demonstrated by Lane N E, et al. New England J Med 2010; 363:1521-1531; Schnitzer T J, et al. Osteoarthritis Cartilage 2011; 19:639-646.

The ability of the compounds of the invention, including, but not limited to, those specified in the examples, to treat lower back pain may be demonstrated by Katz N, et al. Pain 2011; 152:2248-2258.

The ability of the compounds of the invention, including, but not limited to, those specified in the examples, to treat the pain of cystitis may be demonstrated by Evans R J, et al. J. Urology 2011; 185:1716-1721.

The ability of the compounds of the invention, including, but not limited to, those specified in the examples, to treat the pain of auto-immune arthritis may be demonstrated by Shelton D L, et al. Pain 2005; 116:8-16.

The ability of the compounds of the invention, including, but not limited to, those specified in the examples, to treat neuropathic pain or inflammatory pain may be demonstrated by Ro L S, et al. Pain 1999; 79:265-274; Ugolini G, et al. Proceedings of the National Academy of Sciences of the USA 2007; 104:2985-2990.

The ability of the compounds of the invention, including, but not limited to, those specified in the examples, to treat the pain of bone fracture may be demonstrated by Ghilardi J R, et al. Bone 2011; 48:389-298.

The ability of the compounds of the invention, including, but not limited to, those specified in the examples, to treat myofascial pain syndrome may be demonstrated by Hayashi K, et al. Journal of Pain 2011; 12:1059-1068.

The ability of the compounds of the invention, including, but not limited to, those specified in the examples, to treat diabetic nephropathy and pathological kidney fibrosis may be demonstrated by Fragiadaki M, et al. Diabetes 2012; 61:2280-2289.

The ability of the compounds of the invention, including, but not limited to, those specified in the examples, to treat cancer may be demonstrated by Albaugh P, et al. ACS Medicinal Chemistry Letters (2012; 3:140-145.

The ability of the compounds of the invention, including, but not limited to, those specified in the examples, to treat neuroblastoma may be demonstrated by Wang T, et al. ACS Medicinal Chemistry Letters 2012; 3:705-709; Thress K, et al. Molecular Cancer Therapeutics 2009; 8:1818-1827.

The ability of the compounds of the invention, including, but not limited to, those specified in the examples, to treat melanoma may be demonstrated by Truzzi F, et al. Journal of Investigative Dermatology 2008; 128:2031-2040.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the invention can be employed in pure form or, where such forms exist, in pharmaceutically acceptable salt or ester, or amide form. Alternatively, the compound can be administered as a pharmaceutical composition containing the compound of interest in combination with one or more pharmaceutically acceptable carriers. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

For treatment or prevention of disease, the total daily dose of the compounds of this invention administered to a human or lower animal may range from about 0.0003 to about 100 mg/kg/day. For purposes of oral administration, more preferable doses can be in the range of from about 0.0003 to about 30 mg/kg/day. If desired, the effective daily dose can be divided into multiple doses for purposes of administration; consequently, single dose compositions may contain such amounts or submultiples thereof to make up the daily dose.

The compounds and processes of the invention will be better understood by reference to the following examples, which are intended as an illustration of and not a limitation upon the scope of the invention.

### EXAMPLES

Abbreviations: DMSO for dimethyl sulfoxide; ESI for electrospray ionization; and HPLC for high performance liquid chromatography.

### Intermediate 1

### 6-bromo-4-(tetrahydro-2H-pyran-4-yloxy)-1H-indazol-3-amine

### Step 1

### 4-bromo-2-fluoro-6-(tetrahydro-2H-pyran-4-yloxy)benzonitrile

Tetrahydro-2H-pyran-4-ol (3 g, 29.4 mmol) was dissolved in tetrahydrofuran (50 mL). Lithium hexamethyldisilazane in dichloromethane (1 M, 29.4 mL) was added in portions over 5 minutes, and the reaction mixture was stirred at ambient temperature for 1 hour. The reaction mixture was then cooled to 0° C. and 4-bromo-2,6-difluorobenzonitrile (5.82 g, 26.7 mmol) was added. The reaction mixture was allowed to warm to ambient temperature with stirring continued for 16 hours. The reaction mixture was concentrated to −10 mL, poured into water (50 mL) and extracted with ethyl acetate (3×100 mL). The combined organic layers were dried over MgSO₄, filtered and concentrated to give the titled compound. ¹H NMR (300 MHz, DMSO-d₆) δ ppm 1.54-1.77 (m, 2H), 1.91-2.05 (m, 2H), 3.52 (ddd, J=11.6, 8.6, 3.0 Hz, 2H), 3.78-3.89 (m, 2H), 4.88-5.00 (m, 1H), 7.47 (dd, J=8.8, 1.5 Hz, 1H), 7.56 (d, J=1.2 Hz, 1H).

### Step 2

### 6-bromo-4-(tetrahydro-2H-pyran-4-yloxy)-1H-indazol-3-amine

To a solution of 4-bromo-2-fluoro-6-(tetrahydro-2H-pyran-4-yloxy)benzonitrile (7.5 g, 25 mmol) in n-butanol (30 mL) was added 65% hydrazine hydrate (7.5 mL, 100 mmol), and the reaction mixture was heated at reflux overnight. The reaction mixture was then concentrated in vacuo and partitioned between water and dichloromethane (50 mL). The organic phase was extracted with additional dichloromethane (2×50 mL). The combined organic layers were dried over MgSO₄ and concentrated. The residue was triturated with ethyl acetate to yield the titled compound. ¹H NMR (300 MHz, DMSO-d₆) δ ppm 1.63-1.79 (m, 2H), 1.95-2.06 (m, 2H), 3.43-3.59 (m, 2H), 3.79-3.91 (m, 2H), 4.71-4.83 (m, 1H), 5.01 (bs, 2H), 6.57 (s, 1H), 6.95 (d, J=1.0 Hz, 1H), 11.50 (s, 1H); MS(ESI+) m/z 312 (M+H)⁺.

### Intermediate 2

### 6-bromo-4-[(1,1,1-trifluoropropan-2-yl)oxy]-1H-indazol-3-amine

The titled compound was prepared using the procedures described for the preparation of Intermediate 1 except 1,1,1-trifluoropropan-2-ol was substituted for tetrahydro-2H-pyran-4-ol in Step 1. ¹H NMR (300 MHz, DMSO-d₆) δ ppm 1.49 (d, J=6.4 Hz, 3H), 4.96 (bs, 2H), 5.51 (p, J=6.4 Hz, 1H), 6.78 (s, 1H), 7.07 (d, J=1.2 Hz, 1H), 11.65 (s, 1H); MS(ESI+) m/z 324 (M+H)⁺.

### Intermediate 3

### 6-bromo-4-(cyclopentyloxy)-1H-indazol-3-amine

The titled compound was prepared using the procedures described for the preparation of Intermediate 1 except cyclopentanol was substituted for tetrahydro-2H-pyran-4-ol in Step 1. ¹H NMR (300 MHz, DMSO-d₆) δ ppm 1.56-2.01 (m, 8H), 4.57-5.20 (m, 3H), 6.41 (d, J=1.3 Hz, 1H), 6.94 (d, J=1.3 Hz, 1H), 11.48 (s, 1H); MS(ESI+) m/z 296 (M+H)⁺.

### Intermediate 4

### tert-butyl 4-[(3-amino-6-bromo-1H-indazol-4-yl)oxy]piperidine-1-carboxylate

The titled compound was prepared using the procedures described for the preparation of Intermediate 1 except tert-butyl 4-hydroxypiperidine-1-carboxylate was substituted for tetrahydro-2H-pyran-4-ol in Step 1. ¹H NMR (300 MHz, DMSO-d₆) δ ppm 1.43 (s, 9H), 1.58-1.77 (m, 2H), 1.86-1.98 (m, 2H), 3.02-3.24 (m, 2H), 3.18-3.29 (m, 2H), 3.60-3.72 (m, 2H), 4.66-4.81 (m, 1H), 5.00 (bs, 2H), 6.56 (d, J=1.3 Hz, 1H), 6.95 (d, J=1.2 Hz, 1H), 11.50 (s, 1H); MS(ESI+) m/z 411 (M+H)⁺.

### Intermediate 5

### 6-bromo-4-[(3S)-tetrahydrofuran-3-yloxy]-1H-indazol-3-amine

The titled compound was prepared using the procedures described for the preparation of Intermediate 1 except (S)-tetrahydrofuran-3-ol was substituted for tetrahydro-2H-pyran-4-ol in Step 1. ¹H NMR (300 MHz, DMSO-d₆) δ ppm 2.04-2.17 (m, 1H), 2.17-2.36 (m, 1H), 3.77 (td, J=8.3, 4.6 Hz, 1H), 3.83-3.90 (m, 1H), 3.92 (d, J=3.0 Hz, 2H), 5.01 (bs, 2H), 5.12-5.20 (m, 1H), 6.42 (d, J=1.3 Hz, 1H), 6.98 (d, J=1.2 Hz, 1H), 11.53 (s, 1H); MS(ESI+) m/z 298 (M+H)⁺.

### Intermediate 6

### 6-bromo-4-[(3R)-tetrahydrofuran-3-yloxy]-1H-indazol-3-amine

The titled compound was prepared using the procedures described for the preparation of Intermediate 1 except (R)-tetrahydrofuran-3-ol was substituted for tetrahydro-2H-pyran-4-ol in Step 1. ¹H NMR (300 MHz, DMSO-d₆) δ ppm 2.04-2.17 (m, 1H), 2.17-2.36 (m, 1H), 3.77 (td, J=8.3, 4.6 Hz, 1H), 3.83-3.90 (m, 1H), 3.92 (d, J=3.0 Hz, 2H), 5.01 (bs, 2H), 5.12-5.20 (m, 1H), 6.42 (d, J=1.3 Hz, 1H), 6.98 (d, J=1.2 Hz, 1H), 11.53 (s, 1H); MS(ESI+) m/z 298 (M+H)⁺.

### Intermediate 7

### 6-bromo-4-{[(2R)-1,1,1-trifluoropropan-2-yl]oxy}-1H-indazol-3-amine

The titled compound was prepared using the procedures described for the preparation of Intermediate 1 except (R)-1,1,1-trifluoropropan-2-ol was substituted for tetrahydro-2H-pyran-4-ol in Step 1. ¹H NMR (300 MHz, DMSO-d₆) δ ppm 1.49 (d, J=6.4 Hz, 3H), 4.96 (bs, 2H), 5.51 (p, J=6.4 Hz, 1H), 6.78 (s, 1H), 7.07 (d, J=1.2 Hz, 1H), 11.65 (s, 1H); MS(ESI+) m/z 324 (M+H)⁺; [α]²⁰_{D}=−4° (c 0.94, CH₃OH).

### Intermediate 8

### 6-bromo-4-{[(2S)-1,1,1-trifluoropropan-2-yl]oxy}-1H-indazol-3-amine

The titled compound was prepared using the procedures described for the preparation of Intermediate 1 except (S)-1,1,1-trifluoropropan-2-ol was substituted for tetrahydro-2H-pyran-4-ol in Step 1. ¹H NMR (300 MHz, DMSO-d₆) δ ppm 1.49 (d, J=6.4 Hz, 3H), 4.96 (bs, 2H), 5.51 (p, J=6.4 Hz, 1H), 6.78 (s, 1H), 7.07 (d, J=1.2 Hz, 1H), 11.65 (s, 1H).); MS(ESI+) m/z 324 (M+H)⁺; [α]²⁰_{D}=+₅° (c 0.97, CH₃OH).

### Intermediate 9

### 6-bromo-4-[(1-methylpiperidin-4-yl)oxy]-1H-indazol-3-amine

The titled compound was prepared using the procedures described for the preparation of Intermediate 1 except 1-methylpiperidin-4-ol was substituted for tetrahydro-2H-pyran-4-ol in Step 1. ¹H NMR (300 MHz, DMSO-d₆) δ ppm 1.65-1.86 (m, 2H), 1.85-2.04 (m, 2H), 2.18 (s, 3H), 2.20-2.37 (m, 2H), 4.53-4.64 (m, 1H), 5.02 (bs, 2H), 6.51 (d, J=1.3 Hz, 1H), 6.94 (d, J=1.2 Hz, 1H), 11.52 (s, 1H); MS(ESI+) m/z 325 (M+H)⁺.

### Intermediate 10

### 6-bromo-4-isopropoxy-1H-indazol-3-amine

The titled compound was prepared using the procedures described for the preparation of Intermediate 1 except propan-2-ol was substituted for tetrahydro-2H-pyran-4-ol in Step 1. ¹H NMR (300 MHz, DMSO-d₆) δ ppm 1.34 (d, J=6.1 Hz, 6H), 4.71-4.79 (m, 1H), 4.99 (s, 2H), 6.45 (d, J=1.0 Hz, 1H), 6.93 (d, J=1.0 Hz, 1H) 11.46 (s, 1H); MS(ESI+) m/z 270 (M+H)⁺.

### Example 1

### 4-[3-amino-4-(tetrahydro-2H-pyran-4-yloxy)-1H-indazol-6-yl]-2-(4-phenylpiperazin-1-yl)benzamide

### Step 1

### 4-[3-amino-4-(tetrahydro-2H-pyran-4-yloxy)-1H-indazol-6-yl]-2-fluorobenzonitrile

To a suspension of 6-bromo-4-(tetrahydro-2H-pyran-4-yloxy)-1H-indazol-3-amine (Intermediate 1, 0.25 g, 0.8 mmol) and 4-cyano-3-fluorophenylboronic acid, (0.2 g, 1.2 mmol) in dimethoxyethane:ethanol (4 mL, 1:1) was added 1 M potassium carbonate (1.21 mL). The mixture was purged with nitrogen and bis(triphenylphosphine)palladium(II) dichloride (0.027 g, 0.038 mmol) was added. The reaction mixture was heated in a microwave reactor (CEM Discover®, ≦300 W) at 160° C. for 20 minutes and then concentrated. The residue was partitioned between water and dichloromethane (3×50 mL). The combined organic layers were concentrated, and the residue was purified chromatographically on silica gel eluting with 50-100% ethyl acetate/hexane gradient to obtain the titled compound. ¹H NMR (300 MHz, DMSO-d₆) δ ppm 1.65-1.88 (m, 2H), 2.07 (dd, J=8.8, 3.8 Hz, 2H), 3.50-3.62 (m, 2H), 3.82-3.93 (m, 2H), 4.88-5.01 (m, 1H), 5.04 (s, 2H), 6.75 (s, 1H), 7.12 (d, J=1.1 Hz, 1H), 7.77 (dd, J=8.1, 1.7 Hz, 1H), 7.92 (dd, J=11.3, 1.6 Hz, 1H), 7.94-8.03 (m, 1H), 11.65 (s, 1H); MS(ESI) m/z 353 (M+H)⁺.

### Step 2

### 4-[3-amino-4-(tetrahydro-2H-pyran-4-yloxy)-1H-indazol-6-yl]-2-(4-phenylpiperazin-1-yl)benzamide

A solution of the product from Step 1 (0.06 g, 0.17 mmol) and 1-phenylpiperazine (0.11 g, 0.68 mmol) in dimethyl sulfoxide (imp was heated at 120° C. for 16 hours. Then the reaction mixture was cooled and diluted with methanol (1 mL). 2 M NaOH (0.1 mL, 0.19 mmol) and 30% hydrogen peroxide (0.03 mL, 0.29 mmol) were added, and the mixture was stirred at ambient temperature for 5 hours. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.68-1.89 (m, 2H), 2.00-2.12 (m, 2H), 3.10-3.22 (m, 4H), 3.32-3.40 (m, 4H), 3.51-3.63 (m, 2H), 3.82-3.93 (m, 2H), 4.86-4.99 (m, 1H), 5.06 (s, 2H), 6.67 (s, 1H), 6.82 (t, J=7.2 Hz, 1H), 6.98-7.05 (m, 3H), 7.21-7.30 (m, 2H), 7.40-7.48 (m, 2H), 7.55 (d, J=2.6 Hz, 1H), 7.82 (d, J=7.8 Hz, 1H), 8.47-8.52 (m, 1H), 11.49 (s, 1H); MS (ESI) m/z 513 (M+H)⁺.

### Example 2

### 4-[3-amino-4-(tetrahydro-2H-pyran-4-yloxy)-1H-indazol-6-yl]-2-[4-(pyridin-3-yl)piperazin-1-yl]benzamide

The titled compound was prepared using the procedures described in Example 1 except 1-(pyridin-3-yl)piperazine was substituted for 1-phenylpiperazine in Step 2. ¹H NMR (300 MHz, DMSO-d₆) δ ppm 1.69-1.84 (m, 2H), 2.00-2.12 (m, 2H), 3.10-3.24 (m, 4H), 3.35-3.50 (m, 4H), 3.51-3.63 (m, 2H), 3.82-3.93 (m, 2H), 4.85-4.98 (m, 1H), 5.03 (bs, 2H), 6.67 (s, 1H), 7.03 (d, J=1.0 Hz, 1H), 7.25 (dd, J=8.5, 4.5 Hz, 1H), 7.37-7.48 (m, 3H), 7.52-7.58 (m, 1H), 7.80 (d, J=7.8 Hz, 1H), 8.03 (dd, J=4.5, 1.2 Hz, 1H), 8.38 (d, J=2.9 Hz, 1H), 8.41-8.47 (m, 1H), 11.49 (s, 1H); MS (ESI) m/z 514 (M+H)⁺.

### Example 3

### 4-[3-amino-4-(tetrahydro-2H-pyran-4-yloxy)-1H-indazol-6-yl]-2-(4-cyclopropylpiperazin-1-yl)benzamide

The titled compound was prepared using the procedures described in Example 1 except 1-cyclopropylpiperazine was substituted for 1-phenylpiperazine in Step 2. ¹H NMR (300 MHz, DMSO-d₆) δ ppm 0.31-0.38 (m, 2H), 0.42-0.50 (m, 2H), 1.65-1.83 (m, 3H), 1.99-2.11 (m, 2H), 2.70-2.76 (m, 4H), 2.96-3.03 (m, 4H), 3.45-3.67 (m, 2H), 3.73-3.92 (m, 2H), 4.87-4.98 (m, 1H), 5.06 (s, 2H), 6.64 (s, 1H), 6.99 (d, J=0.9 Hz, 1H), 7.37 (d, J=1.7 Hz, 1H), 7.41 (dd, J=8.0, 1.6 Hz, 1H), 7.55-7.63 (m, 1H), 7.83 (d, J=8.0 Hz, 1H), 8.60-8.65 (m, 1H); MS (ESI) m/z 477 (M+H)⁺.

### Example 4

### 2-(4-acetylpiperazin-1-yl)-4-[3-amino-4-(tetrahydro-2H-pyran-4-yloxy)-1H-indazol-6-yl]benzamide

The titled compound was prepared using the procedures described in Example 1 except 1-(piperazin-1-yl)ethanone was substituted for 1-phenylpiperazine in Step 2. ¹H NMR (300 MHz, DMSO-d₆) δ ppm 1.59-1.86 (m, 2H), 1.85-1.98 (m, 2H), 2.05 (s, 3H),), 3.02 (d, J=21.4 Hz, 4H), 3.51-3.60 (m, 2H), 3.64 (m, 4H), 3.70-3.96 (m, 2H), 4.92 (m, 1H), 5.00 (s, 2H), 6.56-6.78 (m, 1H), 7.00 (s, 1H), 7.29-7.46 (m, 1H), 7.54 (bs, 1H), 7.77 (d, J=7.9 Hz, 1H), 8.37-8.43 (m, 1H); MS (ESI) m/z 479 (M+H)⁺.

### Example 5

### 4-[3-amino-4-(tetrahydro-2H-pyran-4-yloxy)-1H-indazol-6-yl]-2-{4-[2-(morpholin-4-yl)ethyl]piperazin-1-yl}benzamide

The titled compound was prepared using the procedures described in Example 1 except 4-(2-(piperazin-1-yl)ethyl)morpholine was substituted for 1-phenylpiperazine in Step 2. ¹H NMR (300 MHz, DMSO-d₆) δ ppm 1.63-1.89 (m, 2H), 1.94-2.11 (m, 2H), 2.19-2.44 (m, 6H), 2.41-2.48 (m, 4H), 2.61 (bs, 4H), 2.94-3.12 (m, 4H), 3.51-3.62 (m, 4H), 3.81-3.92 (m, 2H), 4.81-5.01 (m, 1H), 5.01 (s, 2H), 6.64 (s, 1H), 7.00 (d, J=1.0 Hz, 1H), 7.36-7.45 (m, 1H), 7.41 (d, J=9.2 Hz, 1H), 7.53-7.59 (m, 1H), 7.82 (d, J=7.9 Hz, 1H), 8.54-8.60 (m, 1H), 11.48 (bs, 1H); MS (ESI) m/z 550 (M+H)⁺.

### Example 6

### 4-[3-amino-4-(tetrahydro-2H-pyran-4-yloxy)-1H-indazol-6-yl]-2-[4-(5-methoxypyrimidin-4-yl)piperazin-1-yl]benzamide

The titled compound was prepared using the procedures described in Example 1 except 5-methoxy-4-(piperazin-1-yl)pyrimidine was substituted for 1-phenylpiperazine in Step 2. ¹H NMR (300 MHz, DMSO-d₆) δ ppm 1.68-1.90 (m, 2H), 1.98-2.10 (m, 2H), 3.15 (dd, J=6.0, 3.4 Hz, 4H), 3.50-3.65 (m, 2H), 3.81-3.92 (m, 9H), 4.88-5.02 (m, 1H), 5.00 (s, 2H), 6.65 (s, 1H), 7.02 (d, J=0.9 Hz, 1H), 7.36-7.46 (m, 2H), 7.52-7.58 (m, 1H), 7.80 (d, J=7.9 Hz, 1H), 8.09 (s, 1H), 8.29 (s, 1H), 8.46-8.52 (m, 1H), 11.47 (bs, 1H); MS (ESI) m/z 545 (M+H)⁺.

### Example 7

### 4-[3-amino-4-(tetrahydro-2H-pyran-4-yloxy)-1H-indazol-6-yl]-2-[4-(3-hydroxypropyl)piperazin-1-yl]benzamide

The titled compound was prepared using the procedures described in Example 1 except 3-(piperazin-1-yl)propan-1-ol was substituted for 1-phenylpiperazine in Step 2. ¹H NMR (300 MHz, DMSO-d₆) δ ppm 1.56-1.69 (m, 2H), 1.70-1.83 (m, 2H), 1.93-2.18 (m, 2H), 2.32-2.51 (m, 2H), 2.49-2.66 (m, 4H), 3.04 (t, J=4.3 Hz, 4H), 3.19-3.40 (m, 6H),), 4.86-4.97 (m, 1H), 5.00 (s, 2H), 6.64 (s, 1H), 7.00 (d, J=0.7 Hz, 1H), 7.41 (dd, J=10.6, 2.6 Hz, 1H), 7.55 (s, 1H), 7.82 (d, J=8.0 Hz, 1H), 8.57 (s, 1H), 11.09-12.15 (m, 1H); MS (ESI) m/z 495 (M+H)⁺.

### Example 8

### 4-[3-amino-4-(tetrahydro-2H-pyran-4-yloxy)-1H-indazol-6-yl]-2-[4-(5-chloropyridin-3-yl)piperazin-1-yl]benzamide

The titled compound was prepared using the procedures described in Example 1 except 1-(5-chloropyridin-3-yl)piperazine was substituted for 1-phenylpiperazine in Step 2. ¹H NMR (300 MHz, DMSO-d₆) δ ppm 1.68-1.85 (m, 2H), 2.01-2.18 (m, 2H), 3.05-3.28 (m, 4H), 3.43-3.68 (m, 4H), 3.80-4.09 (m, 2H), 4.88-5.02 (m, 1H), 5.02 (s, 2H), 6.66 (s, 1H), 7.02 (s, 1H), 7.44 (dd, J=16.5, 8.4 Hz, 1H), 7.47-7.61 (m, 2H), 7.79 (d, J=8.0 Hz, 2H), 8.01 (d, J=1.9 Hz, 1H), 8.32-8.42 (m, 1H), 11.47 (s, 1H); MS (ESI) m/z 548 (M+H)⁺.

### Example 9

### 4-[3-amino-4-(tetrahydro-2H-pyran-4-yloxy)-1H-indazol-6-yl]-2-[4-(1-methylpiperidin-4-yl)piperazin-1-yl]benzamide

The titled compound was prepared using the procedures described in Example 1 except 1-(1-methylpiperidin-4-yl)piperazine was substituted for 1-phenylpiperazine in Step 2. ¹H NMR (300 MHz, DMSO-d₆) δ ppm 1.35-1.51 (m, 2H), 1.71-1.83 (m, 4H), 2.0-2.1 (m, 2H), 2.14 (s, 3H), 2.63-2.70 (m, 4H), 2.70-2.75 (m, 1H), 2.75-2.89 (m, 2H)), 2.99-3.06 (m, 4H), 3.46-3.63 (m, 2H), 3.75-3.96 (m, 2H), 4.03 (bs, 2H), 4.86-4.94 (m, 1H), 5.00 (s, 2H), 6.65 (s, 1H), 7.00 (s, 1H), 7.37-7.45 (m, 2H), 7.54 (d, J=2.4 Hz, 1H), 7.82 (d, J=7.9 Hz, 1H), 8.61 (s, 1H), 11.48 (s, 1H); MS (ESI) m/z 534 (M+H)⁺.

### Example 10

### 4-[3-amino-4-(tetrahydro-2H-pyran-4-yloxy)-1H-indazol-6-yl]-2-{4-[2-(pyridin-2-yl)ethyl]piperazin-1-yl}benzamide

The titled compound was prepared using the procedures described in Example 1 except 1-(2-(pyridin-2-yl)ethyl)piperazine was substituted for 1-phenylpiperazine in Step 2. ¹H NMR (500 MHz, DMSO-d₆) δ ppm 1.67-1.82 (m, 2H), 1.98-2.10 (m, 2H), 2.61-2.70 (m, 4H), 2.75 (t, J=7.8 Hz, 2H), 2.94 (t, J=7.6 Hz, 2H), 3.01-3.08 (m, 4H), 3.51-3.67 (m, 2H), 3.76-4.01 (m, 2H), 4.88-4.96 (m, 1H), 5.02 (s, 2H), 6.65 (s, 1H), 7.01 (s, 1H), 7.20 (dd, J=7.5, 4.9 Hz, 1H), 7.25-7.38 (m, 1H), 7.36-7.50 (m, 2H), 7.55-7.63 (m, 1H), 7.70 (td, J=7.6, 1.9 Hz, 1H), 7.77-7.87 (m, 1H), 8.43-8.51 (m, 1H), 8.56-8.64 (m, 1H), 10.79-12.56 (m, 1H); MS (ESI) m/z 542 (M+H)⁺.

### Example 11

### 4-[3-amino-4-(tetrahydro-2H-pyran-4-yloxy)-1H-indazol-6-yl]-2-{4-[2-(2-hydroxyethoxy)ethyl]piperazin-1-yl}benzamide

The titled compound was prepared using the procedures described in Example 1 except 2-(2-(piperazin-1-yl)ethoxy)ethanol was substituted for 1-phenylpiperazine in Step 2. ¹H NMR (500 MHz, DMSO-d₆) δ ppm 1.60-1.84 (m, 2H), 1.99-2.16 (m, 2H), 2.55 (t, J=5.9 Hz, 2H), 2.60-2.66 (m, 4H), 3.00-3.06 (m, 4H), 3.42 (ddd, J=16.2, 14.9, 5.2 Hz, 4H), 3.46-3.54 (m, 2H), 3.53-3.63 (m, 2H), 3.80-3.91 (m, 2H), 4.86-4.97 (m, 1H), 4.99 (s, 2H), 6.69 (d, J=42.2 Hz, 1H), 7.04 (d, J=35.7 Hz, 1H), 7.33-7.51 (m, 2H), 7.54-7.63 (m, 1H), 7.79-7.90 (m, 1H), 8.53-8.61 (m, 1H); MS (ESI) m/z 525 (M+H)⁺.

### Example 12

### 4-[3-amino-4-(tetrahydro-2H-pyran-4-yloxy)-1H-indazol-6-yl]-2-[4-(3-chlorophenyl)piperazin-1-yl]benzamide

The titled compound was prepared using the procedures described in Example 1 except 1-(3-chlorophenyl)piperazine was substituted for 1-phenylpiperazine in Step 2. ¹H NMR (300 MHz, DMSO-d₆) δ ppm 1.71-1.82 (m, 2H), 2.03-2.09 (m, 2H), 3.17-3.20 (m, 4H), 3.38-3.40 (m, 4H), 3.53-3.60 (m, 2H), 3.83-3.90 (m, 2H), 4.89-4.97 (m, 2H), 5.02 (s, 2H), 6.67 (s, 1H), 682 (dd, J=7.9, 1.2 Hz, 1H), 6.96-7.04 (m, 3H), 7.25 (t, J=8.1 Hz, 1H), 7.40-7.45 (m, 2H), 7.58 (d, J=2.8 Hz, 1H), 7.80 (d, J=8.3 Hz, 1H), 8.44 (d, J=2.8 Hz, 1H), 11.51 (s, 1H); MS (ESI) m/z 547 (M+H)⁺.

### Example 13

### 1-{4-[3-amino-4-(tetrahydro-2H-pyran-4-yloxy)-1H-indazol-6-yl]-2-(4-phenylpiperazin-1-yl)phenyl}ethanone

### Step 1

### 1-{4-[3-amino-4-(tetrahydro-2H-pyran-4-yloxy)-1H-indazol-6-yl]-2-fluorophenyl}ethanone

To a suspension of 6-bromo-4-(tetrahydro-2H-pyran-4-yloxy)-1H-indazol-3-amine (Intermediate 1, 0.5 g, 1.62 mmol) and 4-acetyl-3-fluorophenylboronic acid (0.35 g, 1.92 mmol) in dimethoxyethane:ethanol (6 mL, 1:1) was added 1 M potassium carbonate (2.4 mL). The reaction mixture was purged with nitrogen and bis(triphenylphosphine)palladium(II) dichloride (0.054 g, 0.077 mmol) was added. The reaction mixture was then heated in a microwave reactor (CEM Discover®, ≦300 W) at 160° C. for 20 minutes and then concentrated. The concentrate was partitioned in water/dichloromethane (3×50 mL). The combined organic layers were concentrated and purified chromatographically on silica gel eluting with a 50-100% ethyl acetate/hexane gradient to obtain the titled compound. ¹H NMR (300 MHz, DMSO-d₆) δ ppm 1.66-1.84 (m, 2H), 1.96-2.13 (m, 2H), 2.61 (d, J=4.2 Hz, 3H), 3.48-3.63 (m, 2H), 3.81-3.94 (m, 2H), 4.96 (dt, J=12.2, 4.1 Hz, 1H), 5.02 (s, 2H), 6.74 (s, 1H), 7.10 (d, J=1.0 Hz, 1H), 7.64-7.76 (m, 2H), 7.88 (t, J=8.0 Hz, 1H), 11.60 (s, 1H); MS (ESI) m/z 370 (M+H)⁺.

### Step 2

### 1-{4-[3-amino-4-(tetrahydro-2H-pyran-4-yloxy)-1H-indazol-6-yl]-2-(4-phenylpiperazin-1-yl)phenyl}ethanone

A solution of the product from Step 1 (0.1 g, 0.27 mmol) and 1-phenylpiperazine (0.176 g, 1.73 mmol) in dimethyl sulfoxide (1 mL) was heated at 120° C. overnight. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (400 MHz, DMSO-d₆) δ ppm 1.76 (dt, J=12.8, 4.5 Hz, 2H), 2.05 (d, J=15.7 Hz, 2H), 2.63 (s, 3H), 3.08-3.20 (m, 4H), 3.38-3.5 (m, 4H), 3.49-3.69 (m, 2H), 3.79-4.00 (m, 2H), 4.85-4.96 (m, 1H), 5.00 (bs, 2H), 5.74 (s, 1H), 6.66 (s, 1H), 6.81 (t, J=7.3 Hz, 1H), 7.01 (dd, J=8.3, 4.3 Hz, 3H), 7.16-7.28 (m, 3H), 7.33-7.39 (m, 1H), 7.44 (d, J=7.7 Hz, 1H), 11.49 (s, 1H); MS (ESI) m/z 512 (M+H)⁺.

### Example 14

### 1-{4-[3-amino-4-(tetrahydro-2H-pyran-4-yloxy)-1H-indazol-6-yl]-2-(4-cyclopropylpiperazin-1-yl)phenyl}ethanone

The titled compound was prepared using the procedures described in Example 13 except 1-cyclopropylpiperazine was substituted for 1-phenylpiperazine in Step 2. ¹H NMR (300 MHz, DMSO-d₆) δ ppm 0.34 (m, 2H), 0.45 (d, J=4.3 Hz, 2H), 1.59-1.84 (m, 2H), 2.05-2.09 (m, 2H), 2.27 (d, J=1.8 Hz, 1H), 2.62 (s, 3H), 2.91-3.04 (m, 4H), 3.56 (ddd, J=11.6, 8.5, 3.1 Hz, 2H), 3.81-3.92 (m, 2H), 3.94 (m, 4H) 4.86-4.9 (m, 1H), 5.01 (s, 2H), 6.63 (s, 1H), 6.98 (s, 1H), 7.27 (s, 1H), 7.32 (d, J=8.0 Hz, 1H), 7.41 (d, J=8.0 Hz, 1H), 11.5 (s, 1H); MS (ESI) m/z 476 (M+H)⁺.

### Example 15

### 1-{4-[3-amino-4-(tetrahydro-2H-pyran-4-yloxy)-1H-indazol-6-yl]-2-[4-(pyrimidin-2-yl)piperazin-1-yl]phenyl}ethanone

The titled compound was prepared using the procedures described in Example 13 except 2-(piperazin-1-yl)pyrimidine was substituted for 1-phenylpiperazine in Step 2. ¹H NMR (500 MHz, DMSO-d₆) δ ppm 1.70-1.81 (m, 2H), 2.00-2.11 (m, 2H), 2.67 (s, 3H), 3.07-3.13 (m, 4H), 3.52-3.61 (m, 2H), 3.82-3.90 (m, 2H), 3.90-3.96 (m, 4H), 4.91 (dp, J=7.9, 4.0 Hz, 1H), 5.00 (bs, 2H), 6.65 (d, J=2.9 Hz, 1H), 6.66 (dd, J=7.7, 3.0 Hz, 1H), 7.02 (s, 1H), 7.35 (s, 1H), 7.36-7.39 (m, 1H), 7.44-7.50 (m, 1H), 8.37-8.42 (m, 2H), 11.46-11.58 (m, 1H); MS (ESI) m/z 514 (M+H)⁺.

### Example 16

### 1-{4-[3-amino-4-(tetrahydro-2H-pyran-4-yloxy)-1H-indazol-6-yl]-2-[4-(pyridin-3-yl)piperazin-1-yl]phenyl}ethanone

The titled compound was prepared using the procedures described in Example 13 except 1-(pyridin-3-yl)piperazine was substituted for 1-phenylpiperazine in Step 2. ¹H NMR (300 MHz, DMSO-d₆) δ ppm 1.66-1.86 (m, 2H), 1.96-2.30 (m, 2H), 2.65 (s, 3H), 3.20 (d, J=4.7 Hz, 4H), 3.39 (d, J=5.1 Hz, 4H), 3.46-3.65 (m, 2H), 3.82-3.93 (m, 2H), 4.84-4.98 (m, 1H), 5.00 (s, 2H), 6.67 (s, 1H), 7.03 (d, J=0.9 Hz, 1H), 7.25 (dd, J=8.4, 4.5 Hz, 1H), 7.31-7.44 (m, 3H), 7.46 (d, J=8.0 Hz, 1H), 8.03 (dd, J=4.5, 1.3 Hz, 1H), 8.37 (d, J=2.9 Hz, 1H), 11.49 (s, 1H); MS (ESI) m/z 513 (M+H)⁺.

### Example 17

### 1-{4-[3-amino-4-(tetrahydro-2H-pyran-4-yloxy)-1H-indazol-6-yl]-2-[4-(4-fluorophenyl)piperazin-1-yl]phenyl}ethanone

The titled compound was prepared using the procedures described in Example 13 except 1-(4-fluorophenyl)piperazine was substituted for 1-phenylpiperazine in Step 2. ¹H NMR (300 MHz, DMSO-d₆) δ ppm 1.60-1.88 (m, 2H), 1.96-2.21 (m, 2H), 2.64 (s, 3H), 3.19 (d, J=4.0 Hz, 4H), 3.27 (d, J=3.8 Hz, 4H), 3.46-3.66 (m, 2H), 3.79-3.95 (m, 2H), 4.92 (ddd, J=10.0, 8.2, 4.0 Hz, 1H), 5.00 (s, 2H), 6.67 (s, 1H), 6.93-7.20 (m, 5H), 7.30-7.49 (m, 3H), 11.56 (d, J=38.0 Hz, 1H); MS (ESI) m/z 528 (M+H)⁺.

### Example 18

### 1-{4-[3-amino-4-(tetrahydro-2H-pyran-4-yloxy)-1H-indazol-6-yl]-2-[4-(3,3-dimethylbutyl)piperazin-1-yl]phenyl}ethanone

The titled compound was prepared using the procedures described in Example 13 except 1-(3,3-dimethylbutyl)piperazine was substituted for 1-phenylpiperazine in Step 2. ¹H NMR (300 MHz, DMSO-d₆) δ ppm 0.90 (s, 9H), 1.38 (dd, J=9.5, 6.6 Hz, 2H), 1.68-1.87 (m, 2H), 1.96-2.14 (m, 2H), 2.35 (t, J=8.0 Hz, 2H), 2.52-2.61 (m, 4H), 2.61 (s, 3H), 3.02 (s, 4H), 3.40-3.64 (m, 2H), 3.77-4.04 (m, 2H), 4.75-4.99 (m, 1H), 4.97 (bs, 2H), 6.64 (s, 1H), 6.99 (d, J=0.9 Hz, 1H), 7.28 (s, 1H), 7.29-7.35 (m, 1H), 7.40 (d, J=7.9 Hz, 1H), 11.48 (s, 1H).

### Example 19

### 1-{4-[3-amino-4-(tetrahydro-2H-pyran-4-yloxy)-1H-indazol-6-yl]-2-(4-cyclohexylpiperazin-1-yl)phenyl}ethanone

The titled compound was prepared using the procedures described in Example 13 except 1-cyclohexylpiperazine was substituted for 1-phenylpiperazine in Step 2. ¹H NMR (300 MHz, DMSO-d₆) δ ppm 1.22 (t, J=9.6 Hz, 4H), 1.54-1.63 (m, 1H), 1.67-1.88 (m, 6H), 1.95-2.14 (m, 3H), 2.30 (t, J=11.7 Hz, 1H), 2.61 (s, 3H), 2.63-2.74 (m, 4H), 3.01 (d, J=3.9 Hz, 4H), 3.56 (ddd, J=11.5, 8.5, 3.0 Hz, 2H), 3.81-3.92 (m, 2H), 4.86-4.96 (m, 1H), 4.99 (s, 2H), 6.64 (s, 1H), 6.99 (d, J=0.9 Hz, 1H), 7.27 (s, 1H), 7.31 (dd, J=8.0, 1.4 Hz, 1H), 7.40 (d, J=7.9 Hz, 1H), 11.48 (s, 1H); MS (ESI) m/z 518 (M+H)⁺.

### Example 20

### 1-{4-[3-amino-4-(tetrahydro-2H-pyran-4-yloxy)-1H-indazol-6-yl]-2-[4-(cyclopropylcarbonyl)piperazin-1-yl]phenyl}ethanone

The titled compound was prepared using the procedures described in Example 13 except cyclopropyl(piperazin-1-yl)methanone was substituted for 1-phenylpiperazine in Step 2. ¹H NMR (300 MHz, DMSO-d₆) δ ppm 0.64-0.82 (m, 4H), 1.57-1.84 (m, 2H), 1.91-2.12 (m, 3H), 2.64 (s, 3H), 3.07 (s, 4H), 3.55 (ddd, J=14.5, 8.7, 4.5 Hz, 2H), 3.63 (d, J=13.6 Hz, 4H), 3.79-3.97 (m, 2H), 4.84-4.98 (m, 1H), 5.00 (s, 1H), 6.65 (s, 1H), 7.02 (s, 1H), 7.33 (s, 1H), 7.35-7.41 (m, 1H), 7.47 (d, J=7.9 Hz, 1H), 11.49 (s, 1H); MS (ESI) m/z 504 (M+H)⁺.

### Example 21

### 1-{4-[3-amino-4-(tetrahydro-2H-pyran-4-yloxy)-1H-indazol-6-yl]-2-[4-(1,3-thiazol-2-yl)piperazin-1-yl]phenyl}ethanone

The titled compound was prepared using the procedures described in Example 13 except 2-(piperazin-1-yl)thiazole was substituted for 1-phenylpiperazine in Step 2. ¹H NMR (300 MHz, DMSO-d₆) δ ppm 1.76 (d, J=8.5 Hz, 2H), 2.05 (ddd, J=9.2, 6.1, 3.7 Hz, 2H), 2.65 (s, 3H), 3.13-3.20 (m, 4H), 3.51-3.64 (m, 6H), 3.79-3.95 (m, 2H), 4.92 (ddd, J=10.8, 6.3, 2.5 Hz, 1H), 5.01 (bs, 2H), 6.66 (s, 1H), 6.89 (d, J=3.5 Hz, 1H), 7.02 (d, J=0.9 Hz, 1H), 7.21 (d, J=3.5 Hz, 1H), 7.37 (d, J=2.4 Hz, 1H), 7.40 (d, J=1.5 Hz, 1H), 7.48 (d, J=7.9 Hz, 1H), 11.50 (s, 1H); MS(ESI) m/z 519 (M+H)⁺.

### Example 22

### 1-{4-[3-amino-4-(tetrahydro-2H-pyran-4-yloxy)-1H-indazol-6-yl]-2-[4-(pyridin-3-yl)piperazin-1-yl]phenyl}ethanol

Sodium borohydride (6 mg, 0.164 mmol) was added to a solution of 1-{4-[3-amino-4-(tetrahydro-2H-pyran-4-yloxy)-1H-indazol-6-yl]-2-[4-(pyridin-3-yl)piperazin-1-yl]phenyl}ethanone (Example 16, 42 mg, 0.082 mmol) in methanol (3 mL), and the mixture was stirred at ambient temperature for 5 hours. Then 1 M citric acid (2 mL) was added followed by stirring for 1 hour. The reaction mixture was concentrated and partition in water/dichloromethane (3×25 mL). The combined organic layers were dried over MgSO₄, filtered, and concentrated. Chromatographic purification on silica gel eluting with 5% methanol/dichloromethane gave the titled compound. ¹H NMR (300 MHz, DMSO-d₆) δ ppm 1.38 (d, J=6.3 Hz, 3H), 1.76 (d, J=8.6 Hz, 2H), 2.06 (d, J=13.9 Hz, 2H), 2.94-3.10 (m, 2H), 3.13-3.29 (m, 2H), 3.37 (m, 4H), 3.56 (ddd, J=11.3, 8.5, 2.9 Hz, 2H), 3.77-3.94 (m, 2H), 4.86-4.95 (m, 1H), 4.97 (s, 2H), 5.02 (d, J=4.5 Hz, 1H), 5.17-5.27 (m, 1H), 6.62 (s, 1H), 6.95 (s, 1H), 7.24 (dd, J=8.5, 4.5 Hz, 1H), 7.30-7.48 (m, 3H), 7.56 (d, J=8.0 Hz, 1H), 8.03 (dd, J=4.5, 1.3 Hz, 1H), 8.37 (d, J=2.9 Hz, 1H), 11.41 (s, 1H).

### Example 23

### 4-[3-amino-4-(tetrahydro-2H-pyran-4-yloxy)-1H-indazol-6-yl]-N-cyclopropyl-2-(4-phenylpiperazin-1-yl)benzamide

### Step 1

### 4-[3-amino-4-(tetrahydro-2H-pyran-4-yloxy)-1H-indazol-6-yl]-N-cyclopropyl-2-fluorobenzamide

To a suspension of 6-bromo-4-(tetrahydro-2H-pyran-4-yloxy)-1H-indazol-3-amine (Intermediate 1, 0.33 g, 1.05 mmol) and 4-(cyclopropylcarbamoyl)-3-fluorophenylboronic acid (0.35 g, 1.57 mmol) in dimethoxyethane:ethanol (5 mL, 1:1) was added 1 M potassium carbonate (2.4 mL). The mixture was purged with nitrogen and bis(triphenylphosphine)palladium(II) dichloride (0.035 g, 0.048 mmol) was added. The resultant mixture was heat in a microwave reactor (CEM Discover®, ≦300 W) at 160° C. for 20 minutes. Then the reaction mixture was concentrated, and the concentrate was partitioned between water and dichloromethane (3×50 mL). The combined organic layers were concentrated, and the concentrate was purified chromatographically on silica gel eluting with a 50-100% ethyl acetate/hexane gradient to obtain the titled compound. ¹H NMR (300 MHz, DMSO-d₆) δ ppm 0.48-0.62 (m, 2H), 0.71 (td, J=7.1, 4.9 Hz, 2H), 1.64-1.87 (m, 2H), 2.05 (dd, J=9.4, 5.4 Hz, 2H)), 2.78-2.92 (m, 1H), 3.48-3.62 (m, 2H), 3.82-3.93 (m, 2H), 4.89-4.98 (m, 1H), 5.01 (s, 2H), 6.48 (s, 1H), 6.69 (s, 1H), 7.04 (d, J=1.0 Hz, 1H), 7.63 (dt, J=9.8, 5.1 Hz, 2H), 8.35 (d, J=5.0 Hz, 1H), 11.54 (s, 1H); MS (ESI) m/z 411 (M+H)⁺.

### Step 2

### 4-[3-amino-4-(tetrahydro-2H-pyran-4-yloxy)-1H-indazol-6-yl]-N-cyclopropyl-2-(4-phenylpiperazin-1-yl)benzamide

A solution of the product from Step 1 (0.075 g, 0.18 mmol) and 1-phenylpiperazine (0.09 g, 0.55 mmol) in dimethyl sulfoxide (imp was heated at 120° C. overnight. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 0.50-0.63 (m, 2H), 0.73 (td, J=7.1, 4.9 Hz, 2H), 1.69-1.84 (m, 2H), 1.98-2.12 (m, 2H), 2.89 (td, J=7.3, 3.9 Hz, 1H), 3.18 (d, J=4.5 Hz, 4H), 3.41 (m, 4H), 3.48-3.64 (m, 2H), 3.81-3.92 (m, 2H), 4.82-5.00 (m, 1H), 5.00 (s, 2H), 6.65 (s, 1H), 6.83 (t, J=7.2 Hz, 1H), 6.98-7.05 (m, 3H), 7.21-7.31 (m, 2H), 7.42-7.49 (m, 2H), 7.76 (d, J=8.4 Hz, 1H), 9.20 (d, J=4.4 Hz, 1H), 11.48 (s, 1H); MS (ESI) m/z 553 (M+H)⁺.

### Example 24

### 4-[3-amino-4-(tetrahydro-2H-pyran-4-yloxy)-1H-indazol-6-yl]-N-cyclopropyl-2-[4-(pyridin-3-yl)piperazin-1-yl]benzamide

The titled compound was prepared using the procedures described in Example 23 except 1-(pyridin-3-yl)piperazine was substituted for 1-phenylpiperazine in Step 2. ¹H NMR (300 MHz, DMSO-d₆) δ ppm 0.50-0.64 (m, 2H), 0.62-0.77 (m, 2H), 1.77 (d, J=8.4 Hz, 2H), 1.99-2.14 (m, 2H), 2.89 (d, J=4.0 Hz, 1H), 3.04-3.26 (m, 4H), 3.46-3.51 (m, 4H), 3.48-3.64 (m, 2H), 3.81-3.92 (m, 2H), 4.96 (d, J=22.9 Hz, 1H), 5.00 (s, 1H), 6.65 (s, 1H), 7.02 (s, 1H), 7.26 (dd, J=8.4, 4.5 Hz, 1H), 7.36-7.51 (m, 2H), 7.74 (d, J=7.8 Hz, 1H), 8.04 (dd, J=4.5, 1.2 Hz, 1H), 8.38 (d, J=2.9 Hz, 1H), 9.11 (d, J=4.4 Hz, 1H), 11.23-11.76 (m, 1H).); MS (ESI) m/z 554 (M+H)⁺.

### Example 25

### 4-[3-amino-4-(tetrahydro-2H-pyran-4-yloxy)-1H-indazol-6-yl]-N-cyclopropyl-2-[4-(2,2-dimethylpropanoyl)piperazin-1-yl]benzamide

The titled compound was prepared using the procedures described in Example 23 except 2,2-dimethyl-1-(piperazin-1-yl)propan-1-one was substituted for 1-phenylpiperazine in Step 2. ¹H NMR (300 MHz, DMSO-d₆) δ ppm 0.51-0.66 (m, 2H), 0.68-0.83 (m, 2H), 1.23 (d, J=3.6 Hz, 9H), 1.76 (dd, J=8.3, 4.3 Hz, 2H), 2.05 (d, J=12.3 Hz, 2H), 2.83-2.94 (m, 1H), 2.99 (m, 4H), 3.56 (t, J=8.5 Hz, 2H), 3.70 (s, 4H), 3.85 (dd, J=11.0, 5.7 Hz, 2H), 4.91 (ddd, J=11.6, 7.8, 3.7 Hz, 1H), 5.00 (s, 2H), 6.62 (s, 1H), 7.02 (d, J=16.8 Hz, 1H), 7.37 (s, 1H), 7.42 (d, J=9.3 Hz, 1H), 7.70 (d, J=8.0 Hz, 1H), 9.04 (d, J=4.3 Hz, 1H), 11.47 (s, 1H); MS (ESI) m/z 561 (M+H)⁺.

### Example 26

### 4-{3-amino-4-[(1,1,1-trifluoropropan-2-yl)oxy]-1H-indazol-6-yl}-N-cyclopropyl-2-(4-phenylpiperazin-1-yl)benzamide

The titled compound was prepared using the procedures described in Example 23 except 6-bromo-4-[(1,1,1-trifluoropropan-2-yl)oxy]-1H-indazol-3-amine (Intermediate 2) was substituted for 6-bromo-4-(tetrahydro-2H-pyran-4-yloxy)-1H-indazol-3-amine (Intermediate 1) in Step 1. ¹H NMR (300 MHz, DMSO-d₆) δ ppm 0.52-0.59 (m, 2H), 0.66-0.88 (m, 2H)), 1.54 (d, J=6.3 Hz, 3H), 2.81-2.99 (m, 1H), 3.15-3.23 (m, 4H), 4.95 (s, 2H), 5.68 (dd, J=12.9, 6.4 Hz, 1H), 6.80-6.89 (m, 2H), 7.02 (d, J=7.9 Hz, 2H), 7.14 (s, 1H), 7.26 (dd, J=8.6, 7.1 Hz, 2H), 7.50 (s, 1H), 7.53 (dd, J=4.5, 2.9 Hz, 1H), 7.78 (d, J=8.2 Hz, 1H), 9.25 (d, J=4.4 Hz, 1H), 11.63 (s, 1H); MS (ESI) m/z 565 (M+H)⁺.

### Example 27

### 4-{3-amino-4-[(1,1,1-trifluoropropan-2-yl)oxy]-1H-indazol-6-yl}-N-cyclopropyl-2-(4-cyclopropylpiperazin-1-yl)benzamide

The titled compound was prepared using the procedures described in Example 23 except 6-bromo-4-[(1,1,1-trifluoropropan-2-yl)oxy]-1H-indazol-3-amine (Intermediate 2) was substituted for 6-bromo-4-(tetrahydro-2H-pyran-4-yloxy)-1H-indazol-3-amine (Intermediate 1) in Step 1 and 1-cyclopropylpiperazine was substituted for 1-phenylpiperazine in Step 2. ¹H NMR (300 MHz, DMSO-d₆) δ ppm 0.28-0.39 (m, 2H), 0.42-0.52 (m, 2H), 0.54-0.64 (m, 2H), 0.78 (td, J=7.0, 4.9 Hz, 2H), 1.53 (d, J=6.3 Hz, 3H), 1.64-1.75 (m, 1H), 2.64-2.80 (m, 4H), 2.91 (td, J=7.3, 4.0 Hz, 1H), 2.92-3.09 (m, 4H), 4.94 (bs, 2H), 5.34-5.85 (m, 1H), 6.82 (s, 1H), 7.11 (d, J=0.9 Hz, 1H), 7.43-7.54 (m, 2H), 7.82 (d, J=8.0 Hz, 1H), 9.48 (d, J=4.5 Hz, 1H), 11.62 (s, 1H); MS (ESI) m/z 529 (M+H)⁺.

### Example 28

### 4-{3-amino-4-[(1,1,1-trifluoropropan-2-yl)oxy]-1H-indazol-6-yl}-N-cyclopropyl-2-[4-(pyridin-3-yl)piperazin-1-yl]benzamide

The titled compound was prepared using the procedures described in Example 23 except 6-bromo-4-[(1,1,1-trifluoropropan-2-yl)oxy]-1H-indazol-3-amine (Intermediate 2) was substituted for 6-bromo-4-(tetrahydro-2H-pyran-4-yloxy)-1H-indazol-3-amine (Intermediate 1) in Step 1 and 1-(pyridin-3-yl)piperazine was substituted for 1-phenylpiperazine in Step 2. The crude reaction mixture was purified by preparative HPLC on a Phenomenex® Luna® C8(2) 5 μm 100 Å AXIA™ column (30 mm×75 mm) A gradient of acetonitrile (A) and 0.1% trifluoroacetic acid in water (B) was used, at a flow rate of 50 mL/minute (0-0.5 minutes 10% A, 0.5-7.0 minutes linear gradient 10-95% A, 7.0-10.0 minutes 95% A, 10.0-12.0 minutes linear gradient 95-10% A). ¹H NMR (500 MHz, DMSO-d₆) δ ppm 0.54-0.64 (m, 2H), 0.66-0.76 (m, 2H), 1.55 (d, J=6.3 Hz, 3H), 2.85-2.94 (m, 1H), 3.19-3.24 (m, 4H), 3.54-3.60 (m, 4H), 4.95 (s, 2H), 5.68 (dt, J=12.7, 6.4 Hz, 1H), 6.90 (s, 1H), 7.17 (s, 1H), 7.45 (d, J=1.6 Hz, 1H), 7.51 (dd, J=8.0, 1.6 Hz, 1H), 7.70 (d, J=7.9 Hz, 1H), 7.86 (dd, J=8.9, 5.3 Hz, 1H), 8.15 (dd, J=9.0, 2.7 Hz, 1H), 8.22-8.28 (m, 1H), 8.53 (bs, 1H), 8.96 (d, J=4.4 Hz, 1H), 11.74 (d, J=239.9 Hz, 1H); MS (ESI) m/z 566 (M+H)⁺.

### Example 29

### 4-[3-amino-4-(tetrahydro-2H-pyran-4-yloxy)-1H-indazol-6-yl]-2-(4-cyclohexylpiperazin-1-yl)-N-methylbenzamide

### Step 1

### 4-[3-amino-4-(tetrahydro-2H-pyran-4-yloxy)-1H-indazol-6-yl]-2-fluoro-N-methylbenzamide

To a suspension of 6-bromo-4-(tetrahydro-2H-pyran-4-yloxy)-1H-indazol-3-amine (Intermediate 1, 0.5 g, 1.62 mmol) and 3-fluoro-4-(methylcarbamoyl)phenylboronic acid (0.35 g, 1.92 mmol) in dimethoxyethane:ethanol (6 mL, 1:1) was added 1 M potassium carbonate (2.4 mL). The reaction mixture was purged with nitrogen and bis(triphenylphosphine)palladium(II) dichloride (0.054 g, 0.077 mmol) was added. The reaction mixture was then heated in a microwave reactor (CEM Discover®, ≦300 W) at 160° C. for 20 minutes and then concentrated. The concentrate was partitioned in water/dichloromethane (3×50 mL). The combined organic layers were concentrated and purified chromatographically on silica gel eluting with a 0-10% methanol/ethyl acetate gradient to obtain the titled compound. ¹H NMR (300 MHz, DMSO-d₆) δ ppm 1.67-1.83 (m, 2H), 2.00-2.13 (m, 2H), 2.80 (d, J=4.5 Hz, 3H), 3.48-3.62 (m, 2H), 3.78-3.93 (m, 2H), 4.94 (dt, J=12.1, 6.2 Hz, 1H), 5.01 (s, 2H), 6.70 (s, 1H), 7.05 (d, J=1.0 Hz, 1H), 7.58-7.74 (m, 3H), 8.19-8.22 (m, 1H), 11.51-11.58 (m, 1H); MS (ESI) m/z 385 (M+H)⁺.

### Step 2

### 4-[3-amino-4-(tetrahydro-2H-pyran-4-yloxy)-1H-indazol-6-yl]-2-(4-cyclohexylpiperazin-1-yl)-N-methylbenzamide

A solution of the product from Step 1 (0.075 g, 0.18 mmol) and 1-cyclohexylpiperazine (0.092 g, 0.546 mmol) in dimethyl sulfoxide (1 mL) was heated at 120° C. overnight. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.18 (dt, J=14.6, 9.4 Hz, 6H), 1.40-1.63 (m, 1H), 1.79 (dtd, J=13.1, 8.3, 4.1 Hz, 6H), 2.00-2.12 (m, 2H), 2.22-2.34 (m, 1H), 2.62-2.78 (m, 4H), 2.85 (d, J=4.7 Hz, 3H), 3.00 (d, J=4.0 Hz, 4H), 3.57 (ddd, J=11.5, 8.4, 3.1 Hz, 2H), 3.81-3.92 (m, 2H), 4.83-4.99 (m, 1H), 4.99 (s, 2H), 6.64 (s, 1H), 6.99 (s, 1H), 7.37 (s, 1H), 7.40 (dd, J=8.0, 1.6 Hz, 1H), 7.77 (d, J=7.9 Hz, 1H), 9.07 (d, J=4.8 Hz, 1H), 11.47 (s, 1H).

### Example 30

### 4-[3-amino-4-(tetrahydro-2H-pyran-4-yloxy)-1H-indazol-6-yl]-N-methyl-2-(4-phenylpiperazin-1-yl)benzamide

The titled compound was prepared as described in Example 29 substituting 1-phenylpiperazine for 1-cyclohexylpiperazine. ¹H NMR (300 MHz, DMSO-d₆) δ ppm 1.69-1.84 (m, 2H), 1.99-2.12 (m, 2H), 2.85 (d, J=4.7 Hz, 3H), 3.06-3.24 (m, 4H), 3.35 (m, 4H), 3.57 (ddd, J=11.5, 8.5, 3.1 Hz, 2H), 3.88 (dd, J=11.6, 5.1 Hz, 2H), 4.93 (ddd, J=10.8, 7.5, 3.4 Hz, 1H), 4.97 (s, 2H), 6.66 (s, 1H), 6.82 (t, J=7.2 Hz, 1H), 6.98-7.06 (m, 3H), 7.21-7.30 (m, 2H), 7.40-7.48 (m, 2H), 7.78 (d, J=7.8 Hz, 1H), 8.95 (d, J=4.8 Hz, 1H), 11.48 (s, 1H); MS (ESI) m/z 527.3 (M+H)⁺.

### Example 31

### 4-[3-amino-4-(tetrahydro-2H-pyran-4-yloxy)-1H-indazol-6-yl]-N-methyl-2-[4-(pyridin-3-yl)piperazin-1-yl]benzamide

The titled compound was prepared as described in Example 29 substituting 1-(pyridin-3-yl)piperazine for 1-cyclohexylpiperazine. ¹H NMR (300 MHz, DMSO-d₆) δ ppm 1.67-1.88 (m, 2H), 2.00-2.16 (m, 2H), 2.86 (d, J=4.7 Hz, 3H), 3.18 (s, 4H), 3.37-3.46 (m, 4H), 3.57 (ddd, J=11.4, 8.7, 3.0 Hz, 2H), 3.86 (dd, J=11.2, 5.8 Hz, 2H), 4.85-4.99 (m, 1H), 5.00 (s, 2H), 6.66 (s, 1H), 7.02 (s, 1H), 7.25 (dd, J=8.4, 4.5 Hz, 1H), 7.42 (td, J=9.2, 1.6 Hz, 3H), 7.75 (t, J=7.3 Hz, 1H), 8.03 (dd, J=4.5, 1.2 Hz, 1H), 8.38 (d, J=2.6 Hz, 1H), 8.90 (d, J=4.9 Hz, 1H), 11.52 (s, 1H); MS (ESI) m/z 528 (M+H)⁺.

### Example 32

### 4-[3-amino-4-(tetrahydro-2H-pyran-4-yloxy)-1H-indazol-6-yl]-2-[4-(cyclopropylcarbonyl)piperazin-1-yl]-N-methylbenzamide

The titled compound was prepared as described in Example 29 substituting cyclopropyl(piperazin-1-yl)methanone for 1-cyclohexylpiperazine. ¹H NMR (300 MHz, DMSO-d₆) δ ppm 0.66-0.88 (m, 4H), 1.76 (dd, J=30.0, 21.2 Hz, 2H), 2.06 (d, J=16.8 Hz, 2H), 2.87 (d, J=4.7 Hz, 3H), 3.05 (m, 4H), 3.55 (dd, J=14.2, 5.7 Hz, 2H), 3.67 (m, 2H), 3.78-3.99 (m, 4H), 4.85-4.99 (m, 1H), 4.99 (s, 2H), 6.64 (s, 1H), 7.01 (s, 1H), 7.37 (d, J=1.5 Hz, 1H), 7.42 (d, J=8.1 Hz, 1H), 7.75 (d, J=8.0 Hz, 1H), 8.90 (d, J=4.7 Hz, 1H), 11.01-12.07 (m, 1H); MS (ESI) m/z 519 (M+H)⁺.

### Example 33

### 4-{3-amino-4-[(1,1,1-trifluoropropan-2-yl)oxy]-1H-indazol-6-yl}-N-methyl-2-(4-phenylpiperazin-1-yl)benzamide

### Step 1

### 4-{3-amino-4-[(1,1,1-trifluoropropan-2-yl)oxy]-1H-indazol-6-yl}-2-fluoro-N-methylbenzamide

To a suspension of 6-bromo-4[(1,1,1-trifluoropropan-2-yl)oxy]-1H-indazol-3-amine (Intermediate 2, 0.328 g, 1.01 mmol) and 3-fluoro-4-(methylcarbamoyl)phenylboronic acid (0.298 g, 1.50 mmol) in dimethoxyethane:ethanol (4 mL, 1:1) was added 1 M potassium carbonate (1.5 mL). The reaction mixture was purged with nitrogen and bis(triphenylphosphine)palladium(II) dichloride (0.026 g, 0.036 mmol) was added. The reaction mixture was then heated in a microwave reactor (CEM Discover®, ≦300 W) at 160° C. for 20 minutes and then concentrated. The concentrate was partitioned in water/dichloromethane (3×50 mL). The combined organic layers were concentrated and purified chromatographically on silica gel eluting with ethyl acetate to obtain the titled compound. ¹H NMR (500 MHz, DMSO-d₆) δ ppm 1.54 (d, J=6.3 Hz, 3H), 2.81 (d, J=4.3 Hz, 3H), 4.96 (bs, 2H), 5.68 (dq, J=12.7, 6.3 Hz, 1H), 6.92 (s, 1H), 7.20 (s, 1H), 7.65-7.76 (m, 3H), 8.22-8.28 (m, 1H), 11.72 (s, 1H); MS (ESI) m/z 397 (M+H)⁺.

### Step 2

### 4-{3-amino-4-[(1,1,1-trifluoropropan-2-yl)oxy]-1H-indazol-6-yl}-N-methyl-2-(4-phenylpiperazin-1-yl)benzamide

A solution of the product from Step 1 (48.2 mg, 0.122 mmol) and 1-phenylpiperazine (98.2 g, 0.605 mmol) in dimethyl sulfoxide (1 mL) was heated at 150° C. for 48 hours. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.54 (d, J=6.1 Hz, 3H), 2.86 (d, J=4.6 Hz, 3H), 3.17-3.20 (m, 4H), 3.36-3.38 (m, 4H), 4.94 (s, 2H), 5.62-5.70 (m, 1H), 6.79-6.85 (m, 2H), 7.01-7.04 (m, 2H), 7.15 (s, 1H), 7.23-7.28 (m, 2H), 7.48-7.51 (m, 2H), 7.79 (d, J=7.8 Hz, 1H), 8.98 (q, J=4.6 Hz, 1H), 11.65 (s, 1H); MS (ESI) m/z 539 (M+H)⁺.

### Example 34

### 4-{3-amino-4-[(1,1,1-trifluoropropan-2-yl)oxy]-1H-indazol-6-yl}-2-(4-cyclopropylpiperazin-1-yl)-N-methylbenzamide

The titled compound was prepared as described in Example 33 except 1-cyclopropylpiperazine was substituted for 1-phenylpiperazine. ¹H NMR (300 MHz, DMSO-d₆) δ ppm 0.32-0.36 (m, 2H), 0.44-0.49 (m, 2H), 1.53 (d, J=6.4 Hz, 3H), 1.67-1.74 (m, 1H), 2.73-2.76 (m, 4H), 3.00-3.03 (m, 4H), 4.94 (s, 2H), 5.62-5.70 (m, 1H), 6.83 (s, 1H), 7.12 (s, 1H), 7.43-7.49 (m, 2H), 7.59 (d, J=2.4 Hz, 1H), 7.84 (d, J=7.9 Hz, 1H), 8.68 (d, J=2.8 Hz, 1H), 11.62 (s, 1H); MS (ESI) m/z 503 (M+H)⁺.

### Example 35

### 4-{3-amino-4-[(1,1,1-trifluoropropan-2-yl)oxy]-1H-indazol-6-yl}-2-(4-cyclohexylpiperazin-1-yl)-N-methylbenzamide

The titled compound was prepared as the acetate salt using the procedure described in Example 33 except 1-cyclohexylpiperazine was substituted for 1-phenylpiperazine. ¹H NMR (300 MHz, DMSO-d₆) δ ppm 1.10-1.30 (m, 5H), 1.52-1.61 (m, 4H), 1.75-1.82 (m, 4H), 1.89 (s, 3H), 2.26-2.34 (m, 1H), 2.68-2.70 (m, 4H), 2.85 (d, J=4.8 Hz, 3H), 2.99-3.02 (m, 4H), 4.94 (s, 2H), 5.62-5.71 (m, 1H), 6.83 (s, 1H), 7.12 (s, 1H), 7.44-7.48 (m, 2H), 7.79 (d, J=7.9 Hz, 1H), 9.13 (q, J=4.5 Hz, 1H), 11.62 (s, 1H); MS (ESI) m/z 545 (M+H)⁺.

### Example 36

### 4-{3-amino-4-[(1,1,1-trifluoropropan-2-yl)oxy]-1H-indazol-6-yl}-N-methyl-2-[4-(pyridin-3-yl)piperazin-1-yl]benzamide

The titled compound was prepared as described in Example 33 except 1-(pyridin-3-yl)piperazine was substituted for 1-phenylpiperazine. ¹H NMR (300 MHz, DMSO-d₆) δ ppm 1.54 (d, J=6.1 Hz, 3H), 2.85 (d, J=4.6 Hz, 3H), 3.18-3.21 (m, 4H), 3.41-3.44 (m, 4H), 4.94 (s, 2H), 5.62-5.70 (m, 1H), 6.85 (s, 1H), 7.15 (s, 1H), 7.01-7.04 (m, 2H), 7.25 (dd, J=8.3, 4.4 Hz, 1H), 7.41 (ddd, J=8.4, 2.9, 1.4 Hz, 1H), 7.46-7.51 (m, 2H), 7.78 (d, J=7.9 Hz, 1H), 8.03 (dd, J=4.4, 1.2 Hz, 1H), 8.38 (d, J=2.8 Hz, 1H), 8.93 (q, J=4.6 Hz, 1H), 11.63 (s, 1H); MS (ESI) m/z 540 (M+H)⁺.

### Example 37

### 4-{3-amino-4-[(1,1,1-trifluoropropan-2-yl)oxy]-1H-indazol-6-yl}-N-methyl-2-{4-[2-(morpholin-4-yl)ethyl]piperazin-1-yl}benzamide

The titled compound as the tris trifluoroacetate salt was prepared as described in Example 33 except 4-(2-(piperazin-1-yl)ethyl)morpholine was substituted for 1-phenylpiperazine. The crude reaction mixture was purified by preparative HPLC on a Phenomenex® Luna® C8(2) 5 um 100 Å AXIA™ column (30 mm×75 mm) A gradient of acetonitrile (A) and 0.1% trifluoroacetic acid in water (B) was used, at a flow rate of 50 mL/minute (0-0.5 minutes 10% A, 0.5-7.0 minutes linear gradient 10-95% A, 7.0-10.0 minutes 95% A, 10.0-12.0 minutes linear gradient 95-10% A). Samples were injected in 1.5 mL dimethyl sulfoxide:methanol (1:1). ¹H NMR (300 MHz, DMSO-d₆) δ ppm 1.55 (d, J=4.4 Hz, 3H), 2.86 (d, J=4.8 Hz, 3H), 2.97-3.15 9 m, 15H), 3.76 (m, 4H), 5.62-5.71 (m, 1H), 6.87 (s, 1H), 7.11 (s, 1H), 7.36 (d, J=1.6 Hz, 1H), 7.50 (dd, J=7.9, 1.2 Hz, 1H), 7.68 (d, J=7.9 Hz, 1H), 8.56 (q, J=4.8 Hz, 1H), 11.84 (s, 1H); MS (ESI) m/z 576 (M+H)⁺.

### Example 38

### 4-{3-amino-4-[(1,1,1-trifluoropropan-2-yl)oxy]-1H-indazol-6-yl}-N-methyl-2-[4-(pyrazin-2-yl)piperazin-1-yl]benzamide

The titled compound was prepared as described in Example 33 except 2-(piperazin-1-yl)pyrazine was substituted for 1-phenylpiperazine. ¹H NMR (300 MHz, DMSO-d₆) δ ppm 1.53 (d, J=6.1 Hz, 3H), 2.87 (d, J=4.8 Hz, 3H), 3.13-3.16 (m, 4H), 3.77-3.90 (m, 4H), 4.93 (s, 2H), 5.59-5.69 (m, 1H), 6.84 (s, 1H), 7.15 (s, 1H), 7.23-7.28 (m, 2H), 7.45-7.51 (m, 2H), 7.78 (d, J=8.1 Hz, 1H), 7.88 (d, J=2.7 Hz, 1H), 8.12 (dd, J=2.5, 1.5 Hz, 1H), 8.41 (d, J=1.4 Hz, 1H), 8.95 (q, J=4.6 Hz, 1H), 11.62 (s, 1H).); MS (ESI) m/z 541 (M+H)⁺.

### Example 39

### 4-(3-amino-4-{[(2S)-1,1,1-trifluoropropan-2-yl]oxy}-1H-indazol-6-yl)-N-methyl-2-(4-phenylpiperazin-1-yl)benzamide

### Step 1

### 4-(3-amino-4-{[(2S)-1,1,1-trifluoropropan-2-yl]oxy}-1H-indazol-6-yl)-2-fluoro-N-methylbenzamide

To a suspension of 6-bromo-4-{[(2S)-1,1,1-trifluoropropan-2-yl]oxy}-1H-indazol-3-amine (Intermediate 8, 0.992 g, 3.06 mmol) and 3-fluoro-4-(methylcarbamoyl)phenylboronic acid (0.892 g, 4.53 mmol) in dimethoxyethane:ethanol (10 mL, 1:1) was added 1 M potassium carbonate (5 mL). The reaction mixture was purged with nitrogen and bis(triphenylphosphine)palladium(II) dichloride (0.063 g, 0.09 mmol) was added. The reaction mixture was then heated in a microwave reactor (CEM Discover®, ≦300 W) at 160° C. for 20 minutes and then concentrated. The concentrate was partitioned in water/dichloromethane (3×50 mL). The combined organic layers were concentrated and purified chromatographically on silica gel eluting with a 0-10% ethanol/ethyl acetate gradient to obtain the titled compound. ¹H NMR (500 MHz, DMSO-d₆) δ ppm 1.54 (d, J=6.3 Hz, 3H), 2.81 (d, J=4.3 Hz, 3H), 4.96 (bs, 2H), 5.68 (dq, J=12.7, 6.3 Hz, 1H), 6.92 (s, 1H), 7.20 (s, 1H), 7.65-7.76 (m, 3H), 8.22-8.28 (m, 1H), 11.72 (s, 1H); MS (ESI) m/z 539 (M+H)⁺; [α]²⁰_{D}=+15° (c 1.01, CH₃OH).

### Step 2

### 4-(3-amino-4-{[(2S)-1,1,1-trifluoropropan-2-yl]oxy}-1H-indazol-6-yl)-N-methyl-2-(4-phenylpiperazin-1-yl)benzamide

A solution of the product from Step 1 (48.4 mg, 0.122 mmol) and 1-phenylpiperazine (82.6 g, 0.5095 mmol) in dimethyl sulfoxide (imp was heated at 150° C. for 48 hours. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.54 (d, J=6.1 Hz, 3H), 2.86 (d, J=4.6 Hz, 3H), 3.17-3.20 (m, 4H), 3.36-3.38 (m, 4H), 4.94 (s, 2H), 5.62-5.70 (m, 1H), 6.79-6.85 (m, 2H), 7.01-7.04 (m, 2H), 7.15 (s, 1H), 7.23-7.28 (m, 2H), 7.48-7.51 (m, 2H), 7.79 (d, J=7.8 Hz, 1H), 8.98 (q, J=4.6 Hz, 1H), 11.65 (s, 1H); MS(ESI) m/z 539 (M+H)⁺.

### Example 40

### 4-(3-amino-4-{[(2S)-1,1,1-trifluoropropan-2-yl]oxy}-1H-indazol-6-yl)-2-[4-(3,3-dimethylbutyl)piperazin-1-yl]-N-methylbenzamide

The titled compound was prepared as described in Example 39 except 1-(3,3-dimethylbutyl)piperazine was substituted for 1-phenylpiperazine. ¹H NMR (300 MHz, DMSO-d₆) δ ppm 0.91 (s, 9H), 1.41-1.36 (m, 2H), 1.54 (d, J=6.4 Hz, 3H), 2.33-2.39 (m, 3H), 2.54-2.59 (m, 4H), 2.86 (d, J=4.8 Hz, 3H), 2.99-3.03 (m, 4H), 4.93 (s, 2H), 5.61-5.69 (m, 1H), 6.83 (s, 1H), 7.12 (s, 1H), 7.43-7.49 (m, 2H), 7.79 (d, 18.1 Hz, 1H), 9.10 (q, J=4.2 Hz, 1H), 11.61 (s, 1H); MS(ESI) m/z 547 (M+H)⁺.

### Example 41

### 4-(3-amino-4-{[(2S)-1,1,1-trifluoropropan-2-yl]oxy}-1H-indazol-6-yl)-N-methyl-2-[4-(pyridin-3-yl)piperazin-1-yl]benzamide

The titled compound was prepared as described in Example 39 except 1-(pyridin-3-yl)piperazine was substituted for 1-phenylpiperazine. ¹H NMR (300 MHz, DMSO-d₆) δ ppm 1.54 (d, J=6.1 Hz, 3H), 2.85 (d, J=4.6 Hz, 3H), 3.18-3.21 (m, 4H), 3.41-3.44 (m, 4H), 4.94 (s, 2H), 5.62-5.70 (m, 1H), 6.85 (s, 1H), 7.15 (s, 1H), 7.25 (dd, J=8.3, 4.4 Hz, 1H), 7.41 (ddd, J=8.4, 2.9, 1.4 Hz, 1H), 7.46-7.51 (m, 2H), 7.78 (d, J=7.9 Hz, 1H), 8.03 (dd, J=4.4, 1.2 Hz, 1H), 8.38 (d, J=2.8 Hz, 1H), 8.93 (q, J=4.6 Hz, 1H), 11.63 (s, 1H); MS(ESI) m/z 540 (M+H)⁺.

### Example 42

### 4-(3-amino-4-{[(2S)-1,1,1-trifluoropropan-2-yl]oxy}-1H-indazol-6-yl)-2-(4-cyclobutylpiperazin-1-yl)-N-methylbenzamide

The titled compound was prepared as described in Example 39 except 1-cyclobutylpiperazine was substituted for 1-phenylpiperazine. ¹H NMR (300 MHz, DMSO-d₆) δ ppm 1.54 (d, J=6.4 Hz, 3H), 1.63-1.70 (m, 2H), 1.75-1.84 (m, 2H), 1.97-2.05 (m, 2H), 2.43-2.46 (m, 4H), 2.85 (d, J=4.8 Hz, 3H), 3.00-3.03 (m, 4H), 4.93 (s, 2H), 5.62-5.69 (m, 1H), 6.83 (s, 1H), 7.12 (s, 1H), 7.45-7.49 (m, 2H), 7.79 (d, J=7.8 Hz, 1H), 9.12 (q, J=4.3 Hz, 1H), 11.62 (s, 1H); MS(ESI) m/z 517 (M+H)⁺.

### Example 43

### 4-(3-amino-4-{[(2S)-1,1,1-trifluoropropan-2-yl]oxy}-1H-indazol-6-yl)-N-methyl-2-[4-(pyrimidin-2-yl)piperazin-1-yl]benzamide

The titled compound was prepared as described in Example 39 except 2-(piperazin-1-yl)pyrimidine was substituted for 1-phenylpiperazine. ¹H NMR (300 MHz, DMSO-d₆) δ ppm 1.53 (d, J=6.4 Hz, 3H), 2.87 (d, J=4.4 Hz, 3H), 3.13-3.16 (m, 4H), 3.77-3.80 (m, 4H), 4.94 (s, 2H), 5.61-5.69 (m, 1H), 6.84 (s, 1H), 7.14 (s, 1H), 7.45-7.51 (m, 2H), 7.78 (d, J=7.9 Hz, 1H), 7.88 (d, J=2.8 Hz, 1H), 8.13 (dd, J=2.4, 1.6 Hz, 1H), 8.41 (d, J=1.2 Hz, 1H), 8.96 (q, J=4.6 Hz, 1H), 11.62 (s, 1H); MS(ESI) m/z 541 (M+H)⁺.

### Example 44

### 4-(3-amino-4-{[(2S)-1,1,1-trifluoropropan-2-yl]oxy}-1H-indazol-6-yl)-2-(4-cyclopentylpiperazin-1-yl)-N-methylbenzamide

The titled compound was prepared as described in Example 39 except 1-cyclopentylpiperazine was substituted for 1-phenylpiperazine. ¹H NMR (300 MHz, DMSO-d₆) δ ppm 1.31-1.42 (m, 2H), 1.49-1.68 (m, 7H), 1.79-1.87 (m, 2H), 2.54-2.67 (m, 5H), 2.85 (d, J=4.8 Hz, 3H), 3.01-3.04 (m, 4H), 4.93 (s, 2H), 5.62-5.70 (m, 1H), 6.83 (s, 1H), 7.1 (s, 1H), 7.44-7.49 (m, 2H), 7.80 (d, J=7.8 Hz, 1H), 9.16 (q, J=4.6 Hz, 1H), 11.62 (s, 1H); MS(ESI) m/z 531 (M+H)⁺.

### Example 45

### 4-(3-amino-4-{[(2S)-1,1,1-trifluoropropan-2-yl]oxy}-1H-indazol-6-yl)-2-[4-(2,2-dimethylpropanoyl)piperazin-1-yl]-N-methylbenzamide

The titled compound was prepared as described in Example 39 except tert-butyl piperazine-1-carboxylate was substituted for 1-phenylpiperazine. ¹H NMR (300 MHz, DMSO-d₆) δ ppm 1.23 (s, 9H), 1.53 (d, J=6.4 Hz, 3H), 2.87 (d, J=4.8 Hz, 3H), 2.98-3.01 (m, 4H), 3.74-3.77 (m, 4H), 4.94 (s, 2H), 5.60-5.68 (m, 1H), 6.83 (s, 1H), 7.13 (s, 1H), 7.40 (d, J=1.6 Hz, 1H), 7.48 (dd, J=7.9, 1.6 Hz, 1H), 7.75 (d, J=7.9 Hz, 1H), 8.90 (q, J=4.6 Hz, 1H), 11.62 (s, 1H); MS(ESI) m/z 547 (M+H)⁺.

### Example 46

### 4-(3-amino-4-{[(2S)-1,1,1-trifluoropropan-2-yl]oxy}-1H-indazol-6-yl)-N-methyl-2-[4-(pyrazin-2-yl)piperazin-1-yl]benzamide

The titled compound was prepared as described in Example 39 except 2-(piperazin-1-yl)pyrazine was substituted for 1-phenylpiperazine. ¹H NMR (300 MHz, DMSO-d₆) δ ppm 1.53 (d, J=6.4 Hz, 3H), 2.87 (d, J=4.4 Hz, 3H), 3.13-3.16 (m, 4H), 3.77-3.80 (m, 4H), 4.94 (s, 2H), 5.61-5.69 (m, 1H), 6.84 (s, 1H), 7.14 (s, 1H), 7.45-7.51 (m, 2H), 7.78 (d, J=7.9 Hz, 1H), 7.88 (d, J=2.8 Hz, 1H), 8.13 (dd, J=2.4, 1.6 Hz, 1H), 8.41 (d, J=1.2 Hz, 1H), 8.96 (q, J=4.6 Hz, 1H), 11.62 (s, 1H); MS(ESI) m/z 541 (M+H)⁺.

### Example 47

### 4-(3-amino-4-{[(2S)-1,1,1-trifluoropropan-2-yl]oxy}-1H-indazol-6-yl)-2-(4-cyclohexylpiperazin-1-yl)-N-methylbenzamide

The titled compound was prepared as described in Example 39 except 1-cyclohexylpiperazine was substituted for 1-phenylpiperazine. ¹H NMR (300 MHz, DMSO-d₆) δ ppm 1.10-1.26 (m, 5H), 1.52-1.61 (m, 4H), 1.76-1.83 (m, 4H), 2.25-2.32 (m, 1H), 2.67-2.70 (m, 4H), 2.85 (d, J=4.8 Hz, 3H), 2.99-3.02 (m, 4H), 4.94 (s, 2H), 5.62-5.71 (m, 1H), 6.83 (s, 1H), 7.12 (s, 1H), 7.44-7.48 (m, 2H), 7.79 (d, J=7.9 Hz, 1H), 9.13 (q, J=4.2 Hz, 1H), 11.62 (s, 1H); [α]²⁰_{D}=+14° (c 0.96, CH₃OH).

### Example 48

### 4-(3-amino-4-{[(2R)-1,1,1-trifluoropropan-2-yl]oxy}-1H-indazol-6-yl)-N-methyl-2-(4-phenylpiperazin-1-yl)benzamide

### Step 1

### 4-(3-amino-4-{[(2R)-1,1,1-trifluoropropan-2-yl]oxy}-1H-indazol-6-yl)-2-fluoro-N-methylbenzamide(

To a suspension of 6-bromo-4-{[(2R)-1,1,1-trifluoropropan-2-yl]oxy}-1H-indazol-3-amine (Intermediate 7, 1.11 g, 3.42 mmol) and 3-fluoro-4-(methylcarbamoyl)phenylboronic acid (1.01 g, 5.08 mmol) in dimethoxyethane:ethanol:water (5 mL:5 mL:1 mL) was added potassium carbonate (844 mg, 6.11 mmol). The reaction mixture was purged with nitrogen and bis(triphenylphosphine)palladium(II) dichloride (0.078 g, 0.11 mmol) was added. The reaction mixture was then heated in a microwave reactor (CEM Discover®, ≦300 W) at 160° C. for 20 minutes and then concentrated. The concentrate was partitioned in water/dichloromethane (3×50 mL). The combined organic layers were concentrated and purified chromatographically on silica gel eluting with a 0-10% ethanol/ethyl acetate gradient to obtain the titled compound. ¹H NMR (500 MHz, DMSO-d₆) δ ppm 1.54 (d, J=6.3 Hz, 3H), 2.81 (d, J=4.3 Hz, 3H), 4.96 (bs, 2H), 5.68 (dq, J=12.7, 6.3 Hz, 1H), 6.92 (s, 1H), 7.20 (s, 1H), 7.65-7.76 (m, 3H), 8.22-8.28 (m, 1H), 11.72 (s, 1H); MS (ESI) m/z 539 (M+H)⁺; [α]²⁰_{D}=−12° (c 1.01, CH₃OH)

### Step 2

### 4-(3-amino-4-{[(2R)-1,1,1-trifluoropropan-2-yl]oxy}-1H-indazol-6-yl)-N-methyl-2-(4-phenylpiperazin-1-yl)benzamide

A solution of the product from Step 1 (48.4 mg, 0.122 mmol) and 1-phenylpiperazine (82.6 g, 0.5095 mmol) in dimethyl sulfoxide (1 mL) was heated at 150° C. for 48 hours. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.54 (d, J=6.1 Hz, 3H), 2.86 (d, J=4.6 Hz, 3H), 3.17-3.20 (m, 4H), 3.36-3.38 (m, 4H), 4.94 (s, 2H), 5.62-5.70 (m, 1H), 6.79-6.85 (m, 2H), 7.01-7.04 (m, 2H), 7.15 (s, 1H), 7.23-7.28 (m, 2H), 7.48-7.51 (m, 2H), 7.79 (d, J=7.8 Hz, 1H), 8.98 (q, J=4.6 Hz, 1H), 11.65 (s, 1H); MS(ESI) m/z 539 (M+H)⁺.

### Example 49

### 4-(3-amino-4-{[(2R)-1,1,1-trifluoropropan-2-yl]oxy}-1H-indazol-6-yl)-N-methyl-2-[4-(pyridin-3-yl)piperazin-1-yl]benzamide

The titled compound was prepared as described in Example 48 except 1-(pyridin-3-yl)piperazine was substituted for 1-phenylpiperazine. ¹H NMR (300 MHz, DMSO-d₆) δ ppm 1.54 (d, J=6.1 Hz, 3H), 2.85 (d, J=4.6 Hz, 3H), 3.18-3.21 (m, 4H), 3.41-3.44 (m, 4H), 4.94 (s, 2H), 5.62-5.70 (m, 1H), 6.85 (s, 1H), 7.15 (s, 1H), 7.25 (dd, J=8.3, 4.4 Hz, 1H), 7.41 (ddd, J=8.4, 2.9, 1.4 Hz, 1H), 7.46-7.51 (m, 2H), 7.78 (d, J=7.9 Hz, 1H), 8.03 (dd, J=4.4, 1.2 Hz, 1H), 8.38 (d, J=2.8 Hz, 1H), 8.93 (q, J=4.6 Hz, 1H), 11.63 (s, 1H); MS(ESI) m/z 540 (M+H)⁺.

### Example 50

### 4-(3-amino-4-{[(2R)-1,1,1-trifluoropropan-2-yl]oxy}-1H-indazol-6-yl)-2-(4-cyclobutylpiperazin-1-yl)-N-methylbenzamide

The titled compound was prepared as described in Example 48 except 1-cyclobutylpiperazine was substituted for 1-phenylpiperazine. ¹H NMR (300 MHz, DMSO-d₆) δ ppm 1.54 (d, J=6.4 Hz, 3H), 1.63-1.70 (m, 2H), 1.75-1.84 (m, 2H), 1.97-2.05 (m, 2H), 2.43-2.46 (m, 4H), 2.85 (d, J=4.8 Hz, 3H), 3.00-3.03 (m, 4H), 4.93 (s, 2H), 5.62-5.69 (m, 1H), 6.83 (s, 1H), 7.12 (s, 1H), 7.45-7.49 (m, 2H), 7.79 (d, J=7.8 Hz, 1H), 9.12 (q, J=4.3 Hz, 1H), 11.62 (s, 1H); MS(ESI) m/z 517 (M+H)⁺.

### Example 51

### 4-(3-amino-4-{[(2R)-1,1,1-trifluoropropan-2-yl]oxy}-1H-indazol-6-yl)-2-[4-(3,3-dimethylbutyl)piperazin-1-yl]-N-methylbenzamide

The titled compound was prepared as described in Example 48 except 1-(3,3-dimethylbutyl)piperazine was substituted for 1-phenylpiperazine. ¹H NMR (300 MHz, DMSO-d₆) δ ppm 0.91 (s, 9H), 1.41-1.36 (m, 2H), 1.54 (d, J=6.4 Hz, 3H), 2.33-2.39 (m, 3H), 2.54-2.59 (m, 4H), 2.86 (d, J=4.8 Hz, 3H), 2.99-3.03 (m, 4H), 4.93 (s, 2H), 5.61-5.69 (m, 1H), 6.83 (s, 1H), 7.12 (s, 1H), 7.43-7.49 (m, 2H), 7.79 (d, 18.1 Hz, 1H), 9.10 (q, J=4.2 Hz, 1H), 11.61 (s, 1H); MS(ESI) m/z 547 (M+H)⁺.

### Example 52

### 4-(3-amino-4-{[(2R)-1,1,1-trifluoropropan-2-yl]oxy}-1H-indazol-6-yl)-N-methyl-2-[4-(pyrimidin-2-yl)piperazin-1-yl]benzamide

The titled compound was prepared as described in Example 48 except 2-(piperazin-1-yl)pyrimidine was substituted for 1-phenylpiperazine. ¹H NMR (300 MHz, DMSO-d₆) δ ppm 1.53 (d, J=6.4 Hz, 3H), 2.87 (d, J=4.4 Hz, 3H), 3.13-3.16 (m, 4H), 3.77-3.80 (m, 4H), 4.94 (s, 2H), 5.61-5.69 (m, 1H), 6.84 (s, 1H), 7.14 (s, 1H), 7.45-7.51 (m, 2H), 7.78 (d, J=7.9 Hz, 1H), 7.88 (d, J=2.8 Hz, 1H), 8.13 (dd, J=2.4, 1.6 Hz, 1H), 8.41 (d, J=1.2 Hz, 1H), 8.96 (q, J=4.6 Hz, 1H), 11.62 (s, 1H); MS(ESI) m/z 541 (M+H)⁺.

### Example 53

### 4-(3-amino-4-{[(2R)-1,1,1-trifluoropropan-2-yl]oxy}-1H-indazol-6-yl)-2-(4-cyclopentylpiperazin-1-yl)-N-methylbenzamide

The titled compound was prepared as described in Example 48 except 1-cyclopentylpiperazine was substituted for 1-phenylpiperazine. ¹H NMR (300 MHz, DMSO-d₆) δ ppm 1.31-1.42 (m, 2H), 1.49-1.68 (m, 7H), 1.79-1.87 (m, 2H), 2.54-2.67 (m, 5H), 2.85 (d, J=4.8 Hz, 3H), 3.01-3.04 (m, 4H), 4.93 (s, 2H), 5.62-5.70 (m, 1H), 6.83 (s, 1H), 7.1 (s, 1H), 7.44-7.49 (m, 2H), 7.80 (d, J=7.8 Hz, 1H), 9.16 (q, J=4.6 Hz, 1H), 11.62 (s, 1H); MS(ESI) m/z 531 (M+H)⁺.

### Example 54

### 4-(3-amino-4-{[(2R)-1,1,1-trifluoropropan-2-yl]oxy}-1H-indazol-6-yl)-2-[4-(2,2-dimethylpropanoyl)piperazin-1-yl]-N-methylbenzamide

The titled compound was prepared as described in Example 48 except tert-butyl piperazine-1-carboxylate was substituted for 1-phenylpiperazine. ¹H NMR (300 MHz, DMSO-d₆) δ ppm 1.23 (s, 9H), 1.53 (d, J=6.4 Hz, 3H), 2.87 (d, J=4.8 Hz, 3H), 2.98-3.01 (m, 4H), 3.74-3.77 (m, 4H), 4.94 (s, 2H), 5.60-5.68 (m, 1H), 6.83 (s, 1H), 7.13 (s, 1H), 7.40 (d, J=1.6 Hz, 1H), 7.48 (dd, J=7.9, 1.6 Hz, 1H), 7.75 (d, J=7.9 Hz, 1H), 8.90 (q, J=4.6 Hz, 1H), 11.62 (s, 1H); MS(ESI) m/z 547 (M+H)⁺.

### Example 55

### 4-(3-amino-4-{[(2R)-1,1,1-trifluoropropan-2-yl]oxy}-1H-indazol-6-yl)-N-methyl-2-[4-(pyrazin-2-yl)piperazin-1-yl]benzamide

The titled compound was prepared as described in Example 48 except 2-(piperazin-1-yl)pyrazine was substituted for 1-phenylpiperazine. ¹H NMR (300 MHz, DMSO-d₆) δ ppm 1.53 (d, J=6.4 Hz, 3H), 2.87 (d, J=4.4 Hz, 3H), 3.13-3.16 (m, 4H), 3.77-3.80 (m, 4H), 4.94 (s, 2H), 5.61-5.69 (m, 1H), 6.84 (s, 1H), 7.14 (s, 1H), 7.45-7.51 (m, 2H), 7.78 (d, J=7.9 Hz, 1H), 7.88 (d, J=2.8 Hz, 1H), 8.13 (dd, J=2.4, 1.6 Hz, 1H), 8.41 (d, J=1.2 Hz, 1H), 8.96 (q, J=4.6 Hz, 1H), 11.62 (s, 1H); MS(ESI) m/z 541 (M+H)⁺.

### Example 56

### 4-(3-amino-4-{[(2R)-1,1,1-trifluoropropan-2-yl]oxy}-1H-indazol-6-yl)-2-(4-cyclohexylpiperazin-1-yl)-N-methylbenzamide

The titled compound was prepared as described in Example 48 except 1-cyclohexylpiperazine was substituted for 1-phenylpiperazine. ¹H NMR (300 MHz, DMSO-d₆) δ ppm 1.10-1.26 (m, 5H), 1.52-1.61 (m, 4H), 1.76-1.83 (m, 4H), 2.25-2.32 (m, 1H), 2.67-2.70 (m, 4H), 2.85 (d, J=4.8 Hz, 3H), 2.99-3.02 (m, 4H), 4.94 (s, 2H), 5.62-5.71 (m, 1H), 6.83 (s, 1H), 7.12 (s, 1H), 7.44-7.48 (m, 2H), 7.79 (d, J=7.9 Hz, 1H), 9.13 (q, J=4.2 Hz, 1H), 11.62 (s, 1H); [α]²⁰_{D}=−13.2° (c 0.92, CH₃OH).

### Example 57

### 4-(3-amino-4-{[(2R)-1,1,1-trifluoropropan-2-yl]oxy}-1H-indazol-6-yl)-N-methyl-2-(octahydro-2H-pyrido[1,2-a]pyrazin-2-yl)benzamide

The titled compound was prepared as described in Example 48 except octahydro-1H-pyrido[1,2-a]pyrazine was substituted for 1-phenylpiperazine. ¹H NMR (300 MHz, DMSO-d₆) δ ppm 1.08-1.39 (m, 2H), 1.37-1.57 (m, 5H), 1.56-1.79 (m, 2H), 1.95-2.27 (m, 2H), 2.32 (td, J=11.0, 3.6 Hz, 1H), 2.65 (t, J=10.5 Hz, 1H), 2.71-2.91 (m, 4H), 2.90-3.13 (m, 3H), 4.93 (s, 2H), 5.65 (p, J=6.4 Hz, 1H), 6.83 (s, 1H), 7.12 (s, 1H), 7.42 (d, J=1.8 Hz, 1H), 7.47 (dd, J=8.1, 1.6 Hz, 1H), 7.79 (d, J=8.0 Hz, 1H), 9.06-9.17 (m, 1H); MS(ESI) m/z 517 (M+H)⁺.

### Example 58

### 4-(3-amino-4-{[(2R)-1,1,1-trifluoropropan-2-yl]oxy}-1H-indazol-6-yl)-2-(2-isobutylmorpholin-4-yl)-N-methylbenzamide

The titled compound was prepared as described in Example 48 except 2-isobutylmorpholine was substituted for 1-phenylpiperazine. ¹H NMR (300 MHz, DMSO-d₆) δ ppm 0.91 (d, J=6.7 Hz, 6H), 1.09-1.29 (m, 2H), 1.35-1.51 (m, 1H), 1.54 (d, J=6.3 Hz, 3H), 1.66-1.81 (m, 1H), 2.55-2.63 (m, 1H), 2.85 (d, J=4.6 Hz, 3H), 2.89-3.22 (m, 3H), 3.62-3.79 (m, 2H), 3.88-4.08 (m, 1H), 4.93 (bs, 2H), 5.58-5.71 (m, 1H), 6.83 (s, 1H), 7.13 (d, J=0.9 Hz, 1H), 7.37 (d, J=1.7 Hz, 1H), 7.46 (dd, J=8.0, 1.6 Hz, 1H), 7.72 (d, J=8.0 Hz, 1H), 8.84 (d, J=4.7 Hz, 1H); MS(ESI) m/z 520 (M+H)⁺.

### Example 59

### 4-(3-amino-4-{[(2R)-1,1,1-trifluoropropan-2-yl]oxy}-1H-indazol-6-yl)-N-methyl-2-[4-(morpholin-4-yl)piperidin-1-yl]benzamide

The titled compound was prepared as described in Example 48 except 4-(piperidin-4-yl)morpholine was substituted for 1-phenylpiperazine. ¹H NMR (300 MHz, DMSO-d₆) δ ppm 1.47-1.71 (m, 4H), 1.87-2.04 (m, 2H), 2.05-2.37 (m, 2H), 2.71-2.94 (m, 5H), 3.21-3.5 (m, 4H), 3.51-3.64 (m, 4H), 4.94 (s, 2H), 5.58-5.82 (m, 1H), 6.82 (s, 1H), 7.11 (d, J=0.9 Hz, 1H), 7.35-7.62 (m, 2H), 7.79 (d, J=8.0 Hz, 1H), 9.01-9.14 (d, J=4.7 Hz, 1H), 11.61 (s, 1H); MS(ESI) m/z 547 (M+H)⁺.

### Example 60

### 4-(3-amino-4-{[(2R)-1,1,1-trifluoropropan-2-yl]oxy}-1H-indazol-6-yl)-2-(4-hydroxy-4-phenylpiperidin-1-yl)-N-methylbenzamide

The titled compound was prepared as described in Example 48 except 4-phenylpiperidin-4-ol was substituted for 1-phenylpiperazine. ¹H NMR (300 MHz, DMSO-d₆) δ ppm 1.54 (d, J=6.1 Hz, 3H), 1.76-1.80 (m, 2H), 2.15-2.26 (m, 3H), 2.89 (d, J=4.6 Hz, 3H), 3.05-3.09 (m, 2H), 3.23-3.26 (m, 2H), 4.94 (s, 2H), 5.02 (s, 1H), 5.63-5.71 (m, 1H), 6.86 (s, 1H), 7.13 (s, 1H), 7.22-7.27 (m, 1H), 7.34-7.39 (m, 2H), 7.45-7.47 (m, 2H), 7.57-7.60 (m, 2H), 7.79 (d, J=8.5 Hz, 1H), 9.12 (q, J=4.2 Hz, 1H), 11.59 (s, 1H); MS(ESI) m/z 554 (M+H)⁺.

### Example 61

### 4-(3-amino-4-{[(2R)-1,1,1-trifluoropropan-2-yl]oxy}-1H-indazol-6-yl)-2-(4-cyclohexylpiperidin-1-yl)-N-methylbenzamide

The titled compound was prepared as described in Example 48 except 4-cyclohexylpiperidine was substituted for 1-phenylpiperazine. ¹H NMR (300 MHz, DMSO-d₆) δ ppm 0.99 (m, 2H), 1.17 (m, 4H), 1.31-1.49 (m, 2H), 1.53 (d, 3H),), 1.62-1.82 (m, 8H) 2.68-2.85 (m, 2H), 2.86 (d, J=4.7 Hz, 3H), 3.25 (m, 2H) 4.93 (s, 2H), 5.60-5.68 (m, 1H), 6.82 (s, 1H), 7.10 (s, 1H), 7.41-7.47 (m, 2H), 7.82 (d, J=8.1 Hz, 1H), 9.23 (q, J=4.2 Hz, 1H), 11.61 (s, 1H); MS(ESI) m/z 544 (M+H)⁺.

### Example 62

### 4-{3-amino-4-[(1-methylpiperidin-4-yl)oxy]-1H-indazol-6-yl}-N-cyclopropyl-2-(4-phenylpiperazin-1-yl)benzamide

The titled compound was prepared as the acetate salt using the procedures described for Example 23 substituting 6-bromo-4-[(1-methylpiperidin-4-yl)oxy]-1H-indazol-3-amine (Intermediate 9) for 6-bromo-4-(tetrahydro-2H-pyran-4-yloxy)-1H-indazol-3-amine (Intermediate 1). ¹H NMR (300 MHz, DMSO-d₆) δ ppm 0.50-0.57 (m, 1H), 0.73 (td, J=7.1, 5.0 Hz, 1H), 1.83 (d, J=5.0 Hz, 1H), 1.92-2.07 (m, 1H), 2.08 (s, 3H), 2.19 (s, 3H), 2.22-2.41 (m, 1H), 2.52-2.64 (m, 1H), 2.79-2.94 (m, 1H), 3.13-3.20 (m, 2H), 4.75 (s, 1H), 5.00 (bs, 1H), 6.61 (s, 1H), 6.83 (t, J=7.3 Hz, 1H), 7.02 (d, J=8.0 Hz, 2H), 7.20-7.33 (m, 1H), 7.41-7.48 (m, 1H), 7.76 (d, J=8.5 Hz, 1H), 9.21 (d, J=4.3 Hz, 1H), 11.47 (s, 1H); MS (ESI) m/z 566 (M+H)⁺.

### Example 63

### 4-[3-amino-4-(piperidin-4-yloxy)-1H-indazol-6-yl]-2-(4-phenylpiperazin-1-yl)benzamide

### Step 1

### tert-butyl 4-({3-amino-6-[4-carbamoyl-3-(4-phenylpiperazin-1-yl)phenyl]-1H-indazol-4-yl}oxy)piperidine-1-carboxylate

The titled compound was prepared as described for Example 1 replacing 6-bromo-4-(tetrahydro-2H-pyran-4-yloxy)-1H-indazol-3-amine (Intermediate 1) with tert-butyl 4-[(3-amino-6-bromo-1H-indazol-4-yl)oxy]piperidine-1-carboxylate (Intermediate 4) to obtain the titled compound; MS (ESI) m/z 612 (M+H)⁺.

### Step 2

### 4-[3-amino-4-(piperidin-4-yloxy)-1H-indazol-6-yl]-2-(4-phenylpiperazin-1-yl)benzamide

To a solution of the product obtained in Step 1 (0.12 g, 0.2 mmol) in dichloromethane (5 mL) was added trifluoroacetic acid (1 mL), and the mixture was stirred at room temperature for 1 hour. The mixture was then concentrated and purified chromatographically eluting with 10% CH₃OH/CH₂Cl₂/1% concentrated NH₄OH) to yield the titled compound. ¹H NMR (300 MHz, DMSO-d₆) δ ppm 1.93-2.14 (m, 2H), 2.66-2.88 (m, 2H), 2.95-3.15 (m, 2H), 3.19 (bs, 4H), 3.3 (bs, 4H), 4.79-4.9 (m, 1H), 5.02 (s, 2H), 6.63 (s, 1H), 6.82 (t, J=7.3 Hz, 1H), 6.98-7.05 (m, 3H), 7.17-7.32 (m, 3H), 7.40-7.47 (m, 2H), 7.56 (s, 1H), 7.82 (d, J=8.1 Hz, 1H), 8.34-8.73 (m, 1H), 11.29-11.85 (m, 1H); MS (ESI) m/z 512 (M+H)⁺.

### Example 64

### 4-{4-[(1-acetylpiperidin-4-yl)oxy]-3-amino-1H-indazol-6-yl}-2-(4-phenylpiperazin-1-yl)benzamide

Acetic anhydride (0.018 ml, 0.19 mmol) and triethylamine (0.075 ml, 0.54 mmol) were added to a solution of 4-[3-amino-4-(piperidin-4-yloxy)-1H-indazol-6-yl]-2-(4-phenylpiperazin-1-yl)benzamide (Example 63, 0.09 g, 0.18 mmol) in dichloromethane (10 mL), and the mixture was then stirred at ambient temperature for 1 hour. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.65-1.89 (m, 2H), 1.97-2.08 (m, 2H), 2.03 (s, 3H), 3.19-3.24 (m, 4H), 3.32-3.39 (m, 4H), 3.38-3.51 (m, 2H), 3.62-3.82 (m, 2H), 4.92-4.97 (m, 1H), 4.99 (s, 2H), 6.69 (s, 1H), 6.82 (t, J=7.2 Hz, 1H), 7.02 (m, 3H), 7.17-7.32 (m, 2H), 7.45 (d, J=9.4 Hz, 2H), 7.56 (d, J=2.2 Hz, 1H), 7.82 (d, J=7.9 Hz, 1H), 8.49 (s, 1H), 11.49 (s, 1H); MS (ESI) m/z 554 (M+H)⁺.

### Example 65

### 4-{3-amino-4-[(3S)-tetrahydrofuran-3-yloxy]-1H-indazol-6-yl}-2-(4-cyclopropylpiperazin-1-yl)benzamide

The titled compound was prepared using the procedures described in Example 1 substituting 6-bromo-4-[(3S)-tetrahydrofuran-3-yloxy]-1H-indazol-3-amine (Intermediate 5) for 6-bromo-4-(tetrahydro-2H-pyran-4-yloxy)-1H-indazol-3-amine (Intermediate 1) in Step 1 and substituting 1-cyclopropylpiperazine for 1-phenylpiperazine in Step 2. ¹H NMR (300 MHz, DMSO-d₆) δ ppm 0.25-0.40 (m, 2H), 0.41-0.52 (m, 2H), 1.65-1.76 (m, 1H), 2.08-2.25 (m, 1H), 2.22-2.37 (m, 1H), 2.68-2.77 (m, 4H), 2.94-3.06 (m, 4H), 3.80 (td, J=8.2, 4.7 Hz, 1H), 3.90 (t, J=7.7 Hz, 1H), 3.96 (d, J=3.2 Hz, 2H), 4.98 (bs, 2H), 5.28-5.35 (m, 1H), 6.52 (s, 1H), 7.02 (d, J=0.9 Hz, 1H), 7.37-7.47 (m, 2H), 7.54-7.60 (m, 1H), 7.83 (d, J=8.0 Hz, 1H), 8.64 (d, J=2.4 Hz, 1H), 11.49 (bs, 1H); MS (ESI) m/z 463 (M+H)⁺.

### Example 66

### 4-{3-amino-4-[(3S)-tetrahydrofuran-3-yloxy]-1H-indazol-6-yl}-2-(4-phenylpiperazin-1-yl)benzamide

The titled compound was prepared using the procedures described in Example 1 substituting 6-bromo-4-[(3S)-tetrahydrofuran-3-yloxy]-1H-indazol-3-amine (Intermediate 5) for 6-bromo-4-(tetrahydro-2H-pyran-4-yloxy)-1H-indazol-3-amine (Intermediate 1) in Step 1. ¹H NMR (300 MHz, DMSO-d₆) δ ppm 2.11-2.24 (m, 1H), 2.24-2.37 (m, 1H), 3.17-3.26 (m, 4H), 3.32-3.36 (m, 4H), 3.80 (td, J=8.2, 4.7 Hz, 1H), 3.86-3.96 (m, 1H), 3.97 (d, J=3.3 Hz, 2H), 4.99 (bs, 2H), 5.28-5.37 (m, 1H), 6.55 (s, 1H), 6.82 (t, J=7.3 Hz, 1H), 7.01 (d, J=7.9 Hz, 2H), 7.06 (s, 1H), 7.25 (dd, J=8.5, 7.1 Hz, 2H), 7.35-7.52 (m, 2H), 7.54 (s, 1H), 7.82 (d, J=8.4 Hz, 1H), 8.51 (d, J=2.1 Hz, 1H), 11.50 (s, 1H); MS (ESI) m/z 499 (M+H)⁺.

### Example 67

### 4-{3-amino-4-[(3S)-tetrahydrofuran-3-yloxy]-1H-indazol-6-yl}-2-[4-(pyridin-3-yl)piperazin-1-yl]benzamide

The titled compound was prepared using the procedures described in Example 1 substituting 6-bromo-4-[(3S)-tetrahydrofuran-3-yloxy]-1H-indazol-3-amine (Intermediate 5) for 6-bromo-4-(tetrahydro-2H-pyran-4-yloxy)-1H-indazol-3-amine (Intermediate 1) in Step 1 and substituting 1-(pyridin-3-yl)piperazine for 1-phenylpiperazine in Step 2. ¹H NMR (300 MHz, DMSO-d₆) δ ppm 2.13-2.39 (m 2H), 3.15-3.25 (m, 4H), 3.36-3.46 (m, 4H), 3.80 (td, J=8.2, 4.6 Hz, m, 1H), 3.90 (t, J=7.7 Hz, 1H), 4.00 (d, J=10.6 Hz, 2H), 5.00 (bs, 2H), 5.32 (dt, J=5.5, 2.8 Hz, 1H), 6.54 (s, 1H), 7.05 (s, 1H), 7.25 (dd, J=8.4, 4.5 Hz, 1H), 7.36-7.49 (m, 2H), 7.55 (d, J=1.9 Hz, 1H), 7.81 (d, J=7.8 Hz, 1H), 8.03 (dd, J=4.5, 1.2 Hz, 1H), 8.38 (d, J=2.9 Hz, 1H), 8.45 (d, J=2.4 Hz, 1H), 11.51 (s, 1H).

### Example 68

### 4-{3-amino-4-[(3R)-tetrahydrofuran-3-yloxy]-1H-indazol-6-yl}-2-[4-(pyridin-3-yl)piperazin-1-yl]benzamide

The titled compound was prepared using the procedures described in Example 1 substituting 6-bromo-4-[(3R)-tetrahydrofuran-3-yloxy]-1H-indazol-3-amine (Intermediate 6) for 6-bromo-4-(tetrahydro-2H-pyran-4-yloxy)-1H-indazol-3-amine (Intermediate 1) in Step 1 and substituting 1-(pyridin-3-yl)piperazine for 1-phenylpiperazine in Step 2. ¹H NMR (300 MHz, DMSO-d₆) δ ppm 2.13-2.39 (m 2H), 3.15-3.25 (m, 4H), 3.36-3.46 (m, 4H), 3.80 (td, J=8.2, 4.6 Hz, m, 1H), 3.90 (t, J=7.7 Hz, 1H), 4.00 (d, J=10.6 Hz, 2H), 5.00 (bs, 2H), 5.32 (dt, J=5.5, 2.8 Hz, 1H), 6.54 (s, 1H), 7.05 (s, 1H), 7.25 (dd, J=8.4, 4.5 Hz, 1H), 7.36-7.49 (m, 2H), 7.55 (d, J=1.9 Hz, 1H), 7.81 (d, J=7.8 Hz, 1H), 8.03 (dd, J=4.5, 1.2 Hz, 1H), 8.38 (d, J=2.9 Hz, 1H), 8.45 (d, J=2.4 Hz, 1H), 11.51 (s, 1H).

### Example 69

### 4-[3-amino-4-(cyclopentyloxy)-1H-indazol-6-yl]-2-[4-(pyridin-3-yl)piperazin-1-yl]benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 1-(pyridin-3-yl)piperazine for 1-phenylpiperazine in Step 2. ¹H NMR (300 MHz, DMSO-d₆) δ ppm 1.61-2.03 (m, 8H), 3.17-3.23 (m, 4H), 3.38-3.43 (m, 4H), 4.94 (s, 2H), 5.09-5.14 (m, 1H), 6.56 (s, 1H), 7.01 (s, 1H), 7.25 (dd, J=8.5, 4.6 Hz, 1H), 7.38-7.46 (m, 3H), 7.54-7.55 (m, 1H), 7.80 (d, J=8.7 Hz, 1H), 8.03 (dd, J=4.6, 1.4 Hz, 1H), 8.38 (d, J=2.8 Hz, 1H), 8.44-8.45 (m, 1H), 11.46 (s, 1H); MS (ESI) m/z 498.3 (M+H)⁺.

### Example 70

### 4-[3-amino-4-(cyclopentyloxy)-1H-indazol-6-yl]-2-{4-[2-(morpholin-4-yl)ethyl]piperazin-1-yl}benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 4-(2-(piperazin-1-yl)ethyl)morpholine for 1-phenylpiperazine in Step 2. ¹H NMR (300 MHz, DMSO-d₆) δ ppm 1.61-2.02 (m, 8H), 2.38-2.41 (m, 4H), 2.44-2.46 (m, 4H), 2.58-2.65 (m, 4H), 3.01-3.04 (m, 4H), 3.55-3.58 (m, 4H), 4.93 (s, 2H), 5.08-5.14 (m, 1H), 6.53 (s, 1H), 6.98 (s, 1H), 7.39-7.42 (m, 2H), 7.52 (d, J=3.1 Hz, 1H), 7.82 (d, J=7.8 Hz, 1H), 8.57 (d, J=3.1 Hz, 1H), 11.46 (s, 1H); MS (ESI) m/z 534 (M+H)⁺.

### Example 71

### 4-[3-amino-4-(cyclopentyloxy)-1H-indazol-6-yl]-2-(4-phenylpiperazin-1-yl)benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1. ¹H NMR (300 MHz, DMSO-d₆) δ ppm 1.59-2.03 (m, 8H), 3.19-3.22 (m, 4H), 3.33-3.36 (m, 4H), 4.94 (s, 2H), 5.09-5.14 (m, 1H), 6.56 (s, 1H), 3.82 (t, J=7.3 Hz, 1H), 7.00-7.02 (m, 3H), 7.22-7.28 (m, 2H), 7.42-7.45 (m, 2H), 7.54 (d, J=2.4 Hz, 1H), 7.82 (d, J=8.5 Hz, 1H), 8.50 (d, J=2.4 Hz, 1H), 11.46 (s, 1H); MS (ESI) m/z 497 (M+H)⁺.

### Example 72

### 4-[3-amino-4-(cyclopentyloxy)-1H-indazol-6-yl]-2-[4-(4-fluorophenyl)piperazin-1-yl]benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 1-(4-fluorophenyl)piperazine for 1-phenylpiperazine in Step 2. ¹H NMR (300 MHz, DMSO-d₆) δ ppm 1.61-2.03 (m, 8H), 3.18-3.23 (m, 4H), 3.27-3.33 (m, 4H), 4.94 (s, 2H), 5.09-5.13 (m, 1H), 6.56 (s, 1H), 7.01-7.11 (m, 5H), 7.43-7.47 (m, 2H), 7.54-7.57 (m, 1H), 7.81 (d, J=8.7 Hz, 1H), 8.47-8.50 (m, 1H), 11.46 (s, 1H); MS (ESI) m/z 515 (M+H)⁺.

### Example 73

### tert-butyl 4-{5-[3-amino-4-(cyclopentyloxy)-1H-indazol-6-yl]-2-carbamoylphenyl}piperazine-1-carboxylate

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting tert-butyl piperazine-1-carboxylate for 1-phenylpiperazine in Step 2. ¹H NMR (300 MHz, DMSO-d₆) δ ppm 1.43 (s, 9H), 1.61-2.02 (m, 8H), 2.99-3.02 (m, 4H), 3.51-3.55 (m, 4H), 4.94 (s, 2H), 5.09-5.14 (m, 1H), 6.53 (s, 1H), 3.99 (s, 1H), 7.34-7.41 (m, 2H), 7.78 (d, J=7.9 Hz, 1H), 11.46 (s, 1H); MS (ESI) m/z 521 (M+H)⁺.

### Example 74

### 4-[3-amino-4-(cyclopentyloxy)-1H-indazol-6-yl]-2-(4-cyclohexylpiperazin-1-yl)benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 4-cyclohexylpiperidine for 1-phenylpiperazine in Step 2. ¹H NMR (300 MHz, DMSO-d₆) δ ppm 1.05-1.26 (m, 5H), 1.57-2.04 (m, 13H), 2.24-2.32 (m, 1H), 2.67-2.70 (m, 4H), 3.01-3.04 (m, 4H), 4.93 (s, 2H), 5.08-5.14 (m, 1H), 6.54 (s, 1H), 6.98 (d, J=1.0 Hz, 1H), 7.36-7.42 (m, 2H), 7.52 (d, J=3.1 Hz, 1H), 7.83 (d, J=8.1 Hz, 1H), 8.63 (d, J=3.1 Hz, 1H), 11.42 (s, 1H); MS (ESI) m/z 503 (M+H)⁺.

### Example 75

### 4-[3-amino-4-(cyclopentyloxy)-1H-indazol-6-yl]-2-[4-(pyridin-2-yl)piperazin-1-yl]benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 1-(pyridin-2-yl)piperazine for 1-phenylpiperazine in Step 2. ¹H NMR (300 MHz, DMSO-d₆) δ ppm 1.58-2.02 (m, 8H), 3.14-3.17 (m, 4H), 3.67-3.70 (m, 4H), 4.94 (s, 2H), 5.09-5.13 (m, 1H), 6.55 (s, 1H), 6.68 (dd, J=6.9, 5.0 Hz, 1H), 6.90 (d, J=8.7 Hz, 1H), 7.00 (s, 1H), 7.34-7.60 (m, 4H), 7.82 (d, J=7.9 Hz, 1H), 8.15 (dd, J=5.0, 1.4 Hz, 1H), 8.50-8.51 (m, 1H), 11.45 (s, 1H); MS (ESI) m/z 498 (M+H)⁺.

### Example 76

### 4-[3-amino-4-(cyclopentyloxy)-1H-indazol-6-yl]-2-(4-phenylpiperidin-1-yl)benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 4-phenylpiperidine for 1-phenylpiperazine in Step 2. ¹H NMR (300 MHz, DMSO-d₆) δ ppm 1.59-2.05 (m, 12H), 2.64-2.75 (m, 1H), 2.92-3.01 (m, 2H), 3.28-3.37 (m, 2H+H₂O), 4.94 (s, 2H), 5.08-5.14 (m, 1H), 6.55 (s, 1H), 7.00 (s, 1H), 7.19-7.25 (m, 1H), 7.30-7.37 (m, 4H), 7.40-7.44 (m, 2H), 7.52 (d, J=2.7 Hz, 1H), 7.86 (d, J=7.8 Hz, 1H), 8.71 (d, J=3.1 Hz, 1H) 11.45 (s, 1H); MS (ESI) m/z 496 (M+H)⁺.

### Example 77

### 2-(4-acetylpiperazin-1-yl)-4-[3-amino-4-(cyclopentyloxy)-1H-indazol-6-yl]benzamide

The titled compound was prepared as a trifluoroacetate salt using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 1-(piperazin-1-yl)ethanone for 1-phenylpiperazine in Step 2. The crude reaction mixture was purified by preparative HPLC on a Phenomenex® Luna® C8(2) 5 um 100 Å AXIA™ column (30 mm×75 mm) A gradient of acetonitrile (A) and 0.1% trifluoroacetic acid in water (B) was used, at a flow rate of 50 mL/minute (0-0.5 minutes 10% A, 0.5-7.0 minutes linear gradient 10-95% A, 7.0-10.0 minutes 95% A, 10.0-12.0 minutes linear gradient 95-10% A) to provide the titled compound. ¹H NMR (300 MHz, DMSO-d₆) δ ppm 1.58-2.02 (m, 8H), 2.05 (s, 3H), 3.00-3.09 (m, 4H), 3.63-3.67 (m, 4H), 5.12-5.17 (m, 1H), 6.65 (s, 1H), 7.08 (s, 1H), 7.39 (d, J=1.4 Hz, 1H), 7.45 (dd, J=8.1, 1.7 Hz, 1H), 7.59 (br s, 1H), 7.80 (d, J=7.8 Hz, 1H), 8.40 (br s, 1H), 12.12 (s, 1H); MS (ESI) m/z 463.5 (M+H)⁺.

### Example 78

### 4-[3-amino-4-(cyclopentyloxy)-1H-indazol-6-yl]-2-(4-cyclopropylpiperazin-1-yl)benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 1-cyclopropylpiperazine for 1-phenylpiperazine in Step 2. ¹H NMR (300 MHz, DMSO-d₆) δ ppm 0.32-0.36 (m, 2H), 0.43-0.49 (m, 2H), 1.60-2.01 (m, 9H), 2.78-2.76 (m, 4H), 2.96-3.04 (m, 4H), 4.94 (s, 2H), 5.09-5.13 (m, 1H), 6.53 (s, 1H), 6.97 (s, 1H), 7.81-7.54 (m, 3H), 7.83 (d, J=7.9 Hz, 1H), 8.62 (s, 1H), 11.45 (s, 1H); MS (ESI) m/z 461 (M+H)⁺.

### Example 79

### 1-{5-[3-amino-4-(cyclopentyloxy)-1H-indazol-6-yl]-2-carbamoylphenyl}piperidine-4-carboxamide

The titled compound was prepared as a trifluoroacetate salt using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting piperidine-4-carboxamide for 1-phenylpiperazine in Step 2. The crude reaction mixture was purified by preparative HPLC on a Phenomenex® Luna® C8(2) 5 um 100 Å AXIA™ column (30 mm×75 mm) A gradient of acetonitrile (A) and 0.1% trifluoroacetic acid in water (B) was used, at a flow rate of 50 mL/minute (0-0.5 minutes 10% A, 0.5-7.0 minutes linear gradient 10-95% A, 7.0-10.0 minutes 95% A, 10.0-12.0 minutes linear gradient 95-10% A) to provide the titled compound. ¹H NMR (300 MHz, DMSO-d₆) δ ppm 1.61-2.04 (m, 12H), 2.31-2.28 (m, 1H), 3.07-3.15 (m, 2H), 3.36-3.40 (m, 3H), 4.51 (s, 2H), 6.53 (s, 1H), 6.86 (s, 1H), 7.09 (s, 1H), 7.38 (s, 1H), 7.59 (d, J=8.1 Hz, 1H), 7.66 (s, 1H), 7.90 (s, 1H), 7.96 (d, J=8.1 Hz, 1H), 8.76 (s, 1H), 11.89 (s, 1H); MS (ESI) m/z 463 (M+H)⁺.

### Example 80

### 4-[3-amino-4-(cyclopentyloxy)-1H-indazol-6-yl]-2-(4-benzoylpiperazin-1-yl)benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting phenyl(piperazin-1-yl)methanone for 1-phenylpiperazine in Step 2. ¹H NMR (300 MHz, DMSO-d₆) δ ppm 1.60-2.02 (m, 8H), 3.04-3.12 (m, 4H), 3.59-3.81 (m, 4H), 4.94 (s, 2H), 5.09-5.12 (m, 1H), 6.53 (s, 1H), 6.99 (s, 1H), 7.41-7.54 (m, 8H), 7.79 (d, J=7.9 Hz, 1H), 8.44 (d, J=2.4 Hz, 1H), 11.46 (s, 1H); MS (ESI) m/z 525 (M+H)⁺.

### Example 81

### 4-[3-amino-4-(cyclopentyloxy)-1H-indazol-6-yl]-2-[4-(cyclopropylcarbonyl)piperazin-1-yl]benzamide

The titled compound was prepared as a trifluoroacetate salt using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting cyclopropyl(piperazin-1-yl)methanone for 1-phenylpiperazine in Step 2. The crude reaction mixture was purified by preparative HPLC on a Phenomenex® Luna® C8(2) 5 um 100 Å AXIA™ column (30 mm×75 mm) A gradient of acetonitrile (A) and 0.1% trifluoroacetic acid in water (B) was used, at a flow rate of 50 mL/minute (0-0.5 minutes 10% A, 0.5-7.0 minutes linear gradient 10-95% A, 7.0-10.0 minutes 95% A, 10.0-12.0 minutes linear gradient 95-10% A) to provide the titled compound. ¹H NMR (300 MHz, DMSO-d₆) δ ppm 0.71-0.78 (m, 4H), 1.58-2.08 (m, 9H), 3.0.-3.10 (m, 4H), 3.70-3.89 (m, 4H), 4.91 (s, 3H), 5.13-5.18 (m, 1H), 6.66 (s, 1H), 7.09 (s, 1H), 7.42 (d, J=1.4 Hz, 1H), 7.45 (dd, J=8.1, 1.4 Hz, 1H), 7.60 (s, 1H), 7.80 (d, J=8.1 Hz, 1H), 8.41 (s, 1H), 12.11 (s, 1H); MS (ESI) m/z 490 (M+H)⁺.

### Example 82

### 4-[3-amino-4-(cyclopentyloxy)-1H-indazol-6-yl]-2-[4-(morpholin-4-ylcarbonyl)piperazin-1-yl]benzamide

The titled compound was prepared as a trifluoroacetate salt using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting morpholino(piperazin-1-yl)methanone for 1-phenylpiperazine in Step 2. The crude reaction mixture was purified by preparative HPLC on a Phenomenex® Luna® C8(2) 5 um 100 Å AXIA™ column (30 mm×75 mm) A gradient of acetonitrile (A) and 0.1% trifluoroacetic acid in water (B) was used, at a flow rate of 50 mL/minute (0-0.5 minutes 10% A, 0.5-7.0 minutes linear gradient 10-95% A, 7.0-10.0 minutes 95% A, 10.0-12.0 minutes linear gradient 95-10% A) to provide the titled compound. ¹H NMR (300 MHz, DMSO-d₆) δ ppm 1.60-2.05 (m, 8H), 3.04-3.08 (m, 4H), 3.17-3.20 (m, 4H), 3.37-3.39 (m, 4H), 3.57-3.60 (m, 4H), 4.57 (s, 3H), 5.12-5.17 (m, 1H), 6.64 (s, 1H), 7.07 (s, 1H), 7.40 (d, J=1.4 Hz, 1H), 7.44 (dd, J=8.0, 1.5 Hz, 1H), 7.57 (s, 1H), 7.79 (d, J=8.1 Hz, 1H), 8.41 (s, 1H), 12.00 (s, 1H); MS (ESI) m/z 534 (M+H)⁺.

### Example 83

### 4-(3-amino-1H-indazol-6-yl)-2-{4-[2-oxo-2-(pyrrolidin-1-yl)ethyl]piperazin-1-yl}benzamide

### Step 1

### 4-(3-amino-1H-indazol-6-yl)-2-fluorobenzonitrile

To a suspension of 6-bromo-1H-indazol-3-amine (0.86 g, 4.1 mmol) and 4-cyano-3-fluorophenylboronic acid, (0.85 g, 5.15 mmol) in dimethoxyethane:ethanol (15 mL, 2:1) was added 1 M potassium carbonate (5.0 mL). The mixture was purged with nitrogen and bis(triphenylphosphine)palladium(II) dichloride (0.090 g, 0.128 mmol) was added. The reaction mixture was heated in a microwave reactor (CEM Discover®, ≦300 W)) at 160° C. for 20 minutes and then concentrated. The reaction mixture was poured into water (50 mL) and extracted with ethyl acetate (2×100 mL). The combined organic layers were dried over MgSO₄, filtered, and concentrated. The residue was triturated with ether to yield the titled compound. ¹H NMR (300 MHz, DMSO-d₆) δ ppm 5.43 (s, 2H), 7.30 (dd, J=8.5, 1.4 Hz, 1H), 7.59-7.60 (m, 1H), 7.76-7.82 (m, 2H), 7.91 (dd, J=11.2, 1.7 Hz, 1H), 7.96-8.01 (m, 1H), 11.62 (s, 1H); MS (ESI) m/z 253 (M+H)⁺.

### Step 2

### 4-(3-amino-1H-indazol-6-yl)-2-{4-[2-oxo-2-(pyrrolidin-1-yl)ethyl]piperazin-1-yl}benzamide

A solution of product from Step 1 (0.072 g, 0.285 mmol) and 1-(pyrrolidinocarbonylmethyl)piperazine (0.205 g, 1.04 mmol) in dimethyl sulfoxide (imp was heated at 120° C. for 16 hours. Then the reaction mixture was cooled and diluted with methanol (1 mL). 2 M NaOH (0.15 mL, 0.3 mmol) and 30% hydrogen peroxide (0.05 mL, 0.5 mmol) were added, and the mixture was stirred at ambient temperature for 5 hours. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.71-1.91 (m, 4H), 2.67-2.71 (m, 4H), 3.03-3.06 (m, 4H), 3.16-3.20 (m, 2H), 3.48-3.52 (m, 2H), 5.37 (s, 2H), 7.25 (dd, J=8.5, 1.4 Hz, 1H), 7.43-7.48 (m, 3H), 7.56 (d, J=2.7 Hz, 1H), 7.77 (d, J=8.1 Hz, 1H), 7.84 (d, J=8.1 Hz, 1H), 8.61 (d, J=2.7 Hz, 1H), 11.45 (s, 1H); MS (ESI) m/z 448 (M+H)⁺.

### Example 84

### 4-(3-amino-1H-indazol-6-yl)-2-(4-phenylpiperazin-1-yl)benzamide

The titled compound was prepared using the procedures described in Example 83 substituting 1-(pyrrolidinocarbonylmethyl)piperazine in Step 2 with 1-phenyl-piperazine. ¹H NMR (300 MHz, DMSO-d₆) δ ppm 3.19-3.22 (m, 4H), 3.33-3.36 (m, 4H), 5.38 (s, 2H), 6.82 (t, J=7.3 Hz, 1H), 7.00-7.02 (m, 3H), 7.22-7.28 (m, 3H), 7.45-7.50 (m, 3H), 7.54 (d, J=2.0 Hz, 1H), 7.79 (d, J=8.5 Hz, 1H), 7.81-7.84 (m, 1H), 8.50 (d, J=2.7 Hz, 1H), 11.46 (s, 1H); MS (ESI) m/z 413 (M+H)⁺. ¹H NMR (300 MHz, DMSO-d₆) δ ppm 3.19-3.22 (m, 4H), 3.33-3.36 (m, 4H), 5.38 (s, 2H), 6.82 (t, J=7.3 Hz, 1H), 7.00-7.02 (m, 3H), 7.22-7.28 (m, 3H), 7.45-7.50 (m, 3H), 7.54 (d, J=2.0 Hz, 1H), 7.79 (d, J=8.5 Hz, 1H), 7.81-7.84 (m, 1H), 8.50 (d, J=2.7 Hz, 1H), 11.46 (s, 1H); MS (ESI) m/z 413 (M+H)⁺.

### Example 85

### 4-(3-amino-1H-indazol-6-yl)-2-(4-cyclohexylpiperazin-1-yl)benzamide

The titled compound was prepared as the acetate using the procedures described in Example 83 substituting 1-(pyrrolidinocarbonylmethyl)piperazine in Step 2 with 1-cyclohexylpiperazine. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.07-1.29 (m, 5H), 1.57-1.60 (M, 1H), 1.74-1.80 (m, 4H), 1.89 (s, 3H), 2.26-2.31 (m, 1H), 2.67-2.70 (m, 4H), 3.01-3.04 (m, H), 5.38 (s, 2H), 7.24 (dd, J=8.3, 1.2 Hz, 1H), 7.42-7.48 (m, 3H), 7.53 (d, J=2.8 Hz, 1H), 7.76 (d, J=8.3 Hz, 1H), 7.84 (d, J=8.7 Hz, 1H), 8.63 (d, J=3.2 Hz, 1H), 11.45 (s, 1H); MS (ESI) m/z 419 (M+H)⁺.

### Example 86

### 4-(3-amino-1H-indazol-6-yl)-2-[4-(pyridin-2-yl)piperazin-1-yl]benzamide

The titled compound was prepared using the procedures described in Example 83 substituting 1-(pyrrolidinocarbonylmethyl)piperazine in Step 2 with 1-(pyridin-2-yl)piperazine. ¹H NMR (300 MHz, DMSO-d₆) δ ppm 3.14-3.17 (m, 4H), 3.68-3.71 (m, 4H), 5.38 (s, 2H), 6.68 (dd, J=6.7, 5.2 Hz, 1H), 6.91 (d, J=8.7 Hz, 1H), 7.25 (dd, J=8.3, 1.6 Hz, 1H), 7.45-7.49 (m, 3H), 7.54-7.60 (m, 2H), 7.77 (d, J=8.3 Hz, 1H), 7.83 (d, J=8.3 Hz, 1H), 8.15 (dd, J=4.8, 1.2 Hz, 1H), 8.52 (d, J=2.4 Hz, 1H), 11.46 (s, 1H); MS (ESI) m/z 414 (M+H)⁺.

### Example 87

### 4-(3-amino-1H-indazol-6-yl)-2-(4-benzylpiperazin-1-yl)benzamide

The titled compound was prepared using the procedures described in Example 83 substituting 1-(pyrrolidinocarbonylmethyl)piperazine in Step 2 with 1-benzylpiperazine. ¹H NMR (300 MHz, DMSO-d₆) δ ppm 2.57-2.60 (m, 4H), 3.04-3.07 (m, 4H), 3.56 (s, 2H), 7.22-7.35 (m, 6H), 7.43-7.48 (m, 3H), 7.55-7.56 (m, 1H), 7.76 (d, J=8.3 Hz, 1H), 7.84 (d, J=8.3 Hz, 1H), 8.62 (d, J=2.9 Hz, 1H), 11.45 (s, 1H); MS (ESI) m/z 427 (M+H)⁺.

### Example 88

### 4-(3-amino-1H-indazol-6-yl)-2-(4-cyclopropylpiperazin-1-yl)benzamide

The titled compound was prepared as the acetate salt using the procedures described in Example 83 substituting 1-(pyrrolidinocarbonylmethyl)piperazine in Step 2 with 1-cyclopropylpiperazine. ¹H NMR (300 MHz, DMSO-d₆) δ ppm 0.31-0.36 (m, 2H), 0.43-0.49 (m, 2H), 1.67-1.74 (m, 1H), 1.87 (s, 3H), 2.72-2.74 (m, 4H), 2.99-3.01 (m, 4H), 5.38 (s, 2H), 7.23 (dd, J=8.5, 1.4 Hz, 1H), 7.39-7.48 (m, 3H), 7.57 (d, J=2.4 Hz, 1H), 7.76 (d, J=8.3 Hz, 1H), 7.84 (d, J=8.0 Hz, 1H), 8.63 (d, J=2.4 Hz, 1H), 11.45 (s, 1H); MS (ESI) m/z 377 (M+H)⁺.

### Example 89

### 4-(3-amino-1H-indazol-6-yl)-2-{4-[2-(morpholin-4-yl)ethyl]piperazin-1-yl}benzamide

The titled compound was prepared as the acetate salt using the procedures described in Example 83 substituting 1-(pyrrolidinocarbonylmethyl)piperazine in Step 2 with 1-(2-morpholinoethyl)piperazine. ¹H NMR (300 MHz, DMSO-d₆) δ ppm 1.88 (s, 3H), 2.39-2.42 (m, 4H), 2.45-2.47 (m, 4H), 2.60-2.62 (m, 4H), 3.02-3.05 (m, 4H), 3.55-3.58 (m, 4H), 5.37 (s, 2H), 7.24 (dd, J=8.5, 1.4 Hz, 1H), 7.43-7.48 (m, 3H), 7.55 (d, J=2.7 Hz, 1H), 7.77 (d, J=8.5 Hz, 1H), 7.83 (d, J=8.5 Hz, 1H), 8.58 (d, J=3.1 Hz, 1H), 11.44 (s, 1H); MS (ESI) m/z 450 (M+H)⁺.

### Example 90

### 4-(3-amino-1H-indazol-6-yl)-2-[4-(pyridin-3-yl)piperazin-1-yl]benzamide

The titled compound was prepared using the procedures described in Example 83 substituting 1-(pyrrolidinocarbonylmethyl)piperazine in Step 2 with 1-(pyridin-3-yl)piperazine. ¹H NMR (300 MHz, DMSO-d₆) δ ppm 3.20-3.23 (m, 4H), 3.41-3.71 (m, 4H), 5.39 (s, 2H), 7.23-7.28 (m, 2H), 7.39-7.54 (m, 5H), 7.76-7.83 (m, 2H), 8.03 (dd, J=4.4, 1.2 Hz, 1H), 8.38 (d, J=2.8 Hz, 1H), 8.38 (d, J=2.0 Hz, 1H), 11.46 (s, 1H); MS (ESI) m/z 414 (M+H)⁺.

### Example 91

### 4-{3-amino-4-[(1,1,1-trifluoropropan-2-yl)oxy]-1H-indazol-6-yl}-2-[4-(pyridin-3-yl)piperazin-1-yl]benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-(pyridin-3-yl)piperazine for 1-phenylpiperazine in Step 2. ¹H NMR (300 MHz, DMSO-d₆) δ ppm 1.55 (d, J=6.4 Hz, 3H), 3.21-3.24 (m, 4H), 3.41-3.44 (m, 4H), 4.94 (s, 2H), 5.61-5.70 (m, 1H), 6.86 (s, 1H), 7.15 (s, 1H), 7.25 (dd, J=8.3, 4.6 Hz, 1H), 7.40 (ddd, J=8.5, 3.1, 1.4 Hz, 1H), 7.46-7.51 (m, 2H), 7.55 (d, J=2.4 Hz, 1H), 7.82 (d, J=8.1 Hz, 1H), 8.03 (dd, J=4.6, 1.2 Hz, 1H), 8.38 (d, J=3.1 Hz, 1H), 8.47 (d, J=2.4 Hz, 1H), 11.62 (s, 1H); MS (ESI) m/z 526 (M+H)⁺.

### Example 92

### 4-{3-amino-4-[(1,1,1-trifluoropropan-2-yl)oxy]-1H-indazol-6-yl}-2-(4-phenylpiperazin-1-yl)benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1. ¹H NMR (300 MHz, DMSO-d₆) δ ppm 1.54 (d, J=6.4 Hz, 3H), 3.18.-3.25 (m, 4H), 3.34-3.38 (m, 4H), 4.95 (s, 2H), 5.62-5.71 (m, 1H), 6.79-6.86 (m, 2H), 7.00-7.03 (m, 2H), 7.15 (s, 1H), 7.22-7.28 (m, 2H), 7.48-7.52 (m, 2H), 7.57 (d, J=2.4 Hz, 1H), 7.83 (d, J=7.9 Hz, 1H), 8.53 (d, J=2.4 Hz, 1H), 11.63 (s, 1H); MS (ESI) m/z 525 (M+H)⁺.

### Example 93

### 4-{3-amino-4-[(1,1,1-trifluoropropan-2-yl)oxy]-1H-indazol-6-yl}-2-(4-cyclohexylpiperazin-1-yl)benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-cyclohexylpiperazine for 1-phenylpiperazine in Step 2. ¹H NMR (300 MHz, DMSO-d₆) δ ppm 1.06-1.29 (m, 5H), 1.53-1.61 (m, 4H), 1.75-1.81 (m, 4H), 2.26-2.34 (m, 1H), 2.66-2.73 (m, 4H), 3.01-3.05 (m, 4H), 4.93 (s, 2H), 5.62-5.71 (m, 1H), 6.83 (s, 1H), 7.12 (s, 1H), 7.45-7.49 (m, 2H), 7.54 (d, J=2.4 Hz, 1H), 7.84 (d, J=8.1 Hz, 1H), 8.67 (d, J=3.1 Hz, 1H), 11.61 (s, 1H); MS (ESI) m/z 531 (M+H)⁺.

### Example 94

### 4-{3-amino-4-[(1,1,1-trifluoropropan-2-yl)oxy]-1H-indazol-6-yl}-2-[4-(pyridin-2-yl)piperazin-1-yl]benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-(pyridin-2-yl)piperazine for 1-phenylpiperazine in Step 2. ¹H NMR (300 MHz, DMSO-d₆) δ ppm 1.54 (d, J=6.4 Hz, 3H), 3.14-3.18 (m, 4H), 3.68-3.71 (m, 4H), 4.93 (s, 2H), 5.60-5.69 (m, 1H), 6.65-6.70 (m, 1H), 6.84 (s, 1H), 6.91 (d, J=8.8 Hz, 1H), 7.15 (s, 1H), 7.45-7.60 (m, 4H), 7.83 (d, J=7.8 Hz, 1H), 8.14-8.16 (m, 1H), 8.53 (d, J=2.4 Hz, 1H), 11.62 (s, 1H); MS (ESI) m/z 526 (M+H)⁺.

### Example 95

### 4-{3-amino-4-[(1,1,1-trifluoropropan-2-yl)oxy]-1H-indazol-6-yl}-2-{4-[2-(morpholin-4-yl)ethyl]piperazin-1-yl}benzamide

The titled compound was prepared as an acetate salt using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 4-(2-(piperazin-1-yl)ethyl)morpholine for 1-phenylpiperazine in Step 2. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.54 (d, J=6.1 Hz, 3H), 1.88 (s, 6H), 2.38-2.41 (m, 6H), 2.60-2.64 (m, 4H), 3.06-3.06 (m, 4H), 3.55-3.58 (m, 4H), 4.93 (s, 2H), 5.61-5.70 (m, 1H), 6.83 (s, 1H), 7.12 (s, 1H), 7.44-7.49 (m, 2H), 7.56 (d, J=3.1 Hz, 1H), 7.83 (d, J=8.1 Hz, 1H), 8.61 (d, J=2.7 Hz, 1H), 11.61 (s, 1H); MS (ESI) m/z 562 (M+H)⁺.

### Example 96

### 2-(4-acetylpiperazin-1-yl)-4-{3-amino-4-[(1,1,1-trifluoropropan-2-yl)oxy]-1H-indazol-6-yl}benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-(piperazin-1-yl)ethanone for 1-phenylpiperazine in Step 2. ¹H NMR (300 MHz, DMSO-d₆) δ ppm 1.53 (d, J=6.4 Hz, 3H), 2.05 (s, 3H), 2.98-3.07 (m, 4H), 3.63-3.65 (m, 4H), 4.93 (s, 2H), 5.60-5.68 (m, 1H), 6.83 (s, 1H), 7.12 (s, 1H), 7.39 (d, J=1.7 Hz, 1H), 7.48 (dd, J=8.1, 1.7 Hz, 1H), 7.54 (d, J=2.4 Hz, 1H), 7.78 (d, J=8.1 Hz, 1H), 8.42 (d, J=2.7 Hz, 1H), 11.62 (s, 1H); MS (ESI) m/z 491 (M+H)⁺.

### Example 97

### 4-{3-amino-4-[(1,1,1-trifluoropropan-2-yl)oxy]-1H-indazol-6-yl}-2-(4-cyclopropylpiperazin-1-yl)benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-cyclopropylpiperazine for 1-phenylpiperazine in Step 2. ¹H NMR (300 MHz, DMSO-d₆) δ ppm 0.32-0.36 (m, 2H), 0.44-0.49 (m, 2H), 1.53 (d, J=6.4 Hz, 3H), 1.67-1.74 (m, 1H), 2.73-2.76 (m, 4H), 3.00-3.03 (m, 4H), 4.94 (s, 2H), 5.62-5.70 (m, 1H), 6.83 (s, 1H), 7.12 (s, 1H), 7.43-7.49 (m, 2H), 7.59 (d, J=2.4 Hz, 1H), 7.84 (d, J=7.9 Hz, 1H), 8.68 (d, J=2.8 Hz, 1H), 11.62 (s, 1H); MS (ESI) m/z 489 (M+H)⁺.

### Example 98

### 4-{3-amino-4-[(1,1,1-trifluoropropan-2-yl)oxy]-1H-indazol-6-yl}-2-[4-(pyridin-4-yl)piperazin-1-yl]benzamide

The titled compound was prepared as an acetate salt using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-(pyridin-4-yl)piperazine for 1-phenylpiperazine in Step 2. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.54 (d, J=6.1 Hz, 3H), 1.88 (s, 6H), 3.17-3.53 (m, 4H), 3.52-3.55 (m, 4H), 4.94 (s, 2H), 5.60-5.69 (m, 1H), 6.84 (s, 1H), 6.89 (m, 2H), 7.14 (s, 1H), 7.43-7.50 (m, 2H), 7.55 (d, J=2.4 Hz, 1H), 7.80 (d, J=8.1 Hz, 1H), 8.16-8.21 (m, 2H), 8.41 (d, J=1.7 Hz, 1H), 11.61 (s, 1H); MS (ESI) m/z 526 (M+H)⁺.

### Example 99

### 4-{3-amino-4-[(1,1,1-trifluoropropan-2-yl)oxy]-1H-indazol-6-yl}-2-[(3aR*,6aS*)-5-benzylhexahydropyrrolo[3,4-c]pyrrol-2(1H)-yl]benzamide

The titled compound was prepared as a trifluoroacetate salt using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting (3aR*,6aS*)-2-benzyloctahydropyrrolo[3,4-c]pyrrole for 1-phenylpiperazine in Step 2. MS (ESI) m/z 565 (M+H)⁺.

### Example 100

### 4-{3-amino-4-[(1,1,1-trifluoropropan-2-yl)oxy]-1H-indazol-6-yl}-2-[4-(3,3-dimethylbutyl)piperazin-1-yl]benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-(3,3-dimethylbutyl)piperazine for 1-phenylpiperazine in Step 2. ¹H NMR (300 MHz, DMSO-d₆) δ ppm 0.91 (s, 9H), 1.36-1.41 (m, 2H), 1.54 (d, J=6.4 Hz, 3H), 2.33-2.38 (m, 2H), 2.55-2.78 (m, 4H), 3.03-3.06 (m, 4H), 4.94 (s, 2H), 5.62-5.72 (m, 1H), 6.83 (s, 1H), 7.12 (s, 1H), 7.43-7.49 (m, 2H), 7.57 (d, J=3.2 Hz, 1H), 7.84 (d, J=7.9 Hz, 1H), 8.63 (d, J=2.8 Hz, 1H), 11.62 (s, 1H); MS (ESI) m/z 533 (M+H)⁺.

### Example 101

### 4-{3-amino-4-[(1,1,1-trifluoropropan-2-yl)oxy]-1H-indazol-6-yl}-2-(4-isopropylpiperazin-1-yl)benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-isopropylpiperazine for 1-phenylpiperazine in Step 2. ¹H NMR (300 MHz, DMSO-d₆) δ ppm 1.02 (d, J=6.4 Hz, 6H), 1.54 (d, J=6.4 Hz, 3H), 2.63-2.66 (m, 4H), 2.68-2.75 (m, 1H), 3.03-3.06 (m, 4H), 4.94 (s, 2H), 5.62-5.70 (m, 1H), 6.83 (s, 1H), 7.13 (s, 1H), 7.45-7.49 (m, 2H), 7.55 (d, J=2.7 Hz, 1H), 7.85 (d, J=7.8 Hz, 1H), 8.68 (d, J=2.7 Hz, 1H), 11.62 (s, 1H); MS (ESI) m/z 491 (M+H)⁺.

### Example 102

### 4-{3-amino-4-[(1,1,1-trifluoropropan-2-yl)oxy]-1H-indazol-6-yl}-2-(4-cyclopentylpiperazin-1-yl)benzamide

The titled compound was prepared as the acetate salt using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting 1-cyclopentylpiperazine for 1-phenylpiperazine in Step 2. ¹H NMR (300 MHz, DMSO-d₆) δ ppm 1.43-1.50 (m, 2H), 1.60-1.64 (m, 5H), 1.68-1.74 (m, 2H), 1.87-1.93 (m, 2H), 1.96 (s, 3H), 2.26-2.34 (m, 1H), 2.70-2.72 (m, 4H), 3.13-3.15 (m, 4H), 5.03 (s, 2H), 5.72-5.78 (m, 1H), 6.82 (s, 1H), 7.22 (s, 1H), 7.54-7.58 (m, 2H), 7.65 (d, J=2.4 Hz, 1H), 7.94 (d, J=7.9 Hz, 1H), 8.78 (d, J=3.1 Hz, 1H), 11.71 (s, 1H); MS (ESI) m/z 531 (M+H)⁺.

### Example 103

### 4-{3-amino-4-[(1,1,1-trifluoropropan-2-yl)oxy]-1H-indazol-6-yl}-2-[4-(cyclopropylcarbonyl)piperazin-1-yl]benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting cyclopropyl(piperazin-1-yl)methanone for 1-phenylpiperazine in Step 2. ¹H NMR (300 MHz, DMSO-d₆) δ ppm 0.71-1.78 (m, 4H), 1.54 (d, J=6.4 Hz, 3H), 2.00-2.06 (m, 1H), 3.02-3.09 (m, 4H), 3.66-3.70 (m, 2H), 3.85-3.89 (m, 2H), 4.94 (s, 2H), 5.61-5.71 (m, 1H), 6.83 (s, 1H), 7.13 (s, 1H), 7.41 (d, J=1.2 Hz, 1H), 7.48 (dd, J=7.9, 1.6 Hz, 1H), 7.55 (d, J=1.6 Hz, 1H), 7.79 (d, J=7.9 Hz, 1H), 8.43 (d, J=2.4 Hz, 1H), 11.62 (s, 1H); MS (ESI) m/z 517 (M+H)⁺.

### Example 104

### 4-{3-amino-4-[(1,1,1-trifluoropropan-2-yl)oxy]-1H-indazol-6-yl}-2-[4-(2,2-dimethylpropanoyl)piperazin-1-yl]benzamide

The titled compound was prepared using the procedures described in Example 1 substituting 6-bromo-4-[(1,1,1-trifluoropropan-2-yl)oxy]-1H-indazol-3-amine (Intermediate 2) for 6-bromo-4-(tetrahydro-2H-pyran-4-yloxy)-1H-indazol-3-amine (Intermediate 1) in Step 1 and substituting tert-butyl piperazine-1-carboxylate for 1-phenylpiperazine in Step 2. ¹H NMR (300 MHz, DMSO-d₆) δ ppm 1.23 (s, 9H), 1.53 (d, J=6.4 Hz, 3H), 3.02-3.05 (m, 4H), 3.74-3.78 (m, 4H), 4.94 (s, 2H), 5.60-5.68 (m, 1H), 6.83 (s, 1H), 7.13 (s, 1H), 7.40 (d, J=1.6 Hz, 1H), 7.48 (dd, J=7.9, 1.6 Hz, 1H), 7.53 (d, J=2.0 Hz, 1H), 7.79 (d, J=7.9 Hz, 1H), 8.43 (d, J=2.4 Hz, 1H), 11.62 (s, 1H); MS (ESI) m/z 533 (M+H)⁺.

### Example 105

### 4-(3-amino-4-{[(2R)-1,1,1-trifluoropropan-2-yl]oxy}-1H-indazol-6-yl)-2-[2-(cyclohexyloxy)ethoxy]-N-methylbenzamide

Potassium t-butoxide (26.4 mg, 0.235 mmol) was dissolved in dimethyl sulfoxide (1 mL). 2-(Cyclohexyloxy)ethanol (75 mg, 0.52 mmol) was added to this solution, and the reaction mixture was purged with nitrogen followed by stirring at ambient temperature for 1 hour. 4-(3-Amino-4-{[(2R)-1,1,1-trifluoropropan-2-yl]oxy}-1H-indazol-6-yl)-2-fluoro-N-methylbenzamide (50.7 mg, 0.128 mmol, Example 48, Step 1) was added, and the resultant mixture was kept at room temperature overnight. The reaction mixture was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound. ¹H NMR (300 MHz, DMSO-d₆) δ ppm 1.15-1.31 (m, 5H), 1.46-1.55 (m, 4H), 1.65-1.73 (m, 2H), 1.84-1.92 (m, 2H), 2.86 (d, J=4.8 Hz, 3H), 3.83-3.86 (m, 2H), 4.39-4.42 (m, 2H), 4.99 (s, 2H), 5.60-5.69 (m, 1H), 6.88 (s, 1H), 7.17 (s, 1H), 7.37-7.45 (m, 2H), 7.75 (d, J=8.7 Hz, 1H), 8.24 (q, J=4.2 Hz, 1H), 11.69 (s, 1H); MS (ESI) m/z 521 (M+H)⁺.

### Example 106

### 4-(3-amino-4-{[(2R)-1,1,1-trifluoropropan-2-yl]oxy}-1H-indazol-6-yl)-N-methyl-2-(2-phenoxyethoxy)benzamide

The titled compound was prepared using the procedures described in Example 105 substituting 2-phenoxyethanol for 2-(cyclohexyloxy)ethanol. ¹H NMR (300 MHz, DMSO-d₆) δ ppm 1.50-1.55 (m, 3H), 2.75 (d, J=4.8 Hz, 3H), 4.44-4.47 (m, 2H), 4.63-4.65 (m, 2H), 4.95 (s, 2H), 5.69-5.59 (m, 1H), 6.89-7.67 (m, 8H), 7.81 (d, J=8.3 Hz, 1H), 7.92 (dd, J=7.9, 2.4 Hz, 1H), 8.17 (q, J=3.7 Hz, 1H), 11.67 (s, 1H); MS(ESI) m/z 515 (M+H)⁺.

### Example 107

### 4-[3-amino-4-(cyclopentyloxy)-1H-indazol-6-yl]-2-{4-[2-oxo-2-(piperidin-1-yl)ethyl]piperazin-1-yl}benzamide

The titled compound was prepared using the procedures described in Example 1 substituting 6-bromo-4-(cyclopentyloxy)-1H-indazol-3-amine (Intermediate 3) for 6-bromo-4-(tetrahydro-2H-pyran-4-yloxy)-1H-indazol-3-amine (Intermediate 1) in Step 1 and substituting 1-(2-oxo-2-piperidin-1-ylethyl)piperazine for 1-phenylpiperazine in Step 2. ¹H NMR (300 MHz, DMSO-d₆) δ ppm 1.32-1.50 (m, 2H), 1.50-1.72 (m, 6H), 1.69-1.84 (m, 2H), 1.82-2.08 (m, 4H), 2.63 (bs, 4H), 3.04 (s, 2H), 3.21 (bs, 4H), 3.41-3.53 (m, 4H), 4.94 (s, 2H), 5.08-5.12 (m, 1H), 6.53 (s, 1H), 6.99 (s, 1H), 7.38-7.41 (m, 2H), 7.57 (d, J=2.4 Hz, 1H), 7.81 (d, J=7.9 Hz, 1H), 8.51 (d, J=2.8 Hz, 1H), 11.45 (s, 1H); MS (ESI) m/z 546.3 (M+H)⁺.

### Example 108

### 4-[3-amino-4-(tetrahydro-2H-pyran-4-yloxy)-1H-indazol-6-yl]-N-methyl-2-[4-(2,2,2-trifluoroethyl)piperazin-1-yl]benzamide

### Step 1

### 4-bromo-N-methyl-2-[4-(2,2,2-trifluoroethyl)piperazin-1-yl]benzamide

4-Bromo-2-fluoro-N-methylbenzamide (1.17 g, 5.04 mmol) was dissolved in dimethyl sulfoxide (8 mL). 1-(2,2,2-Trifluoroethyl)piperazine (1.69 g, 10.05 mmol) and K₂CO₃ (1.55 g, 11.22 mmol) were added, and the reaction mixture was heated to 120° C. for 17 hours. The reaction mixture was partition between water (100 mL) and ethyl acetate (3×100 mL). The combined organic layers were washed with brine (100 mL), dried over MgSO₄, filtered and concentrated. The residue was purified by silica gel chromatography (ethyl acetate, R_{f}=0.51) to give the titled compound (1.60 g, 83%). ¹H NMR (300 MHz, DMSO-d₆) δ ppm 2.75-2.80 (m, 7H), 2.92-2.95 (m, 4H), 3.24 (q. J=10.3 Hz, 2H), 7.25-7.28 (m, 2H), 7.48 (d, J=8.5 Hz, 1H), 8.58 (q, J=4.4 Hz, 1H); MS(ESI+) m/z 380 & 382 (M+H)⁺.

### Step 2

### N-methyl-4-(4,4,5,5-tetramethyl-1,3,2-dioxaborolan-2-yl)-2-[4-(2,2,2-trifluoroethyl)piperazin-1-yl]benzamide

4-Bromo-N-methyl-2-[4-(2,2,2-trifluoroethyl)piperazin-1-yl]benzamide (Step 1, 1.59 g, 4.18 mmol) was dissolved in dioxane (25 mL). Bis(pinacolato)diboron (1.61 g, 6.34 mmol), potassium acetate (1.65 g, 6.34 mmol) and [1,1′-bis(diphenylphosphino)ferrocene]dichloro palladium(II) (90.8 mg, 0.124 mmol) were added to the reaction mixture. The reaction mixture was purged with nitrogen for 15 minutes and then heated to 85° C. for 3 hours. The reaction mixture was concentrated and partition between water (100 mL) and EtOAc (3×100 mL). The combined organics were washed with brine (100 mL), dried over MgSO₄, filtered and concentrated. The residue was purified by silica get chromatography (20% ethyl acetate in dichloromethane, R_{f}=0.30) to give the titled compound (1.30 g, 73%). ¹H NMR (300 MHz, DMSO-d₆) δ ppm 1.30 (s, 12H), 2.77-2.82 (m, 7H), 2.91-2.94 (m, 4H), 3.25 (q. J=10.3 Hz, 2H), 7.38-7.41 (m, 2H), 7.63 (d, J=7.5 Hz, 1H), 8.81 (q, J=4.4 Hz, 1H); MS(ESI+) m/z 428 (M+H)⁺.

### Step 3

### 4-[3-amino-4-(tetrahydro-2H-pyran-4-yloxy)-1H-indazol-6-yl]-N-methyl-2-[4-(2,2,2-trifluoroethyl)piperazin-1-yl]benzamide

To a suspension of 6-bromo-4-(tetrahydro-2H-pyran-4-yloxy)-1H-indazol-3-amine (Intermediate 1, 1.43 g, 4.58 mmol) and N-methyl-4-(4,4,5,5-tetramethyl-1,3,2-dioxaborolan-2-yl)-2-[4-(2,2,2-trifluoroethyl)piperazin-1-yl]benzamide (Step 2, 1.62 g, 3.79 mmol) in dimethoxyethane:ethanol (12 mL, 1:1) was added 1 M potassium carbonate (4.5 mL). The mixture was purged with nitrogen and bis(triphenylphosphine)palladium(II) dichloride (78.0 mg, 0.111 mmol) was added. The reaction mixture was heated in a microwave reactor (CEM Discover®, ≦300 W) at 160° C. for 20 minutes and then concentrated. The residue was poured into water (50 mL) and extracted with EtOAc (3×50 mL). The combined organic layers were washed with brine (50 mL), dried over MgSO₄, filtered and concentrated. The residue was purified by silica gel chromatography (0%->10% ethanol in ethyl acetate, R_{f}=0.10->0.29) to give the titled compound (1.50 g, 74%). ¹H NMR (300 MHz, DMSO-d₆) δ ppm 1.72-1.80 (m, 2H), 2.03-2.07 (m, 2H), 2.81-2.86 (m, 7H), 3.02-3.04 (m, 4H), 3.27 (q, J=10.2 Hz, 2H), 3.54-3.60 (m, 2H), 3.84-3.89 (m, 2H), 4.90-4.96 (m, 1H), 5.00 (s, 2H), 6.65 (s, 1H), 7.01 (s, 1H), 7.38-7.42 (m, 2H), 7.75 (d, J=7.9 Hz, 1H), 8.95 (q, J=4.6 Hz, 1H), 11.48 (s, 1H); MS(ESI) m/z 533 (M+H)⁺.

### Example 109

### 4-(3-amino-4-isopropoxy-1H-indazol-6-yl)-2-(4-cyclohexylpiperazin-1-yl)-N-methylbenzamide

### Step 1

### 2-(4-cyclohexylpiperazin-1-yl)-N-methyl-4-(4,4,5,5-tetramethyl-1,3,2-dioxaborolan-2-yl)benzamide

The titled compound was prepared using the procedures described for the preparation of Example 108, Steps 1 and 2 except 1-cyclohexylpiperazine was substituted for 1-(2,2,2-trifluoroethyl)piperazine in Step 1; MS(ESI+) m/z 428 (M+H)⁺.

### Step 2

### 4-(3-amino-4-isopropoxy-1H-indazol-6-yl)-2-(4-cyclohexylpiperazin-1-yl)-N-methylbenzamide

To a suspension of 6-bromo-4-isopropoxy-1H-indazol-3-amine (Intermediate 10, 98.9 mg, 0.366 mmol) and 2-(4-cyclohexylpiperazin-1-yl)-N-methyl-4-(4,4,5,5-tetramethyl-1,3,2-dioxaborolan-2-yl)benzamide (Step 1, 218.8 mg, 0.512 mmol) in dimethoxyethane:ethanol (2 mL, 1:1) was added 1 M potassium carbonate (0.6 mL). The mixture was purged with nitrogen and bis(triphenylphosphine)palladium(II) dichloride (9.9 mg, 0.014 mmol) was added. The reaction mixture was heated in a microwave reactor (CEM Discover, ≦300 W) at 160° C. for 20 minutes and then concentrated. The reaction was poured into water (35 mL) and extracted with ethyl acetate (3×35 mL). The combined organic layers were washed with brine (35 mL), dried over MgSO₄, filtered and concentrated. The residue was purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound as the acetate salt (158.1 mg, 78%). ¹H NMR (300 MHz, DMSO-d₆) δ ppm 1.16-1.30 (m, 5H), 1.38 (d, J=6.1 Hz, 6H), 1.57-1.60 (m, 1H), 1.75-1.83 (m, 4H), 1.87 (s, 3H), 2.25-2.31 (m, 1H), 2.67-2.70 (m, 4H), 2.8 (d, J=4.8 Hz, 3H), 2.98-3.01 (m, 4H), 4.87-4.93 (m, 1H), 4.97 (s, 2H), 6.58 (s, 1H), 6.97 (s, 1H), 7.39-7.42 (m, 2H), 7.77 (d, J=7.8 Hz, 1H), 9.09 (q, J=4.4 Hz, 1H), 11.39 (s, 1H); MS (ESI) m/z 491 (M+H)⁺.

### Example 110

### 4-[3-amino-4-(tetrahydro-2H-pyran-4-yloxy)-1H-indazol-6-yl]-2-[4-(cyclopentylmethyl)piperazin-1-yl]-N-methylbenzamide

The titled compound was prepared as described in Example 29 substituting 1-(cyclopentylmethyl)piperazine for 1-cyclohexylpiperazine. ¹H NMR (300 MHz, DMSO-d₆) δ ppm 1.15-1.25 (m, 2H), 1.44-1.58 (m, 4H), 1.66-1.80 (m, 4H), 1.88 (s, 3H), 2.02-2.16 (m, 4H), 2.26 (d, J=7.5 Hz, 2H), 2.54-2.58 (m, 3H), 2.85 (d, J=4.8 Hz, 3H), 2.99-3.02 (m, 4H), 3.53-3.60 (m, 2H), 3.83-3.90 (m, 2H), 4.89-4.97 (m, 1H), 4.99 (s, 2H), 6.64 (s, 1H), 7.00 (s, 1H), 7.39-7.43 (m, 2H), 7.77 (d, J=7.8 Hz, 1H), 9.06 (q, J=4.3 Hz, 1H), 11.46 (s, 1H); MS (ESI) m/z 533 (M+H)⁺.

### Example 111

### tert-butyl 4-{5-[3-amino-4-(tetrahydro-2H-pyran-4-yloxy)-1H-indazol-6-yl]-2-(methylcarbamoyl)phenyl}piperazine-1-carboxylate

### Step 1

### tert-butyl 4-[2-(methylcarbamoyl)-5-(4,4,5,5-tetramethyl-1,3,2-dioxaborolan-2-yl)phenyl]piperazine-1-carboxylate

The titled compound was prepared using the procedures described for the preparation of Example 108, Steps 1 and 2 except tert-butyl 1-piperazinecarboxylate was substituted for 1-(2,2,2-trifluoroethyl)piperazine in Step 1. ¹H NMR (300 MHz, DMSO-d₆) δ ppm 1.30 (s, 12H), 1.42 (s, 9H), 2.82 (d, J=4.6 Hz, 3H), 2.85-2.87 (m, 4H), 3.46-3.49 (m, 4H), 7.35 (s, 1H), 7.41 (d, J=7.6 Hz, 1H), 7.63 (d, J=7.6 Hz, 1H), 8.75 (q, J=4.5 Hz, 1H); MS(ESI+) m/z 446 (M+H)⁺.

### Step 2

### tert-butyl 4-{5-[3-amino-4-(tetrahydro-2H-pyran-4-yloxy)-1H-indazol-6-yl]-2-(methylcarbamoyl)phenyl}piperazine-1-carboxylate

The titled compound was prepared as described in Example 108, Step 3 substituting tert-butyl 4-[2-(methylcarbamoyl)-5-(4,4,5,5-tetramethyl-1,3,2-dioxaborolan-2-yl)phenyl]piperazine-1-carboxylate (Step 1) for N-methyl-4-(4,4,5,5-tetramethyl-1,3,2-dioxaborolan-2-yl)-2-[4-(2,2,2-trifluoroethyl)piperazin-1-yl]benzamide. ¹H NMR (300 MHz, DMSO-d₆) δ ppm 1.43 (s, 9H), 1.70-1.82 (m, 2H), 1.87 (s, 3H), 2.02-2.09 (m, 2H), 2.85 (d, J=4.8 Hz, 3H), 2.95-2.99 (m, 4H) 3.51-3.60 (m, 6H), 3.83-3.89 (m, 2H), 4.89-4.96 (m, 1H), 4.99 (s, 2H), 6.64 (s, 1H), 7.01 (s, 1H), 7.37 (d, J=1.4 Hz, 1H), 7.72 (dd, J=8.0, 1.5 Hz, 1H), 7.76 (d, J=8.1 Hz, 1H), 8.86 (q, J=4.4 Hz, 1H), 11.48 (s, 1H); MS (ESI) m/z 551 (M+H)⁺.

### Example 112

### 4-[3-amino-4-(tetrahydro-2H-pyran-4-yloxy)-1H-indazol-6-yl]-N-methyl-2-(piperazin-1-yl)benzamide

A solution of product from Example 29, Step 1 (780.3 mg, 2.03 mmol) and piperazine (919.2 mg, 10.67 mmol) in dimethyl sulfoxide (4 mL) was heated at 130° C. for 21 hours. The reaction was partitioned between 1 M NaOH (100 mL) and 10% isopropanol in dichloromethane (3×100 mL). The combined organic layers were washed with water (100 mL) and concentrated to provide the titled compound (907.4 mg, 99%). ¹H NMR (300 MHz, DMSO-d₆) δ ppm 1.71-1.82 (m, 2H), 2.02-2.09 (m, 2H), 2.85-2.92 (m, 11H), 3.53-3.60 (m, 2H), 3.83-3.90 (m, 2H), 4.88-4.96 (m, 1H), 5.02 (s, 2H), 6.83 (s, 1H), 6.99 (s, 1H), 7.35 (d, J=1.7 Hz, 1H), 7.41 (dd, J=8.1, 1.7 Hz, 1H), 7.79 (d, J=8.1 Hz, 1H), 9.17 (q, J=4.8 Hz, 1H), 11.46 (s, 1H); MS (ESI) m/z 451 (M+H)⁺.

### Example 113

### 4-[3-amino-4-(tetrahydro-2H-pyran-4-yloxy)-1H-indazol-6-yl]-2-[4-(5-fluoropyrimidin-2-yl)piperazin-1-yl]-N-methylbenzamide

A solution of product from Example 112 (238.7 mg, 0.53 mmol), 2-chloro-5-fluoropyrimidine (196.3 mg, 1.481 mmol) and triethylamine (0.15 mL, 1.08 mmol) in dimethyl sulfoxide (1 mL) was heated at 40° C. for 20 hours. The reaction mixture was diluted with methanol (1 mL) and purified by preparative HPLC on a Waters Nova-Pak® HR C18 6 μm 60 Å Prep-Pak® cartridge column (40 mm×100 mm) using a gradient of 10% to 100% acetonitrile in 10 mM aqueous ammonium acetate over 12 minutes at a flow rate of 70 mL/minute to provide the titled compound (204.3 mg, 71%). ¹H NMR (300 MHz, DMSO-d₆) δ ppm 1.73-1.79 (m, 2H), 2.03-2.03 (m, 2H, 2.87 (d, J=4.9 Hz, 3H), 3.08-3.10 (m, 4H), 3.54-3.58 (m, 2H), 3.84-3.91 (m, 6H), 4.89-4.94 (m, 1H), 5.00 (s, 2H), 6.65 (s, 1H), 7.01 (s, 1H), 7.39 (d, J=1.5 Hz, 1H), 7.44 (dd, J=7.9, 1.5 Hz, 1H), 7.78 (d, J=7.9 Hz, 1H), 8.05 (s, 2H), 9.00 (q, J=4.6 Hz, 1H), 11.49 (s, 1H); MS (ESI) m/z 547 (M+H)⁺.

### Example 114

### isopropyl 4-{5-[3-amino-4-(tetrahydro-2H-pyran-4-yloxy)-1H-indazol-6-yl]-2-(methylcarbamoyl)phenyl}piperazine-1-carboxylate

A solution of product from Example 112 (452.3 mg, 1.00 mmol), N,N-diisopropylethylamine (0.5 mL, 2.86 mmol) and isopropyl chloroformate (1 M in toluene, 1.0 mL, 1.0 mmol) in dichloromethane (5 mL) was stirred at ambient temperature for 2 hours. The reaction mixture was quenched with methanol (0.1 mL) and concentrated. The residue was purified by silica gel chromatography (gradient 0% to 10% ethanol in ethyl acetate, R_{f}=0.07->0.25) to give the titled compound (256.2 mg, 48%). ¹H NMR (300 MHz, DMSO-d₆) δ ppm 1.21 (d, J=6.1 Hz, 6H), 1.70-1.82 (m, 2H), 2.02-2.09 (m, 2H), 2.86 (d, J=4.8 Hz, 3H), 2.97-3.00 (m, 4H) 3.53-3.60 (m, 6H), 3.83-3.90 (m, 2H), 1.77-1.85 (m, 1H), 4.89-4.96 (m, 1H), 4.99 (s, 2H), 6.64 (s, 1H), 7.00 (s, 1H), 7.37 (d, J=1.4 Hz, 1H), 7.43 (dd, J=8.0, 1.5 Hz, 1H), 7.76 (d, J=8.1 Hz, 1H), 8.90 (q, J=4.8 Hz, 1H), 11.45 (s, 1H); MS(ESI) m/z 537 (M+H)⁺.

### Example 115

### N-ethyl-4-[3-methyl-4-(tetrahydro-2H-pyran-4-yloxy)-1H-indazol-6-yl]-2-(4-phenylpiperazin-1-yl)benzamide

### Step 1

### 4-bromo-2-fluoro-6-(tetrahydro-2H-pyran-4-yloxy)benzaldehyde

Diisobutylaluminum hydride (1 M in CH₂Cl₂, 75 mL, 75.0 mmol) was added, drop wise, over 20 minutes at room temperature to a solution of 4-bromo-2-fluoro-6-(tetrahydro-2H-pyran-4-yloxy)benzonitrile (18 g, 60 mmol, Intermediate 1, Step 1) in CH₂Cl₂ (250 mL). After 15 minutes, the reaction was quenched with saturated aqueous NH₄Cl followed by the addition of CH₂Cl₂ (500 mL) and 1 M HCl (500 mL; to break up aluminum salts) and stirred for 15 minutes. The organic phase was separated, dried (MgSO₄) and concentrated under vacuum. The residue was taken up in ether, stirred to pulverize, filtered and dried to provide 13.83 g (76%) of the titled compound as a white solid. ¹H NMR (300 MHz, CDCl₃) δ ppm 1.75-1.95 (m, 2H), 1.98-2.15 (m, 2H), 3.53-3.71 (m, 2H), 3.91-4.05 (m, 2H), 4.64 (tt, J=7.4, 3.8 Hz, 1H), 6.80-6.99 (m, 2H), 10.40 (d, J=1.1 Hz, 1H).

### Step 2

### 1-[4-bromo-2-fluoro-6-(tetrahydro-2H-pyran-4-yloxy)phenyl]ethanol

To a solution of 4-bromo-2-fluoro-6-(tetrahydro-2H-pyran-4-yloxy)benzaldehyde (13.8 g, 45.6 mmol, Step 1) in tetrahydrofuran (150 mL) at room temperature was slowly added methylmagnesium bromide (60.8 mL, 182 mmol) as a 3 M solution in ether (slight exotherm) and stirring was continued overnight. The mixture was quenched with water (250 mL). Saturated NH₄Cl (250 mL) was added, and the mixture was stirred for 15 minutes. The reaction was partitioned between ether (500 mL) and water (250 mL). The organic phase was washed with brine (2×250 mL), dried (Na₂SO₄), filtered and concentrated. This material was used directly in the next reaction without further purification. ¹H NMR (300 MHz, CDCl₃) δ ppm 1.56 (d, J=6.8 Hz, 3H), 1.76-1.90 (m, 2H), 2.03-2.15 (m, 2H), 3.62 (ddd, J=11.8, 5.8, 2.3 Hz, 2H), 3.92-4.04 (m, 2H), 4.56 (tt, J=8.0, 3.9 Hz, 1H), 5.12-5.27 (m, 1H), 6.82 (t, J=1.2 Hz, 1H), 6.87 (dd, J=9.5, 1.7 Hz, 1H).

### Step 3

### 1-[4-bromo-2-fluoro-6-(tetrahydro-2H-pyran-4-yloxy)phenyl]ethanone

To a mixture of 1-[4-bromo-2-fluoro-6-(tetrahydro-2H-pyran-4-yloxy)phenyl]ethanol (14.6 g, 45.6 mmol, Step 2), 4-methylmorpholine 4-oxide (8.02 g, 68.4 mmol) and 4 angstrom molecular sieves (20 g, 46 mmol) in CH₂Cl₂ (200 mL) at room temperature was added tetrapropylammonium perruthenate (0.802 g, 2.28 mmol) and stirring was continued overnight (for convenience). The dark reaction mixture was filtered through a 2000 mL sintered glass funnel with a 1-inch pad of diatomaceous earth (top) and 1-inch pad of silica (bottom); eluted with ethyl acetate (1500 mL). The filtrate was concentrated to an oil, and the residue was purified by silica gel chromatography (330 g column, 0% to 30% ethyl acetate:heptane) to provide 12.52 g (87% from Step 1) of the titled compound as a colorless oil that solidified to a white solid upon standing. ¹H NMR (300 MHz, CDCl₃) δ ppm 1.72-1.86 (m, 2H), 1.94-2.13 (m, 2H), 2.52 (d, J=0.9 Hz, 3H), 3.60 (ddd, J=11.5, 7.8, 3.4 Hz, 2H), 3.77-4.05 (m, 2H), 4.53 (tt, J=7.6, 3.8 Hz, 1H), 6.86 (t, J=1.3 Hz, 1H), 6.91 (dd, J=8.7, 1.5 Hz, 1H); MS (ESI) m/z 317/319 (M+H)⁺.

### Step 4

### 6-bromo-3-methyl-4-(tetrahydro-2H-pyran-4-yloxy)-1H-indazole

To a solution of 1-[4-bromo-2-fluoro-6-(tetrahydro-2H-pyran-4-yloxy)phenyl]ethanone (12.5 g, 39.5 mmol, Step 3) in dimethoxyethane (120 mL) at room temperature was added hydrazine (6.26 mL, 197 mmol), and the reaction was heated to 90° C. After stirring overnight, the mixture was cooled to room temperature. The supernatant solution was carefully decanted from an insoluble oil and concentrated. The residue was concentrated from ethyl acetate (2×250 mL) to remove trace hydrazine and triturated with 50% heptane/ether (150 mL). The solid was filtered, washed with heptane and dried in a vacuum oven at 50° C. overnight to give 11.43 g (93%) of the titled compound as a white solid. ¹H NMR (300 MHz, CDCl₃) δ ppm 1.76-1.97 (m, 2H), 2.06-2.27 (m, 2H), 2.68 (s, 3H), 3.54-3.74 (m, 2H), 4.00 (ddd, J=11.2, 7.2, 3.6 Hz, 2H), 4.69 (tt, J=7.1, 3.7 Hz, 1H), 6.53 (d, J=0.8 Hz, 1H), 7.14 (d, J=1.1 Hz, 1H); MS (ESI) m/z 311/313 (M+H)⁺.

### Step 5

### 4-bromo-N-ethyl-2-fluorobenzamide

A mixture of 4-bromo-2-fluorobenzoic acid (3.52 g, 16.07 mmol) and thionyl chloride (10 mL, 137 mmol) was heated at 80° C. for 80 minutes, then cooled to room temperature and concentrated under vacuum. The residue was taken up in CH₂Cl₂ (15 mL) and concentrated under vacuum. This was repeated once more to ensure removal of excess thionyl chloride. The residue was dissolved in CH₂Cl₂ (15 mL) and treated with a solution of ethylamine (2 M in tetrahydrofuran, 5 mL, 10 mmol). The mixture was stirred at room temperature for 2 hours and then concentrated under vacuum. The residue was taken up with 8% aqueous H₂SO₄ (5 mL) and extracted with CH₂Cl₂ (2×10 mL). The combined organic phase was filtered through diatomaceous earth and concentrated to a white solid. The residue was crystallized from ethanol-water (1:1, 25 mL) to provide the titled compound (849 mg in two crops, 90%). ¹H NMR (400 MHz, DMSO-d₆) δ ppm 1.10 (t, J=7.2 Hz, 3H), 3.14-3.32 (m, 2H), 7.49 (dd, J=8.3, 1.8 Hz, 1H), 7.52-7.58 (m, 1H), 7.65 (dd, J=10.0, 1.7 Hz, 1H), 8.35 (s, 1H); MS (ESI) m/z 246/248 (M+H)⁺.

### Step 6

### 4-bromo-N-ethyl-2-(4-phenylpiperazin-1-yl)benzamide

A mixture of 1-phenylpiperazine (1098 mg, 6.77 mmol) and 4-bromo-N-ethyl-2-fluorobenzamide (555 mg, 2.255 mmol, Step 5) in dimethyl sulfoxide (1 mL) was heated at 120° C. for 150 minutes. The mixture was diluted with hot water (30 mL), and cooled to room temperature. The precipitate was collected by filtration, and recrystallized from ethanol-water (ca. 2:1, 30 mL) to provide the titled compound (694 mg). ¹H NMR (300 MHz, CD₃OD) δ ppm 1.24 (t, J=7.3 Hz, 3H), 3.11-3.21 (m, 4H), 3.26-3.34 (m, 4H), 3.43 (q, J=7.2 Hz, 2H), 6.66-6.93 (m, 1H), 6.97-7.10 (m, 2H), 7.19-7.29 (m, 2H), 7.33 (dd, J=8.3, 1.9 Hz, 1H), 7.42 (d, J=1.9 Hz, 1H), 7.66 (d, J=8.3 Hz, 1H); MS (ESI) m/z 388/390 (M+H)⁺.

### Step 7

### N-ethyl-2-(4-phenylpiperazin-1-yl)-4-(4,4,5,5-tetramethyl-1,3,2-dioxaborolan-2-yl)benzamide

A mixture of bis(pinacolato)diboron (395 mg, 1.56 mmol), 4-bromo-N-ethyl-2-(4-phenylpiperazin-1-yl)benzamide (300 mg, 0.773 mmol, Step 6) and potassium acetate (379 mg, 3.86 mmol) in dioxane (3 mL) was purged with a nitrogen stream for 5 minutes. [1,1′-Bis(diphenylphosphino)ferrocene)palladium dichloride (5.6 mg, 7.65 μmol) was added, and the mixture was heated at 100° C. for 90 minutes, then cooled to room temperature. The reaction was diluted with water (15 mL) and extracted with CH₂Cl₂ (30 mL). The organic phase was concentrated and the residue purified by flash chromatography (silica gel eluted with hexanes-ethyl acetate, 90:10-65:35) to provide the titled compound (262 mg). ¹H NMR (300 MHz, CDCl₃) δ ppm 1.21 (t, J=7.2 Hz, 3H), 1.35 (s, 12H), 3.19-3.30 (m, 4H), 3.34-3.41 (m, 4H), 3.44-3.62 (m, 2H), 6.96 (t, J=7.4 Hz, 1H), 7.04 (d, J=7.4 Hz, 2H), 7.28-7.43 (m, 2H), 7.64-7.74 (m, 2H), 8.16 (d, J=7.9 Hz, 1H), 9.67 (s, 1H); MS (ESI) m/z 436 (M+H)⁺.

### Step 8

### N-ethyl-4-[3-methyl-4-(tetrahydro-2H-pyran-4-yloxy)-1H-indazol-6-yl]-2-(4-phenylpiperazin-1-yl)benzamide

Bis(triphenylphosphine)palladium(II)dichloride (2.8 mg, 3.99 μmol), N-ethyl-2-(4-phenylpiperazin-1-yl)-4-(4,4,5,5-tetramethyl-1,3,2-dioxaborolan-2-yl)benzamide (40.0 mg, 0.092 mmol, Step 7) and 6-bromo-3-methyl-4-(tetrahydro-2H-pyran-4-yloxy)-1H-indazole (25.2 mg, 0.081 mmol, Step 4) were combined in a microwave vial with stir bar. Ethanol (2 mL) and 1,2 dimethoxyethane (2 mL) were added, followed by 1 M aqueous K₂CO₃ (0.16 mL, 0.160 mmol). The vial was capped and the mixture was irradiated (Biotage Personal Chemistry unit, 300 W) at 120° C. for 20 minutes. The mixture was concentrated under vacuum, and the residue was diluted with water (4 mL) and extracted with CH₂Cl₂ (2×6 mL). The combined organic phase was dried (MgSO₄) and concentrated under vacuum. The residue was purified by flash chromatography (silica gel eluted with hexanes-ethyl acetate, 50:50-0:100) to provide the titled compound (34 mg, 78%). ¹H NMR (300 MHz, DMSO-d₆) δ ppm 1.16 (t, J=7.2 Hz, 3H), 1.76 (tdd, J=10.7, 7.1, 3.4 Hz, 2H), 2.07 (dtd, J=9.3, 6.2, 3.0 Hz, 2H), 2.61 (s, 3H), 3.16-3.24 (m, 4H), 3.26-3.42 (m, 6H), 3.53-3.67 (m, 2H), 3.87 (ddd, J=10.8, 6.9, 3.6 Hz, 2H), 4.91-5.04 (m, 1H), 6.74-6.86 (m, 2H), 6.98-7.06 (m, 2H), 7.16-7.30 (m, 3H), 7.45-7.51 (m, 2H), 7.82 (d, J=8.4 Hz, 1H), 9.17 (t, J=5.6 Hz, 1H), 12.60 (s, 1H); MS (ESI) m/z 540 (M+H)⁺.

### Example 116

### 2-(4-cyclohexylpiperazin-1-yl)-N-methyl-4-[4-(tetrahydro-2H-pyran-4-yloxy)-1H-indazol-6-yl]benzamide

### Step 1

### 4-bromo-2-(4-cyclohexylpiperazin-1-yl)-N-methylbenzamide

A mixture of 1-cyclohexylpiperazine (1.043 g, 6.20 mmol) and 4-bromo-2-fluoro-N-methylbenzamide (1.00 g, 4.31 mmol) in dimethyl sulfoxide (2 mL) was heated at 120° C. for 2 hours, then removed from the heat and added gradually to rapidly stirring water (30 mL). After 20 minutes, the light yellow suspension was filtered and the cake washed well with water. The solid was recrystallized from ethanol-water (15 mL, 2:1) to provide the titled compound as off-white needles (1.08 g, 66%). ¹H NMR (300 MHz, DMSO-d₆) δ ppm 1.01-1.31 (m, 5H), 1.53-1.63 (m, 1H), 1.67-1.85 (m, 4H), 2.19-2.34 (m, 1H), 2.57-2.68 (m, 4H), 2.79 (d, J=4.7 Hz, 3H), 2.86-2.98 (m, 4H), 7.22-7.29 (m, 2H), 7.46-7.53 (m, 1H), 8.65 (q, J=4.4 Hz, 1H); MS (ESI) m/z 380/382 (M+H)⁺.

### Step 2

### 2-(4-cyclohexylpiperazin-1-yl)-N-methyl-4-(4,4,5,5-tetramethyl-1,3,2-dioxaborolan-2-yl)benzamide

The procedure of Example 115, Step 7 was used, substituting 4-bromo-2-(4-cyclohexylpiperazin-1-yl)-N-methylbenzamide (Step 1) for 4-bromo-N-ethyl-2-(4-phenylpiperazin-1-yl)benzamide as described therein, to prepare the titled compound. ¹H NMR (300 MHz, CDCl₃) δ ppm 1.09-1.32 (m, 5H), 1.34 (s, 12H), 1.57-1.74 (m, 1H), 1.78-2.48 (m, 5H), 2.48-2.93 (m, 4H), 3.00 (d, J=4.8 Hz, 3H), 3.02-3.16 (m, 4H), 7.63 (d, J=7.5 Hz, 2H), 8.03-8.25 (m, 1H), 9.79-10.04 (m, 1H); MS (ESI) m/z 428 (M+H)⁺.

### Step 3

### 6-bromo-4-(tetrahydro-2H-pyran-4-yloxy)-1H-indazole

Anhydrous hydrazine (4 mL, 127 mmol) was added dropwise over 30 minutes to a stirring solution of 4-bromo-2-fluoro-6-(tetrahydro-2H-pyran-4-yloxy)benzaldehyde (1.00 g, 3.30 mmol, Example 115, Step 1) in 1,2-dimethoxyethane (8 mL). The mixture was heated under nitrogen at 100° C. for 16 hours, then cooled to room temperature and concentrated under vacuum. The residue was purified by flash chromatography (silica gel eluted with hexanes-ethyl acetate, 80:20-60:40) to provide the titled compound as a white solid (0.82 g, 84%). ¹H NMR (300 MHz, DMSO-d₆) δ ppm 1.59-1.75 (m, 2H), 1.95-2.09 (m, 2H), 3.54 (ddd, J=11.7, 9.1, 2.9 Hz, 2H), 3.88 (dt, J=9.3, 4.4 Hz, 2H), 4.84 (tt, J=8.3, 4.0 Hz, 1H), 6.80 (d, J=1.3 Hz, 1H), 7.30 (t, J=1.1 Hz, 1H), 8.04 (s, 1H), 13.13 (bs, 1H); MS (ESI) m/z 297/299 (M+H)⁺.

### Step 4

### 2-(4-cyclohexylpiperazin-1-yl)-N-methyl-4-[4-(tetrahydro-2H-pyran-4-yloxy)-1H-indazol-6-yl]benzamide

The procedure of Example 115, Step 8 was used, substituting 2-(4-cyclohexylpiperazin-1-yl)-N-methyl-4-(4,4,5,5-tetramethyl-1,3,2-dioxaborolan-2-yl)benzamide (Step 2) for N-ethyl-2-(4-phenylpiperazin-1-yl)-4-(4,4,5,5-tetramethyl-1,3,2-dioxaborolan-2-yl)benzamide, and substituting 6-bromo-4-(tetrahydro-2H-pyran-4-yloxy)-1H-indazole (Step 3) for 6-bromo-3-methyl-4-(tetrahydro-2H-pyran-4-yloxy)-1H-indazole as described therein, to prepare the titled compound. ¹H NMR (300 MHz, DMSO-d₆) δ ppm 1.04-1.32 (m, 5H), 1.51-1.93 (m, 7H), 1.92-2.21 (m, 2H), 2.23-2.33 (m, 1H), 2.64-2.73 (m, 4H), 2.85 (d, J=4.5 Hz, 3H), 3.01 (s, 4H), 3.50-3.62 (m, 2H), 3.84-3.96 (m, 2H), 4.92-5.02 (m, 1H), 6.88 (s, 1H), 7.32 (s, 1H), 7.40-7.49 (m, 2H), 7.79 (d, J=7.9 Hz, 1H), 8.07 (s, 1H), 9.07 (d, J=4.7 Hz, 1H), 13.11 (bs, 1H); MS (ESI) m/z 518 (M+H)⁺.

### Example 117

### N-cyclopropyl-4-[3-methyl-4-(tetrahydro-2H-pyran-4-yloxy)-1H-indazol-6-yl]-2-(4-phenylpiperazin-1-yl)benzamide

### Step 1

### 4-bromo-N-cyclopropyl-2-fluorobenzamide

The procedure of Example 115, Step 5 was used, substituting cyclopropylamine for ethylamine as described therein, to prepare the titled compound. ¹H NMR (300 MHz, DMSO-d₆) δ ppm 0.44-0.62 (m, 2H), 0.62-0.78 (m, 2H), 2.82 (tq, J=8.0, 4.1 Hz, 1H), 7.45-7.54 (m, 2H), 7.63 (d, J=9.6 Hz, 1H), 8.41 (d, J=3.4 Hz, 1H); MS (ESI) m/z 258/260 (M+H)⁺.

### Step 2

### 4-bromo-N-cyclopropyl-2-(4-phenylpiperazin-1-yl)benzamide

The procedure of Example 115, Step 6 was used, substituting 4-bromo-N-cyclopropyl-2-fluorobenzamide for 4-bromo-N-ethyl-2-fluorobenzamide as described therein, to prepare the titled compound. ¹H NMR (300 MHz, DMSO-d₆) δ ppm 0.47-0.60 (m, 2H), 0.60-0.74 (m, 2H), 2.77-2.90 (m, 1H), 3.05-3.12 (m, 4H), 3.22-3.27 (m, 4H), 6.82 (t, J=7.2 Hz, 1H), 6.95-7.03 (m, 2H), 7.21-7.27 (m, 2H), 7.28-7.36 (m, 2H), 7.48 (d, J=8.0 Hz, 1H), 8.80 (d, J=4.3 Hz, 1H); MS (ESI) m/z 400/402 (M+H)⁺.

### Step 3

### 3-methyl-4-(tetrahydro-2H-pyran-4-yloxy)-6-(4,4,5,5-tetramethyl-1,3,2-dioxaborolan-2-yl)-1H-indazole

The procedure of Example 115, Step 7 was used, substituting 6-bromo-3-methyl-4-(tetrahydro-2H-pyran-4-yloxy)-1H-indazole (Example 115, Step 4) for 4-bromo-N-ethyl-2-(4-phenylpiperazin-1-yl)benzamide as described therein, to prepare the titled compound. ¹H NMR (300 MHz, CDCl₃) δ ppm 1.36 (s, 12H), 1.85-1.98 (m, 2H), 2.07-2.18 (m, 2H), 2.76 (s, 3H), 3.63-3.75 (m, 2H), 3.96-4.05 (m, 2H), 4.85 (tt, J=7.0, 3.5 Hz, 1H), 6.79 (s, 1H), 7.52 (s, 1H); MS (DCI) m/z 359 (M+H)⁺.

### Step 4

### N-cyclopropyl-4-[3-methyl-4-(tetrahydro-2H-pyran-4-yloxy)-1H-indazol-6-yl]-2-(4-phenylpiperazin-1-yl)benzamide

The procedure of Example 115, Step 8 was used, substituting 3-methyl-4-(tetrahydro-2H-pyran-4-yloxy)-6-(4,4,5,5-tetramethyl-1,3,2-dioxaborolan-2-yl)-1H-indazole (Step 3) for N-ethyl-2-(4-phenylpiperazin-1-yl)-4-(4,4,5,5-tetramethyl-1,3,2-dioxaborolan-2-yl)benzamide, and substituting 4-bromo-N-cyclopropyl-2-(4-phenylpiperazin-1-yl)benzamide (Step 2) for 6-bromo-3-methyl-4-(tetrahydro-2H-pyran-4-yloxy)-1H-indazole as described therein, to prepare the titled compound. ¹H NMR (300 MHz, DMSO-d₆) δ ppm 0.54 (m. 2H), 0.73 (td, J=6.9, 4.9 Hz, 2H), 1.68-1.83 (m, 2H), 2.00-2.17 (m, 2H), 2.61 (s, 3H), 2.83-2.94 (m, 1H), 3.13-3.22 (m, 4H), 3.25-3.33 (m, 4H), 3.60 (ddd, J=11.3, 7.5, 3.2 Hz, 2H), 3.87 (ddd, J=11.3, 6.8, 3.7 Hz, 2H), 4.97 (tt, J=7.1, 4.1, 3.7 Hz, 1H), 6.78 (s, 1H), 6.83 (t, J=7.3 Hz, 1H), 7.02 (d, J=8.1 Hz, 2H), 7.21 (s, 1H), 7.22-7.29 (m, 2H), 7.45-7.51 (m, 1H), 7.46 (s, 1H), 7.77 (d, J=8.5 Hz, 1H), 9.20 (d, J=4.4 Hz, 1H), 12.59 (s, 1H); MS (DCI) m/z 552 (M+H)⁺.

### Example 118

### N-cyclobutyl-2-(4-cyclohexylpiperazin-1-yl)-4-[3-methyl-4-(tetrahydro-2H-pyran-4-yloxy)-1H-indazol-6-yl]benzamide

### Step 1

### 4-bromo-N-cyclobutyl-2-fluorobenzamide

The procedure of Example 115, Step 5 was used, substituting cyclobutylamine for ethylamine as described therein, to prepare the titled compound. ¹H NMR (300 MHz, DMSO-d₆) δ ppm 1.56-1.74 (m, 2H), 1.92-2.09 (m, 2H), 2.13-2.29 (m, 2H), 4.27-4.45 (m, 1H), 7.44-7.55 (m, 2H), 7.64 (dd, J=10.4, 1.2 Hz, 1H), 8.59 (d, J=7.4 Hz, 1H); MS (DCI) m/z 272/274 (M+H)⁺.

### Step 2

### 4-bromo-N-cyclobutyl-2-(4-cyclohexylpiperazin-1-yl)benzamide

The procedure of Example 116, Step 1 was used, substituting 4-bromo-N-cyclobutyl-2-fluorobenzamide (Step 1) for 4-bromo-2-fluoro-N-methylbenzamide as described therein, to prepare the titled compound. ¹H NMR (300 MHz, DMSO-d₆) δ ppm 1.01-1.31 (m, 5H), 1.51-1.63 (m, 1H), 1.63-1.83 (m, 6H), 1.90-2.08 (m, 2H), 2.16-2.36 (m, 3H), 2.61-2.69 (m, 4H), 2.86-2.94 (m, 4H), 4.27-4.46 (m, 1H), 7.25-7.32 (m, 1H), 7.30 (s, 1H), 7.53 (d, J=8.3 Hz, 1H), 9.18 (d, J=7.5 Hz, 1H); MS (DCI) m/z 420/422 (M+H)⁺.

### Step 3

### N-cyclobutyl-2-(4-cyclohexylpiperazin-1-yl)-4-[3-methyl-4-(tetrahydro-2H-pyran-4-yloxy)-1H-indazol-6-yl]benzamide

The procedure of Example 115, Step 8 was used, substituting 3-methyl-4-(tetrahydro-2H-pyran-4-yloxy)-6-(4,4,5,5-tetramethyl-1,3,2-dioxaborolan-2-yl)-1H-indazole (Example 117, Step 3) for N-ethyl-2-(4-phenylpiperazin-1-yl)-4-(4,4,5,5-tetramethyl-1,3,2-dioxaborolan-2-yl)benzamide, and substituting 4-bromo-N-cyclobutyl-2-(4-cyclohexylpiperazin-1-yl)benzamide (Step 2) for 6-bromo-3-methyl-4-(tetrahydro-2H-pyran-4-yloxy)-1H-indazole as described therein, to prepare the titled compound, which was purified by crystallization from ethyl acetate. ¹H NMR (300 MHz, DMSO-d₆) δ ppm 1.03-1.36 (m, 5H), 1.59 (br d, J=11.5 Hz, 1H), 1.68-1.87 (m, 8H), 1.89-2.13 (m, 4H), 2.21-2.40 (m, 3H), 2.61 (s, 3H), 2.67-2.78 (m, 4H), 2.89-3.11 (m, 4H), 3.49-3.72 (m, 2H), 3.75-3.95 (m, 2H), 4.43 (sext, J=7.8 Hz, 1H), 4.92-5.04 (m, 1H), 6.77 (s, 1H), 7.19 (s, 1H), 7.39-7.51 (m, 2H), 7.82 (d, J=8.5 Hz, 1H), 9.72 (d, J=7.5 Hz, 1H), 12.59 (s, 1H); MS (DCI) m/z 572 (M+H)⁺.

### Example 119

### N-{2-(4-cyclohexylpiperazin-1-yl)-4-[3-methyl-4-(tetrahydro-2H-pyran-4-yloxy)-1H-indazol-6-yl]phenyl}acetamide

### Step 1

### 1-(5-bromo-2-nitrophenyl)-4-cyclohexylpiperazine

A mixture of 4-bromo-2-fluoronitrobenzene (204 mg, 0.927 mmol), 1-cyclohexylpiperazine (328 mg, 1.947 mmol) and ethanol (4 mL) was irradiated at 120° C. (Biotage Personal Chemistry unit, 300 W) for 10 minutes. The solution was cooled to room temperature and concentrated under vacuum. The residue was purified by flash chromatography (silica gel eluted with hexanes-ethyl acetate, 90:10-70:30) to provide the titled compound (300 mg, 88%). ¹H NMR (300 MHz, methanol-d₄) δ ppm 1.08-1.39 (m, 5H), 1.60-1.70 (m, 1H), 1.79-1.89 (m, 2H), 1.90-2.01 (m, 2H), 2.33 (tt, J=10.7, 3.2 Hz, 1H), 2.65-2.77 (m, 4H), 3.03-3.10 (m, 4H), 7.23 (dd, J=8.8, 2.0 Hz, 1H), 7.41 (d, J=2.0 Hz, 1H), 7.68 (d, J=8.8 Hz, 1H); MS (ESI) m/z 368/370 (M+H)⁺.

### Step 2

### 4-bromo-2-(4-cyclohexylpiperazin-1-yl)aniline

Solid ammonium chloride (436 mg, 8.15 mmol) was added to a solution of 1-(5-bromo-2-nitrophenyl)-4-cyclohexylpiperazine (300 mg, 0.815 mmol, Step 1) in acetone (15 mL) and water (3 mL). The mixture was stirred at room temperature as zinc powder (533 mg, 8.15 mmol) was added in portions over 2 minutes. The resulting suspension was stirred at room temperature for 30 minutes, then filtered through diatomaceous earth which was subsequently rinsed with ethyl acetate (30 mL). The filtrate was concentrated under vacuum, and the residue was partitioned between CH₂Cl₂ (30 mL) and 1 M NaOH (10 mL). The organic phase was dried (MgSO₄) and concentrated under vacuum to provide the titled compound (261 mg, 95%). ¹H NMR (300 MHz, methanol-d₄) δ ppm 1.11-1.40 (m, 5H), 1.61-1.72 (m, 1H), 1.80-1.90 (m, 2H), 1.92-2.04 (m, 2H), 2.28-2.40 (m, 1H), 2.73-2.82 (m, 4H), 2.85-2.94 (m, 4H), 6.65 (d, J=8.5 Hz, 1H), 6.96 (dd, J=8.5, 2.4 Hz, 1H), 7.04 (d, J=2.4 Hz, 1H); MS (DCI) m/z 338/340 (M+H)⁺.

### Step 3

### N-[4-bromo-2-(4-cyclohexylpiperazin-1-yl)phenyl]acetamide

Acetic anhydride (150 μL, 1.59 mmol) was added to a solution of 4-bromo-2-(4-cyclohexylpiperazin-1-yl)aniline (261 mg, 0.772 mmol, Step 2) in CH₂Cl₂ (5 mL), and the resulting solution was stirred at room temperature for 3 hours. The solution was concentrated under vacuum and the residue was crystallized from ethanol-water (2:1, 4 mL) to provide the titled compound (215 mg, 73%). ¹H NMR (300 MHz, DMSO-d₆) δ ppm 1.03-1.32 (m, 5H), 1.51-1.64 (m, 1H), 1.69-1.86 (m, 4H), 2.09 (s, 3H), 2.20-2.33 (m, 1H), 2.63-2.70 (m, 4H), 2.76-2.83 (m, 4H), 7.15-7.26 (m, 2H), 7.77 (d, J=9.2 Hz, 1H), 8.78 (s, 1H); MS (DCI) m/z 380/382 (M+H)⁺.

### Step 4

### N-{2-(4-cyclohexylpiperazin-1-yl)-4-[3-methyl-4-(tetrahydro-2H-pyran-4-yloxy)-1H-indazol-6-yl]phenyl}acetamide

The procedure of Example 115, Step 8 was used, substituting 3-methyl-4-(tetrahydro-2H-pyran-4-yloxy)-6-(4,4,5,5-tetramethyl-1,3,2-dioxaborolan-2-yl)-1H-indazole (Example 117, Step 3) for N-ethyl-2-(4-phenylpiperazin-1-yl)-4-(4,4,5,5-tetramethyl-1,3,2-dioxaborolan-2-yl)benzamide, and substituting N-[4-bromo-2-(4-cyclohexylpiperazin-1-yl)phenyl]acetamide (Step 3) for 6-bromo-3-methyl-4-(tetrahydro-2H-pyran-4-yloxy)-1H-indazole as described therein, to prepare the titled compound. ¹H NMR (400 MHz, DMSO-d₆) δ ppm 1.06-1.31 (m, 6H), 1.55-1.64 (m, 1H), 1.69-1.88 (m, 5H), 2.00-2.09 (m, 2H), 2.13 (s, 3H), 2.25-2.34 (m, 1H), 2.59 (s, 3H), 2.69-2.76 (m, 4H), 2.86-2.95 (m, 4H), 3.60 (ddd, J=11.3, 7.6, 3.4 Hz, 2H), 3.81-3.90 (m, 2H), 4.96 (tt, J=7.2, 3.9 Hz, 1H), 6.73 (s, 1H), 7.11 (d, J=0.6 Hz, 1H), 7.32-7.37 (m, 1H), 7.36 (s, 1H), 7.95 (d, J=7.9 Hz, 1H), 8.82 (s, 1H), 12.52 (s, 1H); MS (ESI) m/z 532 (M+H)⁺.

### Example 120

### 4-[4-(cyclopentyloxy)-1H-indazol-6-yl]-N-ethyl-2-(4-phenylpiperazin-1-yl)benzamide

### Step 1

### 4-bromo-2-(cyclopentyloxy)-6-fluorobenzonitrile

Cyclopentanol (1.25 mL, 13.8 mmol) was added over 1 minute to an ice-cooled solution of potassium tert-butoxide (1.62 g, 14.5 mmol) in tetrahydrofuran (25 mL). The resulting solution was stirred at 0° C. for 30 minutes, then cooled to −70° C. under nitrogen. A solution of 4-bromo-2,6-difluorobenzonitrile (3.0 g, 13.8 mmol) in tetrahydrofuran (13 mL) was added over 30 minutes, and the mixture was allowed to gradually warm to room temperature with continued stirring for 12 hours. Saturated aqueous NH₄Cl (15 mL) and water (30 mL) were added, and the mixture was extracted with ethyl acetate (2×50 mL). The combined extracts were concentrated under vacuum, and the residue was purified by flash chromatography (silica gel eluted with hexanes-ethyl acetate, 100:0-90:10) to provide the titled compound (3.52 g, 90%). ¹H NMR (300 MHz, CDCl₃) δ ppm 1.58-2.01 (m, 8H), 4.81-4.87 (m, 1H), 6.90 (s, 1H), 6.94 (dd, J=8.1, 1.4 Hz, 1H).

### Step 2

### 4-bromo-2-(cyclopentyloxy)-6-fluorobenzaldehyde

The procedure of Example 115, Step 1 was used, substituting 4-bromo-2-(cyclopentyloxy)-6-fluorobenzonitrile (Step 1) for 4-bromo-2-fluoro-6-(tetrahydro-2H-pyran-4-yloxy)benzonitrile as described therein, to prepare the titled compound. ¹H NMR (300 MHz, DMSO-d₆) δ ppm 1.54-1.85 (m, 6H), 1.86-2.02 (m, 2H), 5.04-5.14 (tt, J=5.3, 2.4 Hz, 1H), 7.22 (ddd, J=10.3, 1.6, 0.8 Hz, 1H), 7.30 (t, J=1.4 Hz, 1H), 10.18-10.20 (m, 1H).

### Step 3

### 6-bromo-4-(cyclopentyloxy)-1H-indazole

The procedure of Example 116, Step 3 was used, substituting 4-bromo-2-(cyclopentyloxy)-6-fluorobenzaldehyde (Step 2) for 4-bromo-2-fluoro-6-(tetrahydro-2H-pyran-4-yloxy)benzaldehyde as described therein, to prepare the titled compound. ¹H NMR (300 MHz, DMSO-d₆) δ ppm 1.52-1.87 (m, 6H), 1.89-2.07 (m, 2H), 5.02 (tt, J=5.9, 2.4 Hz, 1H), 6.64 (d, J=1.2 Hz, 1H), 7.27 (d, J=1.2 Hz, 1H), 7.98 (s, 1H), 13.11 (s, 1H); MS (ESI) m/z 281/283 (M+H)⁺.

### Step 4

### 4-[4-(cyclopentyloxy)-1H-indazol-6-yl]-N-ethyl-2-(4-phenylpiperazin-1-yl)benzamide

The procedure of Example 115, Step 8 was used, substituting 6-bromo-4-(cyclopentyloxy)-1H-indazole (Step 3) for 6-bromo-3-methyl-4-(tetrahydro-2H-pyran-4-yloxy)-1H-indazole as described therein, to prepare the titled compound. ¹H NMR (300 MHz, DMSO-d₆) δ ppm 1.16 (t, J=7.3 Hz, 3H), 1.59-2.09 (m, 8H), 3.17-3.24 (m, 4H), 3.32-3.41 (m, 6H), 5.17 (tt, J=5.8, 2.9 Hz, 1H), 6.78 (s, 1H), 6.82 (t, J=7.3 Hz, 1H), 7.02 (d, J=7.8 Hz, 2H), 7.22-7.29 (m, 2H), 7.32 (s, 1H), 7.47-7.55 (m, 2H), 7.82 (d, J=7.8 Hz, 1H), 8.01 (s, 1H), 9.16 (t, J=5.6 Hz, 1H), 13.08 (s, 1H); MS (DCI) m/z 510 (M+H)⁺.

### Example 121

### 2-(4-cyclohexylpiperazin-1-yl)-4-[4-(cyclopentyloxy)-3-methyl-1H-indazol-6-yl]-N-methylbenzamide

### Step 1

### 1-[4-bromo-2-(cyclopentyloxy)-6-fluorophenyl]ethanone

A solution of methylmagnesium bromide, (3 M in ethyl ether, 12.0 mL, 36.0 mmol) was added dropwise to a cold (≦−60° C.) solution of 4-bromo-2-(cyclopentyloxy)-6-fluorobenzaldehyde (Example 120, Step 2, 1.92 g, 6.69 mmol) in tetrahydrofuran (50 mL). The reaction mixture was stirred at ≦−60° C. for 2 hours, then allowed to warm to −20° C. over 15 minutes. Saturated aqueous NH₄Cl (6 mL) was added gradually (foaming), and the mixture was diluted with water (40 mL) and extracted with ethyl acetate (2×30 mL). The combined organic phases were washed with saturated aqueous NaCl (20 mL), dried (MgSO₄) and concentrated under vacuum. The residual oil (2.02 g) was dissolved in CH₂Cl₂ (100 mL) and stirred at room temperature as pyridinium chlorochromate (5.03 g, 23.32 mmol) was added in portions over 5 minutes. The mixture was stirred at room temperature for 14 hours, then poured onto a column of silica gel and eluted with CH₂Cl₂ (150 mL), then hexanes-ethyl acetate (90:10, 600 mL) to provide the titled compound (1.75 g, 87%). ¹H NMR (400 MHz, CDCl₃) δ ppm 1.59-1.98 (m, 8H), 2.48 (s, 3H), 4.78 (tt, J=5.4, 2.7 Hz, 1H), 6.82-6.89 (m, 2H); MS (DCI) m/z 301/303 (M+H)⁺.

### Step 2

### 6-bromo-4-(cyclopentyloxy)-3-methyl-1H-indazole

Anhydrous hydrazine (4 mL, 127 mmol) was added dropwise over 30 minutes to a stirring solution of 1-[4-bromo-2-(cyclopentyloxy)-6-fluorophenyl]ethanone (Step 1, 1.75 g, 5.81 mmol) in dioxane (12 mL), and the resulting mixture was heated at 100° C. under nitrogen for 14 hours, then cooled to room temperature and concentrated under vacuum. The residue was purified by flash chromatography (silica gel, eluted with hexanes-ethyl acetate, 90:10-50:50) to provide the titled compound (1.40 g, 82%). ¹H NMR (400 MHz, DMSO-d₆) δ ppm 1.58-1.86 (m, 6H), 1.87-2.00 (m, 2H), 2.49 (s, 3H), 4.97 (tt, J=5.2, 2.6 Hz, 1H), 6.54 (s, 1H), 7.14 (d, J=1.2 Hz, 1H), 12.59 (s, 1H).

### Step 3

### 2-(4-cyclohexylpiperazin-1-yl)-4-[4-(cyclopentyloxy)-3-methyl-1H-indazol-6-yl]-N-methylbenzamide

The procedure of Example 115, Step 8 was used, substituting 2-(4-cyclohexylpiperazin-1-yl)-N-methyl-4-(4,4,5,5-tetramethyl-1,3,2-dioxaborolan-2-yl)benzamide (Example 116, Step 2) for N-ethyl-2-(4-phenylpiperazin-1-yl)-4-(4,4,5,5-tetramethyl-1,3,2-dioxaborolan-2-yl)benzamide, and substituting 6-bromo-4-(cyclopentyloxy)-3-methyl-1H-indazole (Step 3) for 6-bromo-3-methyl-4-(tetrahydro-2H-pyran-4-yloxy)-1H-indazole as described therein, to prepare the titled compound. ¹H NMR (300 MHz, DMSO-d₆) δ ppm 1.09-1.33 (m, 5H), 1.50-2.06 (m, 13H), 2.19-2.35 (m, 1H), 2.54 (s, 3H), 2.64-2.72 (m, 4H), 2.85 (d, J=4.6 Hz, 3H), 2.96-3.04 (m, 4H), 5.14 (tt, J=5.3, 2.5 Hz, 1H), 6.67 (s, 1H), 7.17 (s, 1H), 7.38-7.46 (m, 2H), 7.78 (d, J=7.8 Hz, 1H), 9.07 (q, J=4.6 Hz, 1H), 12.55 (s, 1H); MS (DCI) m/z 516 (M+H)⁺.

### Example 122

### tert-butyl 4-{2-(methylcarbamoyl)-5-[3-methyl-4-(tetrahydro-2H-pyran-4-yloxy)-1H-indazol-6-yl]phenyl}piperazine-1-carboxylate

The procedure of Example 115, Step 8 was used, substituting tert-butyl 4-[2-(methylcarbamoyl)-5-(4,4,5,5-tetramethyl-1,3,2-dioxaborolan-2-yl)phenyl]piperazine-1-carboxylate (Example 111, Step 1) for N-ethyl-2-(4-phenylpiperazin-1-yl)-4-(4,4,5,5-tetramethyl-1,3,2-dioxaborolan-2-yl)benzamide as described therein, to prepare the titled compound. ¹H NMR (300 MHz, DMSO-d₆) δ ppm 1.43 (s, 9H), 1.69-1.83 (m, 2H), 2.00-2.11 (m, 2H), 2.60 (s, 3H), 2.86 (d, J=4.7 Hz, 3H), 2.93-3.02 (m, 4H), 3.47-3.57 (m, 4H), 3.60 (ddd, J=11.4, 7.6, 3.4 Hz, 2H), 3.86 (ddd, J=11.2, 7.1, 3.7 Hz, 2H), 4.97 (tt, J=7.1, 3.6 Hz, 1H), 6.76 (s, 1H), 7.19 (s, 1H), 7.39 (d, J=1.4 Hz, 1H), 7.45 (dd, J=8.1, 1.7 Hz, 1H), 7.77 (d, J=7.8 Hz, 1H), 8.89 (q, J=4.7 Hz, 1H), 12.59 (s, 1H); MS (ESI) m/z 550 (M+H)⁺.

### Example 123

### N-methyl-4-[3-methyl-4-(tetrahydro-2H-pyran-4-yloxy)-1H-indazol-6-yl]-2-[4-(pyridin-3-ylmethyl)piperazin-1-yl]benzamide

### Step 1

### N-methyl-4-[3-methyl-4-(tetrahydro-2H-pyran-4-yloxy)-1H-indazol-6-yl]-2-(piperazin-1-yl)benzamide hydrochloride

A solution of HCl in dioxane (4 M, 3 mL, 12 mmol) was added at room temperature to a solution of tert-butyl 4-{2-(methylcarbamoyl)-5-[3-methyl-4-(tetrahydro-2H-pyran-4-yloxy)-1H-indazol-6-yl]phenyl}piperazine-1-carboxylate (165 mg, 0.300 mmol, Example 122) in ethyl acetate (10 mL), and the resulting slurry was stirred for 16 hours. The mixture was concentrated under vacuum, and the residue was crystallized from ethanol-ethyl acetate (2:1, ca. 20 mL) to provide the titled compound (104 mg, 69%). ¹H NMR (300 MHz, DMSO-d₆) δ ppm 1.67-1.83 (m, 2H), 2.00-2.12 (m, 2H), 2.61 (s, 3H), 2.85 (d, J=4.7 Hz, 3H), 3.18-3.32 (m, 8H), 3.61 (m, 2H), 3.87 (ddd, J=11.1, 7.0, 3.6 Hz, 2H), 4.95 (tt, J=7.1, 3.4 Hz, 2H), 6.76 (s, 1H), 7.19 (d, J=1.0 Hz, 1H), 7.30 (d, J=1.4 Hz, 1H), 7.44 (dd, J=8.0, 1.5 Hz, 1H), 7.66 (d, J=8.1 Hz, 1H), 8.46 (q, J=4.4 Hz, 1H), 9.10 (s, 2H); MS (ESI) m/z 450 (M+H)⁺.

### Step 2

### N-methyl-4-[3-methyl-4-(tetrahydro-2H-pyran-4-yloxy)-1H-indazol-6-yl]-2-[4-(pyridin-3-ylmethyl)piperazin-1-yl]benzamide hydrochloride

Sodium cyanoborohydride (37.5 mg, 0.597 mmol) was added to a mixture of N-methyl-4-[3-methyl-4-(tetrahydro-2H-pyran-4-yloxy)-1H-indazol-6-yl]-2-(piperazin-1-yl)benzamide hydrochloride (29 mg, 0.060 mmol, Step 1) and pyridine-3-carboxaldehyde (32.0 mg, 0.298 mmol) in ethanol (4 mL). The reaction mixture was stirred at room temperature for 90 minutes, then concentrated under vacuum. The residue was diluted with 20% aqueous K₂CO₃ (10 mL) and extracted with ethyl acetate (2×15 mL). The combined extract was washed with saturated brine (5 mL), dried (MgSO₄) and concentrated under vacuum. The residue was purified by flash chromatography (silica gel, eluted with 5% CH₃OH/CH₂Cl₂). The product was dissolved in ethyl acetate (3 mL) and treated with 4 N HCl/dioxane (0.1 mL), and the resulting white suspension was concentrated under vacuum. The residual solid was crystallized from ethyl acetate-ethanol (ca 1:1, 2 mL), to provide the titled compound (8 mg, 22%). ¹H NMR (300 MHz, DMSO-d₆) δ ppm 1.63-1.87 (m, 2H), 1.97-2.12 (m, 2H), 2.60 (s, 3H), 2.85 (d, J=4.7 Hz, 3H), 3.19-3.31 (m, 4H), 3.35-3.50 (m, 4H), 3.59 (ddd, J=11.4, 7.6, 3.4 Hz, 2H), 3.79-3.92 (m, 2H), 4.50 (s, 2H), 4.94 (tt, J=7.4, 3.8 Hz, 1H), 6.75 (s, 1H), 7.17 (s, 1H), 7.31 (s, 1H), 7.45 (dd, J=8.1, 1.4 Hz, 1H), 7.62-7.68 (m, 2H), 8.23 (d, J=8.1 Hz, 1H), 8.45 (q, J=4.7 Hz, 1H), 8.74 (d, J=3.7 Hz, 1H), 8.88 (s, 1H), 10.98 (s, 1H); MS (ESI) m/z 541 (M+H)⁺.

### Example 124

### 2-[4-(cyclopropylcarbonyl)piperazin-1-yl]-N-methyl-4-[3-methyl-4-(tetrahydro-2H-pyran-4-yloxy)-1H-indazol-6-yl]benzamide

A solution of cyclopropanecarbonyl chloride (6.5 mg, 0.062 mmol) in CH₂Cl₂ (1 mL) was added to a mixture of N-methyl-4-[3-methyl-4-(tetrahydro-2H-pyran-4-yloxy)-1H-indazol-6-yl]-2-(piperazin-1-yl)benzamide hydrochloride (25 mg, 0.051 mmol, Example 123, Step 1) and triethylamine (0.018 mL, 0.132 mmol). The mixture was stirred at room temperature for 40 minutes, then diluted with methanol (2 mL) and allowed to stir at room temperature for 12 hours. The solution was concentrated under vacuum, and the residue was purified by flash chromatography (silica gel eluted with 5% CH₃OH/CH₂Cl₂) to provide the titled compound (9 mg, 33%). ¹H NMR (300 MHz, methanol-d₄) δ ppm 0.80-0.88 (m, 2H), 0.88-0.95 (m, 2H), 1.82-1.96 (m, 2H), 1.96-2.06 (m, 1H), 2.09-2.22 (m, 2H), 2.69 (s, 3H), 3.00 (s, 3H), 3.03-3.18 (m, 4H), 3.70 (ddd, J=11.4, 7.6, 3.4 Hz, 2H), 3.76-3.86 (m, 2H), 3.92-4.01 (m, 2H), 4.00 (ddd, J=11.6, 7.8, 3.4 Hz, 2H), 4.93 (tt, J=7.2, 3.7 Hz, 1H), 6.74 (s, 1H), 7.19 (d, J=1.0 Hz, 1H), 7.43-7.49 (m, 2H), 7.86 (d, J=7.8 Hz, 1H); MS (ESI) m/z 518 (M+H)⁺.

### Example 125

### isopropyl 4-{2-(methylcarbamoyl)-5-[3-methyl-4-(tetrahydro-2H-pyran-4-yloxy)-1H-indazol-6-yl]phenyl}piperazine-1-carboxylate

### Step 1

### N-methyl-4-[3-methyl-4-(tetrahydro-2H-pyran-4-yloxy)-1H-indazol-6-yl]-2-(piperazin-1-yl)benzamide

Trifluoroacetic acid (3 mL, 38.9 mmol) was added to an ice-cooled solution of tert-butyl 4-{2-(methylcarbamoyl)-5-[3-methyl-4-(tetrahydro-2H-pyran-4-yloxy)-1H-indazol-6-yl]phenyl}piperazine-1-carboxylate (Example 122, 356 mg, 0.65 mmol) in CH₂Cl₂ (5 mL). The resulting pale yellow solution was stirred with ice cooling for 3 hours. The solution was concentrated under vacuum, and the residue taken up in CH₂Cl₂ (5 mL) and concentrated again to remove excess trifluoroacetic acid. The residue was taken up with 20% aqueous K₂CO₃ (15 mL) and 1 M NaOH (5 mL) and extracted with CH₂Cl₂ (3×20 mL). The combined extract was dried (MgSO₄) and concentrated under vacuum to provide the titled compound (285 mg, 98%). ¹H NMR (300 MHz, DMSO-d₆) δ ppm 1.65-1.83 (m, 2H), 1.96-2.14 (m, 2H), 2.61 (s, 3H), 2.86 (d, J=4.7 Hz, 3H), 2.92-2.97 (m, 8H), 3.60 (ddd, J=11.2, 7.4, 3.4 Hz, 2H), 3.87 (ddd, J=10.8, 6.8, 3.6 Hz, 2H), 4.96 (tt, J=7.2, 3.6 Hz, 1H), 6.76 (s, 1H), 7.18 (d, J=1.0 Hz, 1H), 7.37 (d, J=1.7 Hz, 1H), 7.44 (dd, J=8.0, 1.7 Hz, 1H), 7.79 (d, J=8.0 Hz, 1H), 9.08 (q, J=4.4 Hz, 1H), 12.59 (s, 1H); MS (ESI) m/z 450 (M+H)⁺.

### Step 2

### isopropyl 4-{2-(methylcarbamoyl)-5-[3-methyl-4-(tetrahydro-2H-pyran-4-yloxy)-1H-indazol-6-yl]phenyl}piperazine-1-carboxylate

A solution of isopropyl chloroformate in toluene (1 M, 0.41 mL, 0.41 mmol) was added at room temperature to a stirring mixture of N-methyl-4-[3-methyl-4-(tetrahydro-2H-pyran-4-yloxy)-1H-indazol-6-yl]-2-(piperazin-1-yl)benzamide (140 mg, 0.311 mmol, Step 1) in CH₂Cl₂ (5 mL) and 20% aqueous K₂CO₃ (3 mL). The mixture was stirred at room temperature for 15 minutes, then methanol (1 mL) was added and the mixture stirred for 30 minutes longer. The mixture was partitioned between water (5 mL) and CH₂Cl₂ (10 mL), and the organic phase was separated, dried (MgSO₄) and concentrated under vacuum. The residue was purified by flash chromatography (silica gel eluted with CH₂Cl₂—CH₃OH, 98:2-95:5) to provide the titled compound (140 mg, 84%). ¹H NMR (300 MHz, DMSO-d₆) δ ppm 1.21 (d, J=6.1 Hz, 6H), 1.67-1.82 (m, 2H), 1.99-2.13 (m, 2H), 2.60 (s, 3H), 2.86 (d, J=4.7 Hz, 3H), 2.94-3.07 (m, 4H), 3.52-3.59 (m, 4H), 3.59-3.66 (m, 2H), 3.86 (ddd, J=11.2, 7.1, 3.7 Hz, 2H), 4.81 (qq, J=6.2 Hz, 1H), 4.97 (tt, J=6.9, 3.9, 3.6 Hz, 1H), 6.76 (s, 1H), 7.19 (s, 1H), 7.37-7.41 (m, 1H), 7.45 (dd, J=8.1, 1.7 Hz, 1H), 7.77 (d, J=8.1 Hz, 1H), 8.90 (q, J=4.4 Hz, 1H), 12.60 (s, 1H); MS (ESI) m/z 536 (M+H)⁺.

### Example 126

### 2-[2-(cyclohexyloxy)ethoxy]-N-methyl-4-[3-methyl-4-(tetrahydro-2H-pyran-4-yloxy)-1H-indazol-6-yl]benzamide

### Step 1

### 4-bromo-2-(2-cyclohexyloxy)ethoxy-N-methylbenzamide

2-Cyclohexyloxyethanol (827 mg, 5.74 mmol) was added at room temperature to a solution of potassium tert-butoxide (551 mg, 4.91 mmol) in dimethyl sulfoxide (10 mL), and the mixture was stirred for 1 hour. 4-Bromo-2-fluoro-N-methylbenzamide (1.00 g, 4.31 mmol) was added, and the mixture was stirred at ambient temperature for 2 hours, then diluted with water (100 mL) and extracted with ethyl acetate (3×100 mL). The combined extracts were washed with brine (100 mL), dried over MgSO₄ and concentrated to provide the titled compound (1.47 g, 96%). ¹H NMR (300 MHz, DMSO-d₆) δ ppm 1.13-1.34 (m, 4H), 1.44-1.51 (m, 1H), 1.61-1.67 (m, 2H), 1.82-1.88 (m, 2H), 2.81 (d, J=4.8 Hz, 3H), 3.33-3.38 (m, 1H), 3.77-3.80 (m, 2H), 4.26-4.29 (m, 2H), 7.25 (dd, J=8.3, 1.9 Hz, 1H), 7.42 (d, J=2.0 Hz, 1H), 7.75 (d, J=8.5 Hz, 1H), 8.11 (q, J=4.8 Hz, 1H) MS (ESI) m/z 356 (M+H)⁺.

### Step 2

### 2-[2-(cyclohexyloxy)ethoxy]-N-methyl-4-(4,4,5,5-tetramethyl-1,3,2-dioxaborolan-2-yl)benzamide

The procedure of Example 115, Step 7 was used, substituting 4-bromo-2-(2-cyclohexyloxy)ethoxy-N-methylbenzamide (Step 1) for 4-bromo-N-ethyl-2-(4-phenylpiperazin-1-yl)benzamide as described therein, to provide the titled compound. ¹H NMR (300 MHz, DMSO-d₆) δ ppm 1.21-1.27 (m, 5H), 1.30 (s, 12H), 1.46-1.51 (m, 1H), 1.64-1.69 (m, 2H), 1.83-1.89 (m, 2H), 2.82 (d, J=4.8 Hz, 3H), 3.34-3.38 (m, 1H), 3.77-3.80 (m, 2H), 4.24-4.27 (m, 2H), 7.33-7.36 (m, 2H), 7.85 (d, J=7.8 Hz, 1H), 8.23 (q, J=4.6 Hz, 1H); MS (ESI) m/z 404 (M+H)⁺.

### Step 3

### 2-[2-(cyclohexyloxy)ethoxy]-N-methyl-4-[3-methyl-4-(tetrahydro-2H-pyran-4-yloxy)-1H-indazol-6-yl]benzamide

The procedure of Example 115, Step 8 was used, substituting 2-[2-(cyclohexyloxy)ethoxy]-N-methyl-4-(4,4,5,5-tetramethyl-1,3,2-dioxaborolan-2-yl)benzamide (Step 2) for N-ethyl-2-(4-phenylpiperazin-1-yl)-4-(4,4,5,5-tetramethyl-1,3,2-dioxaborolan-2-yl)benzamide as described therein, to provide the titled compound after purification by preparative HPLC (30×100 mm Waters C18 XBridge™ column eluted with 25 mM aqueous (NH₄)₂CO₃—CH₃OH, 80:20-0:100, 40 mL/min over 15 minutes). ¹H NMR (300 MHz, methanol-d₄) δ ppm 1.21-1.43 (m, 5H), 1.51-1.64 (m, 1H), 1.70-1.82 (m, 2H), 1.83-2.03 (m, 4H), 2.10-2.23 (m, 2H), 2.69 (s, 3H), 2.98 (s, 3H), 3.38-3.48 (m, 1H), 3.70 (ddd, J=11.4, 7.5, 3.7 Hz, 2H), 3.88-3.95 (m, 2H), 4.00 (ddd, J=11.2, 7.1, 3.7 Hz, 2H), 4.37-4.43 (m, 2H), 4.94 (tt, J=7.1, 3.4 Hz, 1H), 6.77 (s, 1H), 7.22 (d, J=1.0 Hz, 1H), 7.33-7.39 (m, 1H), 7.38 (s, 1H), 8.05 (d, J=8.1 Hz, 1H); MS (ESI) m/z 508 (M+H)⁺.

### Example 127

### N-{4-[4-(cyclopentyloxy)-3-methyl-1H-indazol-6-yl]-2-[(2-phenoxyethyl)amino]phenyl}acetamide

### Step 1

### 2-[(5-bromo-2-nitrophenyl)amino]ethanol

A solution of 2-aminoethanol (350 mg, 5.73 mmol) and 4-bromo-2-fluoro-1-nitrobenzene (315 mg, 1.432 mmol) in ethanol (4 mL) was irradiated (Biotage Personal Chemistry unit, 300 W) at 110° C. for 20 minutes. The red-orange solution was concentrated under vacuum, and the residue purified by flash chromatography (silica gel eluted with hexanes-ethyl acetate, 90:10-60:40) to provide the titled compound (331 mg, 89%). ¹H NMR (300 MHz, DMSO-d₆) δ ppm 3.41 (q, J=5.6 Hz, 2H), 3.63 (q, J=5.6 Hz, 2H), 4.99 (t, J=5.2 Hz, 1H), 6.83 (dd, J=9.1, 2.0 Hz, 1H), 7.30 (d, J=2.0 Hz, 1H), 7.99 (d, J=9.1 Hz, 1H), 8.29 (t, J=5.0 Hz, 1H).

### Step 2

### 5-bromo-2-nitro-N-(2-phenoxyethyl)aniline

Diisopropyl azodicarboxylate (394 mg, 1.95 mmol) was added to an ice-cooled solution of phenol (178 mg, 1.90 mmol), 2-[(5-bromo-2-nitrophenyl)amino]ethanol (330 mg, 1.26 mmol, Step 1) and triphenylphosphine (398 mg, 1.52 mmol) in tetrahydrofuran (10 mL). The resulting yellow solution was stirred with ice cooling for 1 hour, then allowed to warm to room temperature overnight (12 hours). The reaction mixture was concentrated under vacuum, and the residue was purified by flash chromatography (silica gel eluted with hexanes-ethyl acetate, 100:0-75:25) to provide the titled compound (430 mg, 92%). ¹H NMR (300 MHz, methanol-d₄) δ ppm 3.70-3.80 (m, 2H), 4.26 (t, J=5.3 Hz, 2H), 6.83 (dd, J=9.2, 2.0 Hz, 1H), 6.90-6.98 (m, 3H), 7.22-7.32 (m, 2H), 7.36 (d, J=2.0 Hz, 1H), 8.03 (d, J=9.2 Hz, 1H).

### Step 3

### N-{4-bromo-2-[(2-phenoxyethyl)amino]phenyl}acetamide

A solution of ammonium chloride (683 mg, 12.8 mmol) in water (4 mL) was added to a solution of 5-bromo-2-nitro-N-(2-phenoxyethyl)aniline (429 mg, 1.27 mmol) in acetone (40 mL). The resulting milky suspension was cooled in ice for 10 minutes, and zinc dust (832 mg, 12.7 mmol) was added in a single portion. After 15 minutes, the grey suspension was filtered with an acetone (40 mL) rinse. The filtrate was concentrated under vacuum, and the residue was taken up in saturated brine (30 mL) and extracted with ethyl acetate (50 mL). The organic phase was dried (MgSO₄) and concentrated. The residue was dissolved in CH₂Cl₂ (10 mL), and the solution stirred at room temperature as a solution of acetic anhydride in CH₂Cl₂ (1.0 M, 1.27 mL, 1.27 mmol) was added. After 25 minutes, methanol (5 mL) was added, and the solution was stirred at room temperature for 12 hours), then concentrated under vacuum. The residue was purified by flash chromatography (silica gel eluted with hexanes-ethyl acetate 80:20-30:70) to provide the titled compound (150 mg, 34%). ¹H NMR (300 MHz, DMSO-d₆) δ ppm 2.02 (s, 3H), 3.46 (q, J=5.6 Hz, 2H), 4.12 (t, J=5.6 Hz, 2H), 5.43 (t, J=5.6 Hz, 1H), 6.72 (dd, J=8.3, 2.4 Hz, 1H), 6.88 (d, J=2.0 Hz, 1H), 6.91-6.98 (m, 1H), 6.96 (d, J=8.7 Hz, 2H), 7.08 (d, J=8.3 Hz, 1H), 7.25-7.35 (m, 2H), 9.15 (s, 1H); MS (ESI) m/z 349/351 (M+H)⁺.

### Step 4

### 4-(cyclopentyloxy)-3-methyl-6-(4,4,5,5-tetramethyl-1,3,2-dioxaborolan-2-yl)-1H-indazole

The procedure of Example 115, Step 7 was used, substituting 6-bromo-4-(cyclopentyloxy)-3-methyl-1H-indazole (Example 121, Step 2) for 4-bromo-N-ethyl-2-(4-phenylpiperazin-1-yl)benzamide as described therein, to provide the titled compound. ¹H NMR (300 MHz, CDCl₃) δ ppm 1.36 (s, 12H), 1.56-1.90 (m, 4H), 1.90-2.06 (m, 4H), 4.93-5.13 (m, 1H), 6.79 (s, 1H), 7.52 (s, 1H); MS (ESI) m/z 343 (M+H)⁺.

### Step 5

### N-{4-[4-(cyclopentyloxy)-3-methyl-1H-indazol-6-yl]-2-[(2-phenoxyethyl)amino]phenyl}acetamide

The procedure of Example 115, Step 8, substituting 4-(cyclopentyloxy)-3-methyl-6-(4,4,5,5-tetramethyl-1,3,2-dioxaborolan-2-yl)-1H-indazole for N-ethyl-2-(4-phenylpiperazin-1-yl)-4-(4,4,5,5-tetramethyl-1,3,2-dioxaborolan-2-yl)benzamide and N-{4-bromo-2-[(2-phenoxyethyl)amino]phenyl}acetamide (Step 3) for 6-bromo-3-methyl-4-(tetrahydro-2H-pyran-4-yloxy)-1H-indazole as described therein, to provide the titled compound, purified by preparative HPLC (30×100 mm Waters C18 XBridge™ column eluted with 25 mM aqueous (NH₄)₂CO₃—CH₃OH, 80:20-0:100, 40 mL/minute over 15 minutes). ¹H NMR (400 MHz, methanol-d₄) δ ppm 1.63-2.00 (m, 8H), 2.16 (s, 3H), 2.61 (s, 3H), 3.62 (t, J=5.6 Hz, 2H), 4.22 (t, J=5.6 Hz, 2H), 5.00 (tt, J=4.0, 3.9, 3.7 Hz, 1H), 6.63 (s, 1H), 6.92 (t, J=7.5 Hz, 1H), 6.94-6.99 (m, 3H), 7.08 (d, J=1.5 Hz, 1H), 7.11 (s, 1H), 7.20 (d, J=7.9 Hz, 1H), 7.23-7.29 (m, 2H); MS (ESI) m/z 485 (M+H)⁺.

### Example 128

### isobutyl 4-{5-[4-(cyclopentyloxy)-3-methyl-1H-indazol-6-yl]-2-(methylcarbamoyl)phenyl}piperazine-1-carboxylate

### Step 1

### tert-butyl 4-{5-[4-(cyclopentyloxy)-3-methyl-1H-indazol-6-yl]-2-(methylcarbamoyl)phenyl}piperazine-1-carboxylate

The procedure of Example 115, Step 8, substituting 4-(cyclopentyloxy)-3-methyl-6-(4,4,5,5-tetramethyl-1,3,2-dioxaborolan-2-yl)-1H-indazole (Example 127, Step 4) for N-ethyl-2-(4-phenylpiperazin-1-yl)-4-(4,4,5,5-tetramethyl-1,3,2-dioxaborolan-2-yl)benzamide, and tert-butyl 4-[5-bromo-2-(methylcarbamoyl)phenyl]piperazine-1-carboxylate (228 mg, 0.573 mmol, Example 172, Step 1) for 6-bromo-3-methyl-4-(tetrahydro-2H-pyran-4-yloxy)-1H-indazole as described therein, to provide the titled compound, purified by preparative HPLC (30×100 mm Waters C18 XBridge™ column eluted with aqueous CF₃CO₂H—CH₃OH, 80:20-0:100, 40 mL/minute over 15 minutes). ¹H NMR (300 MHz, DMSO-d₆) δ ppm 1.43 (s, 9H), 1.57-2.08 (m, 8H), 2.54 (s, 3H), 2.86 (d, J=4.7 Hz, 3H), 2.94-3.03 (m, 4H), 3.50-3.57 (m, 4H), 5.09-5.18 (m, 1H), 6.67 (s, 1H), 7.18 (s, 1H), 7.41 (d, J=1.4 Hz, 1H), 7.45 (dd, J=8.1, 1.4 Hz, 1H), 7.77 (d, J=8.1 Hz, 1H), 8.90 (q, J=4.5 Hz, 1H), 12.56 (s, 1H); MS (ESI) m/z 534 (M+H)⁺.

### Step 2

### 4-[4-(cyclopentyloxy)-3-methyl-1H-indazol-6-yl]-N-methyl-2-(piperazin-1-yl)benzamide trifluoroacetate

Trifluoroacetic acid (2 mL) was added to an ice-cooled solution of tert-butyl 4-{5-[4-(cyclopentyloxy)-3-methyl-1H-indazol-6-yl]-2-(methylcarbamoyl)phenyl}piperazine-1-carboxylate (100 mg, 0.187 mmol, Step 1) in CH₂Cl₂ (4 mL). The solution was stirred with ice cooling for 4 hours, then concentrated under vacuum to leave the titled compound (100 mg, 97%), which was used directly in the next step.

### Step 3

### isobutyl 4-{5-[4-(cyclopentyloxy)-3-methyl-1H-indazol-6-yl]-2-(methylcarbamoyl)phenyl}piperazine-1-carboxylate

A solution of isobutyl chloroformate (12 mg, 0.088 mmol) in ethyl acetate (0.12 mL) was added to a mixture of 4-[4-(cyclopentyloxy)-3-methyl-1H-indazol-6-yl]-N-methyl-2-(piperazin-1-yl)benzamide trifluoroacetate (26 mg, 0.060 mmol) in ethyl acetate (2 mL) and saturated aqueous NaHCO₃ (1 mL). The mixture was stirred vigorously at room temperature for 1 hour. The organic layer was separated, and the aqueous phase was extracted once with ethyl acetate (1 mL). The combined organic phases were purified by flash chromatography (silica gel eluted with hexanes-ethyl acetate, 80:20-20:80) to provide titled compound (12.5 mg, 60%). ¹H NMR (300 MHz, DMSO-d₆) δ ppm 0.91 (d, J=6.8 Hz, 6H), 1.57-2.06 (m, 9H), 2.54 (s, 3H), 2.86 (d, J=4.7 Hz, 3H), 2.97-3.04 (m, 4H), 3.55-3.64 (m, 4H), 3.82 (d, J=6.4 Hz, 2H), 5.13 (tt, J=5.3, 2.7 Hz, 1H), 6.67 (s, 1H), 7.17 (s, 1H), 7.40 (d, J=1.7 Hz, 1H), 7.44 (dd, J=8.1, 1.7 Hz, 1H), 7.76 (d, J=8.1 Hz, 1H), 8.89 (q, J=4.7 Hz, 1H), 12.56 (s, 1H); MS (ESI) m/z 534 (M+H)⁺.

### Example 129

### benzyl 4-{5-[4-(cyclopentyloxy)-3-methyl-1H-indazol-6-yl]-2-(methylcarbamoyl)phenyl}piperazine-1-carboxylate

The procedure of Example 128, Step 3 was used, substituting benzyl chloroformate for isobutyl chloroformate as described therein, to provide the titled compound. ¹H NMR (300 MHz, DMSO-d₆) δ ppm 1.56-2.04 (m, 8H), 2.54 (s, 3H), 2.86 (d, J=4.7 Hz, 3H), 2.95-3.06 (m, 4H), 3.57-3.70 (m, 4H), 5.12 (s, 2H), 5.12-5.17 (m, 1H), 6.66 (s, 1H), 7.17 (s, 1H), 7.28-7.41 (m, 6H), 7.44 (dd, J=8.0, 1.5 Hz, 1H), 7.76 (d, J=8.1 Hz, 1H), 8.88 (q, J=4.3 Hz, 1H), 12.56 (s, 1H); MS (ESI) m/z 568 (M+H)⁺.

### Example 130

### N-{4-[3-methyl-4-(tetrahydro-2H-pyran-4-yloxy)-1H-indazol-6-yl]-2-(2-phenoxyethoxy)phenyl}acetamide

### Step 1

### 4-bromo-1-nitro-2-(2-phenoxyethoxy)benzene

2-Phenoxyethanol (345 mg, 2.500 mmol) was added to a suspension of potassium tert-butoxide (306 mg, 2.73 mmol) in tetrahydrofuran (10 mL). The suspension was stirred for 10 minutes, then 4-bromo-2-fluoronitrobenzene (500 mg, 2.273 mmol) was added, and stirring was continued at room temperature for 41 hours. The mixture was concentrated under vacuum, and the residue was diluted with water (15 mL) and extracted with ethyl acetate (2×30 mL). The combined extract was dried (MgSO₄), concentrated under vacuum and purified by flash chromatography (silica gel eluted with hexanes-ethyl acetate, 100:0-90:10) to provide the titled compound (546 mg, 71%). ¹H NMR (300 MHz, DMSO-d₆) δ ppm 4.35-4.42 (m, 2H), 4.44-4.50 (m, 2H), 6.91-6.95 (m, 2H), 6.96-7.02 (m, 1H), 7.21 (dd, J=8.8, 1.7 Hz, 1H), 7.27-7.35 (m, 2H), 7.38 (d, J=1.7 Hz, 1H), 7.74 (d, J=8.8 Hz, 1H).

### Step 2

### N-[4-bromo-2-(2-phenoxyethoxy)phenyl]acetamide

The procedure of Example 127, Step 3 was used, substituting 4-bromo-1-nitro-2-(2-phenoxyethoxy)benzene (Step 1) for 5-bromo-2-nitro-N-(2-phenoxyethyl)aniline as described therein, to provide the titled compound. ¹H NMR (300 MHz, DMSO-d₆) δ ppm 2.04 (s, 3H), 4.32-4.39 (m, 2H), 4.39-4.45 (m, 2H), 6.92-7.02 (m, 3H), 7.11 (dd, J=8.8, 2.0 Hz, 1H), 7.26-7.34 (m, 2H), 7.34 (d, J=2.0 Hz, 1H), 7.91 (d, J=8.5 Hz, 1H), 8.98 (s, 1H); MS (ESI) m/z 348/350 (M+H)⁺.

### Step 3

### N-[2-(2-phenoxyethoxy)-4-(4,4,5,5-tetramethyl-1,3,2-dioxaborolan-2-yl)phenyl]acetamide

The procedure of Example 115, Step 7 was used, substituting N-[4-bromo-2-(2-phenoxyethoxy)phenyl]acetamide for 4-bromo-N-ethyl-2-(4-phenylpiperazin-1-yl)benzamide as described therein, to provide the titled compound. ¹H NMR (500 MHz, CDCl₃) δ ppm 1.34 (s, 12H), 2.08 (s, 3H), 4.31-4.36 (m, 2H), 4.41-4.48 (m, 2H), 6.93-7.02 (m, 3H), 7.32 (dd, J=8.5, 7.3 Hz, 2H), 7.38 (s, 1H), 7.47 (d, J=7.9 Hz, 1H), 7.95 (s, 1H), 8.40 (d, J=7.9 Hz, 1H); MS (ESI) m/z 398 (M+H)⁺.

### Step 4

### N-{4-[3-methyl-4-(tetrahydro-2H-pyran-4-yloxy)-1H-indazol-6-yl]-2-(2-phenoxyethoxy)phenyl}acetamide

The procedure of Example 115, Step 8 was used, substituting N-[2-(2-phenoxyethoxy)-4-(4,4,5,5-tetramethyl-1,3,2-dioxaborolan-2-yl)phenyl]acetamide for N-ethyl-2-(4-phenylpiperazin-1-yl)-4-(4,4,5,5-tetramethyl-1,3,2-dioxaborolan-2-yl)benzamide as described therein, to provide the titled compound. ¹H NMR (300 MHz, DMSO-d₆) δ ppm 1.66-1.83 (m, 2H), 1.98-2.12 (m, 2H), 2.07 (s, 3H), 2.60 (s, 3H), 3.59 (ddd, J=11.4, 7.6, 3.4 Hz, 2H), 3.86 (ddd, J=11.1, 6.9, 3.7 Hz, 2H), 4.37-4.43 (m, 2H), 4.47-4.55 (m, 2H), 4.95 (tt, J=7.2, 3.7 Hz, 1H), 6.77 (s, 1H), 6.96 (t, J=7.5 Hz, 1H), 7.01 (d, J=7.8 Hz, 2H), 7.16 (s, 1H), 7.23-7.29 (m, 1H), 7.28-7.35 (m, 2H), 7.40 (d, J=1.7 Hz, 1H), 8.04 (br d, J=8.1 Hz, 1H), 8.98 (s, 1H), 12.54 (s, 1H); MS (ESI) m/z 502 (M+H)⁺.

### Example 131

### N-{2-(4-cyclohexylpiperazin-1-yl)-4-[3-methyl-4-(tetrahydro-2H-pyran-4-yloxy)-1H-indazol-6-yl]phenyl}acrylamide

### Step 1

### tert-butyl [4-bromo-2-(4-cyclohexylpiperazin-1-yl)phenyl]carbamate

Di(tert-butyl)dicarbonate (1.55 g, 7.09 mmol) was added to a solution of 4-bromo-2-(4-cyclohexylpiperazin-1-yl)aniline (2.0 g, 5.91 mmol, Example 119, Step 2) and 4-(N,N-dimethylamino)pyridine (0.144 g, 1.182 mmol) in CH₂Cl₂ (30 mL), and the resulting solution was stirred at room temperature. After 54 hours, the solution was concentrated under vacuum, and the residue was purified by flash chromatography (silica gel eluted with hexanes-ethyl acetate, 100:0-80:20) to provide the titled compound (720 mg, 28%). ¹H NMR (300 MHz, DMSO-d₆) δ ppm 1.03-1.31 (m, 5H), 1.46 (s, 9H), 1.58 (br d, J=11.9 Hz, 1H), 1.69-1.84 (m, 4H), 2.22-2.32 (m, 1H), 2.59-2.67 (m, 4H), 2.74-2.81 (m, 4H), 7.23 (dd, J=8.6, 2.2 Hz, 1H), 7.27 (d, J=2.0 Hz, 1H), 7.69 (d, J=8.7 Hz, 1H), 7.75 (s, 1H); MS (ESI) m/z 438/440 (M+H)⁺.

### Step 2

### tert-butyl [2-(4-cyclohexylpiperazin-1-yl)-4-(4,4,5,5-tetramethyl-1,3,2-dioxaborolan-2-yl)phenyl]carbamate

The procedure of Example 115, Step 7 was used, substituting tert-butyl [4-bromo-2-(4-cyclohexylpiperazin-1-yl)phenyl]carbamate (Step 1) for 4-bromo-N-ethyl-2-(4-phenylpiperazin-1-yl)benzamide as described therein, to provide the titled compound. ¹H NMR (300 MHz, CDCl₃) δ ppm 1.32 (s, 12H), 1.54 (s, 9H), 1.58-1.98 (m, 10H), 2.27-2.38 (m, 1H), 2.69-2.79 (m, 4H), 2.85-2.94 (m, 4H), 7.56 (dd, J=8.1, 1.4 Hz, 1H), 7.62 (d, J=1.4 Hz, 1H), 7.88 (s, 1H), 8.06 (d, J=8.5 Hz, 1H); MS (DCI) m/z 486 (M+H)⁺.

### Step 3

### tert-butyl {2-(4-cyclohexylpiperazin-1-yl)-4-[3-methyl-4-(tetrahydro-2H-pyran-4-yloxy)-1H-indazol-6-yl]phenyl}carbamate

A mixture of tert-butyl [2-(4-cyclohexylpiperazin-1-yl)-4-(4,4,5,5-tetramethyl-1,3,2-dioxaborolan-2-yl)phenyl]carbamate (345 mg, 0.711 mmol, Step 2) and 6-bromo-3-methyl-4-(tetrahydro-2H-pyran-4-yloxy)-1H-indazole (230 mg, 0.739 mmol, Example 115, Step 4) in ethanol (7 mL) and 1,2-dimethoxyethane (7 mL) was purged with nitrogen for 5 minutes. Aqueous K₂CO₃ (1 M, 1.4 mL, 1.4 mmol) was added, followed by bis(triphenylphosphine)palladium(II)dichloride (25 mg, 0.036 mmol), and the mixture was warmed to 90° C. under nitrogen. After 19 hours, the solution was cooled to room temperature and concentrated under vacuum. The residue was partitioned with water (7 mL) and CH₂Cl₂ (35 mL). The organic layer was dried (MgSO₄) and concentrated under vacuum. The residue was purified by flash chromatography (silica gel eluted with CH₂Cl₂—CH₃OH, 95:5) to provide the titled compound (310 mg, 74%). ¹H NMR (300 MHz, DMSO-d₆) δ ppm 1.13-1.31 (m, 5H), 1.49 (s, 9H), 1.54-1.86 (m, 7H), 1.99-2.12 (m, 2H), 2.20-2.33 (m, 1H), 2.59 (s, 3H), 2.64-2.74 (m, 4H), 2.81-2.91 (m, 4H), 3.60 (ddd, J=11.2, 7.5, 3.4 Hz, 2H), 3.86 (ddd, J=11.4, 7.4, 3.9 Hz, 2H), 4.96 (tt, J=6.8, 4.1 Hz, 1H), 6.72 (s, 1H), 7.10 (s, 1H), 7.40 (dd, J=8.1, 2.0 Hz, 1H), 7.43 (d, J=2.0 Hz, 1H), 7.80 (s, 1H), 7.87 (d, J=8.5 Hz, 1H), 12.50 (s, 1H); MS (ESI) m/z 590 (M+H)⁺.

### Step 4

### 2-(4-cyclohexylpiperazin-1-yl)-4-[3-methyl-4-(tetrahydro-2H-pyran-4-yloxy)-1H-indazol-6-yl]aniline

Trifluoroacetic acid (1.2 mL, 15.6 mmol) was added to an ice-cooled solution of tert-butyl {2-(4-cyclohexylpiperazin-1-yl)-4-[3-methyl-4-(tetrahydro-2H-pyran-4-yloxy)-1H-indazol-6-yl]phenyl}carbamate (310 mg, 0.53 mmol, Step 3) in CH₂Cl₂ (3 mL). The solution was stirred with ice cooling under nitrogen for 2 hours, then allowed to warm gradually to room temperature for 5 hours. The solution was concentrated under vacuum, and the residue was taken up in 20% aqueous K₂CO₃ (20 mL) and extracted with CH₂Cl₂ (2×20 mL). The combined extract was dried (MgSO₄) and concentrated to provide the titled compound which was used directly in the next step (313 mg).

### Step 5

### N-{2-(4-cyclohexylpiperazin-1-yl)-4-[3-methyl-4-(tetrahydro-2H-pyran-4-yloxy)-1H-indazol-6-yl]phenyl}acrylamide

A solution of acryloyl chloride (24 mg, 0.27 mmol) in CH₂Cl₂ (0.4 mL) was added to an ice cooled solution of 2-(4-cyclohexylpiperazin-1-yl)-4-[3-methyl-4-(tetrahydro-2H-pyran-4-yloxy)-1H-indazol-6-yl]aniline (42 mg, 0.086 mmol, Step 4) and triethylamine (0.048 mL, 0.343 mmol) in CH₂Cl₂ (3 mL). The resulting yellow solution was stirred at 0° C. for 5 hours. Methanol (1 mL) was added, and the mixture was allowed to warm to room temperature, then concentrated under vacuum. The residue was purified by HPLC (30×100 mm Waters C18 XBridge™ column eluted with aqueous 0.1% CF₃CO₂H—CH₃CN, 80:20-0:100, 40 mL/minute over 15 minutes) to provide the titled compound (22 mg, 47%). ¹H NMR (300 MHz, methanol-d₄) δ ppm 1.14-1.42 (m, 6H), 1.65-1.75 (m, 1H), 1.83-1.96 (m, 4H), 1.98-2.09 (m, 2H), 2.09-2.23 (m, 2H), 2.44-2.58 (m, 1H), 2.68 (s, 3H), 2.89-3.00 (m, 4H), 3.00-3.09 (m, 4H), 3.70 (ddd, J=11.4, 7.5, 3.7 Hz, 2H), 4.01 (ddd, J=11.3, 7.2, 3.6 Hz, 2H), 4.87-4.96 (m, 1H), 5.83 (dd, J=10.0, 1.7 Hz, 1H), 6.38 (dd, J=17.0, 1.7 Hz, 1H), 6.55 (dd, J=17.0, 9.9 Hz, 1H), 6.71 (s, 1H), 7.14 (d, J=1.0 Hz, 1H), 7.43 (dd, J=8.1, 2.0 Hz, 1H), 7.47 (d, J=2.0 Hz, 1H), 8.16 (d, J=8.1 Hz, 1H); MS (ESI) m/z 544 (M+H)⁺.

### Example 132

### 4-[3-methyl-4-(tetrahydro-2H-pyran-4-yloxy)-1H-indazol-6-yl]benzamide

### Step 1

### 4-[3-methyl-4-(tetrahydro-2H-pyran-4-yloxy)-1H-indazol-6-yl]benzonitrile

The procedure of Example 115, Step 8 was used, substituting 4-(4,4,5,5-tetramethyl-1,3,2-dioxaborolan-2-yl)benzonitrile for N-ethyl-2-(4-phenylpiperazin-1-yl)-4-(4,4,5,5-tetramethyl-1,3,2-dioxaborolan-2-yl)benzamide as described therein, to provide the titled compound. ¹H NMR (300 MHz, DMSO-d₆) δ ppm 1.66-1.86 (m, 2H), 2.00-2.16 (m, 2H), 2.60 (s, 3H), 3.59 (ddd, J=11.4, 7.9, 3.4 Hz, 2H), 3.77-3.93 (m, 2H), 4.97 (tt, J=7.5, 3.7 Hz, 1H), 6.83 (s, 1H), 6.94 (none, 1H), 7.25 (s, 1H), 7.87-7.97 (m, 4H), 12.69 (s, 1H); MS (ESI) m/z 334 (M+H)⁺.

### Step 2

### 4-[3-methyl-4-(tetrahydro-2H-pyran-4-yloxy)-1H-indazol-6-yl]benzamide

Aqueous NaOH (1 M, 0.21 mL, 0.21 mmol) and then 50% aqueous H₂O₂ (0.068 mL, 1.1 mmol) were added to a solution of 4-[3-methyl-4-(tetrahydro-2H-pyran-4-yloxy)-1H-indazol-6-yl]benzonitrile (46 mg, 0.138 mmol, Step 1) in dimethyl sulfoxide (1 mL). The resulting suspension was stirred at room temperature for 2 hours, then diluted with water (20 mL) and heated to boiling for 5 minutes. The mixture was allowed to cool to room temperature for 2 hours, the precipitate was collected by filtration and washed well with water (15 mL total), and then dried under vacuum. The solid (39 mg) was crystallized from ethanol-water (60:40, 3 mL) to provide the titled compound. ¹H NMR (300 MHz, DMSO-d₆) δ ppm 1.57-1.72 (m, 2H), 1.90-2.03 (m, 2H), 2.49 (s, 3H), 3.49 (ddd, J=11.1, 7.9, 3.1 Hz, 2H), 3.70-3.80 (m, 2H), 4.79-4.91 (m, 1H), 6.71 (s, 1H), 7.10 (s, 1H), 7.26 (s, 1H), 7.67 (d, J=8.2 Hz, 2H), 7.85 (d, J=8.2 Hz, 2H), 7.90 (br s, 1H), 12.51 (s, 1H); MS (ESI) m/z 352 (M+H)⁺.

### Example 133

### N-{2-(4-cyclohexylpiperazin-1-yl)-4-[3-methyl-4-(tetrahydro-2H-pyran-4-yloxy)-1H-indazol-6-yl]phenyl}-2-oxopropanamide

The procedure of Example 131, Step 5 was used, substituting pyruvoyl chloride (prepared as described by Ottenheijm, H C J, et al. Synthesis 1975; 163-164) in place of acryloyl chloride as described therein, to provide the titled compound. ¹H NMR (300 MHz, CHCl₃) δ ppm 1.18-1.35 (m, 4H), 1.62-1.72 (m, 2H), 1.78-2.03 (m, 6H), 2.07-2.20 (m, 2H), 2.29-2.44 (m, 1H), 2.60 (s, 3H), 2.73 (s, 3H), 2.79-2.90 (m, 4H), 2.93-3.04 (m, 4H), 3.71 (ddd, J=11.3, 7.0, 3.7 Hz, 2H), 4.03 (ddd, J=11.5, 7.6, 3.6 Hz, 2H), 4.81 (tt, J=6.8, 3.7, 3.4 Hz, 1H), 6.58 (s, 1H), 7.08 (d, J=1.0 Hz, 1H), 7.36-7.44 (m, 2H), 8.47 (d, J=8.1 Hz, 1H), 10.06 (s, 1H); MS (ESI) m/z 560 (M+H)⁺.

### Example 134

### ethyl 4-{2-(methylcarbamoyl)-5-[3-methyl-4-(tetrahydro-2H-pyran-4-yloxy)-1H-indazol-6-yl]phenyl}piperazine-1-carboxylate

The procedure of Example 125 was used, substituting ethyl chloroformate for isopropyl chloroformate as described therein, to provide the titled compound. ¹H NMR (400 MHz, DMSO-d₆) δ ppm 1.09 (t, J=7.0 Hz, 3H), 1.58-1.70 (m, 2H), 1.87-2.02 (m, 2H), 2.49 (s, 3H), 2.74 (d, J=4.3 Hz, 3H), 2.83-2.92 (m, 4H), 3.40-3.53 (m, 6H), 3.69-3.80 (m, 2H), 3.96 (q, J=7.0 Hz, 2H), 4.79-4.91 (m, 1H), 6.65 (s, 1H), 7.08 (s, 1H), 7.28 (s, 1H), 7.33 (d, J=8.2 Hz, 1H), 7.65 (d, J=8.2 Hz, 1H), 8.77 (q, J=4.3 Hz, 1H), 12.49 (s, 1H); MS (ESI) m/z 522 (M+H)⁺.

### Example 135

### propyl 4-{2-(methylcarbamoyl)-5-[3-methyl-4-(tetrahydro-2H-pyran-4-yloxy)-1H-indazol-6-yl]phenyl}piperazine-1-carboxylate

The procedure of Example 125 was used, substituting propyl chloroformate for isopropyl chloroformate as described therein, to provide the titled compound. ¹H NMR (400 MHz, DMSO-d₆) δ ppm 0.79 (t, J=7.3 Hz, 3H), 1.48 (sextet, J=7.1 Hz, 2H), 1.58-1.69 (m, 2H), 1.88-2.00 (m, 2H), 2.49 (s, 3H), 2.74 (d, J=4.3 Hz, 3H), 2.84-2.91 (m, 4H), 3.40-3.54 (m, 6H), 3.70-3.78 (m, 2H), 3.87 (t, J=6.6 Hz, 2H), 4.79-4.91 (m, 1H), 6.65 (s, 1H), 7.08 (s, 1H), 7.28 (s, 1H), 7.33 (d, J=7.9 Hz, 1H), 7.65 (d, J=7.9 Hz, 1H), 8.78 (q, J=4.6 Hz, 1H), 12.49 (s, 1H); MS (ESI) m/z 536 (M+H)⁺.

### Example 136

### 2,2,2-trifluoroethyl 4-{2-(methylcarbamoyl)-5-[3-methyl-4-(tetrahydro-2H-pyran-4-yloxy)-1H-indazol-6-yl]phenyl}piperazine-1-carboxylate

The procedure of Example 125 was used, substituting 2,2,2-trifluoroethyl chloroformate for isopropyl chloroformate as described therein, to provide the titled compound. ¹H NMR (300 MHz, DMSO-d₆) δ ppm 1.68-1.83 (m, 2H), 1.99-2.12 (m, 2H), 2.61 (s, 3H), 2.86 (d, J=4.7 Hz, 3H), 2.96-3.10 (m, 4H), 3.54-3.66 (m, 6H), 3.86 (ddd, J=11.2, 7.1, 3.7 Hz, 2H), 4.75 (q, J=9.0 Hz, 2H), 4.97 (tt, J=7.0, 3.5 Hz, 1H), 6.77 (s, 1H), 7.20 (s, 1H), 7.40 (s, 1H), 7.45 (dd, J=7.8, 1.5 Hz, 1H), 7.76 (d, J=7.8 Hz, 1H), 8.84 (q, J=4.3 Hz, 1H), 12.60 (s, 1H); MS (ESI) m/z 576 (M+H)⁺.

### Example 137

### 2,2-dimethylpropyl 4-{2-(methylcarbamoyl)-5-[3-methyl-4-(tetrahydro-2H-pyran-4-yloxy)-1H-indazol-6-yl]phenyl}piperazine-1-carboxylate

The procedure of Example 125 was used, substituting neopentyl chloroformate for isopropyl chloroformate as described therein, to provide the titled compound. ¹H NMR (300 MHz, DMSO-d₆) δ ppm 0.93 (s, 9H), 1.69-1.82 (m, 2H), 2.00-2.11 (m, 2H), 2.60 (s, 3H), 2.86 (d, J=4.7 Hz, 3H), 2.98-3.04 (m, 4H), 3.54-3.66 (m, 6H), 3.74 (s, 2H), 3.81-3.91 (m, 2H), 4.97 (tt, J=7.1, 3.6 Hz, 1H), 6.77 (s, 1H), 7.19 (s, 1H), 7.40 (d, J=1.4 Hz, 1H), 7.45 (dd, J=7.9, 1.5 Hz, 1H), 7.76 (d, J=7.9 Hz, 1H), 8.88 (q, J=4.7 Hz, 1H), 12.59 (s, 1H); MS (ESI) m/z 564 (M+H)⁺.

### Example 138

### 2-methoxyethyl 4-{2-(methylcarbamoyl)-5-[3-methyl-4-(tetrahydro-2H-pyran-4-yloxy)-1H-indazol-6-yl]phenyl}piperazine-1-carboxylate

The procedure of Example 125 was used, substituting 2-methoxyethyl chloroformate for isopropyl chloroformate as described therein, to provide the titled compound. ¹H NMR (300 MHz, DMSO-d₆) δ ppm 1.68-1.83 (m, 2H), 2.00-2.13 (m, 2H), 2.60 (s, 3H), 2.86 (d, J=4.7 Hz, 3H), 2.96-3.04 (m, 4H), 3.28 (s, 3H), 3.50-3.66 (m, 8H), 3.86 (ddd, J=11.2, 7.1, 3.7 Hz, 2H), 4.12-4.18 (m, 2H), 4.97 (tt, J=7.1, 3.6 Hz, 1H), 6.76 (s, 1H), 7.19 (s, 1H), 7.39 (d, J=1.4 Hz, 1H), 7.45 (dd, J=7.9, 1.5 Hz, 1H), 7.77 (d, J=7.9 Hz, 1H), 8.88 (q, J=4.3 Hz, 1H), 12.60 (s, 1H); MS (ESI) m/z 552 (M+H)⁺.

### Example 139

### tert-butyl 4-{2-cyano-5-[3-methyl-4-(tetrahydro-2H-pyran-4-yloxy)-1H-indazol-6-yl]phenyl}piperazine-1-carboxylate

### Step 1

### tert-butyl 4-(5-bromo-2-cyanophenyl)piperazine-1-carboxylate

A mixture of piperazine (115 mg, 1.34 mmol) and 4-bromo-2-fluorobenzonitrile (267 mg, 1.34 mmol) in ethanol (4 mL) was irradiated (Biotage Personal Creator 300 W unit) at 120° C. for 300 minutes. The reaction mixture was concentrated under vacuum, and the residue was diluted with CH₂Cl₂ (20 mL) and 1 M NaOH (3 mL). Di-tert-butyl dicarbonate (353 mg, 1.62 mmol) was added, and the mixture was stirred at room temperature for 80 minutes. The layers were separated, and the organic phase was concentrated and purified by flash chromatography (silica gel eluted with heptanes-ethyl acetate, 100:0-70:30) to provide the titled compound (245 mg, 50%). MS (DCI) m/z 366/368 (M+H)⁺.

### Step 2

### tert-butyl 4-[2-cyano-5-(4,4,5,5-tetramethyl-1,3,2-dioxaborolan-2-yl)phenyl]piperazine-1-carboxylate

The procedure of Example 115, Step 7 was used, substituting tert-butyl 4-(5-bromo-2-cyanophenyl)piperazine-1-carboxylate (Step 1) for 4-bromo-N-ethyl-2-(4-phenylpiperazin-1-yl)benzamide as described therein, to provide the titled compound. ¹H NMR (500 MHz, methanol-d₄) δ ppm 1.35 (s, 12H), 1.49 (s, 9H), 3.12-3.17 (m, 4H), 3.57-3.67 (m, 4H), 7.45 (d, J=7.6 Hz, 1H), 7.47 (s, 1H), 7.63 (d, J=7.6 Hz, 1H).

### Step 3

### tert-butyl 4-{2-cyano-5-[3-methyl-4-(tetrahydro-2H-pyran-4-yloxy)-1H-indazol-6-yl]phenyl}piperazine-1-carboxylate

The procedure of Example 115, Step 8 was used, substituting tert-butyl 4-[2-cyano-5-(4,4,5,5-tetramethyl-1,3,2-dioxaborolan-2-yl)phenyl]piperazine-1-carboxylate (Step 2) for N-ethyl-2-(4-phenylpiperazin-1-yl)-4-(4,4,5,5-tetramethyl-1,3,2-dioxaborolan-2-yl)benzamide as described therein, to provide the titled compound. ¹H NMR (300 MHz, DMSO-d₆) δ ppm 1.43 (s, 9H), 1.68-1.80 (m, 2H), 2.00-2.11 (m, 2H), 2.61 (s, 3H), 3.16-3.27 (m, 4H), 3.50-3.55 (m, 4H), 3.60 (ddd, J=11.4, 7.7, 3.6 Hz, 2H), 3.86 (ddd, J=11.2, 7.1, 3.7 Hz, 2H), 4.97 (tt, J=7.1, 3.9 Hz, 1H), 6.78 (s, 1H), 7.24 (s, 1H), 7.36 (d, J=1.4 Hz, 1H), 7.43 (dd, J=8.1, 1.4 Hz, 1H), 7.79 (d, J=8.1 Hz, 1H), 12.67 (s, 1H); MS (ESI) m/z 518 (M+H)⁺.

### Example 140

### tert-butyl 4-{2-carbamoyl-5-[3-methyl-4-(tetrahydro-2H-pyran-4-yloxy)-1H-indazol-6-yl]phenyl}piperazine-1-carboxylate

The procedure of Example 132, Step 2 was used, substituting tert-butyl 4-{2-cyano-5-[3-methyl-4-(tetrahydro-2H-pyran-4-yloxy)-1H-indazol-6-yl]phenyl}piperazine-1-carboxylate (Example 139) for 4-[3-methyl-4-(tetrahydro-2H-pyran-4-yloxy)-1H-indazol-6-yl]benzonitrile as described therein, to provide the titled compound. ¹H NMR (300 MHz, DMSO-d₆) δ ppm 1.43 (s, 9H), 1.66-1.83 (m, 2H), 1.97-2.13 (m, 2H), 2.61 (s, 3H), 2.91-3.08 (m, 4H), 3.45-3.56 (m, 4H), 3.55-3.66 (m, 2H), 3.77-3.94 (m, 2H), 4.97 (tt, J=7.2, 3.9, 3.7 Hz, 1H), 6.76 (s, 1H), 7.20 (s, 1H), 7.38 (d, J=1.4 Hz, 1H), 7.44 (dd, J=7.9, 1.5 Hz, 1H), 7.51 (d, J=2.0 Hz, 1H), 7.79 (d, J=7.9 Hz, 1H), 8.39 (d, J=2.4 Hz, 1H), 12.59 (s, 1H); MS (ESI) m/z 536 (M+H)⁺.

### Example 141

### tert-butyl 4-{2-acetamido-5-[3-methyl-4-(tetrahydro-2H-pyran-4-yloxy)-1H-indazol-6-yl]phenyl}piperazine-1-carboxylate

### Step 1

### tert-butyl 4-(5-bromo-2-nitrophenyl)piperazine-1-carboxylate

A solution of 4-bromo-2-fluoronitrobenzene (1.084 g, 4.93 mmol) and tert-butyl piperazine-1-carboxylate (1.835 g, 9.85 mmol) in ethanol (15 mL) was heated in a microwave (Biotage Personal Creator 300 W unit) at 120° C. for 10 minutes. The solution was cooled to room temperature, concentrated under vacuum, and purified by flash chromatography (silica gel eluted with heptanes-ethyl acetate, 100:0-80:20) to provide the titled compound (1.68 g, 85%). ¹H NMR (300 MHz, methanol-d₄) δ ppm 1.47 (s, 9H), 2.98-3.07 (m, 4H), 3.50-3.58 (m, 4H), 7.29 (dd, J=8.8, 2.0 Hz, 1H), 7.45 (d, J=2.0 Hz, 1H), 7.72 (d, J=8.8 Hz, 1H).

### Step 2

### tert-butyl 4-(2-acetamido-5-bromophenyl)piperazine-1-carboxylate

The procedure of Example 127, Step 3 was used, substituting tert-butyl 4-(5-bromo-2-nitrophenyl)piperazine-1-carboxylate (Step 1) for 5-bromo-2-nitro-N-(2-phenoxyethyl)aniline as described therein, to provide the titled compound. ¹H NMR (400 MHz, methanol-d₄) δ ppm 1.48 (s, 9H), 2.19 (s, 3H), 2.78-2.91 (m, 4H), 3.55-3.68 (m, 4H), 7.23 (dd, J=8.5, 2.1 Hz, 1H), 7.28 (d, J=2.1 Hz, 1H), 7.84 (d, J=8.5 Hz, 1H).

### Step 3

### tert-butyl 4-[2-acetamido-5-(4,4,5,5-tetramethyl-1,3,2-dioxaborolan-2-yl)phenyl]piperazine-1-carboxylate

The procedure of Example 115, Step 7 was used, substituting tert-butyl 4-(2-acetamido-5-bromophenyl)piperazine-1-carboxylate (Step 2) for 4-bromo-N-ethyl-2-(4-phenylpiperazin-1-yl)benzamide as described therein, to provide the titled compound. ¹H NMR (300 MHz, methanol-d₄) δ ppm 1.34 (s, 12H), 1.49 (s, 9H), 2.21 (s, 3H), 2.79-2.89 (m, 4H), 3.57-3.69 (m, 4H), 7.45-7.52 (m, 1H), 7.53 (s, 1H), 8.05 (d, J=8.1 Hz, 1H); MS (ESI) m/z 446 (M+H)⁺.

### Step 4

### tert-butyl 4-{2-acetamido-5-[3-methyl-4-(tetrahydro-2H-pyran-4-yloxy)-1H-indazol-6-yl]phenyl}piperazine-1-carboxylate

The procedure of Example 115, Step 8 was used, substituting tert-butyl 4-[2-acetamido-5-(4,4,5,5-tetramethyl-1,3,2-dioxaborolan-2-yl)phenyl]piperazine-1-carboxylate (Step 3) for N-ethyl-2-(4-phenylpiperazin-1-yl)-4-(4,4,5,5-tetramethyl-1,3,2-dioxaborolan-2-yl)benzamide as described therein, to provide the titled compound, which was purified by crystallization from ethanol. ¹H NMR (300 MHz, DMSO-d₆) δ ppm 1.43 (s, 9H), 1.67-1.84 (m, 2H), 1.98-2.11 (m, 2H), 2.15 (s, 3H), 2.59 (s, 3H), 2.82-2.91 (m, 4H), 3.52-3.59 (m, 4H), 3.57-3.67 (m, 2H), 3.86 (m, 2H), 4.95 (m, 1H), 6.73 (s, 1H), 7.12 (s, 1H), 7.31-7.44 (m, 2H), 8.00 (d, J=8.5 Hz, 1H), 8.91 (s, 1H), 12.52 (s, 1H); MS (ESI) m/z 550 (M+H)⁺.

### Example 142

### isopropyl 4-{2-carbamoyl-5-[3-methyl-4-(tetrahydro-2H-pyran-4-yloxy)-1H-indazol-6-yl]phenyl}piperazine-1-carboxylate

Trifluoroacetic acid (1 mL, 13 mmol) was added to an ice-cooled solution of tert-butyl 4-{2-carbamoyl-5-[3-methyl-4-(tetrahydro-2H-pyran-4-yloxy)-1H-indazol-6-yl]phenyl}piperazine-1-carboxylate (135 mg, 0.25 mmol, Example 140) in CH₂Cl₂ (2.5 mL). The solution was stirred with ice cooling for 1 hour, and then allowed to warm to room temperature. After 90 minutes, the solution was concentrated under vacuum. The residue was dissolved in CH₂Cl₂ (10 mL), and 20% aqueous K₂CO₃ (10 mL) was added. Isopropyl chloroformate (1 M in toluene, 0.50 mL, 0.50 mmol) was added to the mixture. The mixture was stirred at room temperature for 40 minutes and then partitioned between CH₂Cl₂ (15 mL) and water (5 mL). The organic phase was separated, dried (MgSO₄) and concentrated under vacuum. The residue was purified by HPLC (30×100 mm Waters C18 XBridge™ column eluted with 25 mM aqueous (NH₄)₂CO₃—CH₃OH, 80:20-0:100, 40 mL/minute over 15 minutes) to provide the titled compound (72 mg, 55%). ¹H NMR (300 MHz, DMSO-d₆) δ ppm 1.21 (d, J=6.4 Hz, 6H), 1.67-1.82 (m, 2H), 1.98-2.11 (m, 2H), 2.61 (s, 3H), 2.99-3.05 (m, 4H), 3.52-3.60 (m, 4H), 3.58-3.65 (m, 2H), 3.86 (ddd, J=11.1, 7.0, 3.6 Hz, 2H), 4.81 (sextet, J=6.2 Hz, 1H), 4.91-5.02 (m, 1H), 6.77 (s, 1H), 7.20 (s, 1H), 7.39 (d, J=1.7 Hz, 1H), 7.44 (dd, J=7.9, 1.7 Hz, 1H), 7.51 (s, 1H), 7.79 (d, J=7.8 Hz, 1H), 8.40 (s, 1H), 12.60 (s, 1H); MS (ESI) m/z 522 (M+H)⁺.

### Example 143

### N-methyl-4-[3-methyl-4-(tetrahydro-2H-pyran-4-yloxy)-1H-indazol-6-yl]-2-[4-(2,2,2-trifluoroethyl)piperazin-1-yl]benzamide

The procedure of Example 115, Step 8 was used, substituting N-methyl-4-(4,4,5,5-tetramethyl-1,3,2-dioxaborolan-2-yl)-2-[4-(2,2,2-trifluoroethyl)piperazin-1-yl]benzamide (Example 108, Step 2) for N-ethyl-2-(4-phenylpiperazin-1-yl)-4-(4,4,5,5-tetramethyl-1,3,2-dioxaborolan-2-yl)benzamide as described therein, to provide the titled compound. ¹H NMR (300 MHz, DMSO-d₆) δ ppm 1.69-1.82 (m, 2H), 1.99-2.13 (m, 2H), 2.61 (s, 3H), 2.79-2.88 (m, 3H), 2.86 (d, J=4.7 Hz, 3H), 3.00-3.07 (m, 4H), 3.32 (dd, 3H), 3.56-3.66 (m, 2H), 3.86 (ddd, J=11.3, 7.2, 3.6 Hz, 2H), 4.98 (tt, J=3.6 Hz, 1H), 6.77 (s, 1H), 7.20 (d, J=0.7 Hz, 1H), 7.41 (d, J=1.7 Hz, 1H), 7.44 (dd, J=8.1, 1.7 Hz, 1H), 7.76 (d, J=7.8 Hz, 1H), 8.96 (q, J=4.6 Hz, 1H), 12.60 (s, 1H); MS (ESI) m/z 532 (M+H)⁺.

### Example 144

### N-{2-[4-(cyclobutylcarbonyl)piperazin-1-yl]-4-[3-methyl-4-(tetrahydro-2H-pyran-4-yloxy)-1H-indazol-6-yl]phenyl}acetamide

### Step 1

### N-{4-[3-methyl-4-(tetrahydro-2H-pyran-4-yloxy)-1H-indazol-6-yl]-2-(piperazin-1-yl)phenyl}acetamide

Trifluoroacetic acid (3 mL, 38.9 mmol) was added to an ice-cooled solution of tert-butyl 4-{2-acetamido-5-[3-methyl-4-(tetrahydro-2H-pyran-4-yloxy)-1H-indazol-6-yl]phenyl}piperazine-1-carboxylate (735 mg, 1.337 mmol, Example 141) in CH₂Cl₂ (10 mL). The solution was stirred with ice cooling for 5 hours, then warmed gradually to room temperature. After 6 hours, the solution was concentrated under vacuum, and the residue was taken up in CH₂Cl₂ (50 mL) and washed with a mixture of 20% aqueous K₂CO₃ (40 mL) and 1 M NaOH (15 mL). The organic phase was dried (MgSO₄) and concentrated under vacuum to provide the titled compound (559 mg, 93%). ¹H NMR (300 MHz, DMSO-d₆) δ ppm 1.06 (m, 1H), 1.68-1.82 (m, 2H), 2.00-2.12 (m, 2H), 2.14 (s, 3H), 2.59 (s, 3H), 2.77-2.85 (m, 4H), 2.88-2.97 (m, 4H), 3.60 (ddd, J=11.3, 7.7, 3.4 Hz, 2H), 3.81-3.91 (m, 2H), 3.91 (s, 1H), 4.94 (tt, J=7.2, 3.7 Hz, 1H), 6.72 (s, 1H), 7.10 (s, 1H), 7.31-7.39 (m, 2H), 7.98 (d, J=7.1 Hz, 1H), 8.84 (s, 1H), 12.51 (s, 1H); MS (ESI) m/z 450 (M+H)⁺.

### Step 2

### N-{2-[4-(cyclobutylcarbonyl)piperazin-1-yl]-4-[3-methyl-4-(tetrahydro-2H-pyran-4-yloxy)-1H-indazol-6-yl]phenyl}acetamide

A solution of cyclobutanecarbonyl chloride (20.97 mg, 0.177 mmol) in CH₂Cl₂ (0.2 mL) was added to a solution of N-{4-[3-methyl-4-(tetrahydro-2H-pyran-4-yloxy)-1H-indazol-6-yl]-2-(piperazin-1-yl)phenyl}acetamide (53 mg, Step 1) and triethylamine (50 mg) in CH₂Cl₂ (1.5 mL), and the mixture was stirred at room temperature for 30 minutes. Methanol (1 mL) was added, and the solution stirred at room temperature for 4 hours. The mixture was concentrated, and the residue was purified by flash chromatography (silica gel eluted with ethyl acetate-ethanol (100:0-97:3) to provide the titled compound (54 mg, 86%). ¹H NMR (300 MHz, DMSO-d₆) δ ppm 1.66-1.83 (m, 3H), 1.84-1.97 (m, 1H), 2.00-2.29 (m, 7H), 2.15 (s, 3H), 2.59 (s, 3H), 2.81-2.90 (m, 4H), 3.50-3.65 (m, 4H), 3.65-3.73 (m, 2H), 3.80-3.92 (m, 2H), 4.94 (tt, J=7.2, 3.3 Hz, 1H), 6.72 (s, 1H), 7.12 (s, 1H), 7.32-7.42 (m, 1H), 7.37 (s, 1H), 8.00 (d, J=7.9 Hz, 1H), 8.96 (s, 1H), 12.52 (s, 1H); MS (ESI) m/z 532 (M+H)⁺.

### Example 145

### N-{2-(4-benzylpiperazin-1-yl)-4-[3-methyl-4-(tetrahydro-2H-pyran-4-yloxy)-1H-indazol-6-yl]phenyl}acetamide

Sodium cyanoborohydride (40 mg, 0.64 mmol) was added at room temperature to a solution of N-{4-[3-methyl-4-(tetrahydro-2H-pyran-4-yloxy)-1H-indazol-6-yl]-2-(piperazin-1-yl)phenyl}acetamide (44 mg, 0.098 mmol, Example 144, Step 1), benzaldehyde (0.14 mL, 1.4 mmol) and acetic acid (0.048 mL, 0.83 mmol) in CH₃OH (5 mL). The resulting solution was stirred at room temperature for 90 minutes, then concentrated under vacuum. The residue was partitioned between CH₂Cl₂ (15 mL) and 0.5 M NaOH (10 mL). The organic phase was separated and concentrated under vacuum. The residue was purified by HPLC (30×100 mm Waters C18 XBridge™ column eluted with 25 mM aqueous (NH₄)₂CO₃—CH₃OH, 70:30-0:100, 40 mL/minute over 15 minutes) to provide the titled compound (32.9 mg, 62%). ¹H NMR (300 MHz, DMSO-d₆) δ ppm 1.65-1.83 (m, 2H), 1.99-2.12 (m, 2H), 2.13 (s, 3H), 2.59 (s, 3H), 2.59-2.68 (m, 4H), 2.89-2.97 (m, 4H), 3.57 (s, 2H), 3.56-3.65 (m, 2H), 3.86 (ddd, J=11.2, 7.1, 3.4 Hz, 2H), 4.95 (tt, J=7.2, 3.7 Hz, 1H), 6.72 (s, 1H), 7.11 (s, 1H), 7.22-7.41 (m, 7H), 7.96 (d, J=7.8 Hz, 1H), 8.82 (s, 1H), 12.51 (s, 1H); MS (ESI) m/z 540 (M+H)⁺.

### Example 146

### isopropyl 4-{2-acetamido-5-[3-methyl-4-(tetrahydro-2H-pyran-4-yloxy)-1H-indazol-6-yl]phenyl}piperazine-1-carboxylate

The procedure of Example 144, Step 2 was used, substituting isopropyl chloroformate for cyclobutane carbonyl chloride as described therein, to provide the titled compound. ¹H NMR (300 MHz, DMSO-d₆) δ ppm 1.21 (d, J=6.1 Hz, 6H), 1.67-1.82 (m, 2H), 1.99-2.12 (m, 2H), 2.15 (s, 3H), 2.59 (s, 3H), 2.83-2.93 (m, 4H), 3.54-3.67 (m, 6H), 3.86 (m, 2H), 4.81 (sept, J=6.2 Hz, 1H), 4.95 (tt, J=7.1, 3.3 Hz, 1H), 6.73 (s, 1H), 7.12 (s, 1H), 7.35-7.43 (m, 2H), 8.01 (d, J=8.1 Hz, 1H), 8.92 (s, 1H), 12.52 (s, 1H); MS (ESI) m/z 536 (M+H)⁺.

### Example 147

### isopropyl (2-{2-acetamido-5-[3-methyl-4-(tetrahydro-2H-pyran-4-yloxy)-1H-indazol-6-yl]phenoxy}ethyl)carbamate

### Step 1

### isopropyl [2-(5-bromo-2-nitrophenoxy)ethyl]carbamate

N-(2-Hydroxyethyl)phthalimide (0.217 g, 1.14 mmol) was added to a suspension of potassium tert-butoxide (0.153 g, 1.36 mmol) in tetrahydrofuran (5 mL). The suspension was stirred for 5 minutes, then 4-bromo-2-fluoronitrobenzene (0.25 g, 1.14 mmol) was added. The mixture was stirred at room temperature for 14 hours and then concentrated under vacuum. The residue was taken up in CHCl₃ (20 mL) and washed with 8% aqueous H₂SO₄ (10 mL). The organic layer was dried (MgSO₄) and filtered. To the filtrate was added anhydrous hydrazine (0.15 mL, 4.8 mmol), and the mixture was stirred at room temperature for 3 hours. The mixture was filtered with a CHCl₃ (10 mL) rinse. The combined filtrate and washing was concentrated under vacuum, and the residue was taken up in aqueous 10% K₂CO₃ (20 mL) and CH₂Cl₂ (20 mL). This mixture was stirred at room temperature as a solution of isopropyl chloroformate in toluene (1 M, 3.42 mL, 3.42 mmol) was added. After 45 minutes, the aqueous layer was separated and extracted with CH₂Cl₂ (10 mL), and the combined organic phases were concentrated under vacuum. The residue was purified by flash chromatography (silica gel eluted with heptanes-ethyl acetate, 90:10-60:40) to provide the titled compound (116 mg, 29%). ¹H NMR (300 MHz, CDCl₃) δ ppm 1.23 (d, J=6.1 Hz, 6H), 3.63 (q, J=5.2 Hz, 2H), 4.18 (t, J=4.9 Hz, 2H), 4.91 (sept, J=6.2 Hz, 1H), 5.19 (br m, 1H), 7.20 (dd, J=8.5, 2.0 Hz, 1H), 7.23 (d, J=2.0 Hz, 1H), 7.77 (d, J=8.5 Hz, 1H); MS (ESI) m/z 347/349 (M+H)⁺.

### Step 2

### isopropyl [2-(2-acetamido-5-bromophenoxy)ethyl]carbamate

The procedure of Example 127, Step 3 was used, substituting isopropyl [2-(5-bromo-2-nitrophenoxy)ethyl]carbamate for 5-bromo-2-nitro-N-(2-phenoxyethyl)aniline as described therein, to provide the titled compound. ¹H NMR (300 MHz, CDCl₃) δ ppm 1.23 (d, J=6.1 Hz, 6H), 2.25 (s, 3H), 3.62 (dt, J=6.5, 4.9 Hz, 2H), 4.03 (t, J=4.7 Hz, 2H), 4.93 (septet, J=6.3 Hz, 1H), 4.89-5.01 (br s, 1H), 6.90 (d, J=2.0 Hz, 1H), 7.07 (dd, J=8.8, 2.0 Hz, 1H), 8.32 (d, J=8.8 Hz, 1H), 8.35 (br s, 1H); MS (ESI) m/z 359/361 (M+H)⁺.

### Step 3

### isopropyl {2-[2-acetamido-5-(4,4,5,5-tetramethyl-1,3,2-dioxaborolan-2-yl)phenoxy]ethyl}carbamate

The procedure of Example 115, Step 7 was used, substituting isopropyl [2-(2-acetamido-5-bromophenoxy)ethyl]carbamate (Step 2) for 4-bromo-N-ethyl-2-(4-phenylpiperazin-1-yl)benzamide as described therein, to provide the titled compound. ¹H NMR (300 MHz, CDCl₃) δ ppm 1.23 (d, J=6.4 Hz, 6H), 1.33 (s, 12H), 2.26 (s, 3H), 3.62 (q, J=5.1 Hz, 2H), 4.12 (t, J=4.9 Hz, 2H), 4.87 (s, 1H), 4.85-4.98 (m, 1H), 7.19 (s, 1H), 7.43 (dd, J=8.1, 1.0 Hz, 1H), 8.43 (d, J=8.1 Hz, 1H), 8.44 (s, 1H); MS (ESI) m/z 407 (M+H)⁺.

### Step 4

### isopropyl (2-{2-acetamido-5-[3-methyl-4-(tetrahydro-2H-pyran-4-yloxy)-1H-indazol-6-yl]phenoxy}ethyl)carbamate

The procedure of Example 115, Step 8 was used, substituting isopropyl {2-[2-acetamido-5-(4,4,5,5-tetramethyl-1,3,2-dioxaborolan-2-yl)phenoxy]ethyl}carbamate (Step 3) for N-ethyl-2-(4-phenylpiperazin-1-yl)-4-(4,4,5,5-tetramethyl-1,3,2-dioxaborolan-2-yl)benzamide as described therein, to provide the titled compound. ¹H NMR (300 MHz, DMSO-d₆) δ ppm 1.17 (d, J=6.4 Hz, 6H), 1.65-1.83 (m, 2H), 2.00-2.13 (m, 2H), 2.16 (s, 3H), 2.59 (s, 3H), 3.45 (q, J=5.2 Hz, 2H), 3.60 (ddd, J=11.2, 7.5, 3.4 Hz, 2H), 3.86 (ddd, J=11.0, 7.0, 3.7 Hz, 2H), 4.13 (t, J=4.9 Hz, 2H), 4.81 (septet, J=6.3, Hz, 1H), 4.95 (tt, J=7.2, 3.4 Hz, 1H), 6.75 (s, 1H), 7.14 (s, 1H), 7.19-7.27 (m, 1H), 7.24 (s, 1H), 7.41 (t, J=5.8 Hz, 1H), 8.16 (d, J=8.1 Hz, 1H), 8.97 (s, 1H), 12.52 (s, 1H); MS (ESI) m/z 511 (M+H)⁺.

### Example 148

### N-{4-[3-methyl-4-(tetrahydro-2H-pyran-4-yloxy)-1H-indazol-6-yl]-2-[4-(pyridin-3-ylmethyl)piperazin-1-yl]phenyl}acetamide

The procedure of Example 145 was used, substituting pyridine-3-carboxaldehyde for benzaldehyde as described therein, to provide the titled compound. ¹H NMR (300 MHz, DMSO-d₆) δ ppm 1.65-1.82 (m, 2H), 1.97-2.10 (m, 2H), 2.13 (s, 3H), 2.59 (s, 3H), 2.61-2.68 (m, 4H), 2.93 (s, 4H), 3.52-3.66 (m, 2H), 3.61 (s, 2H), 3.78-3.92 (m, 2H), 4.88-5.01 (m, 1H), 6.72 (s, 1H), 7.11 (s, 1H), 7.32-7.42 (m, 2H), 7.38 (s, 1H), 7.76 (d, J=7.8 Hz, 1H), 7.96 (d, J=7.5 Hz, 1H), 8.49 (d, J=3.4 Hz, 1H), 8.55 (s, 1H), 8.83 (s, 1H), 12.51 (s, 1H); MS (ESI) m/z 541 (M+H)⁺.

### Example 149

### N-{2-[4-(3-fluorobenzyl)piperazin-1-yl]-4-[3-methyl-4-(tetrahydro-2H-pyran-4-yloxy)-1H-indazol-6-yl]phenyl}acetamide

The procedure of Example 145 was used, substituting 3-fluorobenzaldehyde for benzaldehyde as described therein, to provide the titled compound. ¹H NMR (300 MHz, DMSO-d₆) δ ppm 1.66-1.83 (m, 2H), 1.99-2.13 (m, 2H), 2.13 (s, 3H), 2.59 (s, 3H), 2.60-2.67 (m, 4H), 2.89-2.98 (m, 4H), 3.53-3.66 (m, 2H), 3.60 (s, 2H), 3.86 (ddd, J=11.2, 7.1, 3.4 Hz, 2H), 4.95 (tt, J=7.0, 3.6 Hz, 1H), 6.73 (s, 1H), 7.07 (dd, J=8.8, 2.4 Hz, 1H), 7.12 (s, 1H), 7.13-7.24 (m, 2H), 7.32-7.43 (m, 3H), 7.96 (d, J=8.1 Hz, 1H), 8.83 (s, 1H), 12.51 (s, 1H); MS (ESI) m/z 558 (M+H)⁺.

### Example 150

### N-(2-{4-[(5-chloro-2-furyl)methyl]piperazin-1-yl}-4-[3-methyl-4-(tetrahydro-2H-pyran-4-yloxy)-1H-indazol-6-yl]phenyl)acetamide

The procedure of Example 145 was used, substituting 5-chlorofurancarboxaldehyde for benzaldehyde as described therein, to provide the titled compound. ¹H NMR (400 MHz, DMSO-d₆) δ ppm 1.69-1.80 (m, 2H), 2.00-2.09 (m, 2H), 2.12 (s, 3H), 2.59 (s, 3H), 2.61-2.69 (m, 4H), 2.90-2.95 (m, 4H), 3.56 (s, 2H), 3.60 (ddd, J=11.4, 7.6, 3.5 Hz, 2H), 3.82-3.89 (m, 2H), 4.95 (tt, J=7.5, 3.7 Hz, 1H), 6.42 (d, J=3.4 Hz, 1H), 6.46 (d, J=3.4 Hz, 1H), 6.72 (s, 1H), 7.11 (s, 1H), 7.33-7.38 (m, 1H), 7.37 (s, 1H), 7.95 (d, J=8.1 Hz, 1H), 8.85 (s, 1H), 12.53 (s, 1H); MS (ESI) m/z 564/566 (M+H)⁺.

### Example 151

### 4-{2-acetamido-5-[3-methyl-4-(tetrahydro-2H-pyran-4-yloxy)-1H-indazol-6-yl]phenyl}-N-isopropylpiperazine-1-carboxamide

Isopropyl isocyanate (10 mg, 0.118 mmol) was combined with N-{4-[3-methyl-4-(tetrahydro-2H-pyran-4-yloxy)-1H-indazol-6-yl]-2-(piperazin-1-yl)phenyl}acetamide (25 mg, 0.056 mmol, Example 144, Step 1) in pyridine (0.102 mL, 1.264 mmol) and CH₂Cl₂ (1 mL). The resulting solution was stirred at room temperature for 3 hours, then concentrated under vacuum. The residue was purified by HPLC (30×100 mm Waters C18 XBridge™ column eluted with 25 mM aqueous (NH₄)₂CO₃—CH₃OH, 80:20-0:100, 40 mL/minute over 15 minutes) to provide the titled compound (8 mg, 27%). ¹H NMR (400 MHz, DMSO-d₆) δ ppm 1.07 (d, J=6.4 Hz, 6H), 1.70-1.80 (m, 2H), 2.01-2.10 (m, 2H), 2.15 (s, 3H), 2.59 (s, 3H), 2.82-2.89 (m, 4H), 3.48-3.54 (m, 4H), 3.60 (ddd, J=11.3, 7.6, 3.4 Hz, 2H), 3.73-3.84 (m, 1H), 3.86 (ddd, J=11.1, 7.2, 3.7 Hz, 2H), 4.95 (tt, J=7.3, 3.6 Hz, 1H), 6.23 (d, J=7.6 Hz, 1H), 6.73 (s, 1H), 7.12 (s, 1H), 7.37 (s, 1H), 7.37-7.41 (m, 1H), 8.00 (d, J=8.1 Hz, 1H), 8.92 (s, 1H), 12.52 (s, 1H; MS (ESI) m/z 535 (M+H)⁺.

### Example 152

### N-methyl-2-{4-[(4-methylphenyl)sulfonyl]piperazin-1-yl}-4-[3-methyl-4-(tetrahydro-2H-pyran-4-yloxy)-1H-indazol-6-yl]benzamide

### Step 1

### N-methyl-2-{4-[(4-methylphenyl)sulfonyl]piperazin-1-yl}-4-(4,4,5,5-tetramethyl-1,3,2-dioxaborolan-2-yl)benzamide

Trifluoroacetic acid (0.8 mL, 10.38 mmol) was added to an ice-cooled solution of tert-butyl 4-[2-(methylcarbamoyl)-5-(4,4,5,5-tetramethyl-1,3,2-dioxaborolan-2-yl)phenyl]piperazine-1-carboxylate (Example 111, Step 1, 49 mg, 0.110 mmol) in CH₂Cl₂ (2 mL). The solution was stirred with ice cooling for 1 hour, then concentrated under vacuum. The residue was dissolved in CH₂Cl₂ (2 mL), and the solution was stirred at room temperature as 4-methylbenzene-1-sulfonyl chloride (0.03 g, 0.157 mmol) and triethylamine (0.2 mL, 1.435 mmol) were added in that order. The mixture was stirred at room temperature for 4 hours, then diluted with CH₂Cl₂ (10 mL) and washed successively with 8% aqueous H₂SO₄ (5 mL) and 20% aqueous K₂CO₃ (5 mL). The organic phase was dried (MgSO₄) and concentrated under vacuum to provide the titled compound (51 mg, 93%). ¹H NMR (400 MHz, DMSO-d₆) δ ppm 1.29 (s, 12H), 2.43 (s, 3H), 2.60 (d, J=4.6 Hz, 3H), 2.95-2.99 (m, 4H), 3.00-3.06 (m, 4H), 7.27 (s, 1H), 7.36 (dd, J=7.5, 0.8 Hz, 1H), 7.45 (d, J=7.3 Hz, 1H), 7.50 (d, J=8.2 Hz, 2H), 7.68 (d, J=8.2 Hz, 2H), 8.27 (q, J=4.6 Hz, 1H); MS (ESI) m/z 500 (M+H)⁺.

### Step 2

### N-methyl-2-{4-[(4-methylphenyl)sulfonyl]piperazin-1-yl}-4-[3-methyl-4-(tetrahydro-2H-pyran-4-yloxy)-1H-indazol-6-yl]benzamide

The procedure of Example 115, Step 8 was used, substituting N-methyl-2-{4-[(4-methylphenyl)sulfonyl]piperazin-1-yl}-4-(4,4,5,5-tetramethyl-1,3,2-dioxaborolan-2-yl)benzamide for N-ethyl-2-(4-phenylpiperazin-1-yl)-4-(4,4,5,5-tetramethyl-1,3,2-dioxaborolan-2-yl)benzamide as described therein, to provide the titled compound. ¹H NMR (300 MHz, DMSO-d₆) δ ppm 1.69-1.81 (m, 2H), 1.99-2.11 (m, 2H), 2.43 (s, 3H), 2.60 (s, 3H), 2.63 (d, J=4.7 Hz, 3H), 3.03-3.14 (m, 8H), 3.60 (ddd, J=11.3, 7.4, 3.4 Hz, 2H), 3.86 (ddd, J=11.3, 7.2, 3.5 Hz, 2H), 4.95 (tt, J=7.2, 3.7 Hz, 1H), 6.74 (s, 1H), 7.17 (s, 1H), 7.31 (d, J=1.7 Hz, 1H), 7.39 (dd, J=7.8, 1.7 Hz, 1H), 7.50 (d, J=7.8 Hz, 2H), 7.58 (d, J=7.8 Hz, 1H), 7.69 (d, J=8.1 Hz, 2H), 8.36 (q, J=4.7 Hz, 1H), 12.59 (s, 1H); MS (ESI) m/z 604 (M+H)⁺.

### Example 153

### methyl 4-{2-(methylcarbamoyl)-5-[3-methyl-4-(tetrahydro-2H-pyran-4-yloxy)-1H-indazol-6-yl]phenyl}piperazine-1-carboxylate

The procedure of Example 125 was used, substituting methyl chloroformate for isopropyl chloroformate as described therein, to provide the titled compound. ¹H NMR (300 MHz, DMSO-d₆) δ ppm 1.68-1.82 (m, 2H), 2.00-2.13 (m, 2H), 2.61 (s, 3H), 2.86 (d, J=4.7 Hz, 3H), 2.95-3.04 (m, 4H), 3.54-3.60 (m, 4H), 3.60-3.64 (m, 2H), 3.63 (s, 3H), 3.86 (ddd, J=11.2, 7.1, 3.7 Hz, 2H), 4.96 (tt, J=7.2, 3.8 Hz, 1H), 6.76 (s, 1H), 7.19 (s, 1H), 7.39 (d, J=1.5 Hz, 1H), 7.45 (dd, J=8.0, 1.5 Hz, 1H), 7.76 (d, J=7.8 Hz, 1H), 8.87 (q, J=4.9 Hz, 1H), 12.61 (br s, 1H); MS (ESI) m/z 508 (M+H)⁺.

### Example 154

### 2-[4-(3-fluorobenzyl)piperazin-1-yl]-N-methyl-4-[3-methyl-4-(tetrahydro-2H-pyran-4-yloxy)-1H-indazol-6-yl]benzamide

The procedure of Example 123, Step 2 was used, substituting 3-fluorobenzaldehyde for pyridine-3-carboxaldehyde as described therein, to provide the titled compound. ¹H NMR (300 MHz, DMSO-d₆) δ ppm 1.66-1.85 (m, 2H), 1.97-2.12 (m, 2H), 2.61 (s, 3H), 2.85 (d, J=4.7 Hz, 3H), 3.06-3.48 (m, 8H), 3.59 (ddd, J=11.4, 7.6, 3.4 Hz, 2H), 3.87 (m, 2H), 4.46 (br s, 2H), 4.93 (tt, J=7.2, 3.9, Hz, 1H), 6.74 (s, 1H), 7.16 (s, 1H), 7.30 (s, 1H), 7.31-7.49 (m, 4H), 7.52-7.69 (m, 2H), 8.41 (m, 1H), 9.76 (br m, 1H), 12.63 (br s, 1H); MS (ESI) m/z 558 (M+H)⁺.

### Example 155

### N-isopropyl-4-{2-(methylcarbamoyl)-5-[3-methyl-4-(tetrahydro-2H-pyran-4-yloxy)-1H-indazol-6-yl]phenyl}piperazine-1-carboxamide

The procedure of Example 151 was used, substituting N-methyl-4-[3-methyl-4-(tetrahydro-2H-pyran-4-yloxy)-1H-indazol-6-yl]-2-(piperazin-1-yl)benzamide (Example 125, Step 1) for N-{4-[3-methyl-4-(tetrahydro-2H-pyran-4-yloxy)-1H-indazol-6-yl]-2-(piperazin-1-yl)phenyl}acetamide as described therein, to provide the titled compound. ¹H NMR (400 MHz, DMSO-d₆) δ ppm 1.07 (d, J=6.4 Hz, 6H), 1.68-1.81 (m, 2H), 1.99-2.14 (m, 2H), 2.61 (s, 3H), 2.86 (d, J=4.6 Hz, 3H), 2.98-3.03 (m, 4H), 3.49-3.55 (m, 4H), 3.57-3.65 (m, 2H), 3.76-3.81 (m, 1H), 3.87 (m, 2H), 4.97 (tt, J=7.2, 3.7 Hz, 1H), 6.27 (d, J=4.6 Hz, 1H), 6.78 (s, 1H), 7.21 (s, 1H), 7.45 (s, 1H), 7.48 (d, J=8.1 Hz, 1H), 7.80 (d, J=8.1 Hz, 1H), 8.99 (q, J=4.7 Hz, 1H), 12.58 (br s, 1H); MS (ESI) m/z 535 (M+H)⁺.

### Example 156

### 4-[3-methyl-4-(tetrahydro-2H-pyran-4-yloxy)-1H-indazol-6-yl]-2-[4-(2,2,2-trifluoroethyl)piperazin-1-yl]benzonitrile

### Step 1

### 4-bromo-2-[4-(2,2,2-trifluoroethyl)piperazin-1-yl]benzonitrile

A mixture of 1-(2,2,2-trifluoroethyl)piperazine (270 mg, 1.606 mmol), 4-bromo-2-fluorobenzonitrile (321 mg, 1.61 mmol) and triethylamine (0.45 mL, 3.21 mmol) in dimethyl sulfoxide (1 mL) was heated in a capped vial at 120° C. for 2 hours, then cooled to room temperature, and added dropwise over 2 minutes to rapidly stirring water (30 mL). The resulting slurry was stirred at room temperature for 20 minutes, and the solid was collected by filtration, washed with water (20 mL) and dried under vacuum to provide the titled compound (431 mg, 77%). ¹H NMR (300 MHz, methanol-d₄) δ ppm 2.84-2.91 (m, 4H), 3.15 (q, J=9.8 Hz, 2H), 3.21-3.28 (m, 4H), 7.24 (dd, J=8.1, 1.7 Hz, 1H), 7.30 (d, J=1.7 Hz, 1H), 7.51 (d, J=8.1 Hz, 1H); MS (ESI) m/z 348/350 (M+H)⁺.

### Step 2

### 4-(4,4,5,5-tetramethyl-1,3,2-dioxaborolan-2-yl)-2-[4-(2,2,2-trifluoroethyl)piperazin-1-yl]benzonitrile

The procedure of Example 115, Step 7 was used, substituting 4-bromo-2-[4-(2,2,2-trifluoroethyl)piperazin-1-yl]benzonitrile (Step 1) for 4-bromo-N-ethyl-2-(4-phenylpiperazin-1-yl)benzamide as described therein, to provide the titled compound. ¹H NMR (300 MHz, CDCl₃) δ ppm 1.34 (s, 12H), 2.86-2.97 (m, 4H), 3.08 (q, J=9.3 Hz, 2H), 3.22-3.34 (m, 4H), 7.42-7.46 (m, 1H), 7.43 (s, 1H), 7.55 (d, J=7.8 Hz, 1H); MS (ESI) m/z 396 (M+H)⁺.

### Step 3

### 4-[3-methyl-4-(tetrahydro-2H-pyran-4-yloxy)-1H-indazol-6-yl]-2-[4-(2,2,2-trifluoroethyl)piperazin-1-yl]benzonitrile

The procedure of Example 115, Step 8 was used, substituting 4-(4,4,5,5-tetramethyl-1,3,2-dioxaborolan-2-yl)-2-[4-(2,2,2-trifluoroethyl)piperazin-1-yl]benzonitrile (Step 2) for N-ethyl-2-(4-phenylpiperazin-1-yl)-4-(4,4,5,5-tetramethyl-1,3,2-dioxaborolan-2-yl)benzamide as described therein, to provide the titled compound, which was purified by crystallization from heptanes-ethyl acetate. ¹H NMR (300 MHz, DMSO-d₆) δ ppm 1.67-1.82 (m, 2H), 2.00-2.12 (m, 2H), 2.61 (s, 3H), 2.78-2.87 (m, 4H), 3.22-3.31 (m, 6H), 3.60 (ddd, J=11.4, 7.6, 3.4 Hz, 2H), 3.86 (ddd, J=11.2, 7.1, 3.4 Hz, 2H), 4.97 (tt, J=7.2, 3.7 Hz, 1H), 6.78 (s, 1H), 7.23 (s, 1H), 7.34 (d, J=1.7 Hz, 1H), 7.41 (dd, J=8.1, 1.7 Hz, 1H), 7.77 (d, J=8.1 Hz, 1H), 12.66 (s, 1H); MS (ESI) m/z 500 (M+H)⁺.

### Example 157

### 4-[3-methyl-4-(tetrahydro-2H-pyran-4-yloxy)-1H-indazol-6-yl]-2-[4-(2,2,2-trifluoroethyl)piperazin-1-yl]benzamide

The procedure of Example 132, Step 2 was used, substituting 4-[3-methyl-4-(tetrahydro-2H-pyran-4-yloxy)-1H-indazol-6-yl]-2-[4-(2,2,2-trifluoroethyl)piperazin-1-yl]benzonitrile (Example 156) for 4-[3-methyl-4-(tetrahydro-2H-pyran-4-yloxy)-1H-indazol-6-yl]benzonitrile as described therein, to provide the titled compound, purified by crystallization from ethanol-water. ¹H NMR (300 MHz, DMSO-d₆) δ ppm 1.66-1.83 (m, 2H), 1.99-2.13 (m, 2H), 2.61 (s, 3H), 2.80-2.87 (m, 4H), 3.03-3.10 (m, 4H), 3.27 (q, J=10.2 Hz, 2H), 3.61 (ddd, J=11.4, 7.5, 3.4 Hz, 2H), 3.87 (ddd, J=11.2, 7.1, 3.4 Hz, 2H), 4.98 (tt, J=7.2, 3.6 Hz, 1H), 6.77 (s, 1H), 7.20 (s, 1H), 7.37-7.49 (m, 2H), 7.57 (d, J=2.7 Hz, 1H), 7.81 (d, J=7.8 Hz, 1H), 8.48 (d, J=2.7 Hz, 1H), 12.59 (s, 1H); MS (ESI) m/z 500 (M+H)⁺.

### Example 158

### tert-butyl 4-{5-[3-ethyl-4-(tetrahydro-2H-pyran-4-yloxy)-1H-indazol-6-yl]-2-(methylcarbamoyl)phenyl}piperazine-1-carboxylate

### Step 1

### 1-[4-bromo-2-fluoro-6-(tetrahydro-2H-pyran-4-yloxy)phenyl]propan-1-ol

The procedure of Example 115, Step 2 was used, substituting ethylmagnesium bromide for methylmagnesium bromide as described therein, to provide the titled compound. ¹H NMR (400 MHz, CDCl₃) δ ppm 0.92 (t, J=7.3 Hz, 3H), 1.73-1.88 (m, 3H), 1.89-2.01 (m, 1H), 2.03-2.14 (m, 2H), 3.62 (ddd, J=11.7, 8.3, 2.9 Hz, 2H), 3.91-4.01 (m, 2H), 4.56 (tt, J=8.0, 4.0 Hz, 1H), 4.91 (dt, J=11.0, 7.3 Hz, 1H), 6.78-6.84 (m, 1H), 6.85-6.89 (m, 1H); MS (ESI) m/z 350/352 (M+H)⁺.

### Step 2

### 1-[4-bromo-2-fluoro-6-(tetrahydro-2H-pyran-4-yloxy)phenyl]propan-1-one

The procedure of Example 115, Step 3 was used, substituting 1-[4-bromo-2-fluoro-6-(tetrahydro-2H-pyran-4-yloxy)phenyl]propan-1-ol (Step 1) for 1-[4-bromo-2-fluoro-6-(tetrahydro-2H-pyran-4-yloxy)phenyl]ethanol as described therein, to provide the titled compound. ¹H NMR (300 MHz, CDCl₃) δ ppm 1.17 (t, J=7.3 Hz, 3H), 1.70-1.84 (m, 2H), 1.94-2.08 (m, 2H), 2.79 (q, J=7.3 Hz, 2H), 3.59 (ddd, J=11.5, 7.8, 3.4 Hz, 2H), 3.84-3.95 (m, 2H), 4.51 (tt, J=7.5, 3.7 Hz, 1H), 6.83-6.87 (m, J=1.0 Hz, 1H), 6.91 (dd, J=8.3, 1.5 Hz, 1H); MS (ESI) m/z 331/333 (M+H)⁺.

### Step 3

### 6-bromo-3-ethyl-4-(tetrahydro-2H-pyran-4-yloxy)-1H-indazole

The procedure of Example 115, Step 4 was used, substituting 1-[4-bromo-2-fluoro-6-(tetrahydro-2H-pyran-4-yloxy)phenyl]propan-1-one (Step 2) for 1-[4-bromo-2-fluoro-6-(tetrahydro-2H-pyran-4-yloxy)phenyl]ethanone as described therein, to provide the titled compound, purified by crystallization from ethanol. ¹H NMR (300 MHz, DMSO-d₆) δ ppm 1.26 (t, J=7.5 Hz, 3H), 1.61-1.76 (m, 2H), 1.97-2.13 (m, 2H), 2.96 (q, J=7.5 Hz, 2H), 3.58 (ddd, J=11.5, 8.1, 3.1 Hz, 2H), 3.84 (m, 2H), 4.83 (tt, J=7.6, 3.7 Hz, 1H), 6.69 (d, J=1.2 Hz, 1H), 7.17 (d, J=1.2 Hz, 1H), 12.61 (s, 1H); MS (ESI) m/z 325/327 (M+H)⁺.

### Step 4

### tert-butyl 4-{5-[3-ethyl-4-(tetrahydro-2H-pyran-4-yloxy)-1H-indazol-6-yl]-2-(methylcarbamoyl)phenyl}piperazine-1-carboxylate

The procedure of Example 115, Step 8 was used, substituting 6-bromo-3-ethyl-4-(tetrahydro-2H-pyran-4-yloxy)-1H-indazole (Step 3) for 6-bromo-3-methyl-4-(tetrahydro-2H-pyran-4-yloxy)-1H-indazole and substituting tert-butyl 4-[2-(methylcarbamoyl)-5-(4,4,5,5-tetramethyl-1,3,2-dioxaborolan-2-yl)phenyl]piperazine-1-carboxylate (Example 111, Step 1) for N-ethyl-2-(4-phenylpiperazin-1-yl)-4-(4,4,5,5-tetramethyl-1,3,2-dioxaborolan-2-yl)benzamide as described therein, to provide the titled compound. ¹H NMR (400 MHz, DMSO-d₆) δ ppm 1.31 (t, J=7.5 Hz, 3H), 1.43 (s, 9H), 1.70-1.81 (m, 2H), 2.02-2.13 (m, 2H), 2.86 (d, J=4.9 Hz, 3H), 2.95-3.00 (m, 4H), 3.02 (q, J=7.5 Hz, 2H), 3.50-3.55 (m, 4H), 3.61 (ddd, J=11.3, 7.6, 3.4 Hz, 2H), 3.86 (ddd, J=11.1, 6.9, 3.7 Hz, 2H), 4.98 (tt, J=7.0, 3.5 Hz, 1H), 6.76 (s, 1H), 7.20 (s, 1H), 7.39 (s, 1H), 7.45 (dd, J=7.9, 1.2 Hz, 1H), 7.77 (d, J=7.9 Hz, 1H), 8.89 (q, J=4.8 Hz, 1H), 12.60 (s, 1H); MS (ESI) m/z 564 (M+H)⁺.

### Example 159

### 2-(4-benzyl-3-oxopiperazin-1-yl)-N-methyl-4-[3-methyl-4-(tetrahydro-2H-pyran-4-yloxy)-1H-indazol-6-yl]benzamide

### Step 1

### tert-butyl 4-benzyl-3-oxopiperazine-1-carboxylate

Di-tert-butyl dicarbonate (750 mg, 3.44 mmol) was added to a solution of piperazine-2-one (344 mg, 3.44 mmol) in CH₂Cl₂ (13 mL). The solution was stirred at room temperature for 16 hours, then concentrated under vacuum. The residue was dissolved in N,N-dimethylformamide (10 mL) and stirred under nitrogen as sodium hydride (60% dispersion in oil, 0.25 g, 6.25 mmol) was added at room temperature. The mixture was stirred for 30 minutes, then benzyl bromide (0.532 mL, 4.47 mmol) was added. The mixture was stirred under nitrogen for 1 hour, and the reaction was quenched by cautious addition of water (3 mL). Finally, the mixture was diluted with water (50 mL) and stirred at room temperature for 2 hours. The resulting precipitate was collected by filtration and washed with water (40 mL), then dried under vacuum to the titled compound (0.82 g, 82%). ¹H NMR (400 MHz, methanol-d₄) δ ppm 1.46 (s, 9H), 3.30-3.33 (m, 2H), 3.60 (t, J=5.2 Hz, 2H), 4.11 (s, 2H), 4.62 (s, 2H), 7.25-7.37 (m, 5H); MS (ESI) m/z 291 (M+H)⁺.

### Step 2

### 1-benzylpiperazin-2-one 4-methylbenzenesulfonate

A solution of tert-butyl 4-benzyl-3-oxopiperazine-1-carboxylate (0.82 g, 2.82 mmol, Step 1) and p-toluenesulfonic acid monohydrate (0.645 g, 3.39 mmol) in ethyl acetate (30 mL) was heated at reflux for 1 hour, then cooled to room temperature and stirred for 16 hours. The precipitate was collected by filtration, washed with ethyl acetate (10 mL) and dried under vacuum to provide the titled compound, ¹H NMR (400 MHz, methanol-d₄) δ ppm 2.37 (s, 3H), 3.46-3.55 (m, 4H), 3.92 (s, 2H), 4.66 (s, 2H), 7.23 (d, J=8.2 Hz, 2H), 7.27-7.39 (m, 5H), 7.70 (d, J=8.2 Hz, 2H); MS (ESI) m/z 191 (M+H)⁺.

### Step 3

### 2-(4-benzyl-3-oxopiperazin-1-yl)-4-bromo-N-methylbenzamide

A mixture of 1-benzylpiperazin-2-one 4-methylbenzenesulfonate (312 mg, 0.862 mmol, Step 2), 4-bromo-2-fluoro-N-methylbenzamide (200 mg, 0.862 mmol) and triethylamine (0.72 mL, 5.2 mmol) in dimethyl sulfoxide (1.5 mL) was heated at 120° C. for 71 hours. The solution was cooled to room temperature and added gradually to rapidly stirring water (40 mL). The resulting mixture was heated to boiling for 5 minutes, then stirred vigorously as it cooled to room temperature. A sticky gum separated. The aqueous supernatant was decanted and the residue was rinsed with water (2×5 mL), then purified by flash chromatography (silica gel eluted with CH₂Cl₂—CH₃OH, 100:0-96:4) to provide the titled compound (99 mg, 29%). ¹H NMR (400 MHz, DMSO-d₆) δ ppm 2.70 (d, J=4.6 Hz, 3H), 3.21-3.30 (m, 4H), 3.73 (s, 2H), 4.58 (s, 2H), 7.24 (s, 1H), 7.24-7.31 (m, 4H), 7.31-7.39 (m, 3H), 8.39 (q, J=4.5 Hz, 1H); MS (ESI) m/z 402/404 (M+H)⁺.

### Step 4

### 2-(4-benzyl-3-oxopiperazin-1-yl)-N-methyl-4-(4,4,5,5-tetramethyl-1,3,2-dioxaborolan-2-yl)benzamide

The procedure of Example 115, Step 7 was used, substituting 2-(4-benzyl-3-oxopiperazin-1-yl)-4-bromo-N-methylbenzamide (Step 3) for 4-bromo-N-ethyl-2-(4-phenylpiperazin-1-yl)benzamide as described therein to provide the titled compound. ¹H NMR (300 MHz, CDCl₃) δ ppm 1.35 (s, 12H), 2.88 (s, 3H), 3.18-3.37 (m, 4H), 3.84 (s, 2H), 4.68-4.73 (m, 2H), 7.27-7.40 (m, 5H), 7.55 (s, 1H), 7.67 (d, J=6.8 Hz, 1H), 8.07 (d, J=6.8 Hz, 1H), 8.68 (s, 1H).

### Step 5

### 2-(4-benzyl-3-oxopiperazin-1-yl)-N-methyl-4-[3-methyl-4-(tetrahydro-2H-pyran-4-yloxy)-1H-indazol-6-yl]benzamide

The procedure of Example 115, Step 8 was used, substituting 2-(4-benzyl-3-oxopiperazin-1-yl)-N-methyl-4-(4,4,5,5-tetramethyl-1,3,2-dioxaborolan-2-yl)benzamide for N-ethyl-2-(4-phenylpiperazin-1-yl)-4-(4,4,5,5-tetramethyl-1,3,2-dioxaborolan-2-yl)benzamide as described therein, to provide the titled compound, which was purified by crystallization from heptanes-ethyl acetate. ¹H NMR (500 MHz, DMSO-d₆) δ ppm 1.71-1.80 (m, 2H), 2.03-2.10 (m, 2H), 2.61 (s, 3H), 2.75 (d, J=4.9 Hz, 3H), 3.31 (s, 4H), 3.60 (ddd, J=11.4, 7.6, 3.4 Hz, 2H), 3.85 (s, 2H), 3.85-3.90 (m, 2H), 4.61 (s, 2H), 4.96 (tt, J=7.1, 3.5 Hz, 1H), 6.77 (s, 1H), 7.22 (s, 1H), 7.22-7.33 (m, 5H), 7.34 (d, J=1.5 Hz, 1H), 7.42 (dd, J=7.9, 1.5 Hz, 1H), 7.60 (d, J=7.9 Hz, 1H), 8.55 (q, J=4.5 Hz, 1H), 12.63 (s, 1H); MS (ESI) m/z 554 (M+H)⁺.

### Example 160

### N-methyl-2-[4-(3-methylbutanoyl)piperazin-1-yl]-4-[3-methyl-4-(tetrahydro-2H-pyran-4-yloxy)-1H-indazol-6-yl]benzamide

The procedure of Example 124 was used, substituting 3-methylbutanoyl chloride for cyclopropanecarbonyl chloride as described therein, to provide the titled compound. ¹H NMR (300 MHz, DMSO-d₆) δ ppm 0.92 (d, J=6.8 Hz, 6H), 1.68-1.82 (m, 2H), 1.93-2.13 (m, 3H), 2.25 (d, J=6.8 Hz, 2H), 2.60 (s, 3H), 2.86 (d, J=4.7 Hz, 3H), 2.91-3.06 (m, 4H), 3.54-3.65 (m, 2H), 3.66 (s, 4H), 3.86 (ddd, J=11.2, 7.1, 3.7 Hz, 2H), 4.96 (tt, J=7.1, 3.4 Hz, 1H), 6.76 (s, 1H), 7.19 (s, 1H), 7.37 (d, J=1.4 Hz, 1H), 7.44 (dd, J=8.1, 1.5 Hz, 1H), 7.75 (d, J=8.1 Hz, 1H), 8.86 (q, J=4.7 Hz, 1H), 12.59 (s, 1H); MS (ESI) m/z 534 (M+H)⁺.

### Example 161

### 4-(cyclopentyloxy)-3-methyl-6-[5-(4-phenylpiperazin-1-yl)pyridin-3-yl]-1H-indazole

### Step 1

### 1-(5-bromopyridin-3-yl)-4-phenylpiperazine

A mixture of 1-phenylpiperazine (2.0 g, 12.3 mmol) and 3-bromo-5-fluoropyridine (220 mg, 1.25 mmol) was irradiated at 220° C. (Biotage Personal Chemistry unit, 300 W) for 20 minutes. The product was purified by flash chromatography (silica gel, eluted with heptanes-ethyl acetate, 90:10-30:70) to provide the titled compound (222 mg, 56%). ¹H NMR (300 MHz, DMSO-d₆) δ ppm 0.71-0.98 (m, 1H), 1.22-1.27 (m, 2H), 3.21-3.30 (m, 4H), 3.35-3.46 (m, 4H), 6.76-6.87 (m, 1H), 6.97-7.04 (m, 2H), 7.20-7.29 (m, 2H), 7.57-7.64 (m, 1H), 8.07 (d, J=1.9 Hz, 1H), 8.36 (d, J=2.6 Hz, 1H).

### Step 2

### 4-(cyclopentyloxy)-3-methyl-6-[5-(4-phenylpiperazin-1-yl)pyridin-3-yl]-1H-indazole

The procedure of Example 115, Step 8 substituting 4-(cyclopentyloxy)-3-methyl-6-(4,4,5,5-tetramethyl-1,3,2-dioxaborolan-2-yl)-1H-indazole (Example 127, Step 4) for N-ethyl-2-(4-phenylpiperazin-1-yl)-4-(4,4,5,5-tetramethyl-1,3,2-dioxaborolan-2-yl)benzamide, and 1-(5-bromopyridin-3-yl)-4-phenylpiperazine for 6-bromo-3-methyl-4-(tetrahydro-2H-pyran-4-yloxy)-1H-indazole as described therein, to provide the titled compound. ¹H NMR (300 MHz, DMSO-d₆) δ ppm 1.57-1.69 (m, 2H), 1.69-1.81 (m, 2H), 1.81-1.91 (m, 2H), 1.93-1.99 (m, 2H), 2.53 (s, 3H), 3.42 (dd, J=21.5, 7.4 Hz, 8H), 5.08-5.21 (m, 1H), 6.69 (s, 1H), 6.81 (t, J=7.2 Hz, 1H), 7.01 (d, J=7.9 Hz, 2H), 7.18 (d, J=0.9 Hz, 1H), 7.24 (dd, J=8.7, 7.3 Hz, 2H), 7.53-7.63 (m, 2H), 8.32 (d, J=1.7 Hz, 1H), 8.36 (d, J=2.7 Hz, 1H), 12.59 (s, 1H); MS (ESI) m/z 454 (M+H)⁺.

### Example 162

### tert-butyl 4-{2-[(methylsulfonyl)amino]-5-[3-methyl-4-(tetrahydro-2H-pyran-4-yloxy)-1H-indazol-6-yl]phenyl}piperazine-1-carboxylate

### Step 1

### tert-butyl 4-{5-bromo-2-[(trifluoroacetyl)amino]phenyl}piperazine-1-carboxylate

The procedure of Example 127, Step 3 was used, substituting tert-butyl 4-(5-bromo-2-nitrophenyl)piperazine-1-carboxylate (Example 141, Step 1) for 5-bromo-2-nitro-N-(2-phenoxyethyl)aniline, and trifluoroacetic anhydride for acetic anhydride as described therein, to provide the titled compound. ¹H NMR (400 MHz, DMSO-d₆) δ ppm 1.42 (s, 9H), 2.80-2.85 (m, 4H), 3.39-3.44 (m, 4H), 7.33 (dd, J=8.5, 2.1 Hz, 1H), 7.35-7.37 (m, 1H), 7.51 (d, J=8.5 Hz, 1H), 10.50 (s, 1H); MS (ESI) m/z 452/454 (M+H)⁺.

### Step 2

### tert-butyl 4-{5-(4,4,5,5-tetramethyl-1,3,2-dioxaborolan-2-yl)-2-[(trifluoroacetyl)amino]phenyl}piperazine-1-carboxylate

The procedure of Example 115, Step 7 was used, substituting tert-butyl 4-{5-bromo-2-[(trifluoroacetyl)amino]phenyl}piperazine-1-carboxylate (Step 1) for 4-bromo-N-ethyl-2-(4-phenylpiperazin-1-yl)benzamide as described therein to provide the titled compound. ¹H NMR (300 MHz, DMSO-d₆) δ ppm 1.30 (s, 12H), 1.42 (s, 9H), 2.76-2.84 (m, 4H), 3.39-3.49 (m, 4H), 7.44 (d, J=1.0 Hz, 1H), 7.48 (dd, J=8.0, 1.2 Hz, 1H), 7.73 (d, J=8.0 Hz, 1H), 10.38 (s, 1H); MS (ESI) m/z 500 (M+H)⁺.

### Step 3

### tert-butyl 4-{2-[(methylsulfonyl)amino]-5-[3-methyl-4-(tetrahydro-2H-pyran-4-yloxy)-1H-indazol-6-yl]phenyl}piperazine-1-carboxylate

The procedure of Example 115, Step 8 was used, substituting tert-butyl 4-{5-(4,4,5,5-tetramethyl-1,3,2-dioxaborolan-2-yl)-2-[(trifluoroacetyl)amino]phenyl}piperazine-1-carboxylate (Step 2) for N-ethyl-2-(4-phenylpiperazin-1-yl)-4-(4,4,5,5-tetramethyl-1,3,2-dioxaborolan-2-yl)benzamide as described therein, to provide tert-butyl 4-{2-amino-5-[3-methyl-4-(tetrahydro-2H-pyran-4-yloxy)-1H-indazol-6-yl]phenyl}piperazine-1-carboxylate. This material (33 mg, 0.065 mmol) was combined with triethylamine (0.05 mL, 0.36 mmol) in CH₂Cl₂ (3 mL) and stirred at room temperature as a solution of methanesulfonyl chloride (22.4 mg, 0.2 mmol) in CH₂Cl₂ (1 mL) was added. The mixture was stirred at room temperature for 4 hours, and then diluted with methanol (3 mL). Concentrated NH₄OH (0.5 mL) was added, and the mixture was stirred at room temperature for 14 hours, and then concentrated under vacuum. The residue was purified by HPLC (30×100 mm Waters C18 XBridge™ column eluted with aqueous 0.1% trifluoroacetic acid-CH₃CN, 80:20-5:95, 40 mL/minute over 15 minutes), then by flash chromatography (silica gel, eluted with ethyl acetate) to provide the titled compound (8.3 mg, 21%). ¹H NMR (500 MHz, DMSO-d₆) δ ppm 1.43 (s, 9H), 1.69-1.80 (m, 2H), 2.02-2.09 (m, 2H), 2.60 (s, 3H), 2.87-2.94 (m, 4H), 3.15 (s, 3H), 3.49-3.56 (m, 4H), 3.60 (ddd, J=11.4, 7.6, 3.4 Hz, 2H), 3.86 (ddd, J=11.1, 7.2, 3.7 Hz, 2H), 4.95 (tt, J=7.1, 3.5 Hz, 1H), 6.73 (s, 1H), 7.14 (s, 1H), 7.40-7.52 (m, 3H), 8.56 (br s, 1H), 12.55 (s, 1H); MS (ESI) m/z 586 (M+H)⁺.

### Example 163

### 1,1,1-trifluoropropan-2-yl 4-{2-(methylcarbamoyl)-5-[3-methyl-4-(tetrahydro-2H-pyran-4-yloxy)-1H-indazol-6-yl]phenyl}piperazine-1-carboxylate

### Step 1

### 4-nitrophenyl (1,1,1-trifluoropropan-2-yl) carbonate

Bis(4-nitrophenyl)carbonate (2.93 g, 9.64 mmol) was added in portions over 10 minutes to an ice-cooled solution of 1,1,1-trifluoro-2-propanol (1.10 g, 9.64 mmol) and triethylamine (1.65 mL, 11.9 mmol) in CH₂Cl₂ (20 mL). The solution was stirred at 0° C. for 1 hour, then diluted with CH₂Cl₂ (20 mL) and washed with 1 M NaOH (30 mL). The organic phase was dried (MgSO₄) and concentrated to provide the titled compound (1.99 g, 74%). ¹H NMR (300 MHz, CDCl₃) δ ppm 1.54-1.60 (m, 3H), 5.24 (septet, J=6.4 Hz, 1H), 7.37-7.45 (m, 2H), 8.26-8.35 (m, 2H).

### Step 2

### 1,1,1-trifluoropropan-2-yl 4-{2-(methylcarbamoyl)-5-[3-methyl-4-(tetrahydro-2H-pyran-4-yloxy)-1H-indazol-6-yl]phenyl}piperazine-1-carboxylate

Solid N-methyl-4-[3-methyl-4-(tetrahydro-2H-pyran-4-yloxy)-1H-indazol-6-yl]-2-(piperazin-1-yl)benzamide (Example 125, Step 1) (37.3 mg, 0.083 mmol) was added to a solution of 4-nitrophenyl (1,1,1-trifluoropropan-2-yl) carbonate (27.8 mg, 0.100 mmol) in CH₂Cl₂ (1 mL). The mixture was stirred at room temperature as triethylamine (0.040 mL, 0.29 mmol) was added, and the resulting solution was stirred for 14 hours. Methanol (1 mL) was added, and stirring was continued for 90 minutes longer. The solution was concentrated under vacuum, and the residue was purified by HPLC (30×100 mm Waters C18 XBridge™ column eluted with aqueous (NH₄)₂CO₃—CH₃CN, 80:20-5:95, 40 mL/minute over 15 minutes), to provide the titled compound (14 mg, 29%). ¹H NMR (300 MHz, DMSO-d₆) δ ppm 1.38 (d, J=6.8 Hz, 3H), 1.68-1.83 (m, 2H), 1.99-2.13 (m, 2H), 2.61 (s, 3H), 2.86 (d, J=4.7 Hz, 3H), 2.97-3.09 (m, 4H), 3.55-3.66 (m, 6H), 3.86 (ddd, J=11.2, 7.1, 3.7 Hz, 2H), 4.96 (tt, J=7.2, 3.9 Hz, 1H), 5.36 (septet, J=6.8 Hz, 1H), 6.76 (s, 1H), 7.20 (s, 1H), 7.40 (d, J=1.4 Hz, 1H), 7.45 (dd, J=8.1, 1.4 Hz, 1H), 7.76 (d, J=8.1 Hz, 1H), 8.85 (q, J=4.7 Hz, 1H), 12.60 (s, 1H); MS (ESI) m/z 590 (M+H)⁺.

### Example 164

### 2-[4-(cyclopropylacetyl)piperazin-1-yl]-N-methyl-4-[3-methyl-4-(tetrahydro-2H-pyran-4-yloxy)-1H-indazol-6-yl]benzamide

Solid 1-[bis(dimethylamino)methylene]-1H-[1,2,3]triazolo[4,5-b]pyridin-1-ium 3-oxide hexafluorophosphate (HATU) (76 mg, 0.200 mmol) was added to a stirring mixture of cyclopropylacetic acid (29 mg, 0.29 mmol), N-methyl-4-[3-methyl-4-(tetrahydro-2H-pyran-4-yloxy)-1H-indazol-6-yl]-2-(piperazin-1-yl)benzamide (Example 125, Step 1) (60 mg, 0.133 mmol) and diisopropylethylamine (0.047 mL, 0.27 mmol) in CH₂Cl₂ (2 mL). The mixture was stirred at room temperature for 2 hours, then methanol (1 mL) and a solution of 25% sodium methoxide/methanol (0.2 mL) was added, and the mixture stirred for 2 hours longer. The reaction mixture was diluted with water (5 mL) and extracted with CH₂Cl₂ (2×5 mL). The extract was concentrated under vacuum, and the residue was purified by HPLC ((30×100 mm Waters C18 XBridge™ column eluted aqueous 0.1% trifluoroacetic acid-CH₃CN, 80:20-5:95, 40 mL/minute over 15 minutes) to provide the titled compound (52 mg, 73%). ¹H NMR (400 MHz, DMSO-d₆) δ ppm −0.02-0.06 (m, 2H), 0.31-0.38 (m, 2H), 0.81-0.94 (m, 1H), 1.58-1.68 (m, 2H), 1.88-1.99 (m, 2H), 2.20 (d, J=6.7 Hz, 2H), 2.49 (s, 3H), 2.75 (d, J=4.5 Hz, 3H), 2.85-2.94 (m, 4H), 3.44-3.59 (m, 6H), 3.70-3.78 (m, 2H), 4.85 (tt, J=7.2, 3.7 Hz, 1H), 6.65 (s, 1H), 7.09 (s, 1H), 7.29 (d, J=1.2 Hz, 1H), 7.35 (dd, J=8.0, 1.2 Hz, 1H), 7.65 (d, J=8.0 Hz, 1H), 8.79 (q, J=4.5 Hz, 1H); MS (ESI) m/z 532 (M+H)⁺.

### Example 165

### 2-[4-(5-fluoropyrimidin-2-yl)piperazin-1-yl]-N-methyl-4-[3-methyl-4-(tetrahydro-2H-pyran-4-yloxy)-1H-indazol-6-yl]benzamide

5-Chloro-2-fluoropyrimidine (73 mg, 0.55 mmol) was added to a solution of N-methyl-4-[3-methyl-4-(tetrahydro-2H-pyran-4-yloxy)-1H-indazol-6-yl]-2-(piperazin-1-yl)benzamide (Example 125, Step 1) (91.3 mg, 0.20 mmol) and triethylamine (73 mg, 0.72 mmol) in dimethyl sulfoxide (1 mL). The mixture was heated at 40° C. for 17 hours. The reaction mixture was cooled to room temperature and subjected to purification by HPLC (Waters Nova-Pak® HR C18 6 μm 60 Å Prep-Pak® cartridge column (40 mm×100 mm) eluted with 10 mM aqueous ammonium acetate-acetonitrile, 90:10-0:100, 70 mL/minute over 12 minutes) to provide the titled compound. ¹H NMR (300 MHz, DMSO-d₆) δ ppm 1.70-1.80 (m, 2H), 2.01-2.10 (m, 2H), 2.60 (s, 3H), 2.87 (d, J=4.8 Hz, 3H), 3.09-3.13 (m, 4H), 3.59-3.63 (m, 2H), 3.82-3.92 (m, 6H), 4.92-5.00 (m, 1H), 6.67 (s, 1H), 7.20 (s, 1H), 7.43 (d, J=1.7 Hz, 1H), 7.47 (dd, J=8.1, 1.7 Hz, 1H), 7.80 (d, J=8.1 Hz, 1H), 8.50 (s, 2H), 9.00 (q, J=4.5 Hz, 1H), 12.58 (s, 1H); MS (ESI) m/z 546 (M+H)⁺.

### Example 166

### 4-[3-methyl-4-(tetrahydro-2H-pyran-4-yloxy)-1H-indazol-6-yl]-2-[6-(2,2,2-trifluoroethyl)-2,6-diazaspiro[3.3]hept-2-yl]benzamide

### Step 1

### 2-(2,2,2-trifluoroethyl)-2,6-diazaspiro[3.3]heptane bis(p-toluenesulfonate)

2,2,2-Trifluoroethyl trifluoromethanesulfonate (313 mg, 1.349 mmol) was added to a stirring suspension of tert-butyl 2,6-diazaspiro[3.3]heptane-2-carboxylate hemioxalate (293 mg, 1.204 mmol, prepared as described by Burkhard, J. et al. Org. Lett. 2008; 10: 3525-3526) and diisopropylethylamine (0.42 mL, 2.4 mmol) in tetrahydrofuran (3 mL). The mixture was heated at 60° C. for 3 hours, then cooled to room temperature and filtered. The solid was washed with tetrahydrofuran (1 mL), and the combined filtrates were concentrated under vacuum. The residue (293 mg) was dissolved in ethyl acetate (10 mL), and treated with a solution of p-toluenesulfonic acid monohydrate (300 mg, 1.577 mmol) in warm ethyl acetate (4 mL). The mixture was heated at reflux for 14 hours, and the resulting suspension was cooled to room temperature and filtered. The solid was washed with cold ethyl acetate (3 mL) and dried under vacuum to provide the titled compound (116 mg, 18%). ¹H NMR (400 MHz, methanol-d₄) δ ppm 2.37 (s, 6H), 4.08 (q, J=8.9 Hz, 2H), 4.35 (s, 4H), 4.50 (s, 4H), 7.25 (d, J=8.1 Hz, 4H), 7.71 (d, J=8.1 Hz, 4H); MS (ESI) m/z 181 (M+H)⁺.

### Step 2

### 4-bromo-2-(6-(2,2,2-trifluoroethyl)-2,6-diazaspiro[3.3]heptan-2-yl)benzonitrile

A solution of 2-(2,2,2-trifluoroethyl)-2,6-diazaspiro[3.3]heptane bis(p-toluenesulfonate) (Step 1, 116 mg, 0.221 mmol), 4-bromo-2-fluorobenzonitrile (44.2 mg, 0.221 mmol) and diisopropylethylamine (0.15 mL, 0.88 mmol) in dimethyl sulfoxide (3 mL) was heated at 120° C. for 45 minutes. The solution was cooled to room temperature, and diluted with water (15 mL). The mixture was extracted with CH₂Cl₂ (2×15 mL), and the organic phase was dried (MgSO₄) and concentrated under vacuum to leave the titled compound (80 mg, 100%). ¹H NMR (300 MHz, methanol-d₄) δ ppm 3.12 (q, J=9.6 Hz, 2H), 3.61 (s, 4H), 4.26 (s, 4H), 6.71 (d, J=1.7 Hz, 1H), 6.87 (dd, J=8.5, 1.7 Hz, 1H), 7.29 (d, J=8.5 Hz, 1H); MS (ESI) m/z 360/362 (M+H)⁺.

### Step 3

### 4-(4,4,5,5-tetramethyl-1,3,2-dioxaborolan-2-yl)-2-[6-(2,2,2-trifluoroethyl)-2,6-diazaspiro[3.3]hept-2-yl]benzonitrile

The procedure of Example 115, Step 7 was used, substituting 4-bromo-2-(6-(2,2,2-trifluoroethyl)-2,6-diazaspiro[3.3]heptan-2-yl)benzonitrile for 4-bromo-N-ethyl-2-(4-phenylpiperazin-1-yl)benzamide as described therein to provide the titled compound. ¹H NMR (300 MHz, CDCl₃) δ ppm 1.33 (s, 12H), 2.98 (q, J=9.5 Hz, 2H), 3.57 (s, 4H), 4.29 (s, 4H), 6.84 (s, 1H), 7.12 (d, J=7.8 Hz, 1H), 7.37 (d, J=7.8 Hz, 1H); MS (ESI) m/z (M+H)⁺.

### Step 4

### 4-[3-methyl-4-(tetrahydro-2H-pyran-4-yloxy)-1H-indazol-6-yl]-2-[6-(2,2,2-trifluoroethyl)-2,6-diazaspiro[3.3]hept-2-yl]benzamide

The procedure of Example 115, Step 8 was used, substituting 4-(4,4,5,5-tetramethyl-1,3,2-dioxaborolan-2-yl)-2-[6-(2,2,2-trifluoroethyl)-2,6-diazaspiro[3.3]hept-2-yl]benzonitrile for N-ethyl-2-(4-phenylpiperazin-1-yl)-4-(4,4,5,5-tetramethyl-1,3,2-dioxaborolan-2-yl)benzamide as described therein, to provide 4-[3-methyl-4-(tetrahydro-2H-pyran-4-yloxy)-1H-indazol-6-yl]-2-[6-(2,2,2-trifluoroethyl)-2,6-diazaspiro[3.3]hept-2-yl]benzonitrile (23 mg, 58%). This material was processed according to the procedure of Example 132, Step 2 to provide the titled compound, purified by crystallization from ethanol-water (2 mL, 50:50) (7.5 mg, 32%). ¹H NMR (400 MHz, DMSO-d₆) δ ppm 1.57-1.70 (m, 2H), 1.87-2.01 (m, 2H), 2.49 (s, 3H), 3.06 (q, J=10.1 Hz, 2H), 3.39 (s, 4H), 3.49 (ddd, J=11.3, 7.6, 3.4 Hz, 2H), 3.75 (ddd, J=11.2, 7.1, 3.7 Hz, 2H), 3.87 (s, 4H), 4.81 (tt, J=7.1, 3.5 Hz, 1H), 6.53 (d, J=1.5 Hz, 1H), 6.58 (s, 1H), 6.87 (dd, J=7.9, 1.5 Hz, 1H), 7.01 (s, 1H), 7.14 (s, 1H), 7.18 (d, J=7.6 Hz, 1H), 7.48 (s, 1H), 12.45 (s, 1H); MS (ESI) m/z 530 (M+H)⁺.

### Example 167

### 4-[3-amino-4-(tetrahydro-2H-pyran-4-yloxy)-1H-indazol-6-yl]-N-methyl-2-[4-(2,2,3,3-tetrafluoropropyl)piperazin-1-yl]benzamide

### Step 1

### 4-bromo-N-methyl-2-[4-(2,2,3,3-tetrafluoropropyl)piperazin-1-yl]benzamide

The titled compound was prepared according to the procedure of Example 108, Step 1, substituting 1-(2,2,3,3-tetrafluoropropyl)piperazine for 1-(2,2,2-trifluoroethyl)piperazine. ¹H NMR (300 MHz, DMSO-d₆) δ ppm 2.70-2.74 (m, 4H), 2.79 (d, J=4.8 Hz, 3H), 2.92-2.95 (m, 4H), 3.00-3.11 (m, 2H), 6.32-6.69 (m, 1H), 7.25 (m, 1H), 7.27 (m, 1H), 7.49 (m, 1H), 8.60 (m, 1H); MS (ESI⁺) m/z 412/414 (M+H)⁺.

### Step 2

### N-methyl-2-[4-(2,2,3,3-tetrafluoropropyl)piperazin-1-yl]-4-(4,4,5,5-tetramethyl-1,3,2-dioxaborolan-2-yl)benzamide

The titled compound was prepared according to the procedure of Example 108, Step 2, substituting the product of Step 1 for the product of Example 108, Step 1. ¹H NMR (300 MHz, DMSO-d₆) δ ppm 1.30 (s, 12H), 2.72-2.75 (m, 4H), 2.82 (d, J=4.8 Hz, 3H), 2.91-2.94 (m, 4H), 3.02-3.13 (m, 2H), 6.39-6.73 (m, 1H), 7.40-7.43 (m, 2H), 7.67 (m, 1H), 8.94 (m, 1H); MS (ESI⁺) m/z 460 (M+H)⁺.

### Step 3

### 4-[3-amino-4-(tetrahydro-2H-pyran-4-yloxy)-1H-indazol-6-yl]-N-methyl-2-[4-(2,2,3,3-tetrafluoropropyl)piperazin-1-yl]benzamide

The titled compound was prepared according to the procedure of Example 108, Step 3, substituting the product of Step 2 for the product of Example 108, Step 2. ¹H NMR (300 MHz, DMSO-d₆) δ ppm 1.72-1.82 (m, 2H), 1.97-2.12 (m, 2H), 2.76-2.79 (m, 4H), 2.85 (d, J=4.8 Hz, 3H), 3.01-3.05 (m, 4H), 3.08 (m, 1H), 3.13 (m, 1H), 3.53-3.59 (m, 2H), 3.84-3.90 (m, 2H), 4.92 (m, 1H), 5.00 (br, 2H), 6.36-6.71 (m, 1H), 6.64 (m, 1H), 7.00 (m, 1H), 7.37-7.43 (m, 2H), 7.76 (m, 1H), 8.95 (m, 1H), 11.47 (m, 1H); MS (ESI⁺) m/z 565 (M+H)⁺.

### Example 168

### 4-[3-amino-4-(tetrahydro-2H-pyran-4-yloxy)-1H-indazol-6-yl]-N-methyl-2-[4-(3,3,3-trifluoropropyl)piperazin-1-yl]benzamide

### Step 1

### 4-bromo-N-methyl-2-[4-(3,3,3-trifluoropropyl)piperazin-1-yl]benzamide

The titled compound was prepared according to the procedure of Example 108, Step 1, substituting 1-(3,3,3-trifluoropropyl)piperazine dihydrochloride for 1-(2,2,2-trifluoroethyl)piperazine. ¹H NMR (300 MHz, DMSO-d₆) δ ppm 2.45 (m, 2H), 2.54-2.61 (m, 6H), 2.80 (d, J=4.8 Hz, 3H), 2.91-2.94 (m, 4H), 7.25-7.28 (m, 2H), 7.49 (m, 1H), 8.59 (m, 1H); MS (ER⁺) m/z 394/396 (M+H)⁺.

### Step 2

### N-methyl-4-(4,4,5,5-tetramethyl-1,3,2-dioxaborolan-2-yl)-2-[4-(3,3,3-trifluoropropyl)piperazin-1-yl]benzamide

The titled compound was prepared according to the procedure of Example 108, Step 2, substituting the product of Step 1 for the product of Example 108, Step 1. ¹H NMR (300 MHz, DMSO-d₆) δ ppm 1.30 (s, 12H), 2.46 (m, 2H), 2.57-2.62 (m, 6H), 2.82 (d, J=4.8 Hz, 3H), 2.90-2.93 (m, 4H), 7.37-7.41 (m, 2H), 7.64 (m, 1H), 8.84 (m, 1H); MS (ESI⁺) m/z 442 (M+H)⁺.

### Step 3

### 4-[3-amino-4-(tetrahydro-2H-pyran-4-yloxy)-1H-indazol-6-yl]-N-methyl-2-[4-(3,3,3-trifluoropropyl)piperazin-1-yl]benzamide

The titled compound was prepared according to the procedure of Example 108, Step 3, substituting the product of Step 2 for the product of Example 108, Step 2. ¹H NMR (300 MHz, DMSO-d₆) δ ppm 1.70-1.82 (m, 2H), 2.01-2.09 (m, 2H), 2.43 (m, 2H), 2.59-2.64 (m, 6H), 2.85 (d, J=4.8 Hz, 3H), 3.00-3.03 (m, 4H), 3.53-3.60 (m, 2H), 3.83-3.90 (m, 2H), 4.92 (m, 1H), 4.99 (br, 2H), 6.64 (m, 1H), 7.00 (m, 1H), 7.35-7.42 (m, 2H), 7.75 (m, 1H), 8.97 (m, 1H), 11.46 (m, 1H); MS (ESI⁺) m/z 547 (M+H)⁺.

### Example 169

### 4-[3-amino-4-(tetrahydro-2H-pyran-4-yloxy)-1H-indazol-6-yl]-2-[4-(2,2-difluoroethyl)piperazin-1-yl]-N-methylbenzamide

### Step 1

### 4-bromo-2-[4-(2,2-difluoroethyl)piperazin-1-yl]-N-methylbenzamide

The titled compound was prepared according to the procedure of Example 108, Step 1, substituting 1-(2,2-difluoroethyl)piperazine for 1-(2,2,2-trifluoroethyl)piperazine. ¹H NMR (300 MHz, DMSO-d₆) δ ppm 2.66-2.69 (m, 4H), 2.75-2.84 (m, 5H), 2.92-2.94 (m, 4H), 6.01-6.31 (m, 1H), 7.25-7.28 (m, 2H), 7.48 (m, 1H), 8.59 (m, 1H); MS (ESI⁺) m/z 362 (M+H)⁺.

### Step 2

### 2-[4-(2,2-difluoroethyl)piperazin-1-yl]-N-methyl-4-(4,4,5,5-tetramethyl-1,3,2-dioxaborolan-2-yl)benzamide

The titled compound was prepared according to the procedure of Example 108, Step 2, substituting the product of Step 1 for the product of Example 108, Step 1 ¹H NMR (300 MHz, DMSO-d₆) δ ppm 1.30 (s, 12H), 2.69-2.71 (m, 4H), 2.76-2.85 (m, 5H), 2.90-2.93 (m, 4H), 6.01-6.31 (h, 1H), 7.37-7.41 (m, 2H), 7.63 (m, 1H), 8.81 (m, 1H); MS (ESI⁺) m/z 410 (M+H)⁺.

### Step 3

### 4-[3-amino-4-(tetrahydro-2H-pyran-4-yloxy)-1H-indazol-6-yl]-2-[4-(2,2-difluoroethyl)piperazin-1-yl]-N-methylbenzamide

The titled compound was prepared according to the procedure of Example 108, Step 3, substituting the product of Step 2 for the product of Example 108, Step 2. ¹H NMR (300 MHz, DMSO-d₆i) δ ppm 1.70-1.82 (m, 2H), 2.01-2.12 (m, 2H), 2.72-2.78 (m, 6H), 2.85 (d, J=4.8 Hz, 3H), 3.01-3.04 (m, 4H), 3.53-3.61 (m, 2H), 3.82-3.90 (m, 2H), 4.92 (m, 1H), 4.99 (br, 2H), 5.99-6.36 (m, 1H), 6.64 (m, 1H), 7.00 (m, 1H), 7.37-7.44 (m, 2H), 7.75 (m, 1H), 8.96 (m, 1H), 11.47 (m, 1H); MS (ESL) m/z 515 (M+H)⁺.

### Example 170

### 4-[3-amino-4-(tetrahydro-2H-pyran-4-yloxy)-1H-indazol-6-yl]-N-methyl-2-[4-(2,2,3,3,3-pentafluoropropyl)piperazin-1-yl]benzamide

### Step 1

### tert-butyl 4-(2,2,3,3,3-pentafluoropropyl)piperazine-1-carboxylate

tert-Butyl piperazine-1-carboxylate (0.289 g, 1.55 mmol), 2,2,3,3,3-pentafluoropropyl trifluoromethanesulfonate (0.700 g, 2.481 mmol), and triethylamine (2.2 mL, 15.78 mmol) were heated in toluene (7.8 mL) overnight at 80° C. The mixture was then cooled to room temperature and concentrated in vacuo. The residue was treated with 1 M aqueous sodium hydroxide (10 mL), and then the mixture was extracted three times with ethyl acetate. The combined organic extracts were washed with brine, dried over Na₂SO₄, and concentrated. The crude product was purified by silica gel chromatography (0 to 10% methanol-ethyl acetate, eluant) to afford the titled compound as a colorless oil, 0.426 g (86%). ¹H NMR (300 MHz, DMSO-d₆) δ ppm 1.46 (s, 9H), 2.60 (m, 4H), 2.99 (m, 2H), 3.43 (m, 4H); MS (ESL) 319 (M+H)⁺.

### Step 2

### 1-(2,2,3,3,3-pentafluoropropyl)piperazine

The product of Step 1 (0.426 g, 1.138 mmol) was dissolved in CH₂Cl₂ (4 mL) and treated with trifluoroacetic acid (4 mL, 51.9 mmol). The mixture stirred at room temperature for 2 hours and was then concentrated in vacuo. The residue was treated with 1 M aqueous potassium carbonate solution (15 mL), and this mixture was then extracted three times with dichloromethane. The combined organic extracts were washed with brine, dried over Na₂SO₄, and concentrated to afford the titled compound as a colorless oil, 0.292 g (100%). ¹H NMR (300 MHz, DMSO-d₆) δ ppm 2.95-2.98 (m, 4H), 3.08 (m, 2H), 3.23-3.26 (m, 4H), 9.40 (br, 1H); MS (DCI⁺) m/z 219 (M+H), 236 (M+NH₄).

### Step 3

### 4-bromo-N-methyl-2-[4-(2,2,3,3,3-pentafluoropropyl)piperazin-1-yl]benzamide

The titled compound was prepared according to the procedure of Example 108, Step 1, substituting the product from Step 2 for 1-(2,2,2-trifluoroethyl)piperazine. ¹H NMR (300 MHz, DMSO-d₆) δ ppm 2.72-2.81 (m, 9H), 2.92-2.95 (m, 4H), 7.25-7.28 (m, 2H), 7.48 (m, 1H), 8.59 (m, 1H); MS (DC) m/z 430/432 (M+H)⁺.

### Step 4

### N-methyl-2-[4-(2,2,3,3,3-pentafluoropropyl)piperazin-1-yl]-4-(4,4,5,5-tetramethyl-1,3,2-dioxaborolan-2-yl)benzamide

The titled compound was prepared according to the procedure of Example 108, Step 2, substituting the product of Step 3 for the product of Example 108, Step 1 ¹H NMR (300 MHz, DMSO-d₆) δ ppm 1.30 (s, 12H), 2.77-2.84 (m, 7H), 2.90-2.93 (m, 4H), 3.20-3.28 (m, 2H), 7.38-7.42 (m, 2H), 7.64 (m, 1H), 8.85 (m, 1H); MS (ESL′) m/z 478 (M+H)⁺.

### Step 5

### 4-[3-amino-4-(tetrahydro-2H-pyran-4-yloxy)-1H-indazol-6-yl]-N-methyl-2-[4-(2,2,3,3,3-pentafluoropropyl)piperazin-1-yl]benzamide

The titled compound was prepared according to the procedure of Example 108, Step 3, substituting the product of Step 4 for the product of Example 108, Step 2. ¹H NMR (300 MHz, DMSO-d₆) δ ppm 1.81-1.84 (m, 2H), 2.04-2.07 (m, 2H), 2.86-2.89 (m, 7H), 3.09-3.11 (m, 4H), 3.30-3.35 (m, 2H), 3.53-3.56 (m, 2H), 3.88-3.92 (m, 2H), 4.87-5.02 (m, 3H), 6.84 (m, 1H), 7.16 (m, 1H), 7.48-7.50 (m, 2H), 7.79 (m, 1H), 8.96 (m, 1H), 11.77 (m, 1H); MS (ESI⁺) 583 (M+H)⁺.

### Example 171

### 4-[3-amino-4-(tetrahydro-2H-pyran-4-yloxy)-1H-indazol-6-yl]-2-{4-[(3,3-difluorocyclobutyl)methyl]piperazin-1-yl}-N-methylbenzamide

### Step 1

### (3,3-difluorocyclobutyl)methanol

A suspension of sodium borohydride (0.556 g, 14.70 mmol) in tetrahydrofuran (50 mL) was cooled to 0° C. and then treated dropwise with a solution of 3,3-difluorocyclobutanecarboxylic acid (2 g, 14.70 mmol) in tetrahydrofuran (50 mL). After completion of the addition, the reaction flask was removed from the cold bath, and the reaction was stirred at room temperature for 1 hour. After this time, the reaction mixture was cooled again to 0° C. Then boron trifluoride-diethyl etherate (1.862 mL, 14.70 mmol) was added slowly, and the reaction was allowed to stir overnight at 0° C. with gradual warming to room temperature. The mixture was then again cooled to 0° C. and quenched with 25 mL of 95% ethanol. The mixture was stirred for 1 hour in the ice bath, and then the mixture was concentrated in vacuo. The residue was partitioned between CH₂Cl₂ and brine, and the phases were separated. The organic layer was dried over Na₂SO₄ and concentrated in vacuo to afford the titled compound as a pale tan oil, 1.629 g (91%). ¹H NMR (300 MHz, CDCl₃) δ ppm 2.31-2.38 (m, 3H), 2.60-2.66 (m, 2H), 3.67 (m, 2H); MS (DCL) m/z 123 (M+H)⁺.

### Step 2

### (3,3-difluorocyclobutyl)methyl 4-methylbenzenesulfonate

The product from Step 1 (1.629 g, 13.34 mmol) in pyridine (40 mL) was cooled to 0° C. and then treated in three portions with p-toluenesulfonyl chloride (6.61 g, 34.7 mmol). The reaction mixture was stirred overnight at 0° C. with gradual warming to room temperature. The reaction mixture was then poured into icy water. The mixture was extracted three times with CH₂Cl₂, then the combined organics were dried over Na₂SO₄ and concentrated in vacuo. The crude material was purified by silica gel chromatography (5 to 50% ethyl acetate-heptane, eluant) to afford the titled compound as a colorless oil, 1.926 g (52%). ¹H NMR (300 MHz, CDCl₃) δ ppm 2.04-2.36 (m, 2H), 2.45-2.71 (m, 3H), 2.46 (s, 3H), 4.06 (d, J=6.4 Hz, 2H), 7.37 (m, 2H), 7.80 (m, 2H); MS (ESL) m/z 294 (M+NH₄).

### Step 3

### N-methyl-2-(piperazin-1-yl)-4-(4,4,5,5-tetramethyl-1,3,2-dioxaborolan-2-yl)benzamide

The titled compound was prepared according to the procedure of Example 170, Step 2 substituting the product of Example 111, Step 1, for the product of Example 170, Step 1. The crude material was taken directly into the next step without purification. MS (ESL) m/z 346 (M+H)⁺.

### Step 4

### 2-{4-[(3,3-difluorocyclobutyl)methyl]piperazin-1-yl}-N-methyl-4-(4,4,5,5-tetra methyl-1,3,2-dioxaborolan-2-yl)benzamide

The titled compound was prepared according to the procedure of Example 170, Step 1, substituting the product of Step 3 for tert-butyl piperazine-1-carboxylate and substituting the product of Step 2 for 2,2,3,3,3-pentafluoropropyl trifluoromethanesulfonate. ¹H NMR (300 MHz, DMSO-d₆) δ ppm 1.30 (s, 12H), 2.21-2.33 (m, 4H), 2.62-2.72 (m, 4H), 2.81 (d, J=6.7 Hz, 3H), 2.88-2.91 (m, 4H), 3.91 (m, 1H), 4.02 (m, 2H), 7.38-7.41 (m, 2H), 7.65 (m, 1H), 8.89 (m, 1H); MS (ES) 450 (M+H)⁺.

### Step 5

### 4-[3-amino-4-(tetrahydro-2H-pyran-4-yloxy)-1H-indazol-6-yl]-2-{4-[(3,3-difluorocyclobutyl)methyl]piperazin-1-yl}-N-methylbenzamide

The titled compound was prepared according to the procedure of Example 108, Step 3, substituting the product of Step 4 for the product of Example 108, Step 2. ¹H NMR (300 MHz, DMSO-d₆) δ ppm 1.71-1.81 (m, 2H), 2.01-2.08 (m, 2H), 2.23-2.37 (m, 4H), 2.56-2.59 (m, 4H), 2.85 (d, J=4.8 Hz, 3H), 2.97-3.04 (m, 4H), 3.47-3.61 (m, 2H), 3.78-4.02 (m, 5H), 4.85-4.99 (m, 3H), 6.64 (m, 1H), 7.00 (m, 1H), 7.39-7.42 (m, 2H), 7.74-7.77 (m, 1H), 9.01 (m, 1H), 11.47 (m, 1H); MS (ES) m/z 555 (M+H)⁺.

### Example 172

### 4-[3-amino-4-(tetrahydro-2H-pyran-4-yloxy)-1H-indazol-6-yl]-2-[4-(2,2-difluoropropyl)piperazin-1-yl]-N-methylbenzamide

### Step 1

### tert-butyl 4-[5-bromo-2-(methylcarbamoyl)phenyl]piperazine-1-carboxylate

The titled compound was prepared according to the procedure of Example 108, Step 1, substituting tert-butyl piperazine-1-carboxylate for 1-(2,2,2-trifluoroethyl)piperazine. ¹H NMR (300 MHz, CDCl₃) δ ppm 1.42 (s, 9H), 2.80 (d, J=4.8 Hz, 3H), 2.89 (m, 4H), 3.47 (m, 4H), 7.27-7.30 (m, 2H), 7.51 (m, 1H), 8.57 (m, 1H); MS (ESI⁺) m/z 398/400 (M+H)⁺.

### Step 2

### 4-bromo-N-methyl-2-(piperazin-1-yl)benzamide

The titled compound was prepared according to the procedure of Example 170, Step 2, substituting the product of Example Step 1 for the product of Step 1. ¹H NMR (300 MHz, DMSO-d₆) δ ppm 2.80-2.92 (m, 11H), 7.23-7.28 (m, 2H), 7.53 (m, 1H), 8.74 (m, 1H); MS (ESI⁺) m/z 298/300 (M+H)⁺.

### Step 3

### 4-bromo-N-methyl-2-[4-(2-oxopropyl)piperazin-1-yl]benzamide

A solution of Step 2 (0.656 g, 2.2 mmol) in acetonitrile (11 mL) was treated with potassium carbonate (0.365 g, 2.64 mmol) and stirred for 5 minutes at room temperature. Chloroacetone (0.18 mL, 2.237 mmol) was then added dropwise. The reaction was stirred at room temperature for 5 minutes and then at reflux overnight. After this time, the mixture was cooled to room temperature, poured into water, and extracted three times with dichloromethane. The extracts were washed with brine, dried over sodium sulfate, and concentrated. The residue was chromatographed on silica gel (0 to 10% methanol-ethyl acetate, eluant) to afford the titled compound as a thick yellow oil, 0.596 g (76%). ¹H NMR (300 MHz, DMSO-d₆) δ ppm 2.09 (s, 3H), 2.55-2.58 (m, 4H), 2.79 (d, J=4.9 Hz, 3H ( ) 2.93-2.95 (m, 4H), 3.25 (s, 2H), 7.25-7.28 (m, 2H), 7.50 (m, 1H), 8.64 (m, 1H); MS (ESI⁺) m/z 354/356 (M+H)⁺.

### Step 4

### 4-bromo-2-[4-(2,2-difluoropropyl)piperazin-1-yl]-N-methylbenzamide

A solution of Step 3 (0.300 g, 0.847 mmol) in dichloromethane (9 mL) was cooled to −78° C., and then treated dropwise with (diethylamino)sulfur trifluoride (DAST) (0.34 mL, 2.57 mmol). The reaction was stirred overnight initially at −78° C. and then with gradual warming to room temperature. After this time, the reaction was quenched with saturated aqueous sodium bicarbonate solution (10 mL), and the mixture was then extracted three times with dichloromethane (20 mL each). The combined organic extracts were washed with brine, dried over Na₂SO₄, and concentrated. The crude material was taken directly into the next reaction without further purification. MS (ESI⁺) m/z 376/378 (M+H)⁺.

### Step 5

### 2-[4-(2,2-difluoropropyl)piperazin-1-yl]-N-methyl-4-(4,4,5,5-tetramethyl-1,3,2-dioxaborolan-2-yl)benzamide

The titled compound was prepared according to the procedure of Example 108, Step 2, substituting the product of Step 4 for the product of Example 108, Step 1. ¹H NMR (300 MHz, DMSO-d₆) δ ppm 1.30 (s, 12H), 1.65 (m, 3H), 2.69-2.93 (m, 13H), 7.39-7.42 (m, 2H), 7.66 (m, 1H), 8.92 (m, 1H); MS (ESI⁺) 424 (M+H)⁺.

### Step 6

### 4-[3-amino-4-(tetrahydro-2H-pyran-4-yloxy)-1H-indazol-6-yl]-2-[4-(2,2-difluoropropyl)piperazin-1-yl]-N-methylbenzamide

The titled compound was prepared according to the procedure of Example 108, Step 3, substituting the product of Step 5 for the product of Example 108, Step 2. ¹H NMR (300 MHz, DMSO-d₆) δ ppm 1.65 (m, 3H), 1.72-1.80 (m, 2H), 2.03-2.08 (m, 2H), 2.75-2.86 (m, 9H), 3.01-3.03 (m, 4H), 3.54-3.59 (m, 2H), 3.84-3.89 (m, 2H), 4.90-5.00 (m, 3H), 6.64 (m, 1H), 7.00 (m, 1H), 7.38-7.42 (m, 2H), 7.76 (m, 1H), 8.99 (m, 1H), 11.48 (m, 1H); MS (ESL) m/z 529 (M+H)⁺.

### Example 173

### 4-[3-amino-4-(tetrahydro-2H-pyran-4-yloxy)-1H-indazol-6-yl]-2-{4-[(2,2-difluorocyclopropyl)methyl]piperazin-1-yl}-N-methylbenzamide

### Step 1

### 2-{4-[(2,2-difluorocyclopropyl)methyl]piperazin-1-yl}-N-methyl-4-(4,4,5,5-tetramethyl-1,3,2-dioxaborolan-2-yl)benzamide

The titled compound was prepared according to the procedure of Example 170, Step 1, substituting the product of Example 171, Step 3 for tert-butyl piperazine-1-carboxylate and substituting 2-(bromomethyl)-1,1-difluorocyclopropane for 2,2,3,3,3-pentafluoropropyl trifluoromethanesulfonate. ¹H NMR (300 MHz, DMSO-d₆) δ ppm 1.18 (m, 1H), 1.32 (s, 12H), 1.60 (m, 1H), 1.84 (m, 1H), 2.56-2.69 (m, 6H), 2.81 (d, J=4.7 Hz, 3H), 2.91-2.94 (m, 4H), 7.38-7.41 (m, 2H), 7.64 (m, 1H), 8.85 (m, 1H); MS (ESL) m/z 436 (M+H)⁺.

### Step 2

### 4-[3-amino-4-(tetrahydro-2H-pyran-4-yloxy)-1H-indazol-6-yl]-2-{4-[(2,2-difluorocyclopropyl)methyl]piperazin-1-yl}-N-methylbenzamide

The titled compound was prepared according to the procedure of Example 108, Step 3, substituting the product of Step 1 for the product of Example 108, Step 2. ¹H NMR (300 MHz, DMSO-d₆) δ ppm 1.20 (m, 1H), 1.61 (m, 1H), 1.71-1.93 (m, 3H), 2.02-2.08 (m, 2H), 2.57-2.68 (m, 6H), 2.85 (d, J=4.7 Hz, 3H), 3.02-3.05 (m, 4H), 3.54-03.60 (m, 2H), 3.83-3.90 (m, 2H), 4.89-4.99 (m, 3H), 6.64 (m, 1H), 7.00 (m, 1H), 7.38-7.43 (m, 2H), 7.77 (m, 1H), 9.01 (m, 1H), 11.47 (m, 1H); MS (ESL) m/z 541 (M+H)⁺.

### Determination of Biological Activity

Abbreviations: ahx for 2-aminohexanoic acid; ATP for adenosine triphosphate; BSA for bovine serum albumin; EDTA for ethylenediaminetetraacetic acid; HEPES for HEPES for 2-(4-(2-hydroxyethyl)piperazin-1-yl)ethanesulfonic acid; LCK for leukocyte specific protein tyrosine kinase; Tween® 20 for polyethylene glycol sorbitan monolaurate.

### Determination of Inhibitory Potency at TrkA Kinase, TrkB Kinase, and TrkC Kinase

TrkA, TrkB, and TrkC enzymes were obtained from Invitrogen (as catalog numbers PV3144, PV3616, and PV3617, respectively). The ability of a compound to inhibit the enzymatic activity of the TrkA, TrkB, and TrkC enzymes was determined in separate assays. Enzyme activity was measured in an HTRF® (homogeneous time resolved fluorescence) assay, which detects enzymatic phosphorylation of a biotinylated synthetic peptide substrate (an LCK peptide analog, biotin-ahx-GAEEEIYAAFFA, from Genemed Synthesis). Phosphorylation is assessed by HTRF® in the presence of an anti-phosphotyrosine antibody conjugated to Eu³⁺ cryptate (Cisbio) as donor fluorophore, and streptavidin conjugated to allophycocyanin (ProZyme) as acceptor fluorophore. The HTRF® signal was detected at two different wavelengths (620 nm and 665 nm) which were used to calculate the fluorescence ratio.

Each enzyme was titrated to a concentration optimized to ensure accurate measurement of the initial reaction rate. To allow the Trk enzymes to undergo activation and auto-phosphorylation, a 20 minute pre-incubation with ATP was carried out (at twice the final targeted enzyme and ATP concentration) prior to addition of test compound and the peptide substrate.

TrkA in vitro assays were performed by pre-incubating TrkA (2-10 nM) with ATP (200 μM) for 20 minutes at ambient temperature in 50 mM HEPES pH 7.4, 10 mM MgCl₂, 2 mM MnCl₂, 100 μM Na₃VO₄, 1 mM dithiothreitol, 0.01% BSA (bovine serum albumin). Then, to the activated Trk enzyme mixture was added the test compound in 2% dimethyl sulfoxide. After 10 minutes, the peptide substrate (125 nM) was added. After 1 hour, enzyme reactions were terminated by addition of equal reaction volumes of detection/stop reagent (containing 0.2 μg/mL anti-phosphotyrosine monoclonal antibody labeled with europium from Cisbio (catalog# PT66-K), and 4 μg/mL PhycoLink® Streptavidin-Allophycocyanin conjugate from ProZyme (catalog# PJ25S) in 60 mM EDTA in 40 mM HEPES pH 7.4, 480 mM KF, 0.01% Tween® 20 and 0.1% BSA bovine serum albumin) Reaction plates were stored at 4° C. overnight before reading the fluorescence ratio signal on a PerkinElmer EnVision™ fluorescence detector. Individual reaction wells were stopped at time points to obtain reaction rates. The test inhibitor compounds were assayed in duplicate, at half-log serial dilutions over a range of concentrations (e.g. starting at 50 μM or 5 μM as the high compound concentration). The percent Trk enzyme inhibition was calculated from the initial rates of the inhibited reactions relative to the uninhibited control. IC₅₀ values were calculated by fitting the inhibition percent to the concentration of inhibitor [I] in the assay in equation 1 below, to solve for the IC₅₀.

Inhibition %=100[I]/([I]+[IC₅₀]) equation 1

The TrkB and TrkC in vitro assays were performed as described above, except that for these enzymes the assay ATP concentration targeted was 5 μM.

| Example | IC₅₀ TrkA [μM] | IC₅₀ TrkB [μM] | IC₅₀ TrkC [μM] |
|---|---|---|---|
| 1 | 0.00266 | 0.000786 | 0.00103 |
| 2 | 0.0074 | 0.00309 | 0.00385 |
| 3 | 0.0553 | 0.0206 | 0.0257 |
| 4 | 0.147 | | |
| 5 | 0.037 | | |
| 6 | 0.0421 | 0.015 | 0.0237 |
| 7 | 0.706 | | |
| 8 | 0.00442 | | |
| 9 | 3.46 | | |
| 10 | 0.136 | | |
| 11 | 0.208 | | |
| 12 | 0.0029 | | |
| 13 | 0.0629 | | |
| 14 | 0.11 | | |
| 15 | 0.00959 | | |
| 16 | 0.0296 | | |
| 17 | 0.0134 | | |
| 18 | 0.0364 | | |
| 19 | 0.0381 | | |
| 20 | 0.101 | | |
| 21 | 0.0333 | | |
| 22 | 0.254 | | |
| 23 | 0.015 | 0.00804 | 0.00853 |
| 24 | 0.0204 | | |
| 25 | 0.188 | | |
| 26 | 0.0581 | | |
| 27 | 0.251 | | |
| 28 | 0.041 | | |
| 29 | 0.0979 | 0.046 | 0.0559 |
| 30 | 0.0104 | 0.0015 | 0.0015 |
| 31 | 0.0421 | | |
| 32 | 0.376 | | |
| 33 | 0.028 | | |
| 34 | 0.318 | 0.33 | 0.318 |
| 35 | 0.0595 | 0.0919 | 0.105 |
| 36 | 0.0258 | | |
| 37 | 0.667 | | |
| 38 | 0.0312 | | |
| 39 | 0.0417 | | |
| 40 | 0.0714 | | |
| 41 | 0.0292 | | |
| 42 | 0.422 | | |
| 43 | 0.0631 | | |
| 44 | 0.0253 | | |
| 45 | 0.30 | | |
| 46 | 0.0325 | | |
| 47 | 0.232 | 0.268 | 0.30 |
| 48 | 0.0385 | | |
| 49 | 0.141 | | |
| 50 | 0.53 | | |
| 51 | 0.0754 | | |
| 52 | 0.0272 | | |
| 53 | 0.0444 | | |
| 54 | 0.101 | | |
| 55 | 0.121 | | |
| 56 | 0.080 | 0.0278 | 0.0371 |
| 57 | 6.32 | | |
| 58 | 0.673 | | |
| 59 | 2.55 | | |
| 60 | 0.223 | | |
| 61 | 0.135 | | |
| 62 | 0.0671 | | |
| 63 | 0.0253 | | |
| 64 | 0.00845 | | |
| 65 | 0.173 | | |
| 66 | 0.00293 | | |
| 67 | 0.00797 | | |
| 68 | 0.00806 | | |
| 69 | 0.00295 | 0.00163 | 0.00309 |
| 70 | 0.0349 | 0.0837 | 0.145 |
| 71 | 0.00492 | 0.0015 | 0.00167 |
| 72 | 0.0175 | 0.00492 | 0.00535 |
| 73 | 0.0137 | 0.00317 | 0.00353 |
| 74 | 0.019 | 0.00389 | 0.00832 |
| 75 | 0.00231 | | |
| 76 | 0.0245 | | |
| 77 | 0.0511 | 0.0844 | 0.158 |
| 78 | 0.0201 | | |
| 79 | 0.221 | | |
| 80 | 0.0175 | | |
| 81 | 0.0174 | | |
| 82 | 0.0223 | | |
| 83 | 8.33 | | |
| 84 | 0.0243 | | |
| 85 | 2.24 | | |
| 86 | 0.0161 | | |
| 87 | 0.0433 | | |
| 88 | 8.44 | | |
| 89 | 13.5 | | |
| 90 | 0.227 | | |
| 91 | 0.00531 | 0.00201 | 0.00289 |
| 92 | 0.00335 | 0.00127 | 0.00172 |
| 93 | 0.027 | | |
| 94 | 0.00241 | | |
| 95 | 0.0898 | | |
| 96 | 0.0892 | | |
| 97 | 0.0273 | | |
| 98 | 0.539 | | |
| 99 | 0.852 | | |
| 100 | 0.00498 | | |
| 101 | 0.377 | 0.317 | 0.305 |
| 102 | 0.0192 | | |
| 103 | 0.0194 | | |
| 104 | 0.014 | | |
| 105 | 0.00323 | | |
| 106 | 0.00267 | | |
| 107 | 0.044 | | |
| 108 | 0.0305 | 0.0413 | 0.0522 |
| 109 | 0.0157 | 0.00258 | 0.00489 |
| 110 | 0.00273 | 0.00163 | 0.00258 |
| 111 | 0.0268 | | |
| 112 | 0.00711 | | |
| 113 | 0.00474 | | |
| 114 | 0.0808 | | |
| 115 | 0.0328 | | |
| 116 | 0.0778 | | |
| 117 | 0.0177 | | |
| 118 | 0.0398 | | |
| 119 | 0.0311 | | |
| 120 | 0.0825 | | |
| 121 | 0.0173 | | |
| 122 | 0.00814 | | |
| 123 | 0.00375 | | |
| 124 | 0.0192 | | |
| 125 | 0.00798 | 0.0032 | 0.00511 |
| 126 | 0.00474 | | |
| 127 | 0.049 | | |
| 128 | 0.214 | | |
| 129 | 0.0508 | | |
| 130 | 0.0137 | 0.0015 | 0.0015 |
| 131 | 0.0469 | | |
| 132 | 0.0341 | | |
| 133 | 0.0159 | | |
| 134 | 0.0127 | | |
| 135 | 0.00829 | | |
| 136 | 0.0181 | | |
| 137 | 0.0573 | | |
| 138 | 0.0251 | | |
| 139 | 0.00686 | | |
| 140 | 0.0319 | | |
| 141 | 0.00377 | | |
| 142 | 0.0232 | | |
| 143 | 0.0958 | | |
| 144 | 0.0254 | | |
| 145 | 0.0126 | | |
| 146 | 0.00443 | | |
| 147 | 0.0309 | | |
| 148 | 0.0346 | | |
| 149 | 0.025 | | |
| 150 | 0.0745 | | |
| 151 | 0.166 | | |
| 152 | 0.00748 | | |
| 153 | 0.00912 | | |
| 154 | 0.0159 | | |
| 155 | 0.00227 | | |
| 156 | 0.0291 | | |
| 157 | 0.0227 | | |
| 158 | 0.317 | | |
| 159 | 0.0215 | | |
| 160 | 0.0593 | | |
| 161 | 0.0737 | | |
| 162 | 0.0281 | | |
| 163 | 0.171 | | |
| 164 | 0.00862 | | |
| 165 | 1.04 | | |
| 166 | 1.04 | | |
| 167 | 0.0115 | | |
| 168 | 0.0116 | | |
| 169 | 0.038 | | |
| 170 | 0.0178 | | |
| 171 | 0.0055 | | |
| 172 | 0.022 | | |
| 173 | 0.0164 | | |

### Determination of the Efficacy of Compounds to Reduce Osteoarthritis Pain

Members of the TrkA inhibitors described above were tested and found effective in reducing osteoarthritis pain. The compounds tested were assessed in an in vivo model well known to those skilled in the art, the rat model of mono-iodoacetic acid induced osteoarthritis pain. A general review of various models of pain can be found in Joshi and Honore, Expert Opinion in Drug Discovery (2004) 1, pp. 323-334, and in the book ‘Drug Discovery and Evaluation, 2^{nd} edition (H. Gerhard Vogel, editor; Springer-Verlag, New York, 2002; pp. 702-706).

### Activity in an Osteoarthritis Model

Pain behavior was assessed by measurement of hind limb grip force (GF) in adult osteoarthritic rats. Male Sprague Dawley rats, generally weighing 125-150 g, were injected in the unilateral knee join with a single intra-articular injection of sodium monoiodoacetate (MIA). Rats were tested at 21-28 days following MIA injection. A behavioral measure of activity-induced pain was carried out. Measurements of the peak hind limb grip force were conducted by recording the maximum compressive force (CFₘₐₓ), in grams of force, exerted on a hind limb strain gauge setup, in a commercially available grip force measurement system (Columbus Instruments, Columbus, Ohio).

During testing, each rat was gently restrained by grasping it around its rib cage and then allowed to grasp the wire mesh frame attached to the strain gauge. The experimenter then moved the animal in a rostral-to-caudal direction until the grip was broken. Each rat was sequentially tested twice at an approximately 2-3 minute interval to obtain a raw mean grip force (CFₘₐₓ). This raw mean grip force data was in turn converted to a maximum hindlimb cumulative compressive force (CFₘₐₓ), as the grams of force/kg of body weight, for each animal.

For evaluating the compound effects, the hind limb grip force testing in the MIA-treated rats was conducted generally 30-60 minutes after dosing with the test compound. A group of age-matched nave (not injected with MIA) animals was added as a comparator to the drug-dosed groups. The vehicle control response for each group of MIA-treated animals was defined as the 0% response (0% effect), whereas the nave control group was defined as the normal response and as 100% effect. The % effect=(Treatment CFₘₐₓ−Vehicle CFₘₐₓ)/Vehicle CFₘₐₓ]×100). Higher % effect numbers indicate increased relief from the pain in the model, with 100% indicating a return to the level of response seen in normal (non-osteoarthritic) animals. All experiments evaluating drug effects in this model were conducted in a randomized blinded fashion.

### Animals, Compounds, and Dosing.

Male Sprague Dawley rats (generally 250-300 g body weight at the time of testing) obtained from Charles River Laboratories (Wilmington, Mass.) were used for all experiments, unless indicated otherwise. The animals were housed in Association for Assessment and Accreditation of Laboratory Animal Care (AAALAC) approved facilities at Abbott Laboratories in a temperature-regulated environment under a controlled 12-hour light-dark cycle, with lights on at 6:00 a.m. Food and water were available ad libitum at all times except during testing. All testing was done following procedures outlined in protocols approved by Abbott Laboratories Institutional Animal Care and Use Committee.

The following table illustrates that compounds of the invention are effective in reducing osteoarthritis pain, with efficacy in the MIA model after oral dosing:

| | dose | |
|---|---|---|
| Compound Name | (mg/kg, | % |
| (Example number) | p.o.) | Effect¹ |
| 4-[3-amino-4-(tetrahydro-2H-pyran-4-yloxy)-1H- | 100 | 77%*** |
| indazol-6-yl]-2-(4-cyclohexylpiperazin-1-yl)-N- | | |
| methylbenzamide | | |
| (Example 29) | | |
| 4-[3-amino-4-(tetrahydro-2H-pyran-4-yloxy)-1H- | 30 | 73%*** |
| indazol-6-yl]-N-methyl-2-[4-(2,2,2- | | |
| trifluoroethyl)piperazin-1-yl]benzamide | | |
| (Example 108) | | |
| 4-[3-amino-4-(tetrahydro-2H-pyran-4-yloxy)-1H- | 30 | 89%*** |
| indazol-6-yl]-2-[4-(5-fluoropyrimidin-2- | | |
| yl)piperazin-1-yl]-N-methylbenzamide | | |
| (Example 113) | | |
| isopropyl 4-{2-(methylcarbamoyl)-5-[3-methyl-4- | 30 | 85%*** |
| (tetrahydro-2H-pyran-4-yloxy)-1H-indazol-6- | | |
| yl]phenyl}piperazine-1-carboxylate | | |
| (Example 125) | | |

¹Data represent mean percent effect. Statistical significance * p <0.05, ** p <0.01, ***p <0.001 as compared to vehicle-treated animals.
Abbreviations:
SEM = standard error of the mean;
p.o. = per os = oral dosed.

Representative compounds of the invention are active in this model, with preferred compounds of the invention active in the model at doses of ranging about 0.1 to 100 mg/kg of body weight.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

What is claimed is:

1. A compound of formula (I): 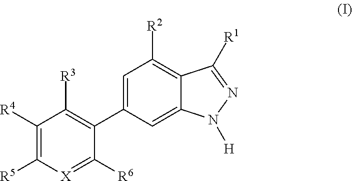
or a pharmaceutically acceptable salt, ester, amide, or radiolabelled form thereof, wherein:
R¹ is hydrogen, C₁-C₄-alkyl, C₁-C₄-fluoroalkyl, C₃-C₆-cycloalkyl or H₂N—;
R² is hydrogen or R⁷O—;
R³ and R⁶ are independently hydrogen or fluorine;
R⁴ is hydrogen, G¹-, G²-, or Y-L¹-(CR^{a}R^{b})_{f}-L²-;
R⁵ is hydrogen, R¹¹C(O)—, R¹⁰N(H)C(O)—, R¹¹C(O)NH—, R¹⁰N(H)SO₂—, R¹¹SO₂NH—, R¹¹CH(OH)—, R¹¹C(O)C(O)NH—, or NC—;
R⁷ is C₁-C₆-alkyl, C₁-C₆-fluoroalkyl, C₃-C₈-cycloalkyl, C₃-C₈-cycloalkyl-C₁-C₄-alkyl, C₄-C₈-cycloalkenyl, C₄-C₈-cycloalkenyl-C₁-C₄-alkyl, M₄-M₇-heterocycle or M₄-M₇-heterocycle-C₁-C₄alkyl, wherein:
the C₁-C₆-alkyl and C₁-C₆-fluoroalkyl are optionally substituted with 1, 2, 3, 4, or 5 substituents selected from halogen, cyano, oxo, O₂N—, R^{s}O—, R^{s}O—(CR^{a}R^{b})ₘ—, R^{s}C(O)O—, (R^{s})(R^{t})NC(O)O—, R^{s}S—, R^{s}S(O)—, R^{s}S(O)₂—, (R^{s})(R^{t})NS(O)₂—, R^{s}C(O)—, R^{s}OC(O)—, (R^{s})(R^{t})NC(O)—, (R^{s})(R^{t})N—, R^{t}C(O)N(R^{s})—, R^{t}OC(O)N(R^{s})—, and (R^{t})S(O)₂N(R^{s})—;
the C₃-C₈-cycloalkyl, C₃-C₈-cycloalkyl-C₁-C₄-alkyl, C₄-C₈-cycloalkenyl, C₄-C₈-cycloalkenyl-C₁-C₄-alkyl, M₄-M₇-heterocycle and M₄-M₇-heterocycle-C₁-C₄alkyl are optionally substituted with 1, 2, 3, 4, or 5 substituents selected from C₁-C₄-alkyl, C₂-C₄-alkenyl, C₂-C₄-alkynyl, halogen, cyano, oxo, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;
R¹⁰ is hydrogen, C₁-C₆-alkyl, or C₃-C₇-cycloalkyl;
R¹¹ is C₁-C₆-alkyl or C₂-C₆-alkenyl;
R^{a} and R^{b}, at each occurrence, are each independently hydrogen, C₁-C₄-alkyl, or C₁-C₄-haloalkyl;
R^{c}, at each occurrence, is independently hydrogen, C₁-C₄-alkyl, C₃-C₆-cycloalkyl or C₁-C₄-haloalkyl;
R^{s}, R^{t}, R^{u}, and R^{v} are, at each occurrence, independently hydrogen, C₁-C₆-alkyl, C₂-C₆-alkenyl, C₂-C₆-alkynyl or C₁-C₆-haloalkyl;
G¹ is monocyclic M₄-M₇-heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, G^{1a}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}-(CR^{a}R^{b})ₚ—C(O)—, G^{1a}-OC(O)—, G^{1a}-(CR^{a}R^{b})ₚ—OC(O)—, G^{1b}-, G^{1b}C(O)—, G^{1b}-(CR^{a}R^{b})ₚ—, G^{1b}C(O)—(CR^{a}R^{b})ₚ, G^{1b}-(CR^{a}R^{b})ₚ—OC(O)—, G^{1b}-(CR^{a}R^{b})ₚ—C(O)—, G^{1c}-, G^{1c}C(O)—, G^{1c}-(CR^{a}R^{b})ₕ—, G^{1c}C(O)—(CR^{a}R^{b})ₚ—, G^{1c}-(CR^{a}R^{b})ₚ—OC(O)—, G^{1c}-(CR^{a}R^{b})ₚ—C(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;
G^{1a} is aryl or heteroaryl wherein the aryl or heteroaryl are optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;
G^{1b} is C₃-C₈-cycloalkyl or C₄-C₈-cycloalkenyl, wherein the C₃-C₈-cycloalkyl or C₄-C₈-cycloalkenyl are optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;
G^{1c} is M₄-M₇-heterocycle M₇-heterocycle wherein the M₄-M₇-heterocycle is optionally substituted with 1, 2, 3, 4, or 5 substituents selected from C₁-C₄-alkyl, C₂-C₄-alkenyl, C₂-C₄-alkynyl, halogen, cyano, O₂N—, R^{u}O—, R^{u}O—(CR^{a}R^{b})ₘ—, R^{u}C(O)O—, (R^{u})(R^{v})NC(O)O—, R^{u}S—, R^{u}S(O)—, R^{u}S(O)₂—, (R^{u})(R^{v})NS(O)₂—, R^{u}C(O)—, R^{u}OC(O)—, (R^{u})(R^{v})NC(O)—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl;
G² is a fused-bicyclic heterocycle or spirocyclic heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₘ—, R^{u}O—(CR^{a}R^{b})ₙ—O—(CR^{a}R^{b})ₘ—, G^{1a}-, G^{1a}C(O)—, G^{1a}-(CR^{a}R^{b})_{q}—, G^{1b}-, G^{1b}C(O)—, G^{1b}-(CR^{a}R^{b})_{q}—, G^{1b}C(O)—(CR^{a}R^{b})_{q}—, (R^{u})(R^{v})N—, R^{v}C(O)N(R^{u})—, (R^{v})OC(O)N(R^{u})—, (R^{v})S(O)₂N(R^{a})—, and C₁-C₄-haloalkyl;
L¹ and L² are independently selected from a bond, —O—, —NR^{c}—, —C(O)—, —R^{c}NC(O)—, —C(O)NR^{c}—, —R^{c}NC(O)O—, —OC(O)NR^{c}—, —NR^{c}C(O)NR^{c}—, —S(O)—, —S(O)₂—, —R^{c}NS(O)₂—, and —S(O)₂NR^{c}—;
X is N, CH, or CF;
Y is monocyclic C₃-C₈-cycloalkyl, monocyclic C₃-C₈-cycloalkenyl, or monocyclic M₄-M₇-heterocycle unsubstituted or optionally substituted with 1, 2, 3, or 4 substituents selected from C₁-C₈-alkyl, C₂-C₈-alkenyl, C₂-C₈-alkynyl, halogen, cyano, oxo, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₚ—, (R^{u})(R^{v})N—, R^{v}C(O)N(R^{u})—, (R^{v})OC(O)N(R^{u})—, (R^{v})S(O)₂N(R^{a})—, and C₁-C₄-haloalkyl; or
Y is aryl or heteroaryl unsubstituted or optionally substituted with 1, 2, 3, or 4 substituents selected from C₁-C₈-alkyl, C₂-C₈-alkenyl, C₂-C₈-alkynyl, halogen, cyano, O₂N—, R^{u}S(O)—, R^{u}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₘ—, (R^{u})(R^{v})N—, R^{v}C(O)N(R^{u})—, (R^{v})OC(O)N(R^{u})—, (R^{v})S(O)₂N(R^{u})—, and C₁-C₄-haloalkyl; or
Y is C₁-C₆-alkyl or C₁-C₆-fluoroalkyl optionally substituted with 1, 2, 3, 4, or 5 substituents selected from halogen, cyano, oxo, O₂N—, R^{s}O—, R^{s}C(O)O—, (R^{s})(R^{t})NC(O)O—, R^{s}S—, R^{s}S(O)—, R^{s}S(O)₂—, (R^{s})(R^{t})NS(O)₂—, R^{s}C(O)—, R^{s}OC(O)—, (R^{s})(R^{t})NC(O)—, (R^{s})(R^{t})N—, R^{t}C(O)N(R^{s})—, (R^{t})OC(O)N(R^{s})—, and (R^{t})S(O)₂N(R^{s})—;
f is 1, 2, 3, or 4; or
f is 2, 3, or 4 when the moieties attaching to each side of —(CR^{a}R^{b})_{f}— are selected from O, NR^{c}, or a ring nitrogen atom of a monocyclic M₄-M₇-heterocycle when L¹ is a bond;
h, j, k and n are independently 2, 3, or 4; and
m, p and q are independently 1, 2, 3, or 4.

2. The compound of claim 1, wherein R¹ is H₂N—.

3. The compound of claim 2, wherein, R² is R⁷O—.

4. The compound of claim 3, wherein R⁴ is G¹- or G²-.

5. The compound of claim 4, wherein
R⁴ is G¹-;
R⁵ is hydrogen, R¹¹C(O)—, R¹⁰N(H)C(O)—, R¹¹C(O)NH—, R¹¹SO₂NH—, R¹⁰N(H)SO₂—, R¹¹CH(OH)—, or NC—;
R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₄-alkyl or R^{u}C(O)—;
G¹ is monocyclic M₄-M₇-heterocycle unsubstituted or optionally substituted with 1 or 2 substituents selected from C₁-C₈-alkyl, C₂-C₈-alkenyl, C₂-C₈-alkynyl, halogen, cyano, oxo, R^{u}S(O)₂—, G^{1a}S(O)₂—, (R^{u})(R^{v})NS(O)₂—, R^{u}C(O)—, R^{u}OC(O)—, (R^{u})(R^{v})NC(O)—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}(CR^{a}R^{b})ₚ—OC(O)—, G^{1b}-, G^{1b}C(O)—, G^{1b}-(CR^{a}R^{b})ₚ—, G^{1b}-(CR^{a}R^{b})ₚ—C(O)—, G^{1c}-, G^{1c}C(O)—, G^{1c}-(CR^{a}R^{b})ₕ—, G^{1c}C(O)—(CR^{a}R^{b})ₚ—, and C₁-C₄-haloalkyl;
G^{1a} is aryl or heteroaryl wherein the aryl or heteroaryl are optionally substituted with 1 substituents selected from C₁-C₄-alkyl, halogen, or R^{u}O—;
G^{1b} is C₃-C₈-cycloalkyl, wherein the C₃-C₈-cycloalkyl is optionally substituted with for 2 halogen; and
G^{1c} is M₄-M₇-heterocycle wherein the M₄-M₇-heterocycle is optionally substituted with 1 C₁-C₄-alkyl.

6. The compound of claim 4, wherein
R⁴ is G²-;
R⁵ is hydrogen, R¹¹C(O)—, R¹⁰N(H)C(O)—, R¹¹C(O)NH—, R¹¹SO₂NH—, R¹¹CH(OH)—, or NC—;
R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₄-alkyl or R^{u}C(O)—; and
G² is a fused-bicyclic heterocycle or spirocyclic heterocycle unsubstituted or optionally substituted with 1 substituent selected from C₁-C₈-alkyl, G^{1a}-(CR^{a}R^{b})_{q}—, and C₁-C₄-haloalkyl.

7. The compound of claim 4, wherein
R⁴ is hydrogen or Y-L¹-(CR^{a}R^{b})_{f}-L²-;
R⁵ is hydrogen, R¹¹C(O)—, R¹⁰N(H)C(O)—, R¹¹C(O)NH—, R¹¹SO₂NH—, R¹¹CH(OH)—, or NC—;
R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₄-alkyl or R^{u}C(O)—;
L¹ and L² are independently selected from —O—, —NR^{c}—, and —OC(O)NR^{c}—; and
Y is C₃-C₈-cycloalkyl, aryl, or C₁-C₆-alkyl.

8. The compound of claim 2, wherein, R² is hydrogen;
R⁴ is G¹-;
R⁵ is hydrogen, R¹¹C(O)—, R¹⁰N(H)C(O)—, R¹¹C(O)NH—, R¹¹SO₂NH—, R¹¹CH(OH)—, or NC—;
G¹ is monocyclic M₄-M₇-heterocycle unsubstituted or optionally substituted with 1 or 2 substituents selected from C₁-C₈-alkyl, C₂-C₈-alkenyl, C₂-C₈-alkynyl, halogen, cyano, oxo, R^{u}S(O)₂—, G^{1a}S(O)₂—, (R^{u})(R^{v})NS(O)₂—, R^{u}C(O)—, R^{u}OC(O)—, (R^{u})(R^{v})NC(O)—, R^{u}O—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}-(CR^{a}R^{b})ₚ—OC(O)—, G^{1b}-, G^{1b}C(O)—, G^{1b}-(CR^{a}R^{b})ₚ—, G^{1b}-(CR^{a}R^{b})ₚ—C(O)—, G^{1c}- , G^{1c}C(O)—, G^{1c}-(CR^{a}R^{b})ₕ—, G^{1c}C(O)—(CR^{a}R^{b})ₚ—, and C₁-C₄-haloalkyl;
G^{1a} is aryl or heteroaryl wherein the aryl or heteroaryl are optionally substituted with 1 substituents selected from C₁-C₄-alkyl, halogen, or R^{u}O—;
G^{1b} is C₃-C₈-cycloalkyl, wherein the C₃-C₈-cycloalkyl is optionally substituted with for 2 halogen; and
G^{1c} is M₄-M₇-heterocycle M₇-heterocycle wherein the M₄-M₇-heterocycle is optionally substituted with 1 C₁-C₄-alkyl.

9. The compound of claim 1, wherein R¹ is hydrogen, C₁-C₄-alkyl, C₁-C₄-fluoroalkyl, or C₃-C₆-cycloalkyl.

10. The compound of claim 9, wherein, R² is R⁷O—.

11. The compound of claim 10, wherein R⁴ is G¹- or G²-.

12. The compound of claim 11, wherein
R⁴ is G¹-;
R⁵ is hydrogen, R¹¹C(O), —R¹⁰N(H)C(O)—, R¹¹C(O)NH—, R¹¹SO₂NH—, R¹¹CH(OH)—, R¹¹C(O)C(O)NH—, or NC—;
R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₄-alkyl or R^{u}C(O)—;
G¹ is monocyclic M₄-M₇-heterocycle unsubstituted or optionally substituted with 1 or 2 substituents selected from C₁-C₈-alkyl, C₂-C₈-alkenyl, C₂-C₈-alkynyl, halogen, cyano, oxo, R^{u}S(O)₂—, G^{1a}S(O)₂—, (R^{u})(R^{v})NS(O)₂—, R^{u}C(O)—, R^{u}OC(O)—, (R^{u})(R^{v})NC(O)—, R^{u}O—(CR^{a}R^{b})ₕ—, R^{u}O—(CR^{a}R^{b})ₖ—O—(CR^{a}R^{b})ⱼ—, R^{u}O—(CR^{a}R^{b})ₖ—OC(O)—, G^{1a}-, G^{1a}C(O)—, G^{1a}-(CR^{a}R^{b})ₚ—, G^{1a}(CR^{a}R^{b})ₚ—OC(O)—, G^{1b}-, G^{1b}C(O)—, G^{1b}-(CR^{a}R^{b})ₚ—, G^{1b}-(CR^{a}R^{b})ₚ—C(O)— G^{1c}- , G^{1c}C(O)—, G^{1c}-(CR^{a}R^{b})ₕ—C(O)—(CR^{a}R^{b})ₚ—, and C₁-C₄-haloalkyl;
G^{1a} is aryl or heteroaryl wherein the aryl or heteroaryl are optionally substituted with 1 substituents selected from C₁-C₄-alkyl, halogen, or R^{u}O—;
G^{1b} is C₃-C₈-cycloalkyl, wherein the C₃-C₈-cycloalkyl is optionally substituted with 1 or 2 halogen; and
G^{1c} is M₄-M₇-heterocycle M₇-heterocycle wherein the M₄-M₇-heterocycle is optionally substituted with 1 C₁-C₄-alkyl.

13. The compound of claim 11, wherein
R⁴ is G²-;
R⁵ is hydrogen, R¹¹C(O)—, R¹⁰N(H)C(O)—, R¹¹C(O)NH—, R¹¹SO₂NH—, R¹¹CH(OH)—, or NC—;
R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₄-alkyl or R^{u}C(O)—; and
G² is a fused-bicyclic heterocycle or spirocyclic heterocycle unsubstituted or optionally substituted with 1 substituent selected from C₁-C₈-alkyl, G^{1a}-(CR^{a}R^{b})_{q}—, and C₁-C₄-haloalkyl.

14. The compound of claim 11, wherein
R⁴ is hydrogen or Y-L¹-(CR^{a}R^{b})_{f}-L²-;
R⁵ is hydrogen, R¹¹C(O)—, R¹⁰N(H)C(O)—, R¹¹C(O)NH—, R¹¹SO₂NH—, R¹¹CH(OH)—, or NC—;
R⁷ is C₁-C₆-alkyl, C₁-C₆-fluoroalkyl, C₃-C₈-cycloalkyl, or M₄-M₇-heterocycle, wherein the M₄-M₇-heterocycle is optionally substituted with C₁-C₄-alkyl or R^{u}C(O)—;
L¹ and L² are independently selected from —O—, —NR^{c}—, and —OC(O)NR^{c}—; and
Y is C₃-C₈-cycloalkyl, aryl, or C₁-C₆-alkyl.

15. The compound of claim 1, selected from:
4-[3-amino-4-(tetrahydro-2H-pyran-4-yloxy)-1H-indazol-6-yl]-2-(4-phenylpiperazin-1-yl)benzamide;
4-[3-amino-4-(tetrahydro-2H-pyran-4-yloxy)-1H-indazol-6-yl]-2-[4-(pyridin-3-yl)piperazin-1-yl]benzamide;
4-[3-amino-4-(tetrahydro-2H-pyran-4-yloxy)-1H-indazol-6-yl]-2-(4-cyclopropylpiperazin-1-yl)benzamide;
2-(4-acetylpiperazin-1-yl)-4-[3-amino-4-(tetrahydro-2H-pyran-4-yloxy)-1H-indazol-6-yl]benzamide;
4-[3-amino-4-(tetrahydro-2H-pyran-4-yloxy)-1H-indazol-6-yl]-2-{4-[2-(morpholin-4-yl)ethyl]piperazin-1-yl}benzamide;
4-[3-amino-4-(tetrahydro-2H-pyran-4-yloxy)-1H-indazol-6-yl]-2-[4-(5-methoxypyrimidin-4-yl)piperazin-1-yl]benzamide;
4-[3-amino-4-(tetrahydro-2H-pyran-4-yloxy)-1H-indazol-6-yl]-2-[4-(3-hydroxypropyl)piperazin-1-yl]benzamide;
4-[3-amino-4-(tetrahydro-2H-pyran-4-yloxy)-1H-indazol-6-yl]-2-[4-(5-chloropyridin-3-yl)piperazin-1-yl]benzamide;
4-[3-amino-4-(tetrahydro-2H-pyran-4-yloxy)-1H-indazol-6-yl]-2-[4-(1-methylpiperidin-4-yl)piperazin-1-yl]benzamide;
4-[3-amino-4-(tetrahydro-2H-pyran-4-yloxy)-1H-indazol-6-yl]-2-[4-[2-(pyridin-2-yl)ethyl]piperazin-1-yl]benzamide;
4-[3-amino-4-(tetrahydro-2H-pyran-4-yloxy)-1H-indazol-6-yl]-2-[4-[2-(2-hydroxyethoxy)ethyl]piperazin-1-yl]benzamide;
4-[3-amino-4-(tetrahydro-2H-pyran-4-yloxy)-1H-indazol-6-yl]-2-[4-(3-chlorophenyl)piperazin-1-yl]benzamide;
1-{4-[3-amino-4-(tetrahydro-2H-pyran-4-yloxy)-1H-indazol-6-yl]-2-(4-phenylpiperazin-1-yl)phenyl}ethanone;
1-{4-[3-amino-4-(tetrahydro-2H-pyran-4-yloxy)-1H-indazol-6-yl]-2-(4-cyclopropylpiperazin-1-yl)phenyl}ethanone;
1-{4-[3-amino-4-(tetrahydro-2H-pyran-4-yloxy)-1H-indazol-6-yl]-2-[4-(pyrimidin-2-yl)piperazin-1-yl]phenyl}ethanone;
1-{4-[3-amino-4-(tetrahydro-2H-pyran-4-yloxy)-1H-indazol-6-yl]-2-[4-(pyridin-3-yl)piperazin-1-yl]phenyl}ethanone;
1-{4-[3-amino-4-(tetrahydro-2H-pyran-4-yloxy)-1H-indazol-6-yl]-2-[4-(4-fluorophenyl)piperazin-1-yl]phenyl}ethanone;
1-{4-[3-amino-4-(tetrahydro-2H-pyran-4-yloxy)-1H-indazol-6-yl]-2-[4-(3,3-dimethylbutyl)piperazin-1-yl]phenyl}ethanone;
1-{4-[3-amino-4-(tetrahydro-2H-pyran-4-yloxy)-1H-indazol-6-yl]-2-(4-cyclohexylpiperazin-1-yl)phenyl}ethanone;
1-{4-[3-amino-4-(tetrahydro-2H-pyran-4-yloxy)-1H-indazol-6-yl]-2-[4-(cyclopropylcarbonyl)piperazin-1-yl]phenyl}ethanone;
1-{4-[3-amino-4-(tetrahydro-2H-pyran-4-yloxy)-1H-indazol-6-yl]-2-[4-(1,3-thiazol-2-yl)piperazin-1-yl]phenyl}ethanone;
1-{4-[3-amino-4-(tetrahydro-2H-pyran-4-yloxy)-1H-indazol-6-yl]-2-[4-(pyridin-3-yl)piperazin-1-yl]phenyl}ethanol;
4-[3-amino-4-(tetrahydro-2H-pyran-4-yloxy)-1H-indazol-6-yl]-N-cyclopropyl-2-(4-phenylpiperazin-1-yl)benzamide;
4-[3-amino-4-(tetrahydro-2H-pyran-4-yloxy)-1H-indazol-6-yl]-N-cyclopropyl-2-[4-(pyridin-3-yl)piperazin-1-yl]benzamide;
4-[3-amino-4-(tetrahydro-2H-pyran-4-yloxy)-1H-indazol-6-yl]-N-cyclopropyl-2-[4-(2,2-dimethylpropanoyl)piperazin-1-yl]benzamide;
4-{3-amino-4-[(1,1,1-trifluoropropan-2-yl)oxy]-1H-indazol-6-yl}-N-cyclopropyl-2-(4-phenylpiperazin-1-yl)benzamide;
4-{3-amino-4-[(1,1,1-trifluoropropan-2-yl)oxy]-1H-indazol-6-yl}-N-cyclopropyl-2-(4-cyclopropylpiperazin-1-yl)benzamide;
4-{3-amino-4-[(1,1,1-trifluoropropan-2-yl)oxy]-1H-indazol-6-yl}-N-cyclopropyl-2-[4-(pyridin-3-yl)piperazin-1-yl]benzamide;
4-[3-amino-4-(tetrahydro-2H-pyran-4-yloxy)-1H-indazol-6-yl]-2-(4-cyclohexylpiperazin-1-yl)-N-methylbenzamide;
4-[3-amino-4-(tetrahydro-2H-pyran-4-yloxy)-1H-indazol-6-yl]-N-methyl-2-(4-phenylpiperazin-1-yl)benzamide;
4-[3-amino-4-(tetrahydro-2H-pyran-4-yloxy)-1H-indazol-6-yl]-N-methyl-2-[4-(pyridin-3-yl)piperazin-1-yl]benzamide;
4-[3-amino-4-(tetrahydro-2H-pyran-4-yloxy)-1H-indazol-6-yl]-2-[4-(cyclopropylcarbonyl)piperazin-1-yl]-N-methylbenzamide;
4-{3-amino-4-[(1,1,1-trifluoropropan-2-yl)oxy]-1H-indazol-6-yl}-N-methyl-2-(4-phenylpiperazin-1-yl)benzamide;
4-{3-amino-4-[(1,1,1-trifluoropropan-2-yl)oxy]-1H-indazol-6-yl}-2-(4-cyclopropylpiperazin-1-yl)-N-methylbenzamide;
4-{3-amino-4-[(1,1,1-trifluoropropan-2-yl)oxy]-1H-indazol-6-yl}-2-(4-cyclohexylpiperazin-1-yl)-N-methylbenzamide;
4-{3-amino-4-[(1,1,1-trifluoropropan-2-yl)oxy]-1H-indazol-6-yl}-N-methyl-2-[4-(pyridin-3-yl)piperazin-1-yl]benzamide;
4-{3-amino-4-[(1,1,1-trifluoropropan-2-yl)oxy]-1H-indazol-6-yl}-N-methyl-2-{4-[2-(morpholin-4-yl)ethyl]piperazin-1-yl}benzamide;
4-{3-amino-4-[(1,1,1-trifluoropropan-2-yl)oxy]-1H-indazol-6-yl}-N-methyl-2-[4-(pyrazin-2-yl)piperazin-1-yl]benzamide;
4-(3-amino-4-{[(2S)-1,1,1-trifluoropropan-2-yl]oxy}-1H-indazol-6-yl)-N-methyl-2-(4-phenylpiperazin-1-yl)benzamide;
4-(3-amino-4-{[(2S)-1,1,1-trifluoropropan-2-yl]oxy}-1H-indazol-6-yl)-2-[4-(3,3-dimethylbutyl)piperazin-1-yl]-N-methylbenzamide;
4-(3-amino-4-{[(2S)-1,1,1-trifluoropropan-2-yl]oxy}-1H-indazol-6-yl)-N-methyl-2-[4-(pyridin-3-yl)piperazin-1-yl]benzamide;
4-(3-amino-4-{[(2S)-1,1,1-trifluoropropan-2-yl]oxy}-1H-indazol-6-yl)-2-(4-cyclobutylpiperazin-1-yl)-N-methylbenzamide;
4-(3-amino-4-{[(2S)-1,1,1-trifluoropropan-2-yl]oxy}-1H-indazol-6-yl)-N-methyl-2-[4-(pyrimidin-2-yl)piperazin-1-yl]benzamide;
4-(3-amino-4-{[(2S)-1,1,1-trifluoropropan-2-yl]oxy}-1H-indazol-6-yl)-2-(4-cyclopentylpiperazin-1-yl)-N-methylbenzamide;
4-(3-amino-4-{[(2S)-1,1,1-trifluoropropan-2-yl]oxy}-1H-indazol-6-yl)-2-[4-(2,2-dimethylpropanoyl)piperazin-1-yl]-N-methylbenzamide;
4-(3-amino-4-{[(2S)-1,1,1-trifluoropropan-2-yl]oxy}-1H-indazol-6-yl)-N-methyl-2-[4-(pyrazin-2-yl)piperazin-1-yl]benzamide;
4-(3-amino-4-{[(2S)-1,1,1-trifluoropropan-2-yl]oxy}-1H-indazol-6-yl)-2-(4-cyclohexylpiperazin-1-yl)-N-methylbenzamide;
4-(3-amino-4-{[(2R)-1,1,1-trifluoropropan-2-yl]oxy}-1H-indazol-6-yl)-N-methyl-2-(4-phenylpiperazin-1-yl)benzamide;
4-(3-amino-4-{[(2R)-1,1,1-trifluoropropan-2-yl]oxy}-1H-indazol-6-yl)-N-methyl-2-[4-(pyridin-3-yl)piperazin-1-yl]benzamide;
4-(3-amino-4-{[(2R)-1,1,1-trifluoropropan-2-yl]oxy}-1H-indazol-6-yl)-2-(4-cyclobutylpiperazin-1-yl)-N-methylbenzamide;
4-(3-amino-4-{[(2R)-1,1,1-trifluoropropan-2-yl]oxy}-1H-indazol-6-yl)-2-[4-(3,3-dimethylbutyl)piperazin-1-yl]-N-methylbenzamide;
4-(3-amino-4-{[(2R)-1,1,1-trifluoropropan-2-yl]oxy}-1H-indazol-6-yl)-N-methyl-2-[4-(pyrimidin-2-yl)piperazin-1-yl]benzamide;
4-(3-amino-4-{[(2R)-1,1,1-trifluoropropan-2-yl]oxy}-1H-indazol-6-yl)-2-(4-cyclopentylpiperazin-1-yl)-N-methylbenzamide;
4-(3-amino-4-{[(2R)-1,1,1-trifluoropropan-2-yl]oxy}-1H-indazol-6-yl)-2-[4-(2,2-dimethylpropanoyl)piperazin-1-yl]-N-methylbenzamide;
4-(3-amino-4-{[(2R)-1,1,1-trifluoropropan-2-yl]oxy}-1H-indazol-6-yl)-N-methyl-2-[4-(pyrazin-2-yl)piperazin-1-yl]benzamide;
4-(3-amino-4-{[(2R)-1,1,1-trifluoropropan-2-yl]oxy}-1H-indazol-6-yl)-2-(4-cyclohexylpiperazin-1-yl)-N-methylbenzamide;
4-(3-amino-4-{[(2R)-1,1,1-trifluoropropan-2-yl]oxy}-1H-indazol-6-yl)-N-methyl-2-(octahydro-2H-pyrido[1,2-a]pyrazin-2-yl)benzamide;
4-(3-amino-4-{[(2R)-1,1,1-trifluoropropan-2-yl]oxy}-1H-indazol-6-yl)-2-(2-isobutylmorpholin-4-yl)-N-methylbenzamide;
4-(3-amino-4-{[(2R)-1,1,1-trifluoropropan-2-yl]oxy}-1H-indazol-6-yl)-N-methyl-2-[4-(morpholin-4-yl)piperidin-1-yl]benzamide;
4-(3-amino-4-{[(2R)-1,1,1-trifluoropropan-2-yl]oxy}-1H-indazol-6-yl)-2-(4-hydroxy-4-phenylpiperidin-1-yl)-N-methylbenzamide;
4-(3-amino-4-{[(2R)-1,1,1-trifluoropropan-2-yl]oxy}-1H-indazol-6-yl)-2-(4-cyclohexylpiperidin-1-yl)-N-methylbenzamide;
4-{3-amino-4-[(1-methylpiperidin-4-yl)oxy]-1H-indazol-6-yl}-N-cyclopropyl-2-(4-phenylpiperazin-1-yl)benzamide;
4-[3-amino-4-(piperidin-4-yloxy)-1H-indazol-6-yl]-2-(4-phenylpiperazin-1-yl)benzamide;
4-{4-[(1-acetylpiperidin-4-yl)oxy]-3-amino-1H-indazol-6-yl}-2-(4-phenylpiperazin-1-yl)benzamide;
4-{3-amino-4-[(3S)-tetrahydrofuran-3-yloxy]-1H-indazol-6-yl}-2-(4-cyclopropylpiperazin-1-yl)benzamide;
4-{3-amino-4-[(3S)-tetrahydrofuran-3-yloxy]-1H-indazol-6-yl}-2-(4-phenylpiperazin-1-yl)benzamide;
4-{3-amino-4-[(3S)-tetrahydrofuran-3-yloxy]-1H-indazol-6-yl}-2-[4-(pyridin-3-yl)piperazin-1-yl]benzamide;
4-{3-amino-4-[(3R)-tetrahydrofuran-3-yloxy]-1H-indazol-6-yl}-2-[4-(pyridin-3-yl)piperazin-1-yl]benzamide;
4-[3-amino-4-(cyclopentyloxy)-1H-indazol-6-yl]-2-[4-(pyridin-3-yl)piperazin-1-yl]benzamide;
4-[3-amino-4-(cyclopentyloxy)-1H-indazol-6-yl]-2-{4-[2-(morpholin-4-yl)ethyl]piperazin-1-yl}benzamide;
4-[3-amino-4-(cyclopentyloxy)-1H-indazol-6-yl]-2-(4-phenylpiperazin-1-yl)benzamide;
4-[3-amino-4-(cyclopentyloxy)-1H-indazol-6-yl]-2-[4-(4-fluorophenyl)piperazin-1-yl]benzamide;
tert-butyl 4-{5-[3-amino-4-(cyclopentyloxy)-1H-indazol-6-yl]-2-carbamoylphenyl}piperazine-1-carboxylate;
4-[3-amino-4-(cyclopentyloxy)-1H-indazol-6-yl]-2-(4-cyclohexylpiperazin-1-yl)benzamide;
4-[3-amino-4-(cyclopentyloxy)-1H-indazol-6-yl]-2-[4-(pyridin-2-yl)piperazin-1-yl]benzamide;
4-[3-amino-4-(cyclopentyloxy)-1H-indazol-6-yl]-2-(4-phenylpiperidin-1-yl)benzamide;
2-(4-acetylpiperazin-1-yl)-4-[3-amino-4-(cyclopentyloxy)-1H-indazol-6-yl]benzamide;
4-[3-amino-4-(cyclopentyloxy)-1H-indazol-6-yl]-2-(4-cyclopropylpiperazin-1-yl)benzamide;
1-{5-[3-amino-4-(cyclopentyloxy)-1H-indazol-6-yl]-2-carbamoylphenyl}piperidine-4-carboxamide;
4-[3-amino-4-(cyclopentyloxy)-1H-indazol-6-yl]-2-(4-benzoylpiperazin-1-yl)benzamide;
4-[3-amino-4-(cyclopentyloxy)-1H-indazol-6-yl]-2-[4-(cyclopropylcarbonyl)piperazin-1-yl]benzamide;
4-[3-amino-4-(cyclopentyloxy)-1H-indazol-6-yl]-2-[4-(morpholin-4-ylcarbonyl)piperazin-1-yl]benzamide;
4-(3-amino-1H-indazol-6-yl)-2-{4-[2-oxo-2-(pyrrolidin-1-yl)ethyl]piperazin-1-yl}benzamide;
4-(3-amino-1H-indazol-6-yl)-2-(4-phenylpiperazin-1-yl)benzamide;
4-(3-amino-1H-indazol-6-yl)-2-(4-cyclohexylpiperazin-1-yl)benzamide;
4-(3-amino-1H-indazol-6-yl)-2-[4-(pyridin-2-yl)piperazin-1-yl]benzamide;
4-(3-amino-1H-indazol-6-yl)-2-(4-benzylpiperazin-1-yl)benzamide;
4-(3-amino-1H-indazol-6-yl)-2-(4-cyclopropylpiperazin-1-yl)benzamide;
4-(3-amino-1H-indazol-6-yl)-2-{4-[2-(morpholin-4-yl)ethyl]piperazin-1-yl}benzamide;
4-(3-amino-1H-indazol-6-yl)-2-[4-(pyridin-3-yl)piperazin-1-yl]benzamide;
4-{3-amino-4-[(1,1,1-trifluoropropan-2-yl)oxy]-1H-indazol-6-yl}-2-[4-(pyridin-3-yl)piperazin-1-yl]benzamide;
4-{3-amino-4-[(1,1,1-trifluoropropan-2-yl)oxy]-1H-indazol-6-yl}-2-(4-phenylpiperazin-1-yl)benzamide;
4-{3-amino-4-[(1,1,1-trifluoropropan-2-yl)oxy]-1H-indazol-6-yl}-2-(4-cyclohexylpiperazin-1-yl)benzamide;
4-{3-amino-4-[(1,1,1-trifluoropropan-2-yl)oxy]-1H-indazol-6-yl}-2-[4-(pyridin-2-yl)piperazin-1-yl]benzamide;
4-{3-amino-4-[(1,1,1-trifluoropropan-2-yl)oxy]-1H-indazol-6-yl}-2-{4-[2-(morpholin-4-yl)ethyl]piperazin-1-yl}benzamide;
2-(4-acetylpiperazin-1-yl)-4-{3-amino-4-[(1,1,1-trifluoropropan-2-yl)oxy]-1H-indazol-6-yl}benzamide;
4-{3-amino-4-[(1,1,1-trifluoropropan-2-yl)oxy]-1H-indazol-6-yl}-2-(4-cyclopropylpiperazin-1-yl)benzamide;
4-{3-amino-4-[(1,1,1-trifluoropropan-2-yl)oxy]-1H-indazol-6-yl}-2-[4-(pyridin-4-yl)piperazin-1-yl]benzamide;
4-{3-amino-4-[(1,1,1-trifluoropropan-2-yl)oxy]-1H-indazol-6-yl}-2-[(3 aR*,6aS*)-5-benzylhexahydropyrrolo[3,4-c]pyrrol-2(1H)-yl]benzamide;
4-{3-amino-4-[(1,1,1-trifluoropropan-2-yl)oxy]-1H-indazol-6-yl}-2-[4-(3,3-dimethylbutyl)piperazin-1-yl]benzamide;
4-{3-amino-4-[(1,1,1-trifluoropropan-2-yl)oxy]-1H-indazol-6-yl}-2-(4-isopropylpiperazin-1-yl)benzamide;
4-{3-amino-4-[(1,1,1-trifluoropropan-2-yl)oxy]-1H-indazol-6-yl}-2-(4-cyclopentylpiperazin-1-yl)benzamide;
4-{3-amino-4-[(1,1,1-trifluoropropan-2-yl)oxy]-1H-indazol-6-yl}-2-[4-(cyclopropylcarbonyl)piperazin-1-yl]benzamide;
4-{3-amino-4-[(1,1,1-trifluoropropan-2-yl)oxy]-1H-indazol-6-yl}-2-[4-(2,2-dimethylpropanoyl)piperazin-1-yl]benzamide;
4-(3-amino-4-{[(2R)-1,1,1-trifluoropropan-2-yl]oxy}-1H-indazol-6-yl)-2-[2-(cyclohexyloxy)ethoxy]-N-methylbenzamide;
4-(3-amino-4-{[(2R)-1,1,1-trifluoropropan-2-yl]oxy}-1H-indazol-6-yl)-N-methyl-2-(2-phenoxyethoxy)benzamide;
4-[3-amino-4-(cyclopentyloxy)-1H-indazol-6-yl]-2-{4-[2-oxo-2-(piperidin-1-yl)ethyl]piperazin-1-yl}benzamide;
4-[3-amino-4-(tetrahydro-2H-pyran-4-yloxy)-1H-indazol-6-yl]-N-methyl-2-[4-(2,2,2-trifluoroethyl)piperazin-1-yl]benzamide;
4-(3-amino-4-isopropoxy-1H-indazol-6-yl)-2-(4-cyclohexylpiperazin-1-yl)-N-methylbenzamide;
4-[3-amino-4-(tetrahydro-2H-pyran-4-yloxy)-1H-indazol-6-yl]-2-[4-(cyclopentylmethyl)piperazin-1-yl]-N-methylbenzamide;
tert-butyl 4-{5-[3-amino-4-(tetrahydro-2H-pyran-4-yloxy)-1H-indazol-6-yl]-2-(methylcarbamoyl)phenyl}piperazine-1-carboxylate;
4-[3-amino-4-(tetrahydro-2H-pyran-4-yloxy)-1H-indazol-6-yl]-N-methyl-2-(piperazin-1-yl)benzamide;
4-[3-amino-4-(tetrahydro-2H-pyran-4-yloxy)-1H-indazol-6-yl]-2-[4-(5-fluoropyrimidin-2-yl)piperazin-1-yl]-N-methylbenzamide;
isopropyl 4-{5-[3-amino-4-(tetrahydro-2H-pyran-4-yloxy)-1H-indazol-6-yl]-2-(methylcarbamoyl)phenyl}piperazine-1-carboxylate;
N-ethyl-4-[3-methyl-4-(tetrahydro-2H-pyran-4-yloxy)-1H-indazol-6-yl]-2-(4-phenylpiperazin-1-yl)benzamide;
2-(4-cyclohexylpiperazin-1-yl)-N-methyl-4-[4-(tetrahydro-2H-pyran-4-yloxy)-1H-indazol-6-yl]benzamide;
N-cyclopropyl-4-[3-methyl-4-(tetrahydro-2H-pyran-4-yloxy)-1H-indazol-6-yl]-2-(4-phenylpiperazin-1-yl)benzamide;
N-cyclobutyl-2-(4-cyclohexylpiperazin-1-yl)-4-[3-methyl-4-(tetrahydro-2H-pyran-4-yloxy)-1H-indazol-6-yl]benzamide;
N-{2-(4-cyclohexylpiperazin-1-yl)-4-[3-methyl-4-(tetrahydro-2H-pyran-4-yloxy)-1H-indazol-6-yl]phenyl}acetamide;
4-[4-(cyclopentyloxy)-1H-indazol-6-yl]-N-ethyl-2-(4-phenylpiperazin-1-yl)benzamide;
2-(4-cyclohexylpiperazin-1-yl)-4-[4-(cyclopentyloxy)-3-methyl-1H-indazol-6-yl]-N-methylbenzamide;
tert-butyl 4-{2-(methylcarbamoyl)-5-[3-methyl-4-(tetrahydro-2H-pyran-4-yloxy)-1H-indazol-6-yl]phenyl}piperazine-1-carboxylate;
N-methyl-4-[3-methyl-4-(tetrahydro-2H-pyran-4-yloxy)-1H-indazol-6-yl]-2-[4-(pyridin-3-ylmethyl)piperazin-1-yl]benzamide;
2-[4-(cyclopropylcarbonyl)piperazin-1-yl]-N-methyl-4-[3-methyl-4-(tetrahydro-2H-pyran-4-yloxy)-1H-indazol-6-yl]benzamide;
isopropyl 4-{2-(methylcarbamoyl)-5-[3-methyl-4-(tetrahydro-2H-pyran-4-yloxy)-1H-indazol-6-yl]phenyl}piperazine-1-carboxylate;
2-[2-(cyclohexyloxy)ethoxy]-N-methyl-4-[3-methyl-4-(tetrahydro-2H-pyran-4-yloxy)-1H-indazol-6-yl]benzamide;
N-{4-[4-(cyclopentyloxy)-3-methyl-1H-indazol-6-yl]-2-[(2-phenoxyethyl)amino]phenyl}acetamide;
isobutyl 4-{5-[4-(cyclopentyloxy)-3-methyl-1H-indazol-6-yl]-2-(methylcarbamoyl)phenyl}piperazine-1-carboxylate;
benzyl 4-{5-[4-(cyclopentyloxy)-3-methyl-1H-indazol-6-yl]-2-(methylcarbamoyl)phenyl}piperazine-1-carboxylate;
N-{4-[3-methyl-4-(tetrahydro-2H-pyran-4-yloxy)-1H-indazol-6-yl]-2-(2-phenoxyethoxy)phenyl}acetamide;
N-{2-(4-cyclohexylpiperazin-1-yl)-4-[3-methyl-4-(tetrahydro-2H-pyran-4-yloxy)-1H-indazol-6-yl]phenyl}acrylamide;
4-[3-methyl-4-(tetrahydro-2H-pyran-4-yloxy)-1H-indazol-6-yl]benzamide;
N-{2-(4-cyclohexylpiperazin-1-yl)-4-[3-methyl-4-(tetrahydro-2H-pyran-4-yloxy)-1H-indazol-6-yl]phenyl}-2-oxopropanamide;
ethyl 4-{2-(methylcarbamoyl)-5-[3-methyl-4-(tetrahydro-2H-pyran-4-yloxy)-1H-indazol-6-yl]phenyl}piperazine-1-carboxylate;
propyl 4-{2-(methylcarbamoyl)-5-[3-methyl-4-(tetrahydro-2H-pyran-4-yloxy)-1H-indazol-6-yl]phenyl}piperazine-1-carboxylate;
2,2,2-trifluoroethyl 4-{2-(methylcarbamoyl)-5-[3-methyl-4-(tetrahydro-2H-pyran-4-yloxy)-1H-indazol-6-yl]phenyl}piperazine-1-carboxylate;
2,2-dimethylpropyl 4-{2-(methylcarbamoyl)-5-[3-methyl-4-(tetrahydro-2H-pyran-4-yloxy)-1H-indazol-6-yl]phenyl}piperazine-1-carboxylate;
2-methoxyethyl 4-{2-(methylcarbamoyl)-5-[3-methyl-4-(tetrahydro-2H-pyran-4-yloxy)-1H-indazol-6-yl]phenyl}piperazine-1-carboxylate;
tert-butyl 4-{2-cyano-5-[3-methyl-4-(tetrahydro-2H-pyran-4-yloxy)-1H-indazol-6-yl]phenyl}piperazine-1-carboxylate;
tert-butyl 4-{2-carbamoyl-5-[3-methyl-4-(tetrahydro-2H-pyran-4-yloxy)-1H-indazol-6-yl]phenyl}piperazine-1-carboxylate;
tert-butyl 4-{2-acetamido-5-[3-methyl-4-(tetrahydro-2H-pyran-4-yloxy)-1H-indazol-6-yl]phenyl}piperazine-1-carboxylate;
isopropyl 4-{2-carbamoyl-5-[3-methyl-4-(tetrahydro-2H-pyran-4-yloxy)-1H-indazol-6-yl]phenyl}piperazine-1-carboxylate;
N-methyl-4-[3-methyl-4-(tetrahydro-2H-pyran-4-yloxy)-1H-indazol-6-yl]-2-[4-(2,2,2-trifluoroethyl)piperazin-1-yl]benzamide;
N-{2-[4-(cyclobutylcarbonyl)piperazin-1-yl]-4-[3-methyl-4-(tetrahydro-2H-pyran-4-yloxy)-1H-indazol-6-yl]phenyl}acetamide;
N-{2-(4-benzylpiperazin-1-yl)-4-[3-methyl-4-(tetrahydro-2H-pyran-4-yloxy)-1H-indazol-6-yl]phenyl}acetamide;
isopropyl 4-{2-acetamido-5-[3-methyl-4-(tetrahydro-2H-pyran-4-yloxy)-1H-indazol-6-yl]phenyl}piperazine-1-carboxylate;
isopropyl (2-{2-acetamido-5-[3-methyl-4-(tetrahydro-2H-pyran-4-yloxy)-1H-indazol-6-yl]phenoxy}ethyl)carbamate;
N-{4-[3-methyl-4-(tetrahydro-2H-pyran-4-yloxy)-1H-indazol-6-yl]-2-[4-(pyridin-3-ylmethyl)piperazin-1-yl]phenyl}acetamide;
N-{2-[4-(3-fluorobenzyl)piperazin-1-yl]-4-[3-methyl-4-(tetrahydro-2H-pyran-4-yloxy)-1H-indazol-6-yl]phenyl}acetamide;
N-(2-{4-[(5-chloro-2-furyl)methyl]piperazin-1-yl}-4-[3-methyl-4-(tetrahydro-2H-pyran-4-yloxy)-1H-indazol-6-yl]phenyl)acetamide;
4-{2-acetamido-5-[3-methyl-4-(tetrahydro-2H-pyran-4-yloxy)-1H-indazol-6-yl]phenyl}-N-isopropylpiperazine-1-carboxamide;
N-methyl-2-{4-[(4-methylphenyl)sulfonyl]piperazin-1-yl}-4-[3-methyl-4-(tetrahydro-2H-pyran-4-yloxy)-1H-indazol-6-yl]benzamide;
methyl 4-{2-(methylcarbamoyl)-5-[3-methyl-4-(tetrahydro-2H-pyran-4-yloxy)-1H-indazol-6-yl]phenyl}piperazine-1-carboxylate;
2-[4-(3-fluorobenzyl)piperazin-1-yl]-N-methyl-4-[3-methyl-4-(tetrahydro-2H-pyran-4-yloxy)-1H-indazol-6-yl]benzamide;
N-isopropyl-4-{2-(methylcarbamoyl)-5-[3-methyl-4-(tetrahydro-2H-pyran-4-yloxy)-1H-indazol-6-yl]phenyl}piperazine-1-carboxamide;
4-[3-methyl-4-(tetrahydro-2H-pyran-4-yloxy)-1H-indazol-6-yl]-2-[4-(2,2,2-trifluoroethyl)piperazin-1-yl]benzamide;
4-[3-methyl-4-(tetrahydro-2H-pyran-4-yloxy)-1H-indazol-6-yl]-2-[4-(2,2,2-trifluoroethyl)piperazin-1-yl]benzonitrile;
tert-butyl 4-{5-[3-ethyl-4-(tetrahydro-2H-pyran-4-yloxy)-1H-indazol-6-yl]-2-(methylcarbamoyl)phenyl}piperazine-1-carboxylate;
2-(4-benzyl-3-oxopiperazin-1-yl)-N-methyl-4-[3-methyl-4-(tetrahydro-2H-pyran-4-yloxy)-1H-indazol-6-yl]benzamide;
N-methyl-2-[4-(3-methylbutanoyl)piperazin-1-yl]-4-[3-methyl-4-(tetrahydro-2H-pyran-4-yloxy)-1H-indazol-6-yl]benzamide;
4-(cyclopentyloxy)-3-methyl-6-[5-(4-phenylpiperazin-1-yl)pyridin-3-yl]-1H-indazole;
tert-butyl 4-{2-[(methylsulfonyl)amino]-5-[3-methyl-4-(tetrahydro-2H-pyran-4-yloxy)-1H-indazol-6-yl]phenyl}piperazine-1-carboxylate;
1,1,1-trifluoropropan-2-yl 4-{2-(methylcarbamoyl)-5-[3-methyl-4-(tetrahydro-2H-pyran-4-yloxy)-1H-indazol-6-yl]phenyl}piperazine-1-carboxylate;
2-[4-(cyclopropylacetyl)piperazin-1-yl]-N-methyl-4-[3-methyl-4-(tetrahydro-2H-pyran-4-yloxy)-1H-indazol-6-yl]benzamide;
2-[4-(5-fluoropyrimidin-2-yl)piperazin-1-yl]-N-methyl-4-[3-methyl-4-(tetrahydro-2H-pyran-4-yloxy)-1H-indazol-6-yl]benzamide;
4-[3-methyl-4-(tetrahydro-2H-pyran-4-yloxy)-1H-indazol-6-yl]-2-[6-(2,2,2-trifluoroethyl)-2,6-diazaspiro[3.3]hept-2-yl]benzamide;
4-[3-amino-4-(tetrahydro-2H-pyran-4-yloxy)-1H-indazol-6-yl]-N-methyl-2-[4-(2,2,3,3-tetrafluoropropyl)piperazin-1-yl]benzamide;
4-[3-amino-4-(tetrahydro-2H-pyran-4-yloxy)-1H-indazol-6-yl]-N-methyl-2-[4-(3,3,3-trifluoropropyl)piperazin-1-yl]benzamide;
4-[3-amino-4-(tetrahydro-2H-pyran-4-yloxy)-1H-indazol-6-yl]-2-[4-(2,2-difluoroethyl)piperazin-1-yl]-N-methylbenzamide;
4-[3-amino-4-(tetrahydro-2H-pyran-4-yloxy)-1H-indazol-6-yl]-N-methyl-2-[4-(2,2,3,3,3-pentafluoropropyl)piperazin-1-yl]benzamide;
4-[3-amino-4-(tetrahydro-2H-pyran-4-yloxy)-1H-indazol-6-yl]-2-{4-[(3,3-difluorocyclobutyl)methyl]piperazin-1-yl}-N-methylbenzamide;
4-[3-amino-4-(tetrahydro-2H-pyran-4-yloxy)-1H-indazol-6-yl]-2-[4-(2,2-difluoropropyl)piperazin-1-yl]-N-methylbenzamide; and
4-[3-amino-4-(tetrahydro-2H-pyran-4-yloxy)-1H-indazol-6-yl]-2-{4-[(2,2-difluorocyclopropyl)methyl]piperazin-1-yl}-N-methylbenzamide.

16. A pharmaceutical composition comprising a therapeutically effective amount of a compound of claim 1 in combination with a pharmaceutically acceptable carrier.

17. A method of selectively modulating the effects of in a mammal comprising administering an effective amount of a compound of claim 1.

18. A method of treating a condition or disorder modulated by TrkA (Tropomyosin receptor kinase isoform A) in a mammal comprising administering an effective amount of a compound of claim 1.

19. The method according to claim 18, wherein the condition or disorder is selected from the group consisting of pain, including osteoarthritis pain, joint pain, neuropathic pain, post-surgical pain, low back pain, and diabetic neuropathy, pain during surgery, cancer pain, chemotherapy induced pain, headaches, including cluster headache, tension headache, migraine pain, trigeminal neuralgia, shingles pain, post-herpetic neuralgia, carpal tunnel syndrome, inflammatory pain, pain from rheumatoid arthritis, colitis, pain of interstitial cystitis, visceral pain, pain from kidney stone, pain from gallstone, angina, fibromyalgia, chronic pain syndrome, thalamic pain syndrome, pain from stroke, phantom limb pain, sunburn, radiculopathy, complex regional pain syndrome, HIV sensory neuropathy, central neuropathic pain syndromes, multiple sclerosis pain, Parkinson disease pain, spinal cord injury pain, menstrual pain, toothache, pain from bone metastasis, pain from endometriosis, pain from uterine fibroids, nociceptive pain, hyperalgesia, and temporomandibular joint pain, inflammation, auto-immune disease, rheumatoid arthritis, psoriasis, psoriatic arthritis, asthma, Crohn's disease, inflammatory bladder cystitis, inflammatory bowel disease, joint swelling, diabetic nephropathy, kidney fibrosis, chronic kidney disease, cancer, neuroblastoma, melanoma, myeloma, cancers of the pancreas, prostate, ovary, colon, thyroid, lung, brain, esophagus, kidney, of bone, and blood.

20. The method according to claim 18, wherein the condition or disorder is selected from the group consisting of pain, including osteoarthritis pain, joint pain, neuropathic pain, post-surgical pain, low back pain, and diabetic neuropathy, pain during surgery, cancer pain, chemotherapy induced pain, headaches, including cluster headache, tension headache, migraine pain, trigeminal neuralgia, shingles pain, post-herpetic neuralgia, carpal tunnel syndrome, inflammatory pain, pain from rheumatoid arthritis, colitis, pain of interstitial cystitis, visceral pain, pain from kidney stone, pain from gallstone, angina, fibromyalgia, chronic pain syndrome, thalamic pain syndrome, pain from stroke, phantom limb pain, sunburn, radiculopathy, complex regional pain syndrome, HIV sensory neuropathy, central neuropathic pain syndromes, multiple sclerosis pain, Parkinson disease pain, spinal cord injury pain, menstrual pain, toothache, pain from bone metastasis, pain from endometriosis, pain from uterine fibroids, nociceptive pain, hyperalgesia, and temporomandibular joint pain.
